SEQUENCE LISTING (134 sequences)

SEQ ID NO: 1 - protein, 616 aa, Mucor hiemalis
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 2 - protein, 616 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGCHGKNGRIDISFPEFQFPQSANWNASLATLDFTHQQDLLCGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 3 - protein, 616 aa, synthetic construct; identical to SEQ ID NO: 1

SEQ ID NO: 4 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSIPNPLLGLDVFQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 5 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVIPNPLLGLDFQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 6 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFIPNPLLGLDQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 7 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKIPNPLLGLDTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 8 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSISASRKLQSVFQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 9 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVISASRKLQSFQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 10 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFISASRKLQSQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 11 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQISASRKLQSNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 12 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNISASRKLQSETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 13 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNEISASRKLQSTLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 14 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNISASRKLQSNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 15 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNISASRKLQSKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 16 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKISASRKLQSTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 17 - protein, 616 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSISASRKLQSEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 18 - protein, 616 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQISASRKLQSQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 19 - protein, 616 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNEISASRKLQSYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 20 - protein, 616 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDGISASRKLQSGGRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 21 - protein, 616 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVGGISASRKLQSGGYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 22 - protein, 616 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQGGISASRKLQSYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 23 - protein, 616 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDIPNPLLGLDGGQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 24 - protein, 616 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDGGIPNPLLGLDGGQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 25 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFISASRKLQLQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 26 - protein, 629 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFKKISASRKLQLKTQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 27 - protein, 632 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFKKISASRKLQLKTLLLQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 28 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKISASRKLQLTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 29 - protein, 628 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKKISASRKLQLKTTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 30 - protein, 631 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKKISASRKLQLKTLLLTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 31 - protein, 630 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFGKPIPNPLLGLDSTQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 32 - protein, 626 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFGIPNPLLGLDQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 33 - protein, 627 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFGGIPNPLLGLDQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 34 - protein, 626 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFIPNPLLGLDGQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 35 - protein, 627 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFIPNPLLGLDGGQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 36 - protein, 627 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFGIPNPLLGLDGQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 37 - protein, 629 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFSGIPNPLLGLDGSQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 38 - protein, 628 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFGIPNPLLGLDGGQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 39 - protein, 628 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFGGIPNPLLGLDGQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 40 - protein, 629 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFGGIPNPLLGLDGGQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 41 - protein, 633 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFSGGGIPNPLLGLDGGGSQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 42 - protein, 635 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFSGGGGIPNPLLGLDGGGGSQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 43 - protein, 630 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKGKPIPNPLLGLDSTTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 44 - protein, 626 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKGIPNPLLGLDTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 45 - protein, 627 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKGGIPNPLLGLDTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 46 - protein, 626 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKIPNPLLGLDGTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 47 - protein, 627 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKIPNPLLGLDGGTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 48 - protein, 627 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKGIPNPLLGLDGTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 49 - protein, 629 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKSGIPNPLLGLDGSTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 50 - protein, 628 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKGIPNPLLGLDGGTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 51 - protein, 628 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKGGIPNPLLGLDGTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 52 - protein, 629 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKGGIPNPLLGLDGGTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 53 - protein, 633 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKSGGGIPNPLLGLDGGGSTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 54 - protein, 635 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKSGGGGIPNPLLGLDGGGGSTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 55 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFYPYDVPDYAQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 56 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKYPYDVPDYATGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 57 - protein, 626 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFEQKLISEEDLQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 58 - protein, 626 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKEQKLISEEDLTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 59 - protein, 624 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFDYKDDDDKQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 60 - protein, 624 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKDYKDDDDKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 61 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQPGEFTLGNQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 62 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKQPGEFTLGNTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 63 - protein, 624 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFETSGLQEQQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 64 - protein, 624 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKETSGLQEQTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 65 - protein, 616 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYAPVGTCAMLPRELGGVVDPNLLVYGTANLRVVDASIMPLEISSALMQPTYGVAEKAADIIKMSRKNNNN

SEQ ID NO: 66 - protein, 621 aa, Mucor prainii
MQDTNSSSTDTYDYVIVGGGVAGLALASRISENKDVTVAVLESGPNANDRFVVYAPGMYGQAVGTDLCPLIPTTPQENMGNRSLTIATGRLLGGGSAINGLVWTRGGLKDYDAWEELGNPGWNGANLFKYFKKVENFTPPTPAQIEYGATYQKSAHGKKGPIDVSFTNYEFSQSASWNASLETLDFTALPDILNGTLAGYSTTPNILDPETVQRVDSYTGYIAPYTSRNNLNVLANHTVSRIQFAPKNGSEPLKATGVEWYPTGNKNQKQIIKARYEVIISSGAIGSPKLLEISGIGNKDIVSAAGVESLIDLPGVGSNMQDHVHAITVSTTNITGYTTNSVFVNETLAQEQREEYEANKTGIWATTPNNLGYPTPEQLFNGTEFVSGKEFADKIRNSTDEWANYYASTNASNVELLKKQYAIVASRYEENYLSPIEINFTPGYEGSGNVDLQNNKYQTVNHVLIAPLSRGYTHINSSDVEDHSVINPQYYSHPMDIDVHIASTKLAREIITASPGLGDINSGEIEPGMNITSEDDLRSWLSNNVRSDWHPVGTCAMLPKELGGVVSPALMVYGTSNLRVVDASIMPLEVSSHLMQPTYGIAEKAADIIKNFYKTQHKNQN

SEQ ID NO: 67 - protein, 614 aa, Mucor guilliermondii
MQSNTDTYDYVIVGGGVGGLALANRLSENKQVTVAVLEAGPNANDEFIVYAPGMYGQAVGTYLAPLRPTVPQENMNNRSLSIATGKLLGGGSAVNGLVWTRGATKDFDAWEELGNPGWNGASMFKYFKKVENFTAPTPYQVNYGATYQKNTHGYKGPVQVSFTNYEFPQSAHWNQSLASLGFDHLPDLLNGTLSGYSTTPNILDPNTDQRCDAYAAYIAPYTARTNLHVLANHTVSRIEFNQTNANQPLVASGVEWYPTGDNTKKQTIKARLEVIVSSGSIGSPKLLEISGIGNKDIVTAAGVKSLLDLPGVGSNMQDHVHAVTVSTTNITGYTTDSVFVNSTLASEQREQYEKDKSGIWTTTPNNLGYPTPAQLFNGTEFMDGKAFAARIRNSSQEWAQYYASKNASTVELLMKQYEIVASRYEENYLSPIEINLTPGYGGVGTVDKTKNKYQTVNHVLIAPLSRGFTHINSSDIEDPVNINPQYYSHPMDIDVHVASTKLARRIINAPGLGDLNSGEVEPGMDITSDSDVRAWLANNVRSDWHPVGTCAMLPKELGGVVDSSLKVYGTANLRVVDASIMPLEVSSHLMQPTFGVAEKAADIIKAEYKKQKAQ

SEQ ID NO: 68 - protein, 616 aa, Mucor hiemalis
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTARRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKTGIWTTTPNNLGYPSPSQLFDGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGEIQPGSNITSDEDVKQWLADNVRSDWHPVGTCAMLPRELGGVVDPNLLVYGTANLRVVDASIMPLEISSHLMQPTYGVAEKAADIIKMSRKNNNN

SEQ ID NO: 69 - protein, 614 aa, Mucor subtillisimus
MQQNGTSNDTYDYVIVGGGVGGLSLASRLSEDKGVTVAVLESGPYADDRFVVYAPGMYGQAVGTELCPLLPTVPQVGMNNRTITIATGRLLGGGSAVNGLVWTRGAMKDFDAWEELGNPGWNGKTMFKYFKKIENFHPPTEEQVQYGATYQKNVHGSGGPIDISFPVFEFPQSANWNASLAYLNFTHQQDLLNGSLHGYSTTPNTLNPETARRADAYAGYIQPNVNRTNLAVLANHTVSRIQFEKSNGSQPLKAIGVEWYTTGGDKSTKQTIKARREVIISSGAIGSPKLLEVSGIGNKQIVTAAGVESLIDLPGVGSNMQDHVHAVTVSTTNIEGYTTNSVFTNETLAQEQKDLYYNNKTGIWTTTPNNLGYPSPSQLFTNTTFRSGKQFAAMIRNSTDKYAQYYASTKNATNIQLLKKQYAIVARRYEEDYISPIEINFTPGYGGTGEVDLQNNKYQTVNHVLVAPLSRGYTHINSSDIEDPVVIDPQYYSHPLDVDVHVASTQLARSILNAPALAAINSGEVEPGEKIQTDQDVRKWLSDNVRSDWHPVGTCAMLPKGLGGVVDSNLKVYGTANLRVVDASIIPLEISSHLMQPVYAVSERAADIIKGSRN

SEQ ID NO: 70 - protein, 625 aa, Mucor circinelloides
MQQDTNNTSSTDTYDYVIVGGGVAGLALASRISENKDVTVAVLESGPNAEDQFVVYAPGMYGQAVGTELAPLVPTTPQENMGNRSLSIATGRLLGGGSAVNGLVWTRGGLKDYDAWEELGNPGWNGSNLFKYFKKVENFHPPTPAQIEYGATYQKSAHGKNGPIDVSFTNFEFPQSAKWNASLESLDFTALPDLLNGTLAGYSTTPNILDPETARRVDAYAGYIVPYMGRNNLNVLANHTVSRIQFAPQNGSEPLKATGVEWYPTGNKDQKQTIKARYEVIISSGAIGSPKLLEISGIGNKDIVTAAGVESLIDLPGVGANMQDHVHAVTVSTTNIDGYTTNSVFTNETLAQEQREQYEANKTGIWTTTPNNLGYPTPEQLFNGTEFVSGKEFAAKIRNSTDEWANYYASTNATNADLLKKQYAIVASRYEENYLSPIEINLTPGYGGTGSPDLQNNKYQTVNHVLIAPLSRGYAHINSSDIEEPSVINPQYYSHPLDIDVHVASTKLAREIITASPGLGDLNSGEVEPGMNVTSEDDLRSWLSNNVRSDWHPVGTCAMLPQELGGVVSPALMVYGTSNLRVVDASIMPLEVSSHLMQPTYGIAEKAADIIKNYYKSQYSGAGKN

SEQ ID NO: 71 - protein, 620 aa, Mucor ambiguous
MQQDTNTSSTDTYDYVIVGGGVAGLALASRISENKDVTVAVLESGPNANDQFVVYAPGMYGQAVGTDLCPLVPTTPQENMGNRSLSIATGRLLGGGSAVNGLVWTRGGLKDYDAWEELGNPGWNGSNLFKYFKKVENFHPPTPAQIEYGATYQKNAHGKNGPIDVSFTNYEFPQSAKWNASLSSLDFTALPDLLNGTLAGYSTTPNILDPETVQRVDSYAGYIAPYTSRSNLNVLANHTVSRIQFAPQNGSEPLKATGVEWYPTGNKDQKQTIKARYEVIISSGAIGSPKLLEISGIGSKDIVSAAGVESLIDLPGVGSNMQDHVHAVTVSTTNITGYTTNSVFVNETLAQEQREEYETNKTGIWTTTPNNLGYPTPEQLFNGTEFVSGKEFADKIRNSTDEWANYYASTNATNVELLKKQYAIVASRYEENYLSPIEINLTPGYGGTGSPDLQNNKYQTVNHVLIAPLSRGYTHINSSDIEEPSVINPQYYSHPMDIDVHVASTKLAREIITASPGLGDLNSGEVEPGMNITSEDDLRSWLSNNVRSDWHPVGTCAMLPKELGGVVSPALMVYGTSNLRVVDASIMPLEVSSHLMQPTYGIAEKAADIIKNAYKNQYKN

SEQ ID NO: 72 - protein, 611 aa, Mucor lusitanicus
MQQDTNNTSSTDTYDYVIVGGGVAGLALASRISENKDVTVAVLESGPNAEDQFVVYAPGMYGQAVGTELAPLVPTTPQENMGNRSLSIATGRLLGGGSAVNGLVWTRGGLKDYDAWEELGNPGWNGSNLFKYFKKVENFHPPTPAQIEYGATYQKSAHGKNGPIDVSFTNFEFPQSAKWNASLESLDFTALPDLLNGTLAGYSTTPNILDPETARRVDAYAGYIVPYMGRNNLNVLANHTVSRIQFAPQNGSEPLKATGVEWYPTGNKDQKQTIKARYEVIISSGAIGSPKLLEISGIGNKDIVTAAGVESLIDLPGVGANMQDHVHAVTVSTTNIDGYTTNSVFTNETLAQEQREQYEANKTGIWTTTPNNLGYPTPEQLFNGTEFVSGKEFAAKIRNSTDEWANYYASTNATNADLLKKQYAIVASRYEENYLSPIEINLTPGYGGTGSPDLQNNKYQTVNHVLIAPLSRGYAHINSSDIEEPSVINPQYYSHPLDIDVHVASTKLAREIITASPGLGDLNSGEVEPGMNVTSEDDLRSWLSNNVRSDWHPVGTCAMLPQELGGVVSPALMVYGTSNLRVVDASIMPLEVSSHLMQPTYGIAEKAADII

SEQ ID NO: 73 - protein, 593 aa, Aspergillus flavus
MLFSLAFLSALSLATASPAGRAKNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPAVNGKEGPLKVGWSGSLASGNLSVALNRTFQAAGVPWVEDVNGGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVLAGASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSA

SEQ ID NO: 74 - protein, 575 aa, Aspergillus niger
MTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

SEQ ID NO: 75 - protein, 630 aa, synthetic construct
MQDTNSSSTDTYDYVIVGGGVAGLALASRISENKDVTVAVLESGPNANDRFVVYAPGMYGQAVGTDLCPLIPTTPQENMGNRSLTIATGRLLGGGSAINGLVWTRGGLKDYDAWEELGNPGWNGANLFKYFKKVENFTPPTPAQIEYGATYQKSAHGKKGPIDVSFTNYEFSQSASWNASLETLDFTALPDILNGTLAGYSTTPNILDPETVQRVDSYTGYIAPYTSRNNLNVLANHTVSRIQFAPKNGSEPLKATGVEWYPTGNKNQKQIIKARYEVIISSGAIGSPKLLEISGIGNKDIVSAAGVESLIDLPGVGSNMQDHVHAITVSTTNITGYTTNSVFYPYDVPDYAVNETLAQEQREEYEANKTGIWATTPNNLGYPTPEQLFNGTEFVSGKEFADKIRNSTDEWANYYASTNASNVELLKKQYAIVASRYEENYLSPIEINFTPGYEGSGNVDLQNNKYQTVNHVLIAPLSRGYTHINSSDVEDHSVINPQYYSHPMDIDVHIASTKLAREIITASPGLGDINSGEIEPGMNITSEDDLRSWLSNNVRSDWHPVGTCAMLPKELGGVVSPALMVYGTSNLRVVDASIMPLEVSSHLMQPTYGIAEKAADIIKNFYKTQHKNQN

SEQ ID NO: 76 - protein, 630 aa, synthetic construct
MQDTNSSSTDTYDYVIVGGGVAGLALASRISENKDVTVAVLESGPNANDRFVVYAPGMYGQAVGTDLCPLIPTTPQENMGNRSLTIATGRLLGGGSAINGLVWTRGGLKDYDAWEELGNPGWNGANLFKYFKKVENFTPPTPAQIEYGATYQKSAHGKKGPIDVSFTNYEFSQSASWNASLETLDFTALPDILNGTLAGYSTTPNILDPETVQRVDSYTGYIAPYTSRNNLNVLANHTVSRIQFAPKNGSEPLKATGVEWYPTGNKNQKQIIKARYEVIISSGAIGSPKLLEISGIGNKDIVSAAGVESLIDLPGVGSNMQDHVHAITVSTTNITGYTTNSVFVNETLAQEQREEYEANKYPYDVPDYATGIWATTPNNLGYPTPEQLFNGTEFVSGKEFADKIRNSTDEWANYYASTNASNVELLKKQYAIVASRYEENYLSPIEINFTPGYEGSGNVDLQNNKYQTVNHVLIAPLSRGYTHINSSDVEDHSVINPQYYSHPMDIDVHIASTKLAREIITASPGLGDINSGEIEPGMNITSEDDLRSWLSNNVRSDWHPVGTCAMLPKELGGVVSPALMVYGTSNLRVVDASIMPLEVSSHLMQPTYGIAEKAADIIKNFYKTQHKNQN

SEQ ID NO: 77 - protein, 630 aa, synthetic construct
MQDTNSSSTDTYDYVIVGGGVAGLALASRISENKDVTVAVLESGPNANDRFVVYAPGMYGQAVGTDLCPLIPTTPQENMGNRSLTIATGRLLGGGSAINGLVWTRGGLKDYDAWEELGNPGWNGANLFKYFKKVENFTPPTPAQIEYGATYQKSAHGKKGPIDVSFTNYEFSQSASWNASLETLDFTALPDILNGTLAGYSTTPNILDPETVQRVDSYTGYIAPYTSRNNLNVLANHTVSRIQFAPKNGSEPLKATGVEWYPTGNKNQKQIIKARYEVIISSGAIGSPKLLEISGIGNKDIVSAAGVESLIDLPGVGSNMQDHVHAITVSTTNITGYTTNSVFVNETLAQEQREEYEANKQPGEFTLGNTGIWATTPNNLGYPTPEQLFNGTEFVSGKEFADKIRNSTDEWANYYASTNASNVELLKKQYAIVASRYEENYLSPIEINFTPGYEGSGNVDLQNNKYQTVNHVLIAPLSRGYTHINSSDVEDHSVINPQYYSHPMDIDVHIASTKLAREIITASPGLGDINSGEIEPGMNITSEDDLRSWLSNNVRSDWHPVGTCAMLPKELGGVVSPALMVYGTSNLRVVDASIMPLEVSSHLMQPTYGIAEKAADIIKNFYKTQHKNQN

SEQ ID NO: 78 - protein, 630 aa, synthetic construct
MQDTNSSSTDTYDYVIVGGGVAGLALASRISENKDVTVAVLESGPNANDRFVVYAPGMYGQAVGTDLCPLIPTTPQENMGNRSLTIATGRLLGGGSAINGLVWTRGGLKDYDAWEELGNPGWNGANLFKYFKKVENFTPPTPAQIEYGATYQKSAHGKKGPIDVSFTNYEFSQSASWNASLETLDFTALPDILNGTLAGYSTTPNILDPETVQRVDSYTGYIAPYTSRNNLNVLANHTVSRIQFAPKNGSEPLKATGVEWYPTGNKNQKQIIKARYEVIISSGAIGSPKLLEISGIGNKDIVSAAGVESLIDLPGVGSNMQDHVHAITVSTTNITGYTTNSVFISASRKLQLVNETLAQEQREEYEANKTGIWATTPNNLGYPTPEQLFNGTEFVSGKEFADKIRNSTDEWANYYASTNASNVELLKKQYAIVASRYEENYLSPIEINFTPGYEGSGNVDLQNNKYQTVNHVLIAPLSRGYTHINSSDVEDHSVINPQYYSHPMDIDVHIASTKLAREIITASPGLGDINSGEIEPGMNITSEDDLRSWLSNNVRSDWHPVGTCAMLPKELGGVVSPALMVYGTSNLRVVDASIMPLEVSSHLMQPTYGIAEKAADIIKNFYKTQHKNQN

SEQ ID NO: 79 - protein, 630 aa, synthetic construct
MQDTNSSSTDTYDYVIVGGGVAGLALASRISENKDVTVAVLESGPNANDRFVVYAPGMYGQAVGTDLCPLIPTTPQENMGNRSLTIATGRLLGGGSAINGLVWTRGGLKDYDAWEELGNPGWNGANLFKYFKKVENFTPPTPAQIEYGATYQKSAHGKKGPIDVSFTNYEFSQSASWNASLETLDFTALPDILNGTLAGYSTTPNILDPETVQRVDSYTGYIAPYTSRNNLNVLANHTVSRIQFAPKNGSEPLKATGVEWYPTGNKNQKQIIKARYEVIISSGAIGSPKLLEISGIGNKDIVSAAGVESLIDLPGVGSNMQDHVHAITVSTTNITGYTTNSVFVNETLAQEQREEYEANKISASRKLQLTGIWATTPNNLGYPTPEQLFNGTEFVSGKEFADKIRNSTDEWANYYASTNASNVELLKKQYAIVASRYEENYLSPIEINFTPGYEGSGNVDLQNNKYQTVNHVLIAPLSRGYTHINSSDVEDHSVINPQYYSHPMDIDVHIASTKLAREIITASPGLGDINSGEIEPGMNITSEDDLRSWLSNNVRSDWHPVGTCAMLPKELGGVVSPALMVYGTSNLRVVDASIMPLEVSSHLMQPTYGIAEKAADIIKNFYKTQHKNQN

SEQ ID NO: 80 - protein, 623 aa, synthetic construct
MQSNTDTYDYVIVGGGVGGLALANRLSENKQVTVAVLEAGPNANDEFIVYAPGMYGQAVGTYLAPLRPTVPQENMNNRSLSIATGKLLGGGSAVNGLVWTRGATKDFDAWEELGNPGWNGASMFKYFKKVENFTAPTPYQVNYGATYQKNTHGYKGPVQVSFTNYEFPQSAHWNQSLASLGFDHLPDLLNGTLSGYSTTPNILDPNTDQRCDAYAAYIAPYTARTNLHVLANHTVSRIEFNQTNANQPLVASGVEWYPTGDNTKKQTIKARLEVIVSSGSIGSPKLLEISGIGNKDIVTAAGVKSLLDLPGVGSNMQDHVHAVTVSTTNITGYTTDSVFVYPYDVPDYANSTLASEQREQYEKDKSGIWTTTPNNLGYPTPAQLFNGTEFMDGKAFAARIRNSSQEWAQYYASKNASTVELLMKQYEIVASRYEENYLSPIEINLTPGYGGVGTVDKTKNKYQTVNHVLIAPLSRGFTHINSSDIEDPVNINPQYYSHPMDIDVHVASTKLARRIINAPGLGDLNSGEVEPGMDITSDSDVRAWLANNVRSDWHPVGTCAMLPKELGGVVDSSLKVYGTANLRVVDASIMPLEVSSHLMQPTFGVAEKAADIIKAEYKKQKAQ

SEQ ID NO: 81 - protein, 623 aa, synthetic construct
MQSNTDTYDYVIVGGGVGGLALANRLSENKQVTVAVLEAGPNANDEFIVYAPGMYGQAVGTYLAPLRPTVPQENMNNRSLSIATGKLLGGGSAVNGLVWTRGATKDFDAWEELGNPGWNGASMFKYFKKVENFTAPTPYQVNYGATYQKNTHGYKGPVQVSFTNYEFPQSAHWNQSLASLGFDHLPDLLNGTLSGYSTTPNILDPNTDQRCDAYAAYIAPYTARTNLHVLANHTVSRIEFNQTNANQPLVASGVEWYPTGDNTKKQTIKARLEVIVSSGSIGSPKLLEISGIGNKDIVTAAGVKSLLDLPGVGSNMQDHVHAVTVSTTNITGYTTDSVFVNSTLASEQREQYEKDKYPYDVPDYASGIWTTTPNNLGYPTPAQLFNGTEFMDGKAFAARIRNSSQEWAQYYASKNASTVELLMKQYEIVASRYEENYLSPIEINLTPGYGGVGTVDKTKNKYQTVNHVLIAPLSRGFTHINSSDIEDPVNINPQYYSHPMDIDVHVASTKLARRIINAPGLGDLNSGEVEPGMDITSDSDVRAWLANNVRSDWHPVGTCAMLPKELGGVVDSSLKVYGTANLRVVDASIMPLEVSSHLMQPTFGVAEKAADIIKAEYKKQKAQ

SEQ ID NO: 82 - protein, 623 aa, synthetic construct
MQSNTDTYDYVIVGGGVGGLALANRLSENKQVTVAVLEAGPNANDEFIVYAPGMYGQAVGTYLAPLRPTVPQENMNNRSLSIATGKLLGGGSAVNGLVWTRGATKDFDAWEELGNPGWNGASMFKYFKKVENFTAPTPYQVNYGATYQKNTHGYKGPVQVSFTNYEFPQSAHWNQSLASLGFDHLPDLLNGTLSGYSTTPNILDPNTDQRCDAYAAYIAPYTARTNLHVLANHTVSRIEFNQTNANQPLVASGVEWYPTGDNTKKQTIKARLEVIVSSGSIGSPKLLEISGIGNKDIVTAAGVKSLLDLPGVGSNMQDHVHAVTVSTTNITGYTTDSVFVQPGEFTLGNNSTLASEQREQYEKDKSGIWTTTPNNLGYPTPAQLFNGTEFMDGKAFAARIRNSSQEWAQYYASKNASTVELLMKQYEIVASRYEENYLSPIEINLTPGYGGVGTVDKTKNKYQTVNHVLIAPLSRGFTHINSSDIEDPVNINPQYYSHPMDIDVHVASTKLARRIINAPGLGDLNSGEVEPGMDITSDSDVRAWLANNVRSDWHPVGTCAMLPKELGGVVDSSLKVYGTANLRVVDASIMPLEVSSHLMQPTFGVAEKAADIIKAEYKKQKAQ

SEQ ID NO: 83 - protein, 623 aa, synthetic construct
MQSNTDTYDYVIVGGGVGGLALANRLSENKQVTVAVLEAGPNANDEFIVYAPGMYGQAVGTYLAPLRPTVPQENMNNRSLSIATGKLLGGGSAVNGLVWTRGATKDFDAWEELGNPGWNGASMFKYFKKVENFTAPTPYQVNYGATYQKNTHGYKGPVQVSFTNYEFPQSAHWNQSLASLGFDHLPDLLNGTLSGYSTTPNILDPNTDQRCDAYAAYIAPYTARTNLHVLANHTVSRIEFNQTNANQPLVASGVEWYPTGDNTKKQTIKARLEVIVSSGSIGSPKLLEISGIGNKDIVTAAGVKSLLDLPGVGSNMQDHVHAVTVSTTNITGYTTDSVFVNSTLASEQREQYEKDKQPGEFTLGNSGIWTTTPNNLGYPTPAQLFNGTEFMDGKAFAARIRNSSQEWAQYYASKNASTVELLMKQYEIVASRYEENYLSPIEINLTPGYGGVGTVDKTKNKYQTVNHVLIAPLSRGFTHINSSDIEDPVNINPQYYSHPMDIDVHVASTKLARRIINAPGLGDLNSGEVEPGMDITSDSDVRAWLANNVRSDWHPVGTCAMLPKELGGVVDSSLKVYGTANLRVVDASIMPLEVSSHLMQPTFGVAEKAADIIKAEYKKQKAQ

SEQ ID NO: 84 - protein, 623 aa, synthetic construct
MQSNTDTYDYVIVGGGVGGLALANRLSENKQVTVAVLEAGPNANDEFIVYAPGMYGQAVGTYLAPLRPTVPQENMNNRSLSIATGKLLGGGSAVNGLVWTRGATKDFDAWEELGNPGWNGASMFKYFKKVENFTAPTPYQVNYGATYQKNTHGYKGPVQVSFTNYEFPQSAHWNQSLASLGFDHLPDLLNGTLSGYSTTPNILDPNTDQRCDAYAAYIAPYTARTNLHVLANHTVSRIEFNQTNANQPLVASGVEWYPTGDNTKKQTIKARLEVIVSSGSIGSPKLLEISGIGNKDIVTAAGVKSLLDLPGVGSNMQDHVHAVTVSTTNITGYTTDSVFVISASRKLQLNSTLASEQREQYEKDKSGIWTTTPNNLGYPTPAQLFNGTEFMDGKAFAARIRNSSQEWAQYYASKNASTVELLMKQYEIVASRYEENYLSPIEINLTPGYGGVGTVDKTKNKYQTVNHVLIAPLSRGFTHINSSDIEDPVNINPQYYSHPMDIDVHVASTKLARRIINAPGLGDLNSGEVEPGMDITSDSDVRAWLANNVRSDWHPVGTCAMLPKELGGVVDSSLKVYGTANLRVVDASIMPLEVSSHLMQPTFGVAEKAADIIKAEYKKQKAQ

SEQ ID NO: 85 - protein, 623 aa, synthetic construct
MQSNTDTYDYVIVGGGVGGLALANRLSENKQVTVAVLEAGPNANDEFIVYAPGMYGQAVGTYLAPLRPTVPQENMNNRSLSIATGKLLGGGSAVNGLVWTRGATKDFDAWEELGNPGWNGASMFKYFKKVENFTAPTPYQVNYGATYQKNTHGYKGPVQVSFTNYEFPQSAHWNQSLASLGFDHLPDLLNGTLSGYSTTPNILDPNTDQRCDAYAAYIAPYTARTNLHVLANHTVSRIEFNQTNANQPLVASGVEWYPTGDNTKKQTIKARLEVIVSSGSIGSPKLLEISGIGNKDIVTAAGVKSLLDLPGVGSNMQDHVHAVTVSTTNITGYTTDSVFVNSTLASEQREQYEKDKISASRKLQLSGIWTTTPNNLGYPTPAQLFNGTEFMDGKAFAARIRNSSQEWAQYYASKNASTVELLMKQYEIVASRYEENYLSPIEINLTPGYGGVGTVDKTKNKYQTVNHVLIAPLSRGFTHINSSDIEDPVNINPQYYSHPMDIDVHVASTKLARRIINAPGLGDLNSGEVEPGMDITSDSDVRAWLANNVRSDWHPVGTCAMLPKELGGVVDSSLKVYGTANLRVVDASIMPLEVSSHLMQPTFGVAEKAADIIKAEYKKQKAQ

SEQ ID NO: 86 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTARRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFYPYDVPDYAQNETLAEEQRQQYYNNKTGIWTTTPNNLGYPSPSQLFDGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGEIQPGSNITSDEDVKQWLADNVRSDWHPVGTCAMLPRELGGVVDPNLLVYGTANLRVVDASIMPLEISSHLMQPTYGVAEKAADIIKMSRKNNNN

SEQ ID NO: 87 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTARRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKYPYDVPDYATGIWTTTPNNLGYPSPSQLFDGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGEIQPGSNITSDEDVKQWLADNVRSDWHPVGTCAMLPRELGGVVDPNLLVYGTANLRVVDASIMPLEISSHLMQPTYGVAEKAADIIKMSRKNNNN

SEQ ID NO: 88 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTARRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQPGEFTLGNQNETLAEEQRQQYYNNKTGIWTTTPNNLGYPSPSQLFDGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGEIQPGSNITSDEDVKQWLADNVRSDWHPVGTCAMLPRELGGVVDPNLLVYGTANLRVVDASIMPLEISSHLMQPTYGVAEKAADIIKMSRKNNNN

SEQ ID NO: 89 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTARRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKQPGEFTLGNTGIWTTTPNNLGYPSPSQLFDGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGEIQPGSNITSDEDVKQWLADNVRSDWHPVGTCAMLPRELGGVVDPNLLVYGTANLRVVDASIMPLEISSHLMQPTYGVAEKAADIIKMSRKNNNN

SEQ ID NO: 90 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTARRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFISASRKLQLQNETLAEEQRQQYYNNKTGIWTTTPNNLGYPSPSQLFDGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGEIQPGSNITSDEDVKQWLADNVRSDWHPVGTCAMLPRELGGVVDPNLLVYGTANLRVVDASIMPLEISSHLMQPTYGVAEKAADIIKMSRKNNNN

SEQ ID NO: 91 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTARRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKISASRKLQLTGIWTTTPNNLGYPSPSQLFDGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGEIQPGSNITSDEDVKQWLADNVRSDWHPVGTCAMLPRELGGVVDPNLLVYGTANLRVVDASIMPLEISSHLMQPTYGVAEKAADIIKMSRKNNNN

SEQ ID NO: 92 - protein, 623 aa, synthetic construct
MQQNGTSNDTYDYVIVGGGVGGLSLASRLSEDKGVTVAVLESGPYADDRFVVYAPGMYGQAVGTELCPLLPTVPQVGMNNRTITIATGRLLGGGSAVNGLVWTRGAMKDFDAWEELGNPGWNGKTMFKYFKKIENFHPPTEEQVQYGATYQKNVHGSGGPIDISFPVFEFPQSANWNASLAYLNFTHQQDLLNGSLHGYSTTPNTLNPETARRADAYAGYIQPNVNRTNLAVLANHTVSRIQFEKSNGSQPLKAIGVEWYTTGGDKSTKQTIKARREVIISSGAIGSPKLLEVSGIGNKQIVTAAGVESLIDLPGVGSNMQDHVHAVTVSTTNIEGYTTNSVFYPYDVPDYATNETLAQEQKDLYYNNKTGIWTTTPNNLGYPSPSQLFTNTTFRSGKQFAAMIRNSTDKYAQYYASTKNATNIQLLKKQYAIVARRYEEDYISPIEINFTPGYGGTGEVDLQNNKYQTVNHVLVAPLSRGYTHINSSDIEDPVVIDPQYYSHPLDVDVHVASTQLARSILNAPALAAINSGEVEPGEKIQTDQDVRKWLSDNVRSDWHPVGTCAMLPKGLGGVVDSNLKVYGTANLRVVDASIIPLEISSHLMQPVYAVSERAADIIKGSRN

SEQ ID NO: 93 - protein, 623 aa, synthetic construct
MQQNGTSNDTYDYVIVGGGVGGLSLASRLSEDKGVTVAVLESGPYADDRFVVYAPGMYGQAVGTELCPLLPTVPQVGMNNRTITIATGRLLGGGSAVNGLVWTRGAMKDFDAWEELGNPGWNGKTMFKYFKKIENFHPPTEEQVQYGATYQKNVHGSGGPIDISFPVFEFPQSANWNASLAYLNFTHQQDLLNGSLHGYSTTPNTLNPETARRADAYAGYIQPNVNRTNLAVLANHTVSRIQFEKSNGSQPLKAIGVEWYTTGGDKSTKQTIKARREVIISSGAIGSPKLLEVSGIGNKQIVTAAGVESLIDLPGVGSNMQDHVHAVTVSTTNIEGYTTNSVFTNETLAQEQKDLYYNNKYPYDVPDYATGIWTTTPNNLGYPSPSQLFTNTTFRSGKQFAAMIRNSTDKYAQYYASTKNATNIQLLKKQYAIVARRYEEDYISPIEINFTPGYGGTGEVDLQNNKYQTVNHVLVAPLSRGYTHINSSDIEDPVVIDPQYYSHPLDVDVHVASTQLARSILNAPALAAINSGEVEPGEKIQTDQDVRKWLSDNVRSDWHPVGTCAMLPKGLGGVVDSNLKVYGTANLRVVDASIIPLEISSHLMQPVYAVSERAADIIKGSRN

SEQ ID NO: 94 - protein, 623 aa, synthetic construct
MQQNGTSNDTYDYVIVGGGVGGLSLASRLSEDKGVTVAVLESGPYADDRFVVYAPGMYGQAVGTELCPLLPTVPQVGMNNRTITIATGRLLGGGSAVNGLVWTRGAMKDFDAWEELGNPGWNGKTMFKYFKKIENFHPPTEEQVQYGATYQKNVHGSGGPIDISFPVFEFPQSANWNASLAYLNFTHQQDLLNGSLHGYSTTPNTLNPETARRADAYAGYIQPNVNRTNLAVLANHTVSRIQFEKSNGSQPLKAIGVEWYTTGGDKSTKQTIKARREVIISSGAIGSPKLLEVSGIGNKQIVTAAGVESLIDLPGVGSNMQDHVHAVTVSTTNIEGYTTNSVFQPGEFTLGNTNETLAQEQKDLYYNNKTGIWTTTPNNLGYPSPSQLFTNTTFRSGKQFAAMIRNSTDKYAQYYASTKNATNIQLLKKQYAIVARRYEEDYISPIEINFTPGYGGTGEVDLQNNKYQTVNHVLVAPLSRGYTHINSSDIEDPVVIDPQYYSHPLDVDVHVASTQLARSILNAPALAAINSGEVEPGEKIQTDQDVRKWLSDNVRSDWHPVGTCAMLPKGLGGVVDSNLKVYGTANLRVVDASIIPLEISSHLMQPVYAVSERAADIIKGSRN

SEQ ID NO: 95 - protein, 623 aa, synthetic construct
MQQNGTSNDTYDYVIVGGGVGGLSLASRLSEDKGVTVAVLESGPYADDRFVVYAPGMYGQAVGTELCPLLPTVPQVGMNNRTITIATGRLLGGGSAVNGLVWTRGAMKDFDAWEELGNPGWNGKTMFKYFKKIENFHPPTEEQVQYGATYQKNVHGSGGPIDISFPVFEFPQSANWNASLAYLNFTHQQDLLNGSLHGYSTTPNTLNPETARRADAYAGYIQPNVNRTNLAVLANHTVSRIQFEKSNGSQPLKAIGVEWYTTGGDKSTKQTIKARREVIISSGAIGSPKLLEVSGIGNKQIVTAAGVESLIDLPGVGSNMQDHVHAVTVSTTNIEGYTTNSVFTNETLAQEQKDLYYNNKQPGEFTLGNTGIWTTTPNNLGYPSPSQLFTNTTFRSGKQFAAMIRNSTDKYAQYYASTKNATNIQLLKKQYAIVARRYEEDYISPIEINFTPGYGGTGEVDLQNNKYQTVNHVLVAPLSRGYTHINSSDIEDPVVIDPQYYSHPLDVDVHVASTQLARSILNAPALAAINSGEVEPGEKIQTDQDVRKWLSDNVRSDWHPVGTCAMLPKGLGGVVDSNLKVYGTANLRVVDASIIPLEISSHLMQPVYAVSERAADIIKGSRN

SEQ ID NO: 96 - protein, 623 aa, synthetic construct
MQQNGTSNDTYDYVIVGGGVGGLSLASRLSEDKGVTVAVLESGPYADDRFVVYAPGMYGQAVGTELCPLLPTVPQVGMNNRTITIATGRLLGGGSAVNGLVWTRGAMKDFDAWEELGNPGWNGKTMFKYFKKIENFHPPTEEQVQYGATYQKNVHGSGGPIDISFPVFEFPQSANWNASLAYLNFTHQQDLLNGSLHGYSTTPNTLNPETARRADAYAGYIQPNVNRTNLAVLANHTVSRIQFEKSNGSQPLKAIGVEWYTTGGDKSTKQTIKARREVIISSGAIGSPKLLEVSGIGNKQIVTAAGVESLIDLPGVGSNMQDHVHAVTVSTTNIEGYTTNSVFISASRKLQLTNETLAQEQKDLYYNNKTGIWTTTPNNLGYPSPSQLFTNTTFRSGKQFAAMIRNSTDKYAQYYASTKNATNIQLLKKQYAIVARRYEEDYISPIEINFTPGYGGTGEVDLQNNKYQTVNHVLVAPLSRGYTHINSSDIEDPVVIDPQYYSHPLDVDVHVASTQLARSILNAPALAAINSGEVEPGEKIQTDQDVRKWLSDNVRSDWHPVGTCAMLPKGLGGVVDSNLKVYGTANLRVVDASIIPLEISSHLMQPVYAVSERAADIIKGSRN

SEQ ID NO: 97 - protein, 623 aa, synthetic construct
MQQNGTSNDTYDYVIVGGGVGGLSLASRLSEDKGVTVAVLESGPYADDRFVVYAPGMYGQAVGTELCPLLPTVPQVGMNNRTITIATGRLLGGGSAVNGLVWTRGAMKDFDAWEELGNPGWNGKTMFKYFKKIENFHPPTEEQVQYGATYQKNVHGSGGPIDISFPVFEFPQSANWNASLAYLNFTHQQDLLNGSLHGYSTTPNTLNPETARRADAYAGYIQPNVNRTNLAVLANHTVSRIQFEKSNGSQPLKAIGVEWYTTGGDKSTKQTIKARREVIISSGAIGSPKLLEVSGIGNKQIVTAAGVESLIDLPGVGSNMQDHVHAVTVSTTNIEGYTTNSVFTNETLAQEQKDLYYNNKISASRKLQLTGIWTTTPNNLGYPSPSQLFTNTTFRSGKQFAAMIRNSTDKYAQYYASTKNATNIQLLKKQYAIVARRYEEDYISPIEINFTPGYGGTGEVDLQNNKYQTVNHVLVAPLSRGYTHINSSDIEDPVVIDPQYYSHPLDVDVHVASTQLARSILNAPALAAINSGEVEPGEKIQTDQDVRKWLSDNVRSDWHPVGTCAMLPKGLGGVVDSNLKVYGTANLRVVDASIIPLEISSHLMQPVYAVSERAADIIKGSRN

SEQ ID NO: 98 - protein, 634 aa, synthetic construct
MQQDTNNTSSTDTYDYVIVGGGVAGLALASRISENKDVTVAVLESGPNAEDQFVVYAPGMYGQAVGTELAPLVPTTPQENMGNRSLSIATGRLLGGGSAVNGLVWTRGGLKDYDAWEELGNPGWNGSNLFKYFKKVENFHPPTPAQIEYGATYQKSAHGKNGPIDVSFTNFEFPQSAKWNASLESLDFTALPDLLNGTLAGYSTTPNILDPETARRVDAYAGYIVPYMGRNNLNVLANHTVSRIQFAPQNGSEPLKATGVEWYPTGNKDQKQTIKARYEVIISSGAIGSPKLLEISGIGNKDIVTAAGVESLIDLPGVGANMQDHVHAVTVSTTNIDGYTTNSVFYPYDVPDYATNETLAQEQREQYEANKTGIWTTTPNNLGYPTPEQLFNGTEFVSGKEFAAKIRNSTDEWANYYASTNATNADLLKKQYAIVASRYEENYLSPIEINLTPGYGGTGSPDLQNNKYQTVNHVLIAPLSRGYAHINSSDIEEPSVINPQYYSHPLDIDVHVASTKLAREIITASPGLGDLNSGEVEPGMNVTSEDDLRSWLSNNVRSDWHPVGTCAMLPQELGGVVSPALMVYGTSNLRVVDASIMPLEVSSHLMQPTYGIAEKAADIIKNYYKSQYSGAGKN

SEQ ID NO: 99 - protein, 634 aa, synthetic construct
MQQDTNNTSSTDTYDYVIVGGGVAGLALASRISENKDVTVAVLESGPNAEDQFVVYAPGMYGQAVGTELAPLVPTTPQENMGNRSLSIATGRLLGGGSAVNGLVWTRGGLKDYDAWEELGNPGWNGSNLFKYFKKVENFHPPTPAQIEYGATYQKSAHGKNGPIDVSFTNFEFPQSAKWNASLESLDFTALPDLLNGTLAGYSTTPNILDPETARRVDAYAGYIVPYMGRNNLNVLANHTVSRIQFAPQNGSEPLKATGVEWYPTGNKDQKQTIKARYEVIISSGAIGSPKLLEISGIGNKDIVTAAGVESLIDLPGVGANMQDHVHAVTVSTTNIDGYTTNSVFTNETLAQEQREQYEANKYPYDVPDYATGIWTTTPNNLGYPTPEQLFNGTEFVSGKEFAAKIRNSTDEWANYYASTNATNADLLKKQYAIVASRYEENYLSPIEINLTPGYGGTGSPDLQNNKYQTVNHVLIAPLSRGYAHINSSDIEEPSVINPQYYSHPLDIDVHVASTKLAREIITASPGLGDLNSGEVEPGMNVTSEDDLRSWLSNNVRSDWHPVGTCAMLPQELGGVVSPALMVYGTSNLRVVDASIMPLEVSSHLMQPTYGIAEKAADIIKNYYKSQYSGAGKN

SEQ ID NO: 100 - protein, 634 aa, synthetic construct
MQQDTNNTSSTDTYDYVIVGGGVAGLALASRISENKDVTVAVLESGPNAEDQFVVYAPGMYGQAVGTELAPLVPTTPQENMGNRSLSIATGRLLGGGSAVNGLVWTRGGLKDYDAWEELGNPGWNGSNLFKYFKKVENFHPPTPAQIEYGATYQKSAHGKNGPIDVSFTNFEFPQSAKWNASLESLDFTALPDLLNGTLAGYSTTPNILDPETARRVDAYAGYIVPYMGRNNLNVLANHTVSRIQFAPQNGSEPLKATGVEWYPTGNKDQKQTIKARYEVIISSGAIGSPKLLEISGIGNKDIVTAAGVESLIDLPGVGANMQDHVHAVTVSTTNIDGYTTNSVFQPGEFTLGNTNETLAQEQREQYEANKTGIWTTTPNNLGYPTPEQLFNGTEFVSGKEFAAKIRNSTDEWANYYASTNATNADLLKKQYAIVASRYEENYLSPIEINLTPGYGGTGSPDLQNNKYQTVNHVLIAPLSRGYAHINSSDIEEPSVINPQYYSHPLDIDVHVASTKLAREIITASPGLGDLNSGEVEPGMNVTSEDDLRSWLSNNVRSDWHPVGTCAMLPQELGGVVSPALMVYGTSNLRVVDASIMPLEVSSHLMQPTYGIAEKAADIIKNYYKSQYSGAGKN

SEQ ID NO: 101 - protein, 634 aa, synthetic construct
MQQDTNNTSSTDTYDYVIVGGGVAGLALASRISENKDVTVAVLESGPNAEDQFVVYAPGMYGQAVGTELAPLVPTTPQENMGNRSLSIATGRLLGGGSAVNGLVWTRGGLKDYDAWEELGNPGWNGSNLFKYFKKVENFHPPTPAQIEYGATYQKSAHGKNGPIDVSFTNFEFPQSAKWNASLESLDFTALPDLLNGTLAGYSTTPNILDPETARRVDAYAGYIVPYMGRNNLNVLANHTVSRIQFAPQNGSEPLKATGVEWYPTGNKDQKQTIKARYEVIISSGAIGSPKLLEISGIGNKDIVTAAGVESLIDLPGVGANMQDHVHAVTVSTTNIDGYTTNSVFTNETLAQEQREQYEANKQPGEFTLGNTGIWTTTPNNLGYPTPEQLFNGTEFVSGKEFAAKIRNSTDEWANYYASTNATNADLLKKQYAIVASRYEENYLSPIEINLTPGYGGTGSPDLQNNKYQTVNHVLIAPLSRGYAHINSSDIEEPSVINPQYYSHPLDIDVHVASTKLAREIITASPGLGDLNSGEVEPGMNVTSEDDLRSWLSNNVRSDWHPVGTCAMLPQELGGVVSPALMVYGTSNLRVVDASIMPLEVSSHLMQPTYGIAEKAADIIKNYYKSQYSGAGKN

SEQ ID NO: 102 - protein, 634 aa, synthetic construct
MQQDTNNTSSTDTYDYVIVGGGVAGLALASRISENKDVTVAVLESGPNAEDQFVVYAPGMYGQAVGTELAPLVPTTPQENMGNRSLSIATGRLLGGGSAVNGLVWTRGGLKDYDAWEELGNPGWNGSNLFKYFKKVENFHPPTPAQIEYGATYQKSAHGKNGPIDVSFTNFEFPQSAKWNASLESLDFTALPDLLNGTLAGYSTTPNILDPETARRVDAYAGYIVPYMGRNNLNVLANHTVSRIQFAPQNGSEPLKATGVEWYPTGNKDQKQTIKARYEVIISSGAIGSPKLLEISGIGNKDIVTAAGVESLIDLPGVGANMQDHVHAVTVSTTNIDGYTTNSVFISASRKLQLTNETLAQEQREQYEANKTGIWTTTPNNLGYPTPEQLFNGTEFVSGKEFAAKIRNSTDEWANYYASTNATNADLLKKQYAIVASRYEENYLSPIEINLTPGYGGTGSPDLQNNKYQTVNHVLIAPLSRGYAHINSSDIEEPSVINPQYYSHPLDIDVHVASTKLAREIITASPGLGDLNSGEVEPGMNVTSEDDLRSWLSNNVRSDWHPVGTCAMLPQELGGVVSPALMVYGTSNLRVVDASIMPLEVSSHLMQPTYGIAEKAADIIKNYYKSQYSGAGKN

SEQ ID NO: 103 - protein, 634 aa, synthetic construct
MQQDTNNTSSTDTYDYVIVGGGVAGLALASRISENKDVTVAVLESGPNAEDQFVVYAPGMYGQAVGTELAPLVPTTPQENMGNRSLSIATGRLLGGGSAVNGLVWTRGGLKDYDAWEELGNPGWNGSNLFKYFKKVENFHPPTPAQIEYGATYQKSAHGKNGPIDVSFTNFEFPQSAKWNASLESLDFTALPDLLNGTLAGYSTTPNILDPETARRVDAYAGYIVPYMGRNNLNVLANHTVSRIQFAPQNGSEPLKATGVEWYPTGNKDQKQTIKARYEVIISSGAIGSPKLLEISGIGNKDIVTAAGVESLIDLPGVGANMQDHVHAVTVSTTNIDGYTTNSVFTNETLAQEQREQYEANKISASRKLQLTGIWTTTPNNLGYPTPEQLFNGTEFVSGKEFAAKIRNSTDEWANYYASTNATNADLLKKQYAIVASRYEENYLSPIEINLTPGYGGTGSPDLQNNKYQTVNHVLIAPLSRGYAHINSSDIEEPSVINPQYYSHPLDIDVHVASTKLAREIITASPGLGDLNSGEVEPGMNVTSEDDLRSWLSNNVRSDWHPVGTCAMLPQELGGVVSPALMVYGTSNLRVVDASIMPLEVSSHLMQPTYGIAEKAADIIKNYYKSQYSGAGKN

SEQ ID NO: 104 - protein, 629 aa, synthetic construct
MQQDTNTSSTDTYDYVIVGGGVAGLALASRISENKDVTVAVLESGPNANDQFVVYAPGMYGQAVGTDLCPLVPTTPQENMGNRSLSIATGRLLGGGSAVNGLVWTRGGLKDYDAWEELGNPGWNGSNLFKYFKKVENFHPPTPAQIEYGATYQKNAHGKNGPIDVSFTNYEFPQSAKWNASLSSLDFTALPDLLNGTLAGYSTTPNILDPETVQRVDSYAGYIAPYTSRSNLNVLANHTVSRIQFAPQNGSEPLKATGVEWYPTGNKDQKQTIKARYEVIISSGAIGSPKLLEISGIGSKDIVSAAGVESLIDLPGVGSNMQDHVHAVTVSTTNITGYTTNSVFYPYDVPDYAVNETLAQEQREEYETNKTGIWTTTPNNLGYPTPEQLFNGTEFVSGKEFADKIRNSTDEWANYYASTNATNVELLKKQYAIVASRYEENYLSPIEINLTPGYGGTGSPDLQNNKYQTVNHVLIAPLSRGYTHINSSDIEEPSVINPQYYSHPMDIDVHVASTKLAREIITASPGLGDLNSGEVEPGMNITSEDDLRSWLSNNVRSDWHPVGTCAMLPKELGGVVSPALMVYGTSNLRVVDASIMPLEVSSHLMQPTYGIAEKAADIIKNAYKNQYKN

SEQ ID NO: 105 - protein, 629 aa, synthetic construct
MQQDTNTSSTDTYDYVIVGGGVAGLALASRISENKDVTVAVLESGPNANDQFVVYAPGMYGQAVGTDLCPLVPTTPQENMGNRSLSIATGRLLGGGSAVNGLVWTRGGLKDYDAWEELGNPGWNGSNLFKYFKKVENFHPPTPAQIEYGATYQKNAHGKNGPIDVSFTNYEFPQSAKWNASLSSLDFTALPDLLNGTLAGYSTTPNILDPETVQRVDSYAGYIAPYTSRSNLNVLANHTVSRIQFAPQNGSEPLKATGVEWYPTGNKDQKQTIKARYEVIISSGAIGSPKLLEISGIGSKDIVSAAGVESLIDLPGVGSNMQDHVHAVTVSTTNITGYTTNSVFVNETLAQEQREEYETNKYPYDVPDYATGIWTTTPNNLGYPTPEQLFNGTEFVSGKEFADKIRNSTDEWANYYASTNATNVELLKKQYAIVASRYEENYLSPIEINLTPGYGGTGSPDLQNNKYQTVNHVLIAPLSRGYTHINSSDIEEPSVINPQYYSHPMDIDVHVASTKLAREIITASPGLGDLNSGEVEPGMNITSEDDLRSWLSNNVRSDWHPVGTCAMLPKELGGVVSPALMVYGTSNLRVVDASIMPLEVSSHLMQPTYGIAEKAADIIKNAYKNQYKN

SEQ ID NO: 106 - protein, 629 aa, synthetic construct
MQQDTNTSSTDTYDYVIVGGGVAGLALASRISENKDVTVAVLESGPNANDQFVVYAPGMYGQAVGTDLCPLVPTTPQENMGNRSLSIATGRLLGGGSAVNGLVWTRGGLKDYDAWEELGNPGWNGSNLFKYFKKVENFHPPTPAQIEYGATYQKNAHGKNGPIDVSFTNYEFPQSAKWNASLSSLDFTALPDLLNGTLAGYSTTPNILDPETVQRVDSYAGYIAPYTSRSNLNVLANHTVSRIQFAPQNGSEPLKATGVEWYPTGNKDQKQTIKARYEVIISSGAIGSPKLLEISGIGSKDIVSAAGVESLIDLPGVGSNMQDHVHAVTVSTTNITGYTTNSVFQPGEFTLGNVNETLAQEQREEYETNKTGIWTTTPNNLGYPTPEQLFNGTEFVSGKEFADKIRNSTDEWANYYASTNATNVELLKKQYAIVASRYEENYLSPIEINLTPGYGGTGSPDLQNNKYQTVNHVLIAPLSRGYTHINSSDIEEPSVINPQYYSHPMDIDVHVASTKLAREIITASPGLGDLNSGEVEPGMNITSEDDLRSWLSNNVRSDWHPVGTCAMLPKELGGVVSPALMVYGTSNLRVVDASIMPLEVSSHLMQPTYGIAEKAADIIKNAYKNQYKN

SEQ ID NO: 107 - protein, 629 aa, synthetic construct
MQQDTNTSSTDTYDYVIVGGGVAGLALASRISENKDVTVAVLESGPNANDQFVVYAPGMYGQAVGTDLCPLVPTTPQENMGNRSLSIATGRLLGGGSAVNGLVWTRGGLKDYDAWEELGNPGWNGSNLFKYFKKVENFHPPTPAQIEYGATYQKNAHGKNGPIDVSFTNYEFPQSAKWNASLSSLDFTALPDLLNGTLAGYSTTPNILDPETVQRVDSYAGYIAPYTSRSNLNVLANHTVSRIQFAPQNGSEPLKATGVEWYPTGNKDQKQTIKARYEVIISSGAIGSPKLLEISGIGSKDIVSAAGVESLIDLPGVGSNMQDHVHAVTVSTTNITGYTTNSVFVNETLAQEQREEYETNKQPGEFTLGNTGIWTTTPNNLGYPTPEQLFNGTEFVSGKEFADKIRNSTDEWANYYASTNATNVELLKKQYAIVASRYEENYLSPIEINLTPGYGGTGSPDLQNNKYQTVNHVLIAPLSRGYTHINSSDIEEPSVINPQYYSHPMDIDVHVASTKLAREIITASPGLGDLNSGEVEPGMNITSEDDLRSWLSNNVRSDWHPVGTCAMLPKELGGVVSPALMVYGTSNLRVVDASIMPLEVSSHLMQPTYGIAEKAADIIKNAYKNQYKN

SEQ ID NO: 108 - protein, 629 aa, synthetic construct
MQQDTNTSSTDTYDYVIVGGGVAGLALASRISENKDVTVAVLESGPNANDQFVVYAPGMYGQAVGTDLCPLVPTTPQENMGNRSLSIATGRLLGGGSAVNGLVWTRGGLKDYDAWEELGNPGWNGSNLFKYFKKVENFHPPTPAQIEYGATYQKNAHGKNGPIDVSFTNYEFPQSAKWNASLSSLDFTALPDLLNGTLAGYSTTPNILDPETVQRVDSYAGYIAPYTSRSNLNVLANHTVSRIQFAPQNGSEPLKATGVEWYPTGNKDQKQTIKARYEVIISSGAIGSPKLLEISGIGSKDIVSAAGVESLIDLPGVGSNMQDHVHAVTVSTTNITGYTTNSVFISASRKLQLVNETLAQEQREEYETNKTGIWTTTPNNLGYPTPEQLFNGTEFVSGKEFADKIRNSTDEWANYYASTNATNVELLKKQYAIVASRYEENYLSPIEINLTPGYGGTGSPDLQNNKYQTVNHVLIAPLSRGYTHINSSDIEEPSVINPQYYSHPMDIDVHVASTKLAREIITASPGLGDLNSGEVEPGMNITSEDDLRSWLSNNVRSDWHPVGTCAMLPKELGGVVSPALMVYGTSNLRVVDASIMPLEVSSHLMQPTYGIAEKAADIIKNAYKNQYKN

SEQ ID NO: 109 - protein, 629 aa, synthetic construct
MQQDTNTSSTDTYDYVIVGGGVAGLALASRISENKDVTVAVLESGPNANDQFVVYAPGMYGQAVGTDLCPLVPTTPQENMGNRSLSIATGRLLGGGSAVNGLVWTRGGLKDYDAWEELGNPGWNGSNLFKYFKKVENFHPPTPAQIEYGATYQKNAHGKNGPIDVSFTNYEFPQSAKWNASLSSLDFTALPDLLNGTLAGYSTTPNILDPETVQRVDSYAGYIAPYTSRSNLNVLANHTVSRIQFAPQNGSEPLKATGVEWYPTGNKDQKQTIKARYEVIISSGAIGSPKLLEISGIGSKDIVSAAGVESLIDLPGVGSNMQDHVHAVTVSTTNITGYTTNSVFVNETLAQEQREEYETNKISASRKLQLTGIWTTTPNNLGYPTPEQLFNGTEFVSGKEFADKIRNSTDEWANYYASTNATNVELLKKQYAIVASRYEENYLSPIEINLTPGYGGTGSPDLQNNKYQTVNHVLIAPLSRGYTHINSSDIEEPSVINPQYYSHPMDIDVHVASTKLAREIITASPGLGDLNSGEVEPGMNITSEDDLRSWLSNNVRSDWHPVGTCAMLPKELGGVVSPALMVYGTSNLRVVDASIMPLEVSSHLMQPTYGIAEKAADIIKNAYKNQYKN

SEQ ID NO: 110 - protein, 602 aa, synthetic construct
MLFSLAFLSALSLATASPAGRAKNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPAVNGKEGPLKVGWSGSLASGNLSVALNRTFQAAGVPWVEDVNGGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVYPYDVPDYALAGASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSA

SEQ ID NO: 111 - protein, 602 aa, synthetic construct
MLFSLAFLSALSLATASPAGRAKNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPAVNGKEGPLKVGWSGSLASGNLSVALNRTFQAAGVPWVEDVNGGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVLAGYPYDVPDYAASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSA

SEQ ID NO: 112 - protein, 602 aa, synthetic construct
MLFSLAFLSALSLATASPAGRAKNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPAVNGKEGPLKVGWSGSLASGNLSVALNRTFQAAGVPWVEDVNGGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVQPGEFTLGNLAGASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSA

SEQ ID NO: 113 - protein, 602 aa, synthetic construct
MLFSLAFLSALSLATASPAGRAKNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPAVNGKEGPLKVGWSGSLASGNLSVALNRTFQAAGVPWVEDVNGGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVLAGQPGEFTLGNASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSA

SEQ ID NO: 114 - protein, 602 aa, synthetic construct
MLFSLAFLSALSLATASPAGRAKNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPAVNGKEGPLKVGWSGSLASGNLSVALNRTFQAAGVPWVEDVNGGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVISASRKLQLLAGASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSA

SEQ ID NO: 115 - protein, 602 aa, synthetic construct
MLFSLAFLSALSLATASPAGRAKNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPAVNGKEGPLKVGWSGSLASGNLSVALNRTFQAAGVPWVEDVNGGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVLAGISASRKLQLASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSA

SEQ ID NO: 116 - protein, 629 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKGSQPGEFTLGNIKTGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 117 - protein, 625 aa, synthetic construct; identical to SEQ ID NO: 62

SEQ ID NO: 118 - protein, 625 aa, synthetic construct
MQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGCHGKNGRIDISFPEFQFPQSANWNASLATLDFTHQQDLLCGSLHGYSTTPNTLDPKTERRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKYPYDVPDYATGIWTTAPNNLGYPSPSQLFNGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGETQPGSNITSDEDVKQWLADNVRSDYHPVGTCAMLPRELGGVVDPNLLVYGTANLRVVDASIMPLEISSHLMQPTYGVAEKAADIIKMSRKNNNN

SEQ ID NO: 119 - protein, 641 aa, unidentified [sequence omitted]

SEQ ID NO: 120 - protein, 641 aa, unidentified
MKITAAIITVATAFASFASAQQDTNSSSTDTYDYVIVGGGVAGLALASRISENKDVTVAVLESGPYAGDRFVVYAPGMYGQAVGTDLAPLIPTTPQENMGNRSLTIATGRLLGGGSAINGLVWTRGGLKDYDAWEELGNPGWNGANLFKYFKKVENFTPPTPAQIEYGATYQKSAHGKKGPIDVSFTNYEFSQSASWNASLETLDFTALPDILNGTLAGYSTTPNILDPETVRRVDSYTGYIAPYTSRNNLNVLANHTVSRIQFAPKNGSEPLKATGVEWYPTGNKNQKQIIKARYEVIISSGAIGSPKLLEISGIGNKDIVSAAGVESLIDLPGVGSNMQDHVHAITVSTTNITGYTTNSVFVNETLAQEQREEYEANKTGIWATCPNNLGYPTPEQLFNGTEFVSGKEFADKIRNSTDEWANYYASTNASNVELLKKQYAIVASRYEENYLSPIEINFTPGYEGSGNVDLQNNKYQTVNHVLIAPLSRGYTHINSSDVEDHSVINPQYYSHPMDIDVHIASTKLAREIITASPGLGDINSGEIEPGMNITSDDDVRKWLSNNVRSDWHPVGTCAMLPKELGGVVSPALMVYGTSNLRVVDASIMPLEVSSHLMQPTYGIAEKAADIIKNFYKTQHKNQN

SEQ ID NO: 121 - protein, 633 aa, unidentified
MKISAAIVTIATAFASLVSAQSNTDTYDYVIVGGGVGGLALANRLSENKQVTVAVLEAGPNANDEFIVYAPGMYGQAVGTYLAPLRPTVPQENMNNRSLSIATGKLLGGGSAVNGLVWTRGATKDFDAWEELGNPGWNGASMFKYFKKVENFTAPTPYQVNYGATYQKNTHGYKGPVQVSFTNYEFPQSAHWNQSLASLGFDHLPDLLNGTLSGYSTTPNILDPNTDQRCDAYAAYIAPYTARTNLHVLANHTVSRIEFNQTNANQPLVASGVEWYPTGDNTKKQTIKARLEVIVSSGSIGSPKLLEISGIGNKDIVTAAGVKSLLDLPGVGSNMQDHVHAVTVSTTNITGYTTDSVFVNSTLASEQREQYEKDKSGIWTTTPNNLGYPTPAQLFNGTEFMDGKAFAARIRNSSQEWAQYYASKNASTVELLMKQYEIVASRYEENYLSPIEINLTPGYGGVGTVDKTKNKYQTVNHVLIAPLSRGFTHINSSDIEDPVNINPQYYSHPMDIDVHVASTKLARRIINAPGLGDLNSGEVEPGMDITSDSDVRAWLANNVRSDWHPVGTCAMLPKELGGVVDSSLKVYGTANLRVVDASIMPLEVSSHLMQPTFGVAEKAADIIKAEYKKQKAQ

SEQ ID NO: 122 - protein, 635 aa, unidentified
MKISVAIVTIAAAFASFANAQKTATSNTYDYVIVGGGVGGLALASRLSEDKSVTVAVLEAGPNADEQFVVYAPGMYGQAVGTDLCPLRPTVPQEAMNNRTLTIATGKLLGGGSAINGLVWTRGALKDFDAWEELGNPGWNGRTMFKYFKKVERFHPPTKAQVQYGATYQKGVHGKNGRIDISFPEFQFPQSANWNASLATLDFTHQQDLLNGSLHGYSTTPNTLDPKTARRVDSYTGYIAPFVSRKNLFVLANHTVSRIQFKPKNGTELLKAVGVEWYTTGDNSNKQTIKARREVIVSSGSIGSPKLLEISGIGNKDIVTAAGVQSLIDLPGVGSNMQDHVHAVTVSTTNITGFTTDSVFQNETLAEEQRQQYYNNKTGIWTTTPNNLGYPSPSQLFDGTSFESGQAFANRIRNSTDQWAEYYASTNATNIELLKKQYAIVASRYEENYLSPIEINFTPGYGGTTDVDLKNNKYQTVNHVLIAPLSRGYTHINSSNIEDPVVINPQYYTHPMDVDVHIASTKLARRILGAEPGLASINSGEIQPGSNITSDEDVKQWLADNVRSDWHPVGTCAMLPRELGGVVDPNLLVYGTANLRVVDASIMPLEISSHLMQPTYGVAEKAADIIKMSRKNNNN

SEQ ID NO: 123 - protein, 631 aa, unidentified
MRLSVAILTLTSALASVTSAQQNNTDTYDYVIVGGGVGGLALASRLSEDKNVTVAVLESGPYADDKFVVYAPGMYGQAVGTDLCPLLPTVPQPSMNNRTITIATGRLLGGGSAVNGLVWTRGAMKDFDAWQELGNPGWNGTTMFKYFKKIENFHPPTEEQIQYGATYNKSVHGFNGPIDIAFPVFEFPQSANWNASLAHLNFTRRQDLLDGSLHGYSTTPNTLNPQTARRADAYAGYIQPNVNRTNLAVLANHTVSRIQFEARNGSQPLKAIGVEWYTTGGDKTSKQTIKARREIILSSGAIGSPKLLEVSGIGNKAIVTAAGVQSLIDLPGVGSNMQDHVHAVTVSTTNIDGYTTNSVFTNETLAQEQKDLYYNNKTGIWTTTPNNLGYPSPSQLFTNTTFKSGKEFAAMIRNSTDKYAQYYAANNATNVELLKKQYSIVARRYEENYISPIEINFTPGYGGTGMADLQNKKYQTVNHVLVAPLSRGYTHINSSDIEDPVVIDPQYYSHPLDVDVHVASTQLARSILNAPGLASINSGEVEPGEKVQSDEDVRKWLSDNVRSDWHPVGTCAMLPRKLGGVVDSKLKVYGTANLRIVDASIIPLEISSHLMQPVYAVSERAADIIKSSSKK

SEQ ID NO: 124 - protein, 633 aa, unidentified
MRLSLAILSLTSALVTVTSAQQNGTSNDTYDYVIVGGGVGGLSLASRLSEDKGVTVAVLESGPYADDRFVVYAPGMYGQAVGTELCPLLPTVPQVGMNNRTITIATGRLLGGGSAVNGLVWTRGAMKDFDAWEELGNPGWNGKTMFKYFKKIENFHPPTEEQVQYGATYQKNVHGSGGPIDISFPVFEFPQSANWNASLAYLNFTHQQDLLNGSLHGYSTTPNTLNPETARRADAYAGYIQPNVNRTNLAVLANHTVSRIQFEKSNGSQPLKAIGVEWYTTGGDKSTKQTIKARREVIISSGAIGSPKLLEVSGIGNKQIVTAAGVESLIDLPGVGSNMQDHVHAVTVSTTNIEGYTTNSVFTNETLAQEQKDLYYNNKTGIWTTTPNNLGYPSPSQLFTNTTFRSGKQFAAMIRNSTDKYAQYYASTKNATNIQLLKKQYAIVARRYEEDYISPIEINFTPGYGGTGEVDLQNNKYQTVNHVLVAPLSRGYTHINSSDIEDPVVIDPQYYSHPLDVDVHVASTQLARSILNAPALAAINSGEVEPGEKIQTDQDVRKWLSDNVRSDWHPVGTCAMLPKGLGGVVDSNLKVYGTANLRVVDASIIPLEISSHLMQPVYAVSERAADIIKGSRN

SEQ ID NO: 125 - protein, 637 aa, unidentified
MKISAAVVTIVTAFASVATAQQQNTSETNTYDYVIVGGGVGGLALASRLSENKGVSVAVLEAGPYAGDQFVVYAPGMYGQAVGTDLCPLLPTTPQENMGNRSLSIATGKLLGGGSSVNGLVWTRGGLKDFDAWEELGNPGWNGASMFNYFKKVENFTPPTPAQAAYGATYQKNAHGTKGPMDVSFTNFEFPQSGNWNASLNAVGFTAVPDLLNGTLHGYSTTPNILDPVNARRADAYAGYIKPYISRNNLAVLANHTVSRIQFAPQSGSQPLRATGVEWYPTGDKSQKQVLNARYEVILSSGAIGSPKLLELSGIGNKDIVAAAGIQSLLDLPGVGSNMQDHVHAVTVSTTNITGYTTNSIFTNDALAAEERQEYDNNKTGIYTTTPNNLGYPSPSQLFRGTSFVSGKQFAARIRNTTDEWAERYAADNATNAELLKKQYAIIASRYEEDYLSPIEINLTPGYGGTADVDLTNNKYQTVNHVLIAPLSRGYTHIKSADIEDAVDINPQYYSHPMDVDVHVASTKLAREIISASPGLGDINSGETEPGKEITSDSDVRKWLADNVRSDWHPVGTCAMLPKELGGVVDPNLKVYGTSNLRVVDASVMPLEVSSHLMQPTFGIAEKAADIIKSANKKRSN

SEQ ID NO: 126 - protein, 637 aa, unidentified
MKISAAVVTIVTAFASVATAQQQNTSETNTYDYVIVGGGVGGLALASRLSENKGVSVAVLEAGPYAGDQFVVYAPGMYGQAVGTDLCPLLPTTPQENMGNRSLSIATGKLLGGGSSVNGLVWTRGGLKDFDAWEELGNPGWNGASMFNYFKKVENFTPPTPAQAAYGATYQKNAHGTKGPMDVSFTNFEFPQSGNWNASLNAVGFTAVPDLLNGTLHGYSTTPNILDPVNARRADAYAGYIKPYISRNNLAVLANHTVSRIQFAPQSGSQPLRATGVEWYPTGDKSQKQVLNARYEVILSSGAIGSPKLLELSGIGNKDIVAAAGIQSLLDLPGVGSNMQDHVHAVTVSTTNITGYTTNSIFTNDALAAEERQEYDNNKTGIYTTTPNNLGYPSPSQLFRGTSFVSGKQFAARIRNTTDEWAERYAADNATNAELLKKQYAIIASRYEEDYLSPIEINLTPGYGGTADVDLTNNKYQTVNHVLIAPLSRGYTHIKSADIEDAVDINPQYYSHPMDVDVHVASTKLAREIISASPGLGDINSGETEPGKEITSDSDVRKWLADNVRSDWHPVGTCAMLPKELDGVVDPNLKVYGTSNLRVVDASVMPLEVSSHLMQPTFGIAEKAADIIKSANKKRSN

SEQ ID NO: 127 - protein, 644 aa, unidentified
MKISAAIITVVTAFASFASAQQDTNNTSSTDTYDYVIVGGGVAGLALASRISENKDVTVAVLESGPNAEDQFVVYAPGMYGQAVGTELAPLVPTTPQENMGNRSLSIATGRLLGGGSAVNGLVWTRGGLKDYDAWEELGNPGWNGSNLFKYFKKVENFHPPTPAQIEYGATYQKSAHGKNGPIDVSFTNFEFPQSAKWNASLESLDFTALPDLLNGTLAGYSTTPNILDPETARRVDAYAGYIVPYMGRNNLNVLANHTVSRIQFAPQNGSEPLKATGVEWYPTGNKDQKQTIKARYEVIISSGAIGSPKLLEISGIGNKDIVTAAGVESLIDLPGVGANMQDHVHAVTVSTTNIDGYTTNSVFTNETLAQEQREQYEANKTGIWTTTPNNLGYPTPEQLFNGTEFVSGKEFAAKIRNSTDEWANYYASTNATNADLLKKQYAIVASRYEENYLSPIEINLTPGYGGTGSPDLQNNKYQTVNHVLIAPLSRGYAHINSSDIEEPSVINPQYYSHPLDIDVHVASTKLAREIITASPGLGDLNSGEVEPGMNVTSEDDLRSWLSNNVRSDWHPVGTCAMLPQELGGVVSPALMVYGTSNLRVVDASIMPLEVSSHLMQPTYGIAEKAADIIKNYYKSQYSGAGKN

SEQ ID NO: 128 - protein, 19 aa, synthetic construct
SGSGPGSQPGEFTLGNIKS

SEQ ID NO: 129 - protein, 19 aa, synthetic construct
SGSGQPGEFTLGNIKSYPG

SEQ ID NO: 130 - protein, 22 aa, synthetic construct
LFSLAFLSALSLATASPAGRAK

SEQ ID NO: 131 - protein, 571 aa, Aspergillus flavus
MNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPAVNGKEGPLKVGWSGSLASGNLSVALNRTFQAAGVPWVEDVNGGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVLAGASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSA

SEQ ID NO: 132 - protein, 580 aa, Aspergillus flavus
MNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPAVNGKEGPLKVGWSGSLASGNLSVALNRTFQAAGVPWVEDVNGGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVISASRKLQLLAGASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSA

SEQ ID NO: 133 - protein, 580 aa, Aspergillus flavus
MNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPAVNGKEGPLKVGWSGSLASGNLSVALNRTFQAAGVPWVEDVNGGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVYPYDVPDYALAGASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSA

SEQ ID NO: 134 - protein, 580 aa, Aspergillus flavus
MNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPAVNGKEGPLKVGWSGSLASGNLSVALNRTFQAAGVPWVEDVNGGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVQPGEFTLGNLAGASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSA

(19) United States
(12) Patent Application Publication
(10) Pub. No.: US 2024/0240159 A1
(43) Pub. Date: Jul. 18, 2024

(54) UNIVERSAL SENSING SYSTEM

(71) Applicant: Abbott Diabetes Care, Inc, Alameda, CA (US)

(72) Inventors: Josie Corby, Milwaukee, WI (US); Barry S. Kreutz, Chicago, IL (US); Hao Lei, Chicago, IL (US); Anthony S. Muerhoff, Kenosha, WI (US); Christopher Marohnic, Kenosha, WI (US); Thomas P. Leary, Peoria, AZ (US)

(21) Appl. No.: 18/521,870

(22) Filed: Nov. 28, 2023

Related U.S. Application Data
(60) Provisional application No. 63/385,147, filed on Nov. 28, 2022.

(51) Int. Cl. C12N 9/04; C12Q 1/00; G01N 33/573

(52) U.S. Cl. CPC C12N 9/0006; C12Q 1/005; G01N 33/573; G01N 2333/904

(57) ABSTRACT

Provided herein are systems and methods for the detection and analysis of biomolecules. In particular, provided herein are sensor systems employing an enzyme that comprises an allosteric site that interacts with an inhibitor to determine the presence of, absence of, or amount of one or more analytes of interest in a sample. In some embodiments, the allosteric site comprises a grafted epitope that corresponds to one or more analytes of interest.

The present application claims priority to U.S. Provisional Application Ser. No. 63/385,147, filed Nov. 28, 2022, the disclosure of which is herein incorporated by reference in its entirety.

### SEQUENCE LISTING

The text of the computer readable sequence listing filed herewith, titled “40764-209_SEQUENCE_LISTING”, created Nov. 28, 2023, having a file size of 197,680 bytes, is hereby incorporated by reference in its entirety.

### FIELD

Provided herein are systems and methods for the detection and analysis of biomolecules. In particular, provided herein are sensor systems employing an enzyme that comprises an allosteric site that interacts with an inhibitor to determine the presence of, absence of, or amount of one or more analytes of interest in a sample. In some embodiments, the allosteric site comprises a grafted epitope that corresponds to one or more analytes of interest.

### BACKGROUND

Molecular diagnostics is a collection of techniques used to analyze biological markers and has emerged as an important component of medical and health care testing. In medicine, the techniques are used to diagnose and monitor disease, detect risk, monitor health status, and decide which therapies will work best for patients. Traditionally, a biological sample is removed from a subject and sent to a laboratory for analysis by one or more molecular diagnostic tests. Many of the tests are time consuming and utilize complicated, expensive equipment that is available in a limited number of locations. The desire to obtain relevant information more quickly has driven the development of technologies that can be used at point-of-care locations or used directly by patients outside of a medical facility. For example, technology has been developed that allows diabetic patients to self-monitor their blood glucose levels at any time at any location. Unfortunately, such technology is not available for the vast majority of the numerous biomarkers that provide important information about the health and well-being of patients. What is needed are new technology platforms that provide the ability to analyze a wide variety of different biomarkers, including large complex molecules such as proteins, that provide results quickly, and that offer convenience for the user.

One approach that has been described previously comprises an enzyme that is engineered to bind an analyte at an allosteric site, resulting in a decrease or increase in enzyme activity when the analyte is bound to the allosteric site (see, WO2021/067608, herein incorporated by reference in its entirety). However, this approach involves enzyme re-design for each different analyte to be detected, often involving molecule evolution approaches. Accordingly, new approaches are needed.

### SUMMARY

Provided herein are compositions and methods related to universal sensing systems.

The present disclosure provides enzymes suitable for use in sensing systems as described herein. As explained herein in more detail, the enzymes comprise a modified allosteric site comprising a grafted epitope corresponding to an analyte of interest. The epitope is selected or designed to bind to an inhibitor capable of also binding to the analyte.

For example, the epitope may have an amino acid sequence which corresponds to the amino acid sequence of an inhibitor-binding site on the analyte. When contacted with an inhibitor capable of binding to the analyte, the enzymatic activity of the enzyme is inhibitable as the inhibitor also binds to the grafted epitope.

The provided enzymes are useful in sensing analyte (e.g. concentrations thereof) in samples such as biological samples. In the absence of the analyte (or in the presence of only low concentrations of the analyte) the enzymatic activity of the enzyme is typically inhibited because the inhibitor is capable of binding to the epitope of the enzyme, thus inhibiting the activity of the enzyme. In the presence of the analyte (or in the presence of elevated analyte concentrations) the enzymatic activity of the enzyme is typically restored (referred to herein as “deinhibition”) because the inhibitor at least partially binds to the analyte. Accordingly, the enzymes provided herein are amenable to being used in sensing systems for detecting an analyte of interest.

The enzymes provided herein, and systems/uses comprising them, offer significant advantages compared to the approaches that have been considered previously. In particular, the enzymes are readily configured for use in sensing a wide variety of analytes. An epitope corresponding to a given analyte can be readily identified and grafted into the allosteric site of an enzyme as described herein. Accordingly, the approach described herein provides a universal sensing paradigm for a wide variety of analytes, including large molecule analytes such as proteins and peptides. Once an inhibitor that binds to the analyte at a graftable epitope has been identified, that epitope can be grafted into an enzyme as described herein and the resulting epitope-grafted enzyme can be used e.g., in sensor systems as described herein in order to detect the analyte.

Sensing systems employing the enzymes may be adapted to detect any analyte of interest in any sample type. In particular, the sensing systems employ an enzyme that modifies a substrate. The modification of the substrate generates a detectable event. The detectable event is detected by a sensor, processed, and reported to a user. The enzyme comprises an epitope-grafted sequence. The epitope grafted sequence corresponds to an epitope of the analyte of interest (e.g., has an amino acid sequence and/or structure corresponding to an epitope of analyte, although, as discussed below “corresponds to” does not have to mean 100% identical). The sensing systems further employs an inhibitor that competitively binds to the analyte of interest and to the epitope-grafted sequence. When the inhibitor is bound to the epitope-grafted sequence of the enzyme, the enzyme is inhibited, decreasing or preventing the enzyme from processing substrate, resulting in a change in detectable signal (e.g., resulting in a lower or undetectable signal). When the inhibitor is not bound to the epitope grafted sequence of the enzyme, the enzyme processes substrate, resulting in a change in the detectable signal (e.g., generating a detectable signal or increasing the amount of detectable signal). In the presence of analyte near the sensor, the inhibitor will bind to analyte, de-inhibiting the enzyme to the extent that inhibitor migrates from an inhibited enzyme to the analyte, resulting in a detectable event based on the change in signal. In the absence of an analyte near the sensor, the inhibitor will more likely be bound to the epitope-grafted sequence of the enzyme, maintaining the enzyme in an inhibited state, and sensor signal decreases or becomes undetectable relative to an established background level.

By utilizing different epitope sequences, the sensing systems are readily designed to detect any analyte of interest or combinations of analytes. Where combinations of analytes are detected together, two or more epitope sequences may be inserted into a single enzyme at the same or different allosteric locations or multiple different enzymes are employed, each having its own distinct epitope-grafted sequence. As demonstrated in the experimental section below, a large number of diverse analytes were detected, including large molecule protein analytes.

The universal sensor system described herein has advantages over enzyme switch technologies, like those described in WO2021/067608, herein incorporated by reference in its entirety. With the enzyme switch approaches, the enzyme must undergo re-design for each different analyte that is detected, often involving molecule evolution approaches. With the universal sensor system described herein, much less engineering work is required to replace one epitope grafted sequence for another.

In some embodiments, provided herein are enzymes comprising an epitope-grafted allosteric site that is inhibited by contact with an inhibitor and is de-inhibited in the presence of an analyte that binds to the inhibitor. In some embodiments, the enzyme is a glucose-metabolizing enzyme. In some embodiments, the glucose-metabolizing enzyme is an FAD dependent glucose dehydrogenase (FAD-GDH) enzyme. In some embodiments, the FAD-GDH enzyme is a fungal FAD-GDH (an FAD-GDH enzyme derived from a fungal organism). In some embodiments, the FAD-GDH enzyme is a genus Mucor FAD-GDH. In some embodiments, the enzyme is an FAD-GDH from an organism selected from the group consisting of: M. hiemalis, M. circinelloides, M. ambiguus, M. lusitanicus, M. guilliermondii, M. subtillissimus, and M. prainii. In some embodiments, the FAD-GDH enzyme is an Aspergillus genus FAD-GDH (e.g., A. flavus). In some embodiments, the enzyme, other than the epitope-graft, is a wild-type enzyme. In some embodiments, the enzyme, in addition to the epitope graft, comprises a synthetic sequence variation. In some embodiments, the synthetic sequence variation comprises a sequence variation that increase enzyme stability relative to a non-variant enzyme. In some embodiments, the enzyme comprises a sequence selected from the group consisting of: SEQ ID NOS: 1-64, 65-72, 75-127, and 132-134 or a sequence at least 70% identical thereto. In some embodiments, the enzyme is an FAD-GDH enzyme and the allosteric site is located on a surface region corresponding to residue ranges 45-70, 335-362, and 439-457 of SEQ ID NO:1. In some embodiments, the epitope-grafted sequence comprises an epitope sequence corresponding to an analyte. In some embodiments, the analyte is a protein. In some embodiments, the analyte is a peptide. In some embodiments, the analyte is selected from the group consisting of: a cardiovascular disease biomarker, a cancer biomarker, an infectious disease biomarker, an inflammation biomarker, a metabolism biomarker, and a transplant rejection biomarker. In some embodiments, the epitope-grafted sequence comprises from 3 to 30 amino acids.

In some embodiments, provided herein are compositions comprising: an enzyme comprising an epitope-grafted allosteric site that is inhibited by contact with an inhibitor and is de-inhibited in the presence of an analyte that binds to said inhibitor. In some embodiments, the enzyme is a glucose-metabolizing enzyme. In some embodiments, the glucose-metabolizing enzyme is an FAD dependent glucose dehydrogenase (FAD-GDH) enzyme. In some embodiments, the FAD-GDH enzyme is a fungal FAD-GDH. In some embodiments, the FAD-GDH enzyme is a genus Mucor FAD-GDH. In some embodiments, the enzyme is an FAD-GDH from an organism selected from the group consisting of: M. hiemalis, M. circinelloides, M. ambiguus, M. lusitanicus, M. guilliermondii, M. subtillissimus, and M. prainii. In some embodiments, the FAD-GDH enzyme is an Aspergillus genus FAD-GDH (e.g., A. flavus). In some embodiments, the enzyme, other than the epitope-graft, is a wild-type enzyme. In some embodiments, the enzyme, in addition to the epitope graft, comprises a synthetic sequence variation. In some embodiments, the synthetic sequence variation comprises a sequence variation that increase enzyme stability relative to a non-variant enzyme. In some embodiments, the enzyme comprises a sequence selected from the group consisting of: SEQ ID NOS: 1-64, 65-72, 75-127, and 132-134 or a sequence at least 70% identical thereto. In some embodiments, the enzyme is an FAD-GDH enzyme and the allosteric site is located on a surface region corresponding to residue ranges 45-70, 335-362, and 439-457 of SEQ ID NO:1. In some embodiments, the epitope-grafted sequence comprises an epitope sequence corresponding to an analyte. In some embodiments, the analyte is a protein. In some embodiments, the analyte is a peptide. In some embodiments, the analyte is selected from the group consisting of: a cardiovascular disease biomarker, a cancer biomarker, an infectious disease biomarker, an inflammation biomarker, a metabolism biomarker, and a transplant rejection biomarker. In some embodiments, the epitope-grafted sequence comprises from 3 to 30 amino acids.

In some embodiments, provided herein is a system comprising any of the enzymes or compositions above and an inhibitor that binds to the analyte and to said epitope-grafted sequence. In some embodiments, the inhibitor binds to the analyte with a greater affinity than the inhibitor binds to the epitope-grated sequence. In some embodiments, the inhibitor is an immunoglobulin. In some embodiments, the immunoglobulin is an antibody. In some embodiments, the immunoglobulin is an antibody fragment. In some embodiments, the system further comprises a substrate for the enzyme. In some embodiments, the substrate is glucose. In some embodiments, the system further comprises a sensor. In some embodiments, the sensor is an electrochemical sensor. In some embodiments, the sensor detects a product of the enzyme reacting with a substrate. In some embodiments, the system further comprises a sample. In some embodiments, the sample is a biological sample. In some embodiments, the biological sample is selected from the group consisting of blood, serum, plasma, interstitial fluid, saliva, and urine.

In some embodiments, provided herein is a reaction mixture comprising any of the above enzymes or compositions. In some embodiments, the reaction mixture comprises an inhibitor that binds to the analyte and to the epitope-grafted sequence. In some embodiments, the inhibitor is an immunoglobulin. In some embodiments, the immunoglobulin is an antibody. In some embodiments, the immunoglobulin is an antibody fragment. In some embodiments, the reaction mixture further comprises a substrate for said enzyme. In some embodiments, the substrate is glucose. In some embodiments, the reaction mixture further comprises a sample. In some embodiments, the sample is a biological sample. In some embodiments, the biological sample is selected from the group consisting of blood, serum, plasma, interstitial fluid, saliva, and urine.

In some embodiments, provided herein are kits comprising any of the above enzymes, compositions, or systems. In some embodiments, the kit comprises an inhibitor that binds to the analyte and to the epitope-grafted sequence. In some embodiments, the inhibitor is an immunoglobulin. In some embodiments, the immunoglobulin is an antibody. In some embodiments, the immunoglobulin is an antibody fragment. In some embodiments, the kit comprises a substrate for the enzyme. In some embodiments, the substrate is glucose. In some embodiments, the kit comprises a sensor. In some embodiments, the sensor is an electrochemical sensor. In some embodiments, the kit comprises a control sample comprising the analyte. In some embodiments, the kit comprises a control sample lacking the analyte.

In some embodiments, provided herein is a use of an enzyme, a composition, system, reaction mixture, or kit as described above. In some embodiments, provided herein is a use of an enzyme, a composition, system, reaction mixture, or kit as described above for detecting the presence of, absence of, or amount of an analyte in a sample.

In some embodiments, provided herein is a method of detecting analyte, comprising: a) contacting a sample suspected of containing an analyte to an enzyme as described above; and b) detecting, directly or indirectly, activity of said enzyme. In some embodiments, the detecting comprises electrochemical measurement of a byproduct of the enzyme reacting with a substrate.

In some embodiments, provided herein is:
i) an enzyme comprising a modified allosteric site, wherein the modified allosteric site comprises a grafted heterologous epitope; wherein the enzyme has an enzymatic activity which is inhibited by the binding of an inhibitor to the grafted epitope.
ii) the enzyme, wherein the epitope comprises an amino acid sequence corresponding to an inhibitor binding site of a polypeptide analyte and the inhibitor is capable of competitively binding to the polypeptide analyte and to the grafted epitope.
iii) any of above enzymes, wherein the epitope comprises an amino acid sequence having at least 70%, at least 80%, or at least 90% sequence identity to a corresponding sequence of the inhibitor binding site of the analyte.
iv) any of above enzymes, wherein the analyte is a peptide, polypeptide or protein.
v) any of above enzymes, wherein the epitope is a linear epitope.
vi) any of above enzymes, wherein the epitope is a conformational epitope.
vii) any of above enzymes, wherein the analyte is selected from a cardiovascular disease biomarker, a cancer biomarker, an infectious disease biomarker, an inflammation biomarker, a metabolism biomarker, or a transplant rejection biomarker.
viii) any of above enzymes, wherein the epitope comprises from about 3 to about 30 amino acids.
ix) any of above enzymes, wherein the epitope comprises from about 5 to about 15 amino acids, preferably from about 8 to about 10 amino acids.
x) any of above enzymes, wherein said enzyme is a glucose-metabolizing enzyme.
xi) any of above enzymes, wherein the enzyme is an FAD-dependent glucose dehydrogenase (FAD-GDH) (e.g., a fungal FAD-GDH).
xii) any of above enzymes, wherein the enzyme is derived from an FAD-GDH of family Mucoraceae or Aspergillaceae, preferably wherein the enzyme is derived from an FAD-GDH of genus Mucor or genus Aspergillus.
xiii) any of above enzymes, wherein the enzyme is derived from the FAD-GDH from M. hiemalis, M. circinelloides, M. ambiguus, M. lusitanicus, M. guilliermondii, M. prainii, M. subtillissimus, and A. flavus.
xiv) any of above enzymes, wherein the enzyme is derived from the FAD-GDH from M. hiemalis, M. circinelloides, M. ambiguus, M. prainii and M. subtillissimus.
xv) any of above enzymes, wherein the enzyme has at least 70%, at least 80% or at least 90% identity to: a) SEQ ID NOs: 1, 66-72, or 119-127; b) 1, 66-72, 119-127, or 132-134; c) 1, 66-72, 110-115, or 119-127; d) 1, 66-72, 110-112, 114, or 119-127; e) 1, 66-72, 110-112, 114, 119-127, or 132-134; f) 1, 66-72, 110-115, 119-127, or 132-134; optionally wherein the sequence identity is assessed over the entire sequence of the enzyme excluding the epitope.
xvi) any of above enzymes, wherein the allosteric site is located on the surface of the enzyme.
xvii) any of above enzymes, wherein the allosteric site is located on a surface corresponding to Surface 2 of the enzyme of SEQ ID NO: 1, wherein Surface 2 comprises residues F341, E344, E348, and K358 of SEQ ID NO: 1; optionally wherein Surface 2 comprises residues T337, D338, V340, F341, N434, E344, L346, E348, E349, Y354 and K358 of SEQ ID NO: 1.
xviii) any of above enzymes, wherein the epitope is grafted at a position corresponding to (i) from about position 330 to about position 370 of SEQ ID NO: 1; optionally from about position 335 to about position 362; further optionally wherein the epitope is grafted at a position corresponding to position T337, D338, V340, F341, N434, E344, L346, E348, E349, Y354, or K358 of SEQ ID NO: 1; optionally wherein the epitope is grafted at a position corresponding to position 341 or 358 of SEQ ID NO: 1; or (ii) from about position 320 to about position 335 of SEQ ID NO: 131, optionally from about position about position 325 to about position 330, further optionally from about position 327 to about position 329 of SEQ ID NO: 131; optionally wherein the epitope is grafted at a position corresponding to position 328 of SEQ ID NO:131.
xix) any of above enzymes, further comprising one or more amino acid modifications that increase the stability of the enzyme.
xx) any of above enzymes, comprising Cys at the position corresponding to positions 153 and 192 of SEQ ID NO: 118.
xxi) a system comprising any of the above enzymes, and an inhibitor; wherein said inhibitor is capable of binding to the grafted epitope of said enzyme and to an analyte comprising said epitope.
xxii) the system, wherein the affinity of the analyte for the inhibitor is greater than the affinity of the epitope-granted enzyme for the inhibitor.
xxiii) any of the above systems, wherein the inhibitor is an immunoglobulin, an antibody, or an antibody fragment.
xxiv) any of the above systems, further comprising a substrate for said enzyme. xxv) any of the above systems, wherein said substrate is glucose.
xxvi) any of the above systems, further comprising an analyte having a binding site for said inhibitor, wherein the amino acid sequence of said binding site corresponds to the amino acid sequence of the grafted epitope of said enzyme.
xxvii) any of the above systems, wherein the analyte is a peptide, polypeptide or protein; preferably wherein the analyte is selected from a cardiovascular disease biomarker, a cancer biomarker, an infectious disease biomarker, an inflammation biomarker, a metabolism biomarker, or a transplant rejection biomarker.
xxviii) any of the above systems, wherein the analyte is present in a biological sample; optionally wherein the biological sample is selected from blood, serum, plasma, interstitial fluid, saliva, and urine.
xxix) a sensor comprising any of the above enzymes or systems.
xxx) the sensor, wherein said sensor is an electrochemical sensor.
xxxi) any of the above sensors, configured to directly or indirectly sense the rate of substrate turnover by said enzyme.
xxxii) any of the above sensors, configured to detect the product of substrate turnover by said enzyme.
xxxiii) a method of detecting an analyte, e.g. a method of determining the presence, absence, or concentration of an analyte in a sample, the method comprising: a) contacting the sample with any of the above enzymes in the presence of an inhibitor capable of binding to the analyte and to the grafted epitope of said enzyme; and b) taking one or more measurements characteristic of the enzymatic activity of the enzyme.
xxxiv) the method, comprising: a) contacting the sample with any of the above enzymes in the presence of an inhibitor capable of binding to the analyte and to the grafted epitope of said enzyme; b) allowing the inhibitor to inhibit the enzyme; c) allowing analyte present in the sample to bind to the inhibitor thereby deinhibiting the enzyme; and d) taking one or more measurements characteristic of the enzymatic activity of the enzyme; optionally wherein the enzymatic activity of the enzyme is proportional to the concentration of the analyte in the sample.
xxxv) any of the above methods, wherein the sample is a biological sample selected from blood, serum, plasma, interstitial fluid, saliva, or urine.
xxxvi) a method of identifying an allosteric site on an enzyme wherein the allosteric site is capable of being inhibited by an inhibitor, the method comprising: a) generating one or more antibodies and/or antibody mimetics that bind to the enzyme; b) screening the ability of said one or more antibodies and/or antibody mimetics to allosterically inhibit the enzymatic activity of the enzyme, thereby identifying antibodies and/or antibody mimetics which allosterically inhibit the enzymatic activity of the enzyme; and c) identifying the amino acids of the enzyme which contact said antibodies and/or antibody mimetics which allosterically inhibit the enzymatic activity of the enzyme.
xxxv) the above method, further comprising determining the retention of enzymatic activity when said amino acids are modified.
xxxvi) any of the above two methods, further comprising the step of grafting an epitope into the amino acid sequence of the enzyme at a position corresponding to the allosteric site, wherein the epitope comprises an amino acid sequence capable of binding to the inhibitor.
xxxvii) any of the above three methods, wherein the enzyme, allosteric site, inhibitor and/or epitope are as defined above.
xxxviii) a method of diagnosing the health of a subject, comprising a) contacting a biological sample from said subject any of the above sensors; and b) determining the presence, absence, or concentration of an analyte associated with the health of the subject in the sample according to any of the above methods.
xxxix) use of any of the above enzymes, systems, or a sensors according to any of the above methods for determining the presence, absence or concentration of an analyte in a sample.

Provided herein, below, are exemplary compositions of matter, methods, devices (e.g., sensors), systems, reaction mixtures, kits, and apparatus for detecting and/or monitoring analytes in a sample. It is to be understood that the teachings of this disclosure are not limited to the particular aspects described, and as such can, of course, vary. It is also to be understood that the terminology used herein is for the purpose of describing particular aspects only and is not intended to be limiting.

For purposes of illustration, and not limitation, sensor systems can be designed to recognize, identify and/or quantify one or more analytes in a sample. The sensor systems can be utilized in a variety of conditions and configurations, including in a sensor for measuring the presence of or levels of the analyte in a subject. The configuration of such a sensor can depend on the analyte measured and the type of sample the system monitors for the analyte. In some embodiments, sensors are configured for detecting and/or measuring analyte in vivo in a subject. The analyte may be present in any type of sample. For example, the sensor can test for analyte in the dermal fluid, interstitial fluid, subcutaneous fluid, urine, or blood (e.g., capillary blood). In some embodiments, the sensors are configured to detect or measure analyte using a handheld or benchtop device. In such embodiments, sample is transferred from its original source to the device for measurement.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety.

The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other aspects “comprising,” “consisting of” and “consisting essentially of,” the aspects or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Ranges include all intermediate ranges, for example, 6-7, 6-8,7-9, 8-9, and 7-8.

The term “sample” is used in its broadest sense. Samples include biological and environmental samples. Biological samples may be obtained from any source including animals, plants, and microorganisms and encompass fluids, solids, tissues, and gases. Materials obtained from clinical or forensic settings that contain analytes of interest are also within the intended meaning of the term “sample.” In some embodiments, the sample is a biological sample derived from an animal (e.g., a human). Biological samples include, but are not limited to, blood, serum, plasma, interstitial fluid, urine, feces, saliva, tissue, cerebrospinal fluid, semen, vaginal fluids, mucus, lymph, transcellular fluid, aqueous humor, bone marrow, bronchoalveolar lavage, buccal swab, earwax, gastric fluid, gastrointestinal fluid, milk, nasal wash, liposuction, peritoneal fluid, sebum, synovial fluid, tears, sweat, and vitreous humor. Environmental samples include, but are not limited to, water, air, snow, and soil. Samples may be in a processed form, including dried (e.g., dried blood spots) and fixed (e.g., formalin-fixed paraffin-embedded (FFPE)) samples. In some embodiments, the sample is located in vivo in an animal.

“Enzyme” refers to a protein or a fragment thereof having activity (alternatively referred to as catalytic activity, enzyme activity, or enzymatic activity) towards one or more reactants (e.g., enzyme substrate). Examples of one or more reactants (e.g., enzyme substrates) are glucose, lactate, glutamate, ascorbic acid, cholesterol, choline acetylcholine, hypoxanthine, norepinephrine, 5-hydroxytryptamine, phenylethylamine and e/e-methylhistamine, a polyphenol, ethanol, an aldehyde, or malate.

As used herein, the term “epitope” refers to a sequence (e.g., an amino acid sequence) that is recognized by a binding molecule. The term epitope includes sequences recognized by antibodies, antibody fragments, and antibody mimetics, including aptamers, affimers, and DARPins. Epitopes include conformational epitopes and linear epitopes.

“Epitope-grafted” as used herein refers to a molecule that contains a heterologous epitope sequence. For example, an epitope-grafted enzyme is an enzyme that has been modified to include an epitope sequence from a different molecule (e.g., from an analyte of interest). In some embodiments, an epitope-grafted enzyme includes an epitope sequence from a different molecule (e.g. from an analyte of interest) that is grafted into the sequence of the enzyme by being inserted into the sequence of the enzyme, e.g. by adding the sequence of the epitope into the sequence of the enzyme or by replacing one or more amino acids (e.g. contiguous amino acids) of the sequence of the enzyme with the sequence of the epitope. In some embodiments wherein an epitope is grafted into the enzyme sequence by replacing one or more amino acids of the sequence of the enzyme with the sequence of the epitope, at least 1, 2, 3, 4, 5, 6, 7, 8, 9, 10, 15, 20, 25 or at least 30 amino acids (e.g. contiguous amino acids) of the sequence of the enzyme are replaced with the sequence of the epitope. In some embodiments the number of amino acids replaced in the sequence of the enzyme is the same as the number of amino acids in the sequence of the epitope.

“Antigen binding molecule” refers to a molecule that binds a specific antigen. Examples include, but are not limited to, proteins, nucleic acids, aptamers, affimers, DARPins, synthetic molecules, etc.

“Antigen binding protein” refers to proteins that bind to a specific antigen. “Antigen binding proteins” include, but are not limited to, immunoglobulins, including polyclonal, monoclonal, chimeric, single chain, camelid, VHH, and humanized antibodies, Fab fragments, F(ab′)2 fragments, and Fab expression libraries.

“Specific binding” or “specifically binding” when used in reference to the interaction of a binding molecule and an antigen means that the interaction is dependent upon the presence of a particular structure (e.g., the antigenic determinant or epitope) on the antigen; in other words, the antibody is recognizing and binding to a specific structure rather than to antigens in general.

“Affimer” as used herein refers to peptides that specifically or selectively bind to a target (e.g,, analyte, epitope-grafted sequence). Generally, affimers can be small peptides or proteins, generally with a molecular weight less than 12 kDa, Affimers can have the capacity to recognize specific epitopes or antigens, and with binding affinities that can be close to those of antibodies (e.g., in the low nanomolar to picomolar range); however, the term “affirmer,” as used herein, does not encompass antibodies, immunoglobulins, Fab regions of antibodies, or Fc regions of antibodies. Affimers can have the same specificity advantage of antibodies, but can be smaller, can be chemically synthesized or chemically modified, and have the advantage of being free from cell culture contaminants.

“Aptamer” as used herein refers to oligonucleotide or peptide molecules that bind to a specific target molecule. Aptamers are usually created by selecting them from a large random sequence pool, but natural aptamers also exist.

“DARPin” (designed ankyrin repeat proteins) as used herein refers to genetically engineered antibody mimetic proteins typically exhibiting highly specific and high-affinity target protein binding. They are typically derived from natural ankyrin repeat proteins, one of the most common classes of binding proteins in nature, which are responsible for diverse functions such as cell signaling, regulation and structural integrity of the cell. DARPins comprise at least three, repeat motifs or modules, of which the most N- and the most C-terminal modules are referred to as “caps”, since they shield the hydrophobic core of the protein.

“Sensor” as used herein refers to a device or molecule configured to detect the presence and/or measure the level (e.g., presence, absence or concentration) of one or more (e.g., multiple) analytes in a sample. Sensors can include biological, mechanical, and electrical components. An “electrochemical sensor” is chemical sensor in which an electrode is used as a transducer element in the presence of an analyte. In some embodiments, a detectable signal is produced via electrochemical oxidation and reduction reactions on the sensor. These reactions are transduced to an electrical signal that can be correlated to an amount, concentration, or level of an analyte or activity of an enzyme in a sample.

“Sensing layer” as used herein refers to a component of a sensor that includes constituents that facilitate electrooxidation or electroreduction of a compound either directly at an electrode or via one or more electron transfer agents, one or more co-factors or a combination of one or more electron transfer agents and one or more co-factors. In some aspects of the sensor, the sensing layer is disposed in proximity to or on the working electrode.

“Sensing region” as used herein refers to the active chemical area of a sensor.

“Identical”, “identity,” or “sequence identity” as used herein in the context of two or more polypeptide or polynucleotide sequences, means that the sequences have a specified percentage of residues that are the same over a specified region that is determined using a standard algorithm, having regard to the extent that sequences are identical over a window of comparison. Typically, identity is assessed over the full length of the sequence. In some embodiments when the sequence is a sequence of an epitope-grafted enzyme as described herein, the sequence is typically assessed over the full length of the sequence excluding the epitope. Identity or sequence identity may be determined using computer algorithms such as GAP, BESTFIT, FASTA and the BLAST family of programs as for example disclosed by Altschul et al., 1997, Nucl. Acids Res. 25:3389. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

“Subject” or “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some aspects, the subject may be a human or a non-human. In some aspects, the subject is a human. The subject or patient may be undergoing one or more forms of treatment.

The term “variant protein,” “protein variant,” or “variant” as used interchangeably herein is meant a protein that differs from that of a parent protein by virtue of at least one amino acid modification. The term “protein variant” may refer to the protein itself, a composition comprising the protein, or the amino sequence that encodes it. In some aspects, the protein variant has at least one amino acid modification compared to the parent or reference protein, e.g. from about one to about one hundred amino acid (e.g., 2-100, 1-50,2-40, 5-30, 10-20, and all ranges between) modifications compared to the parent protein. In some aspects, the protein variant has from about one to about forty amino acid modifications compared to the parent protein. In some aspects, the protein variant has from about one to about thirty amino acid modifications compared to the parent protein. In some aspects, the protein variant has from about one to about twenty amino acid modifications compared to the parent protein. In some aspects, the protein variant has from about one to about ten amino acid modifications compared to the parent protein. In some aspects, the protein variant has from about one to about five amino acid modifications compared to the parent protein. In some aspects, a protein variant sequence herein will possess at least about 70%, or at least about 80% identity with a parent or reference protein sequence. In other aspects, a protein variant sequence herein will possess at least about 90% identity. In still other aspects, a protein variant sequence will possess least about 95%, 96%, 97%, 98%, or 99% identity. As those skilled in the art will appreciate, any of the sequence identity levels provided herein can be applied to any of the proteins disclosed herein.

Variant proteins typically retain properties of the unmodified parent or reference sequence, or in some instances may have improved properties. For example, a variant enzyme typically retains the enzymatic activity of the unmodified sequence. In some instances, the enzymatic activity may be comparable to that of the unmodified sequence. In some instances, enzymatic activity may be improved. Typically, a variant sequence retains not less than 70%, 80% or 90% enzymatic activity of the unmodified sequence.

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids include, for example, pyrolysine (found in methanogenic organisms and other eukaryotes), selenocysteine (present in many non-eukaryotes as well as most eukaryotes), and N-formylmethionine (encoded by the start codon AUG in bacteria, mitochondria, and chloroplasts). “Unnatural” or “non-natural” amino acids are non-proteinogenic amino acids (e.g., those not naturally encoded or found in the genetic code) that either occur naturally or are chemically synthesized. Over 140 unnatural amino acids are known and thousands of more combinations are possible. Examples of “unnatural” amino acids include β-amino acids (β3 and β2), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975).

In the present disclosure, reference is made to amino acids. In addition to the name of amino acids, the three-letter and one-letter codes are also used herein. For clarity purposes, the amino acids referred to in this disclosure are referred to as follows: alanine (Ala, A), arginine (Arg, R), asparagine (Asn, N), Aspartic acid (Asp, D), cysteine (Cys, C), glutamine (Qln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y) and valine (Val, V).

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). The term “L-amino acid,” as used herein, refers to the “L” isomeric form of a peptide, and conversely the term “D-amino acid” refers to the “D” isomeric form of a peptide (e.g., Dphe, (D)Phe, D-Phe, or DF for the D isomeric form of Phenylalanine). Amino acid residues in the D isomeric form can be substituted for any L-amino acid residue, as long as the desired function is retained by the peptide.

In the case of less common or non-naturally occurring amino acids, unless they are referred to by their full name (e.g. sarcosine, ornithine, etc.), frequently employed three- or four-character codes are employed for residues thereof, including, Sar or Sarc (sarcosine, i.e. N-methylglycine), Aib (α-aminoisobutyric acid), Dab (2,4-diaminobutanoic acid), Dapa (2,3-diaminopropanoic acid), γ-Glu (γ-glutamic acid), GABA (γ-aminobutanoic acid), β-Pro (pyrrolidine-3-carboxylic acid), and 8Ado (8-amino-3,6-dioxaoctanoic acid), Abu (2-amino butyric acid), βhPro (β-homoproline), βhPhe (β-homophenylalanine) and Bip (β,β diphenylalanine), and Ida (Iminodiacetic acid).

An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In some embodiments, a variant enzyme lacks one or more amino acids (e.g., 1, 2, 3, 4, 5, 6, 7, 8, 9, 10, 11, 12, 13, 14, 15, 16, 17, 18, 19, 20, 21, 22, 23, 24, 25, etc.) from the N-terminal end, compared to a corresponding wild-type enzyme. In some such embodiments, a methionine is added at the new N-terminal end of the truncated enzyme.

The term “analyte” as used herein refers to a substance or chemical constituent that is of interest in an analytical procedure, for example, to be identified and/or measured.

Analytes include, but are not limited to, proteins, nucleic acids, lipids, carbohydrates, and minerals. Analytes include “biomarkers,” which are measurable indicators of some biological state or condition. As used herein a “large molecule” analyte refers to an analyte having a molecular mass of greater than 1000 daltons.

### BRIEF DESCRIPTION OF THE FIGURES

FIG. 1 shows a table of reaction rates of sera from mice inoculated with Mucor mutant FAD-GDH (−ΔA600/min measured with addition of either 1:50 or 1:500 diluted sera) measured as the slope of linear regression of the trace from 640-1280 seconds. The ranking of the 1:50 or 1:500 rates were listed from 1-27, with higher ranked sera (higher inhibition) shown in darker grays and lower ranked sera (lower inhibition) in lighter grays. ND, sera were not tested within this experiment.

FIG. 2 is a graph of the calculated percent inhibition in FAD-GDH colorimetric screening assays with selected hybridoma supernatants. Negative values of percent inhibition indicate observed stimulation of GDH activity with these samples.

FIG. 3A is a graph of dose-dependent inhibition of the measured rate of GDH activity using varying concentrations of mAb 1-286 (x-axis, shown in log scale). FIG. 3B is a graph of residual absorbance of reactions after 45 minutes, showing endpoint inhibition due to varying concentrations of mAb 1-286 (x-axis, shown in log scale). Derived parameters from the curve fitting in FIGS. 3A and 3B are shown in the respective insets.

FIG. 4 is a graph of the initial reaction velocity plotted against glucose concentration for each of the indicated antibody dilutions. Vmax and Km parameters were calculated from the curve fitting and listed in the inset table.

FIG. 5 is a Lineweaver-Burk plot of the data presented in FIG. 4. The intersection of the various lines at a common point on the x-axis left of the origin indicate an allosteric mechanism of inhibition.

FIGS. 6A-6C show zoomed-in views of the structure of the non-glycosylated, Mucor FAD-GDH in complex with rFab 286. FIG. 6A shows the interface formed between rFab286 (cartoon representation) and FAD-GDH (surface representation) in the x-ray crystal structure. Surfaces 1 (white), 2 (black), and 3 (gray) are shown as numerals, and the substrate access pore is labelled. FIG. 6B is a rotation of 45° of the view in FIG. 6A to visualize Surface 3. FIG. 6C is a top-down view of the epitope for rFab 286 on FAD-GDH, with the rFab removed for clarity. Bound FAD is visible deep within the active site (arrow).

FIG. 7 is a graph of percent inhibition of GDH activity for select alanine-scanning mutants of non-glycosylated, Mucor FAD-GDH measured in the absence or presence of mAb 1-286 (1 nM). The inhibition was calculated for each replicate and the mean±S.D. is presented. Surface 1 mutations exhibited defective GDH activity as well as a range of blunted inhibitory responses. Surface 2 mutants F341A, E344A, and E348A illustrate these residues are associated with functional responses to the inhibitory antibody. The DQETAAAA mutant combined D338A, Q342A, E344A, and T345A; DQTAAA, DQAA, DTAA, and QTAA include combinations of alanine mutations made at these four positions.

FIG. 8 is SDS-PAGE analysis of purified, recombinant FAD-GDH mutant proteins. Lanes 1, 13, 23: non-glycosylated, wild-type FAD-GDH (lacking alanine mutations); lane 2, Q48A; lane 3, F49A; lane 4, V50A; lane 5, M56A; lane 6, Y57A; lane 7, Q59A; lane 8, T63A; lane 9, D64A; lane 10, L65A; lane 11, C66A; lane 12, R69A; lane 14, E348A; lane 15, E349A; lane 16, Y354A; lane 17, K358A; lane 18, Y442A; lane 19, T446A; lane 20, D447A; lane 21, L450A; lane 22, N452A; lane 24, M56A (repeat expression and purification successful); lane 25, C66A (repeat successful); lane 26, T337A; lane 27, V340A; lane 28, N343A; lane 29, L346A; lane 30, L65A; lane 31, D338A; lane 32, F341A; lane 33, E344A; lane 34, L450A. Purified, but not analyzed by SDS-PAGE: V61A. Black arrows indicate the expected position of the band of FAD-GDH at approximately 70 kDa based on standard proteins run on the same gels.

FIG. 9 is a graph and curve fitting of the GDH activity of wild-type (WT) or each of three mutant FAD-GDH enzymes measured with titration of inhibitory mAb 1-286 concentration as shown in Table 3.

FIG. 10 is a graph of the percent inhibition of HA grafted FAD-GDH enzymes with titration of an anti-HA antibody, anti-Myc antibody or mAb 1-286.

FIG. 11 is a graph of the percent inhibition by VHH-1, VHH-10, VHH-859, and VHH-898 raised against ungrafted 19031 FAD-GDH with titration of enzyme concentration.

FIG. 12 is a graph of the percent inhibition of V5 epitope grafted FAD-GDH enzymes in the presence of an anti-V5 monoclonal antibody or mAb 1-286.

FIG. 13 is a graph of the percent inhibition of TnI epitope grafted FAD-GDH enzymes in the presence of an anti-TnI monoclonal antibody or mAb 1-286.

FIG. 14A shows rates of DCPIP reduction by FAD-GDH and a plot of the assay concentration of FAD-GDH versus a blank-subtracted rate. The data points correspond to 0, 8, 44, 80, and 116 ng/ml final concentration. The linear regression of the data points is shown as a dashed line with the trendline equation and quality of fit (R2) in bold. FIG. 14B is graph of the kinetic absorbance for blank or an exemplary single concentration of FAD-GDH assayed in duplicate.

FIG. 15A is a graph of the concentration of concentration of D-glucose in the serially-diluted per-minute reaction rate. The data points correspond to 6, 12.1, 24.2, 48.5, 97, and 194 mM glucose. FIG. 15B is a graph for the estimation of the Km of the FAD-GDH enzyme for glucose. Double-reciprocal plot of the data from FIG. 15A using the four highest concentrations tested. The x-intercept was calculated from the equation and corresponds to an estimated apparent Km of 64.7 mM.

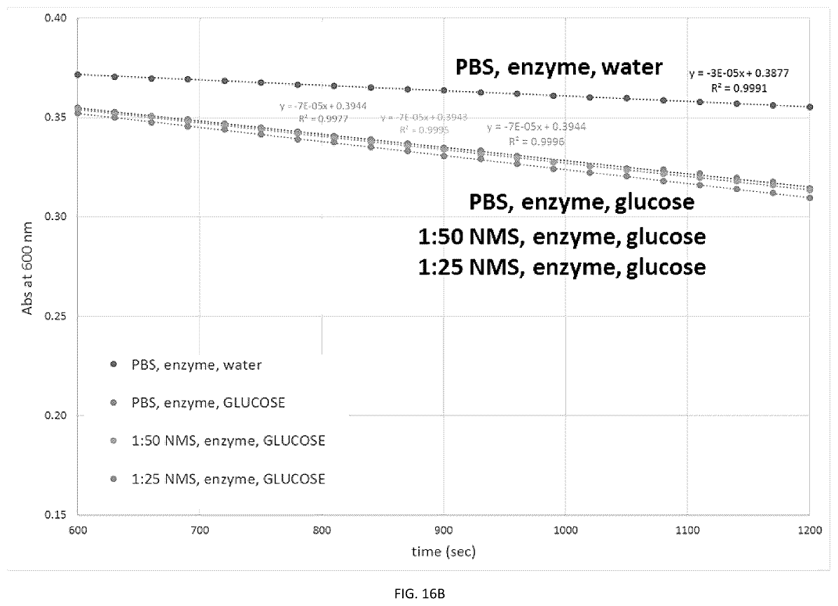

FIG. 16A is a graph of the absorbance of DCPIP reduction reactions containing either PBS or two dilutions of normal mouse serum (NMS). The NMS does not inhibit the rate of the glucose-driven FAD-GDH reaction. FIG. 16B is the linear regression analysis of reactions containing either PBS or two dilutions of normal mouse serum (NMS). The NMS does not inhibit the rate of the glucose-driven FAD-GDH reaction as all three traces are overlapping and have similar rates.

FIG. 17A is a graph of percent inhibition by top-ranking inhibitory sera. The reaction rate of PBS+enzyme+no glucose (control) reaction rate was subtracted from the rates of reactions including inhibitory serum at each dilution. The percent inhibition is plotted as the difference from the NMS reading at each dilution. FIG. 17B is a summary table of the percent inhibition by top-ranking inhibitory sera.

FIG. 18 is a graph of the de-inhibition of WT and 358HA epitope grafted FAD-GDH.

FIGS. 19A-19F are graphs showing the percent inhibition of Mucor (M. prainii, M. guilliermondii, M. hiemalis, M. subtillissimus, M. circinelloides, and M. ambiguus, respectively) epitope grafts by 1-286 antibody and anti-epitope antibodies.

FIGS. 20A and 20B are graphs showing the inhibition of FAD-GDH at various VHH doses. FIG. 20C is a graph of the percent inhibition of either glycosylated or non-glycosylated FAD-GDH in the presence of IgG IO3 and its fragment Fab IO3.

FIG. 21A is a graph of the percent inhibition of ungrafted or V5 epitope-grafted FAD-GDH using various α-V5 antibody concentrations. FIGS. 21B and 21C are graphs of the percent inhibition in the presence of V5 peptide for two versions of V5 epitope graft FAD-GDH enzyme.

FIG. 22 is a graph of the percent inhibition at various α-TnI antibody concentrations for three TnI epitope grafted (358TN1, 358TN4, and 358TN8) FAD-GDH enzymes.

FIG. 23 is a graph of the percent inhibition of FAD-GDH grafted enzymes with various epitopes (V5/TnI/Flag/HA/Myc) in response to the corresponding anti-epitope antibody at 100 nM concentration.

FIG. 24A is a graph of the percent inhibition at various α-HNL antibody concentrations for purified 358HNL-H3 enzyme. FIG. 24B is a graph of the de-inhibition of the enzyme in the presence of HNL peptides.

FIG. 25 is a graph of FAD-GDH inhibition assays using epitope grafted enzymes 341BP and 358BP and various α-NTproBNP antibodies and de-inhibition with NT-ProBNP antigen.

FIG. 26 is a graph of FAD-GDH de-inhibition using differing concentrations of inhibitor and antigen.

FIG. 27 is a graph of stability comparison in between ungrafted FAD-GDH, 358HA epitope graft and 358HACC epitope graft with additional disulfide bond.

FIG. 28 is a graph of percent inhibition for the periplasmic extracts resulting from a phage display of non-glycosylated ungrafted FAD-GDH binding proteins.

FIG. 29 is a graph of a competitive binding assay for four identified and re-formatted anti-FAD-GDH IgGs and 1-286 antibody epitope.

FIG. 30A is a schematic showing two formats for a competitive binding assay of inhibitory IgG IO-3 (clone 3) and 1-286 for ungrafted FAD-GDH. FIG. 30B is a graph of the results of the assay format shown in FIG. 30A, right, having IO-3 coated on the plate and 1-286 antibody titrated. FIG. 30C is a graph of the results of the assay format shown in FIG. 30A, left, having 1-286 coated on the plate and IO-3 antibody titrated.

FIG. 31 is a graph of the calculated percent inhibition for inhibitory IgGs of ungrafted FAD-GDH plotted as a function of concentration for IC50 determination, quantified in the table below.

FIG. 32 shows samples of purified A. flavus FAD-GDH with epitope grafting at position 328 of TNI, HA, or HNL epitopes that were resolved by SDS-PAGE and Coomassie Brilliant Blue staining. Arrows indicate the migratory position of the purified enzymes. The position of molecular weight (M.W.) standards is marked in kilodaltons (kDa) for the four replicated standard lanes (not labeled).

FIG. 33 shows either wild-type, ungrafted Mucor FAD-GDH 19-031 (negative control) or various A. flavus epitope graft constructs made at amino acid position 328 tested for inhibition with various commercial antibodies at indicated concentrations.

FIG. 34 shows epitope-grafted A. flavus FAD-GDH enzymes that were tested for inhibition with various antibodies at the final concentrations indicated. HNL 2-6128 is a negative control for all three grafted A. flavus enzymes, as this antibody recognizes a sequence different from the HNL epitope that was grafted into the enzyme.

FIG. 35A shows ungrafted, wild-type A. flavus FAD-GDH or the epitope-grafted, A. flavus 328HA constructs that were tested for inhibition by anti-HNL (control) or anti-HA ab182009 antibody in a dose-response experiment. FIG. 35B shows ungrafted, wild-type A. flavus FAD-GDH or the epitope-grafted, A. flavus 328HA constructs that were tested for inhibition by anti-HNL (control) or anti-HA ab236632 antibody in a dose-response experiment.

### DETAILED DESCRIPTION

As discussed above, provided herein are systems, methods, and compositions, including kits, devices, and reaction mixtures that utilize an epitope-grafted enzyme for analysis of analytes in samples. Provided below are illustrative embodiments of the technology. It is to be understood that the teachings of this disclosure are not limited to these exemplary embodiments.

### I) Enzymes

A) Sensor enzymes

In some embodiments, the technology provided herein uses one or more enzymes. When exposed to a substrate, the enzyme generates reaction products. The reaction products are directly or indirectly detected to determine the activity of the enzyme. The enzyme is designed or configured such that the enzyme activity varies in response to presence of, absence of, or amount of an analyte in the sample. As such, by measuring the activity of the enzyme, a measure of the presence of, absence of, or amount of an analyte in a sample is achieved.

In some embodiments, the enzyme is any enzyme having an enzyme activity that is detectably altered in the presence of an analyte of interest. In some embodiments, the enzyme comprises one or more allosteric sites that when bound by an inhibiter or inhibitors, alters (e.g., decreases) the activity of the enzyme. In some embodiments, the allosteric site comprises a heterologous sequence. In some embodiments, the heterologous sequence is an epitope graft. In some embodiments, the inhibitor or inhibitors specifically bind to the epitope graft sequence contained within the enzyme and, when bound, inhibit the enzyme activity. Accordingly, in some embodiments, provided herein is an enzyme comprising a modified allosteric site, wherein the modified allosteric site comprises a grafted heterologous epitope; wherein the enzyme has an enzymatic activity which is inhibited by the binding of an inhibitor to the grafted epitope.

In some embodiments, the enzyme is a glucose metabolism enzyme (i.e., an enzyme that utilizes glucose as a substrate). For example, in some embodiments, the enzyme is a glucose dehydrogenase (GDH) (i.e., an enzyme that catalyzes the oxidation of glucose in the presence of a cofactor such as nicotinamide adenine dinucleotide (NAD), nicotinamide adenine dinucleotide phosphate (NADP), flavine adenine dinucleotide (FAD), or pyrroloquinoline quinone (PQQ)) or a glucose oxidase (GO) (i.e., an enzyme that catalyzes the oxidation of glucose to hydrogen peroxide). Ferri et al., Diabetes Sci. Technol., Glucose Electrochemistry, 5(5), 1068-76 (2011), herein incorporated by reference in its entirety, provides a review of exemplary glucose metabolizing enzymes suitable for use in sensors. In some embodiments, the enzyme is a flavin-adenine-dinucleotide-dependent glucose dehydrogenase (FAD-GDH). In some embodiments, the GDH is a pyrroloquinoline quinone glucose dehydrogenase (PQQ-GDH). In some embodiments, the GDH is a nicotine adenine dinucleotide (phosphate)-dependent glucose dehydrogenase (NAD(P)-GDH).

In some embodiments, the enzyme is derived from a microbial source. In some embodiments, the enzyme is derived from a bacterial or fungal source. In some embodiments, the enzyme is derived from a mold. In some embodiments, the enzyme is derived from an organism of the divisional Mucoromycota or Ascomycota. In some embodiments, the enzyme is derived from an organism of the order Mucorales or Eurotiales. In some embodiments, the enzyme is derived from the family Mucoraceae or Aspergillaceae. In some embodiments, the enzyme is derived from the genus Mucor or Aspergillus (e.g., sub-genus Circumdati, e.g., section Flavi). In some embodiments, the enzyme is derived from the species M. hiemalis, M. circinelloides, M. ambiguus, M. lusitanicus, M. guilliermondii, M. subtillissimus, M. prainii, A. Flavus and/or A. oryzae. In some embodiments the enzyme is an FAD-GDH derived from the genus Mucor (e.g., derived from the species M. hiemalis, M. circinelloides, M. ambiguus, M. lusitanicus, M. guilliermondii, M. subtillissimus, and/or M. prainii). In some embodiments, the enzyme is derived from the FAD-GDH from M. hiemalis, M. circinelloides, M. ambiguus, M. prainii and M. subtillissimus.

In some embodiments, the enzyme is a wild-type enzyme. Examples of such wild-type enzymes into which an epitope graft may be inserted are shown in SEQ ID NOS: 66-72, 119-127, and 131. In some embodiments, the enzyme is a modified enzyme (e.g., a synthetically modified enzyme) comprising one or more (e.g., 1, 2, 3, 4, 5, 6, 7, 8, 9, 10, 11, 12, 13, 14, 15, 16, 17, 18, 19, 20, etc.) variations compared to a wild-type enzyme. In some embodiments, the enzyme has at least 70% sequence identity (at least 80%, at least 85%, at least 90%, at least 95%, at least 96%, at least 97%, at least 98%, at least 99%, at least 99.5%) to any one of the sequences associated with accession numbers UCW69416.1 (SEQ ID NO:119), UCW69417.1 (SEQ ID NO:120), UCW69418.1 (SEQ ID NO:121), UCW69419.1 (SEQ ID NO:122), UCW69420.1 (SEQ ID NO:123), UCW69421.1 (SEQ ID NO:124), UCW69422.1 (SEQ ID NO:125), UCW69423.1 (SEQ ID NO:126), or UCW69424.1 (SEQ ID NO:127), or to any of SEQ ID NOS:1-118 or 132-134 (excluding any epitope grafted sequences there, identified by underlining in Table 1). Sequence variations include point mutations, insertions, and deletions as well as chimeric enzymes (i.e., enzyme having sequences derived from two or more different enzymes). As noted above, amino acid modifications are typically conservative substitutions.

One or more synthetic sequences may be added to the enzyme to facility expression or purification of the enzyme. In some such embodiments, the sequence used to facilitate expression or purification are removed prior to use of the enzyme in a sensor.

In some embodiments, one or more amino acids are modified, compared to a wild-type enzyme, to increase a desired property of the enzyme. Desired properties, include, but are not limited to, enzyme activity (e.g., specific activity, turnover rate, Km for substrate, ability to titrate), allosteric inhibitability, de-inhibitability, stability (e.g., thermostability, shelf-life stability, stability when embedded in or otherwise associated with a sensor surface, etc.), engineerability, ability to absorb onto a sensor surface, ability to make fusion proteins (e.g., fusion with an inhibitor), immobilizability (e.g., compatibility with addition of a binding moiety), ability to orient on a surface, compatibility with a sensor layer, biocompatibility with sensing conditions (e.g., sample, pH, salts), resistance to interferants, avoidance of generation of interfering byproducts (e.g., peroxide), affinity to inhibitor, and substrate specificity.

In some embodiments, one or more variants is made to increase the stability of the enzyme. For example, one or more cysteine substitution may be made in the enzyme to allow for stabilizing disulfide bond formation (see, Example 15). In some embodiments, cysteine mutation pairs that are spatially close to each other are introduced, forming a disulfide bond to stabilize the enzyme structure.

Enzymes may be produced in a host cell. Thus, in some embodiments, included herein are nucleic acids and expression systems for recombinant expression of an enzyme in a host cell or organism. The nucleic acid sequences may be altered, compared to a wild-type nucleic acid sequence, to generate a variant enzyme as described above, as well as to facilitate expression of the protein. For example, nucleic acid variants may encode for the same amino acid, but result in a different expression profile in a given host expression system. Nucleic acid sequences encoding an enzyme may be provided in an expression vector suitable for expression in a desired host cell. Alternatively, nucleic acid sequence may be integrating into a genome of a host cell or organism. Suitable host cells include, but are not limited to, bacterial cells (e.g., E. coli), yeast (e.g., Saccharomyces cerevisiae, Pichia pastoris), baculovirus, plant, and animal cells. In some embodiments, enzymes are produced in cell-free systems.

### B) Substrates for the Sensor Enzyme

In some embodiments, enzyme activity is directly or indirectly assessed by measuring the presence of or amount of substrate processed by the enzyme. Any suitable natural or synthetic substrate may be used with a selected enzyme. For example, where the enzyme is a glucose-metabolizing enzyme, glucose can be used as the substrate. In some embodiments, a substrate is modified to facilitate detection of the processing by the enzyme. For example, in some embodiments, a detectable label (e.g., fluorescent, luminescent, radioactive, chemical, affinity tag, etc.) is added to the substrate such that a product of the reaction having or lacking the label is assessed. In some embodiments, a byproduct of substrate processing by the enzyme is detected, directly or indirectly, as a measure of enzyme activity. For example, the enzyme glucose oxidase is comprised of two identical protein subunits and a cofactor at its active site (i.e., flavin adenine dinucleotide (FAD)). With FAD, glucose oxidase catalyzes the oxidation of its reactant glucose at its first hydroxyl group, utilizing molecular oxygen as the electron acceptor, to produce the products gluconolactone and hydrogen peroxide. The hydrogen peroxide product produced can be detected (such as, for example, by electrochemical oxidation at an electrode and the number of electron transfers detected). Alternatively, oxygen consumption can be measured. Glucose dehydrogenase can utilize a number of different co-factors (e.g., NAD, PQQ, etc.). When FAD is used as a co-factor, glucose dehydrogenase catalyzes the oxidation of glucose to produce gluconolactone and FADH₂. The FADH₂ can be electrochemically oxidized at an electrode and the number of electron transfers detected.

### C) Allosteric Sites

In some embodiments, epitope-grafted sequences (e.g. an amino acid sequence) are inserted into an allosteric site on an enzyme. Some substances bind enzymes at a site other than the active site. This other site is called the allosteric site. The allosteric site allows molecules to either activate or inhibit (wholly or partially), enzyme activity. Such molecules bind to the allosteric site and change the confirmation, or shape, of the enzyme. The epitope-grafted sequence provides an allosteric site for altering the activity of the enzyme when an agent (e.g., inhibitor) binds to the epitope grafted sequence located at the allosteric site.

In some embodiments, two or more allosteric site may be utilized in an enzyme.

In some embodiments, an enzyme has one or more surface regions amenable to grafting of a heterologous epitope. Such surface regions may comprise the allosteric site. A surface region amenable to addition of a heterologous sequence is a region on the surface of enzyme that, when modified to insert a heterologous sequence, does not eliminate measurable enzyme activity. In some embodiments, at least 5% (e.g., 10%, 20%, 30%, 40%, 50%, 60%, 70%, 80%, 90%) of the enzyme activity is maintained after addition of a heterologous sequence to the surface region compared to the enzyme without addition of the heterologous sequence. Enzyme activity may be assayed, for example, by measuring an amount of substrate processed by the enzyme during a given time period. Assays for assessing enzyme activity are provided in the Example section below. In some embodiments, the surface region is located in an allosteric region of the enzyme.

For example, in some embodiments, provided herein are enzymes having a surface region comprising amino acids 45-70, 335-362, and/or 439-457 SEQ ID NO:1, or a variant thereof, or corresponding regions in SEQ ID NOS: 66-72 or 119-127. An epitope graft may be inserted at any position within these surface regions.

In some embodiments, the enzyme has an allosteric site located on a surface that comprises residues F341, E344, E348, and K358 of SEQ ID NO: 1 (or corresponding residues in SEQ ID NOS: 66-72 or 119-127, or in a variant sequence). In some embodiments, the surface comprises residues T337, D338, V340, F341, N434, E344, L346, E348, E349, Y354 and K358 of SEQ ID NO: 1 (or corresponding residues in SEQ ID NOS: 66-72 or 119-127, or in a variant sequence). Again, the epitope may be grafted in at any one of these positions (see below in relation to “at”).

In some embodiments, the epitope is grafted at a position corresponding to from about position 330 to about position 370 of SEQ ID NO: 1 or corresponding regions in SEQ ID NOS: 66-72 or 119-127 or in a variant sequence. In some embodiments, the epitope is grafted at a position corresponding to from about position 335 to about position 362 of SEQ ID NO: 1 or corresponding regions in SEQ ID NOS: 66-72 or 119-127 or in a variant sequence. In some embodiments, epitope is grafted at a position corresponding to position T337, D338, V340, F341, N434, E344, L346, E348, E349, Y354, or K358 of SEQ ID NO: 1 or corresponding regions in SEQ ID NOS: 66-72 or 119-127 or a variant sequence. In some embodiments, the epitope is grafted at a position corresponding to position 341 or 358 of SEQ ID NO: 1 or corresponding regions in SEQ ID NOS: 66-72 or 119-127 or a variant sequence. In some embodiments, the epitope is grafted at a position corresponding to position 341 of SEQ ID NO: 1 or corresponding regions in SEQ ID NOS: 66-72 or 119-127 or a variant sequence. In some embodiments, the epitope is grafted at a position corresponding to position 358 of SEQ ID NO: 1 or corresponding regions in SEQ ID NOS: 66-72 or 119-127 or a variant sequence. In some embodiments, the epitope is grafted at a position corresponding to from about position 320 to about 335 of SEQ ID NO: 131 (or corresponding regions in a variant sequence), such as from about position 325 to about position 330 of SEQ ID NO: 1 (or corresponding regions in a variant sequence), e.g. from about position 327 to about position 329 of SEQ ID NO: 131 (or corresponding regions in a variant sequence). In some embodiments, the epitope is grafted at a position corresponding to position 328 of SEQ ID NO:131 (or the corresponding position in a variant sequence). In some embodiments the epitope is grafted at a sequence corresponding to position 328 of SEQ ID NO:131. With respect to an epitope grafted “at” a recited position, the added epitope sequence may be (i) a replacement of the relevant amino acid(s) by the epitope; (ii) insertion of the epitope N-terminal to the relevant amino acid(s); or (iii) insertion of the epitope C-terminal to the relevant amino acid(s). With respect to positions 328, 341, and 358, “at” typically means that the epitope is grafted in after that residue (i.e., C-terminal to position 328, 341, or 358).

In some embodiments, allosteric sites are identified and modified as discussed in the Examples, below.

### D) Epitope Grafting

Epitope sequences are provided in the regions of the enzyme that are suitable for allosteric regulation of enzyme activity. The epitope sequence provides a recognition sequence for interaction with an inhibitor. When the inhibitor interacts with the epitope sequence, the activity of the enzyme is altered. For example, in some embodiments, interaction of the inhibitor with the epitope sequence located in an allosteric site of the enzyme reversibly inhibits enzyme activity. In such a state, the enzyme can be considered “inhibited.” Inhibition need not eliminate all enzyme activity. A detectable reduction in enzyme activity is suitable for many sensor applications. If an analyte, that is also recognized by the inhibitor, is present in proximity to the enzyme, the inhibitor has less association with the epitope grafted sequence on the enzyme and enzyme activity increases. The introduction of the analyte, and the association of the inhibitor with the analyte rather than the epitope-graft sequence in the enzyme, “de-inhibits” the enzyme.

Epitope sequences may be selected based on one or more of several parameters. First, an epitope sequence should provide sufficient structure to allow association (e.g., binding) of an inhibitor with the allosteric site of the enzyme containing the epitope-grafted sequence. Second, the association of the inhibitor with the allosteric site containing the epitope-grafted sequence should inhibit enzyme activity. Third, the strength of association of the inhibitor with the epitope-grafted sequence should be such that presence of analyte in a sample introduced to the enzyme should de-inhibit the enzyme. In some such embodiments, the epitope sequence and the inhibitor are selected such that the inhibitor preferentially binds to an analyte, when present, over the allosteric site containing the epitope-grafted sequence. This can be achieved, for example, by using an epitope-grafted sequence that provides a sequence/confirmation that has weaker affinity for the inhibitor than the corresponding sequence/confirmation found in the analyte. One or more amino acid differences in the epitope-grafted sequence, relative to the corresponding sequence in the analyte, may be used to provide differential binding of the inhibitor to the epitope-grafted enzyme relative to the analyte. In some embodiments, the affinity of the analyte for the inhibitor is greater than the affinity of the epitope-granted enzyme for the inhibitor. The affinity of the analyte for the inhibitor and/or the epitope may be determined as K_{D} values, as can be determined using standard methods known in the art. In some embodiments the affinity of the analyte for the inhibitor is at least 2 times, at least 3 times, at least 5 times, at least 10 times or at least 50 times greater than the affinity of the epitope-granted enzyme for the inhibitor. In some embodiments, inhibitors are designed, selected for, or screened for the property of having a high dissociation rate (k_{off}) from the enzyme.

In some embodiments, the epitope comprises an amino acid sequence corresponding to an inhibitor binding site of an analyte such as a peptide, polypeptide or protein. Inhibitor binding sites on polypeptides can be identified by those skilled in the art. For example, an analyte can be contacted with an inhibitor and the binding site of the inhibitor can be deduced e.g., by X-ray crystallography. This and other methods are described in the examples.

In some embodiments the epitope comprises an amino acid sequence having at least 70%, at least 80%, or at least 90% sequence identity to a corresponding sequence of the inhibitor binding site of the analyte. The epitope may be designed or configured to bind to an inhibitor more weakly than the inhibitor binds to an analyte, e.g. more weakly than the inhibitor binds to the inhibitor-binding-site of the analyte. The strength of binding of the inhibitor to the epitope may be controlled by varying the sequence of the epitope graft compared to the sequence of the inhibitor binding site of the analyte. For example, a grafted epitope having a sequence which comprises 1, 2, 3, 4 or 5 or more modifications (e.g., substitutions, e.g., conservative substitutions) compared to the sequence of an inhibitor-biding site of an analyte may have an altered (e.g., decreased) binding strength for the inhibitor compared to the analyte.

In some embodiments the inhibitor is capable of competitively binding to the polypeptide analyte and to the grafted epitope. In some embodiments the epitope thus comprises an amino acid sequence corresponding to an inhibitor binding site of a polypeptide analyte and the inhibitor competitively binds to the grafted epitope and to the analyte.

In some embodiments, the epitope grafted sequence comprises from 3 to 30 amino acids. The lower end of the range should include a sufficient structure to permit recognition by the inhibitor. In some embodiments, the epitope grafted sequence comprises at least 3, 4, 5, 6, 7, 8, 9, 10, 11, 12, 13, 14, or 15 amino acids. In some embodiments, the epitope grafted sequence has 5 to 15 amino acids (e.g., 8 to 10). In other words, in some embodiments the epitope grafted sequence comprises from 3 to 30 amino acids, e.g. from 5 to 15 amino acids such as from 8 to 10 amino acids. In some embodiments, epitope grafted sequences are selected to have one or more or all polar amino acids (serine (Ser), threonine (Thr), cysteine (Cys), asparagine (Asn), glutamine (Gln), and tyrosine (Tyr)).

In some embodiments, epitope-grafted sequences are linear epitopes. In some embodiments, epitope-grafted sequences are conformation epitopes. A linear or a sequential epitope is an epitope that is recognized by a binding molecule (e.g., antibody, antibody fragment, or antibody mimetic such as an aptamer, affimer, DARPin, etc.) by its linear sequence of amino acids, or primary structure. In contrast, a conformational epitope is recognized by its three-dimensional shape. In some embodiments, the epitope-grafted sequence is a discontinuous epitope, i.e. an epitope that consists of multiple, distinct segments from the primary amino-acid sequence.

In some embodiments an epitope-grafted sequence is a linear epitope having a length of from 3 to 30 amino acids, e.g., from 5 to 15 amino acids such as from 8 to 10 amino acids.

In some embodiments an epitope-grafted sequence is a discontinuous epitope comprising multiple (e.g., 2, 3 or 4) segments each having a length of from 3 to 15 amino acids such as from 5 to 12 amino acids, e.g. from 8 to 10 amino acids. Typically the total length is as above.

In some embodiments, the epitope grafted sequence is inserted into the enzyme while retaining the original amino acids of the enzyme. In other embodiments, one or more amino acids (e.g., 1, 2, 3, 4, 5, 6, 7, 8, 9, 10, 11, 12, 13, 14, 15) from the enzyme are removed and replaced by the epitope grafted sequence. In some embodiments, the removed amino acids reside, prior to removal, on the N-terminal side of the selected epitope grafted sequence insertion site identified in the allosteric site of the enzyme. In some embodiments, an optimal location for a given epitope grafted sequence within an allosteric site of an enzyme is determined by a screening method. In some embodiments, the screening method comprises inserting the graft sequence at staggered locations throughout the allosteric site or within sub-regions of an allosteric site to identify an optimal location (see e.g., SEQ ID Nos 8-13 and 14-16 showing staggered placement of the V5 epitope sequence IPNPLLGLD in staggered locations within an enzyme). In some embodiments, the screening method comprises testing linker sequences on one or both sides of the epitope graft sequence. In some embodiments, the screening methods identify impact of design features on enzyme activity, inhibition of enzyme activity, and/or de-inhibition of enzyme activity.

### II) Inhibitors

In some embodiments, an inhibitor is employed that interacts with one or more epitope-grafted allosteric sites on an enzyme to inhibit enzyme activity. The inhibitor also interacts with at least a portion of an analyte of interest that corresponds to the epitope-grafted sequence such that the enzyme, when bound to inhibitor and in an inhibited state, is de-inhibited in the presence of analyte, which competes with the enzyme for binding of the inhibitor. Any agent may be employed that recognizes the epitope-grafted sequence to inhibit the enzyme and that recognizes the analyte or a portion thereof (e.g., recognizes a corresponding epitope present in the analyte) to de-inhibit the enzyme when analyte is present.

In some embodiments, the inhibitor is an antigen binding protein. In some embodiments the inhibitor is an antibody or an antibody mimetic. In some embodiments, the inhibitor is an immunoglobulin (e.g., antibody or antibody fragment). In some embodiments, the inhibitor is an antibody. As used herein, the term “antibody” is used in its broadest sense to refer to whole antibodies, monoclonal antibodies (including human, humanized, or chimeric antibodies), polyclonal antibodies, and antibody fragments that can bind antigen (e.g., Fab′, F(ab′)2, Fv, single chain antibodies), comprising complementarity determining regions (CDRs) of the foregoing as long as they exhibit the desired biological activity. As used herein, “antibody fragments” comprise a portion of an intact antibody, preferably the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multi-specific antibodies formed from antibody fragments. In some embodiments, the inhibitor is a nanobody (e.g., VHH). In some embodiments, the inhibitor is a camelid single-domain antibody. In some embodiments, the inhibitor is a bi-specific antibody that is configured to bind to two or more different analytes, such that the presence of either analyte results in competition for the inhibitor and partial or complete de-inhibition of the sensor enzyme.

In some embodiments, the inhibitor is an aptamer. Aptamers are oligonucleotide or peptide molecules that bind to a specific target molecule. Aptamers are usually created by selecting them from a large random sequence pool, but natural aptamers also exist.

In some embodiments, the inhibitor is an affimer. Affimers are small proteins that bind to target proteins with affinity typically in the nanomolar range. They are engineered non-antibody binding proteins designed to mimic the molecular recognition characteristics of monoclonal antibodies. These affinity reagents can be optimized to increase their stability, make them tolerant to a range of temperatures and pH, reduce their size, and to increase their expression in host cells.

In some embodiments, the inhibitor is a DARPin. DARPins (an acronym for designed ankyrin repeat proteins) are genetically engineered antibody mimetic proteins typically exhibiting highly specific and high-affinity target protein binding. They are derived from natural ankyrin repeat proteins, one of the most common classes of binding proteins in nature, which are responsible for diverse functions such as cell signaling, regulation and structural integrity of the cell. DARPins comprise at least three, repeat motifs or modules, of which the most N- and the most C-terminal modules are referred to as “caps”, since they shield the hydrophobic core of the protein.

In some embodiments, the binding of an inhibitor to an epitope-grafted enzyme as described herein decreases the enzyme activity of the epitope-grafted enzyme by at least 5% (e.g., at least 10%, 20%, 30%, 40%, 50%, 60%, 70%, 80%, 90%, 95%, 97%, 98%, 99% or 100%) relative to the enzyme activity of the epitope-grafted enzyme in the absence of the inhibitor. In some embodiments, unbinding an inhibitor from an inhibitor-bound epitope-grafted enzyme as described herein (i.e., deinhibiting the enzyme) restores at least 5% (e.g., at least 10%, 20%, 30%, 40%, 50%, 60%, 70%, 80%, 90%, 95%, 97%, 98%, 99% or 100%) of the enzyme activity of the enzyme activity of the epitope-grafted enzyme in the absence of the inhibitor. In some embodiments the inhibition of enzyme activity resulting from the binding of an inhibitor to an epitope-grafted enzyme as described herein is at least 70%, 80%, 90%, 95%, 97%, 98%, 99% or 100% reversible.

In some embodiments the inhibitor is employed at a concentration which decreases the enzyme activity of the epitope-grafted enzyme by at least 5% (e.g., at least 10%, 20%, 30%, 40%, 50%, 60%, 70%, 80%, 90%, 95%, 97%, 98%, 99% or 100%) relative to the enzyme activity of the epitope-grafted enzyme in the absence of the inhibitor. It is routine for those skilled in the art to determine an appropriate inhibitor concentration based on a desired level of enzyme inhibition and the inhibitor being used. For example, in some embodiments the concentration of the inhibitor is from about 0.1 nM to about 10 μM, such as from about 1 nM to about 1 μM. In some embodiments the concentration is from about 0.1 nM to about 1 μM, such as from about 1 nM to about 100 nM.

### III) Analytes

As demonstrated in the Example section below, the universal sensor system technology provided herein can detect and analyze a wide range of diverse analytes, including large molecule proteins.

In some embodiments, the analyte is a prognostic or diagnostic analyte for a patient's health and/or well-being. For example, the analyte can be any molecule of interest for diagnosis, screening, disease staging, forensic analysis, pregnancy testing, drug testing, and other reasons. An analyte may be a biopolymer marker of a physiological state including health, disease, drug response, efficacy, safety, injury, trauma, traumatic brain injury, pain, chronic pain, pregnancy, atherosclerosis, myocardial infarction, diabetes type I or type II, sepsis, cancer, Alzheimer's dementia, multiple sclerosis, and the like. The analyte can include a protein, a peptide, a polypeptide, an amino acid, a hormone, a steroid, a vitamin, a drug including those administered for therapeutic purposes as well as those administered for illicit purposes, a bacterium, a virus, and metabolites of or antibodies to any of the above substances.

In some embodiments, the analyte is one or more of TnI, TnT, BNP, NTproBNP, proBNP, HCG, TSH, NGAL (also known as LCN2), theophylline, digoxin, and phenytoin. In some embodiments, the analyte is one or more of acid phosphatase, alanine aminotransferase, albumin (BCG/BCP), alkaline phosphatase, alanine aminotransferase, alpha-1-acid glycoprotein, alpha-1-antitrypsin, alpha-Fetoprotein, amikacin, amphetamine/methamphetamine, amylase, apolipoprotein A1, apolipoprotein B, anti-HBC (IgG and IgM) antibodies, aspartate aminotransferase, barbiturates, benzodiazepines, beta2-Microglobulin, beta-hCG, bilirubin, cancer antigen 15-3, cancer antigen 125, cancer antigen 19-9XR, carcinoembryonic antigen (CEA), cannabinoids, carbamazepine, ceruloplasmin, cholesterol, cocaine, complement C3, complement C4, cortisol, creatine kinase, creatine, CRP Vario, C-Peptide, cyclic citrullinated peptide, cyclosporine, dehydroepiandrosterone-sulfate (DHEA-S), ecstasy, estradiol, ferritin, folate, follicle-stimulating hormone (FSH), free prostate-specific antigen (PSA), free triiodothyronine (T3), free thyroxine (T4), gamma-glutamyl transferase, gentamicin, haptoglobin, HCV (antibodies to the hepatitis C virus), HDL, hemoglobin A1c, hepatitis B surface antigen, HIV Antigen and/or Antibody, homocysteine, holotranscobalamin (B-12 marker), human epididymis protein 4 (HE4), immunoglobulin A, immunoglobulin G, immunoglobulin M, insulin, IgM antibodies, LDL, lactase dehydrogenase, lipase, lipoprotein A, luteinizing hormone, methadone, microalbumin, myoglobin, opiates, parathyroid hormone, phencyclidine, phenobarbital, phenytoin, prealbumin, procalcitonin (PCT), protein (Urine/CSF), progesterone, prolactin, prostate-specific antigen (PSA), propoxyphene, rheumatoid factor, salicylate, serum benzodiazepines, SHBG, sirolimus, T3, T4, tacrolimus, testosterone, theophylline, thyroglobulin antibodies, tobramycin, TPO antibodies, transferrin, tricyclic antidepressant, triglycerides, vancomycin, glial fibrillary acidic protein (GFAP), and Ubiquitin carboxy-terminal Hydrolase L1 (UCH-L1).

In some embodiments, two or more analytes are detected. In some embodiments, the two or more analytes are detected in an “and” format, where the presence of or amount of each analyte is independently determined. In other embodiments, the two or more analytes are detected in an “or” format, where the presence of any one of the analytes generates a detectable signal identifying that at least one of the analytes is present, but not distinguishing between the analytes.

### IV) Samples

The enzyme and sensor systems described herein find use in the analysis of analytes in any desired sample types. Samples include both biological and environmental samples. Sample may be detected in a laboratory setting, in the field, or any other suitable location. The samples may be brought to the sensors for testing, or the sensor may be applied at the source of the samples. For example, in some embodiments, the sensors are physically proximal to, attached to, or contained within a sample source (e.g., on or in a subject or environmental sample).

In some embodiments, the sample is a biological sample. Biological samples may be obtained from any source including animals, plants, and microorganisms and encompass fluids, solids, tissues, and gases. Materials obtained from clinical or forensic settings that contain analytes of interest are also within the intended meaning of the term sample.

Biological samples include, but are not limited to, whole blood, serum, plasma, saliva, ocular lens fluid, amniotic fluid, synovial fluid, cerebrospinal fluid, lacrimal fluid, lymph fluid, interstitial fluid, peritoneal fluid, bronchial lavage, ascites fluid, bone marrow aspirate, pleural effusion, urine, milk, sweat, sputum, semen, mucus, feces, tissue (skeletal muscle tissue, liver tissue, lung tissue, kidney tissue, myocardial tissue, brain tissue, bone marrow, cervix tissue, skin, etc.), organ (such as biopsy sample), vaginal fluids, aqueous humor, earwax, gastric fluid, gastrointestinal fluid, nasal wash, liposuction, sebum, tears, breath, and vitreous humor. Such samples may be assessed in vitro, ex vivo, or in vivo.

In some embodiments, the sample is an environmental sample. Environmental samples include, but are not limited to, water, air, snow, and soil.

Samples may be in a processed form, including dried (e.g., dried blood spots) and fixed (e.g., formalin-fixed paraffin-embedded (FFPE)) samples. In some embodiments, the sample is located in vivo in an animal.

Where a sensor is used to measure one or more analytes in vivo, it may be placed on or in a subject such that a desired sample within the subject comes into contact with the sensor chemistry. For example, the sensor may be placed in a wearable device that facilitates contact between the sensor chemistry and interstitial fluid or blood of the subject. Such systems, and technology employed in the systems, are described in U.S. Pat. Nos. 6,932,894; 7,620,438; 7,670,470; 7,826,382; 7,920,907; 8,106,780; 8,115,635; 8,147,666; 8,223,021; 8,280,474; 8,358,210; 8,377,271; 8,380,274; 8,390,455; 8,409,093; 8,410,939; 8,437,829; 8,542,122; 8,617,069; 8,688,188; 8,737,259; 8,760,297; 8,816,862; 8,915,850; 9,000,929; 9,007,781; 9,008,743; 9,014,774; 9,042,955; 9,060,805; 9,184,875; 9,186,098; 9,186,113; 9,215,992; 9,226,714; 9,232,916; 9,265,453; 9,271,670; 9,314,198; 9,336,423; 9,351,669; 9,402,544; 9,402,570; 9,414,778; 9,474,475; 9,532,737; 9,549,694; 9,636,068; 9,687,183; 9,693,713; 9,713,443; 9,750,444; 9,808,186; 9,831,985; 9,895,091; 9,907,470; 9,931,066; 9,980,669; 9,993,188; 10,010,280; 10,028,680; 10,136,816; 10,136,845; 10,178,954; 10,201,301; 10,213,139; 10,349,877; 10,492,685; 10,653,344; 10,736,547; 10,765,351; 10,820,842; 10,923,218; 10,952,611; 11,051,724; 11,119,090; 11,179,068; 11,202,591; and 11,213,229, each of which is herein incorporated by reference in its entirety. In some embodiments, the sensors are placed in a wearable mouthpiece that facilitates contact between the sensor chemistry and saliva. In some embodiments, the sensors are placed in line with an instrument that collects biological fluids, such as a syringe, dialysis tubing, breathing tube, catheter channel, and the like. In some embodiments, the sensors are included within an implant (e.g., a stent, a transplant, an artificial joint or limb, etc.).

In some embodiments, the sensor is directly exposed to a sample without any modification or alteration of the sample. In some embodiments, the sample is pre-processed to remove one or more components prior to exposure of the sample to the sensor chemistry.

### V) Detection/Sensor Systems

In some embodiments, provided herein is a system comprising an epitope grafted enzyme and an inhibitor capable of binding thereto. The enzyme and inhibitor are typically as described herein.

In some embodiments, the system further comprises a substrate for the epitope-grafted enzyme. In some embodiments the enzyme is an epitope-grafted FAD-GDH and the substrate is glucose.

In some embodiments the system further comprises an analyte having a binding site for the inhibitor. The analyte may be an analyte as described in more detail herein; for example, the analyte may be a peptide, polypeptide or protein as described herein. In some embodiments the analyte is present in a biological sample as described herein.

Also provided is a sensor comprising an enzyme as described herein. The sensor may comprise a system as described herein. The sensor may be an electrochemical sensor.

In some embodiments, enzymes are integrated within an electrochemical sensor. A general description of suitable sensor configurations and sensor systems employing these sensors utilizing the enzymes of the present disclosure are provided. However, this description should be understood as being non-limiting of the aspects disclosed herein and that alternative sensors and systems are contemplated as remaining within the scope of the present disclosure.

Typically, multiple copies of an enzyme are employed on a sensor surface. The concentration and spacing of the enzymes may be selected based on the desired sensor performance. For example, in some embodiments, a lower concentration of enzyme allows detection of a lower amount of analyte. In some embodiments, where maximal sensitivity is desired, a more diluted, greater spread of enzyme on the sensor surface is employed. In some embodiments, two or more different enzymes, that detect different analytes are employed in a single sensor system. In some embodiments, a monolayer of enzymes is employed.

In some embodiments, the sensors contain or comprises one or more (e.g., multiple) enzymes upon the active area(s) of a single working electrode or upon two or more separate working electrodes. Single working electrode configurations for a sensor may employ two-electrode or three-electrode detection motifs.

In some embodiments, an electron transfer agent may be present in one or more of the sensing regions (e.g., active areas) of any of the sensors or sensor configuration. Suitable electron transfer agents/mediator compounds may facilitate conveyance of electrons to the working electrode when a reactant undergoes an oxidation-reduction reaction. Choice of the electron transfer agent within each active area may dictate the oxidation-reduction potential observed for each. When multiple active areas are present, the electron transfer agent within each active area may be the same or different.

Suitable electron transfer agents may include electroreducible and electrooxidizable ions, complexes or molecules (e.g., quinones) having oxidation-reduction potentials that are a few hundred millivolts above or below the oxidation-reduction potential of an electrode. According to some aspects, suitable electron transfer agents may include low-potential osmium complexes, such as those described in U.S. Pat. Nos. 6,134,461 and 6,605,200, which are incorporated herein by reference in their entirety. Additional examples include those described in U.S. Pat. Nos. 6,736,957, 7,501,053 and 7,754,093, the disclosures of each of which are incorporated herein by reference in their entirety. Other suitable electron transfer agents may comprise metal compounds or complexes of ruthenium, osmium, iron (e.g., polyvinylferrocene or hexacyanoferrate), or cobalt, including metallocene compounds thereof, for example. Suitable examples of electron transfer mediators and polymer-bound electron transfer mediators may include those described in U.S. Pat. Nos. 8,444,834, 8,268,143 and 6,605,201, the disclosures of which are incorporated herein by reference in their entirety. Suitable ligands for the metal complexes may also include, for example, bidentate or higher denticity ligands such as, for example, bipyridine, biimidazole, phenanthroline, or pyridyl(imidazole). Other suitable bidentate ligands may include, for example, amino acids, oxalic acid, acetylacetone, diaminoalkanes, or o-diaminoarenes. Any combination of monodentate, bidentate, tridentate, tetradentate, or higher denticity ligands may be present in a metal complex to achieve a full coordination sphere.

In some embodiments, the active area or sensing region may also include a co-factor that is capable of catalyzing a reaction of the reactant associated with the at least one oxidase or dehydrogenase domain portion of the enzyme. In some aspects, the co-factor is a non-protein organic molecule such as, pyrroquinolinequinone (PQQ), flavine adenine dinucleotide (FAD), nicotinamide adenine dinucleotide (NAD), nicotinamide adenine dinucleotide phosphate (NADP), flavin mononucleotide (FMN), etc.). In certain aspects, a co-factor may be attached to a polymer, cross linking the co-factor with an electron transfer agent. A second co-factor may also be used in certain aspects. In some embodiments, where the enzyme turns over NAD or NADP, diaphorase is included.

In some embodiments, sensors are provided as a component of a benchtop instrument. In some embodiments, sensors are provided as part of a handheld instrument. In some embodiments, sensors are provided as part of a wearable device. In some embodiments, sensors are incorporated into or attached to a medical device, such as a catheter (e.g., indwelling catheter), endoscope, or the like.

### VI) Data Analysis/Software

In some embodiments, a system is provided that comprises a computer processor comprising or running software that controls one or more or all of: sensor control, sensor monitoring, data collection from the sensor, data analysis, data reporting (e.g., display), data storage, data transfer (e.g., to a cloud or communication network), and generation of an alarm or other signal to notify a user (e.g., user, patient, health care worker, etc.) of a notable event (e.g., the presence of an analyte, a change in concentration of an analyte, a threshold concentration of an analyte that corresponds to a need for an intervention, etc.). These processes may be embodied in software, firmware, hardware, or any combinations thereof.

Certain steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the technology may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

In some embodiments, the system tracks, analyzes, and/or reports on one or more of each of the following: a) sensor operational status (power status, battery status, etc.), b) raw signal from a sensor (e.g., electrochemical signal, fluorescent signal, etc.), c) presence or absence of detected analyte(s), d) analyte concentration or change in concentration, e) indication of health status change.

In some embodiments, the processor and/or software is located on a personal computing device (e.g., a handheld or wearable computing device, a tablet, a laptop computer, a desktop computer) associated with the user of the sensor (e.g., a patient, caretaker, healthcare worker, family member). In some embodiments, the processor and/or software is located on a computing device distant from the user (e.g., remote server) and is in electronic communication with the sensor or an intermediary device that receives information from the sensor.

### VII) Further Methods

In some embodiments, provided herein is a method of determining the presence, absence, or concentration of an analyte in a sample, such as a sample (e.g., a biological sample) as described herein. In some embodiments the method comprises the steps of:
a) contacting the sample with an enzyme as described herein in the presence of an inhibitor capable of binding to the analyte and to the grafted epitope of said enzyme; and
b) taking one or more measurements characteristic of the enzymatic activity of the enzyme.

In some embodiments, the method may comprise the steps of:
a) contacting the sample with an enzyme as described herein in the presence of an inhibitor capable of binding to the analyte and to the grafted epitope of said enzyme;
b) allowing the inhibitor to inhibit the enzyme;
c) allowing analyte present in the sample to bind to the inhibitor thereby de-inhibiting the enzyme; and
d) taking one or more measurements characteristic of the enzymatic activity of the enzyme.

In some embodiments the measurements are electrical measurements. Electrical measurements may be made in some embodiments when the enzyme is comprised in a system or sensor as described herein.

In some embodiments the enzymatic activity of the enzyme is proportional to the concentration of the analyte in the sample. For example, the presence, absence, or concentration of an analyte in a sample may be associated with a health condition as described herein. A health condition may be for example a pathological condition or a lifestyle condition. For example, the presence of a disease biomarker may be associated with the existence of a disease. By monitoring the presence, absence or concentration of a given analyte over time the associated health condition may be monitored. This can be useful, for example, to inform a physician in prescribing suitable medication; or to inform a subject in making appropriate lifestyle choices.

Accordingly, in some embodiments provided herein is a method of diagnosing the health of a subject, comprising (a) contacting a biological sample from said subject with an enzyme or sensor as described herein; and (b) determining the presence, absence, or concentration of an analyte associated with the health of the subject in the sample according to the provided methods. In some embodiments, provided herein is an epitope grafted enzyme as described herein, for use in a method of diagnosing the health of a subject, such use comprising contacting a biological sample from said subject with the enzyme; and (b) determining the presence, absence, or concentration of an analyte associated with the health of the subject in the sample as described herein.

In some further embodiments, provided herein is a method of identifying an allosteric site on an enzyme wherein the allosteric site is capable of being inhibited by an inhibitor, the method comprising:
a) generating one or more antibodies and/or antibody mimetics that bind to the enzyme;
b) screening the ability of said one or more antibodies and/or antibody mimetics to allosterically inhibit the enzymatic activity of the enzyme, thereby identifying antibodies and/or antibody mimetics which allosterically inhibit the enzymatic activity of the enzyme; and
c) identifying the amino acids of the enzyme which contact said antibodies and/or antibody mimetics which allosterically inhibit the enzymatic activity of the enzyme.

In some embodiments, said methods further comprising determining the retention of enzymatic activity when said amino acids are modified.

In some embodiments, said methods further comprising the step of grafting an epitope into the amino acid sequence of the enzyme at a position corresponding to the allosteric site, wherein the epitope comprises an amino acid sequence capable of binding to the inhibitor. In some embodiments the epitope is an epitope as described in more detail herein.

In some embodiments these methods can be used to identify, design, or improve an enzyme as described herein. Methods for identifying an allosteric site on an enzyme are described in more detail in the examples.

Also provided is an epitope-modified enzyme obtainable by such methods.

### Sequences

The following sequences are referenced throughout the examples. In addition to the sequences shown below, the sequence may contain a C-terminal G4S linker followed by a 5 His8 tag. The sequences may further optionally include a secretion signal (e.g., LFSLAFLSALSLATASPAGRAK (SEQ ID NO:130), which are recited below in certain of the sequences for illustrative purposes (shown with double underline); while in some embodiments the recited sequence omits the secretion signal peptide sequence). For glycosylated, secreted expression in Pichia culture, each protein sequence is appended at its 10 N-terminus with an AKS signal sequence prior to the listed sequences. The sequences also include an N-terminal methionine residue.

TABLE 1

| | | |
|---|---|---|
| 19031_sequence_ | 1 | Mqktatsntydyvivgggvgglalasrlsedksvtvavleagpnadeqfvvyapgmygqavgtdlcplrptvpqeamnnr |
| untagged | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvfqnetlaeeqrqqyynnktgiwttapnnlgypspsqlfngtsfesgqafanrirnstdqwaeyyastnatniellkkqy |
| | | aivasryeenylspieinftpgyggttdvdlknnkyqtvnhvliaplsrgythinssniedpvvinpqyythpmdvdvhiast |
| | | klarrilgaepglasinsgetqpgsnitsdedvkqwladnvrsdyhpvgtcamlprelggvvdpnllvygtanlrvvdasimp |
| | | leisshlmqptygvaekaadiikmsrknnnn |
| 19031_CC_mutant_ | 2 | Mqktatsntydyvivgggvgglalasrlsedksvtvavleagpnadeqfvvyapgmygqavgtdlcplrptvpqeamnnr |
| no_tag | | tltiatgkllgggsainglvwtrgalkdfdaweelgnpgwngrtmfkyfkkverfhpptkaqvqygatyqkgChgkngrid |
| | | isfpefqfpqsanwnaslatldfthqqdllCgslhgysttpntldpkterrvdsytgyiapfvsrknlfvlanhtvsriqfkpkng |
| | | tellkavgvewyttgdnsnkqtikarrevivssgsigspklleisgignkdivtaagvqslidlpgvgsnmqdhvhavtvsttni |
| | | tgfttdsvfqnetlaeeqrqqyynnktgiwttapnnlgypspsqlfngtsfesgqafanrirnstdqwaeyyastnatniellkk |
| | | qyaivasryeenylspieinftpgyggttdvdlknnkyqtvnhvliaplsrgythinssniedpvvinpqyythpmdvdvhia |
| | | stklarrilgaepglasinsgetqpgsnitsdedvkqwladnvrsdyhpvgtcamlprelggvvdpnllvygtanlrvvdasi |
| | | mpleisshlmqptygvaekaadiikmsrknnnn |
| 19031_sequence_ | 3 | Mqktatsntydyvivgggvgglalasrlsedksvtvavleagpnadeqfvvyapgmygqavgtdlcplrptvpqeamnnr |
| with_C-His6-tag | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvfqnetlaeeqrqqyynnktgiwttapnnlgypspsqlfngtsfesgqafanrirnstdqwaeyyastnatniellkkqy |
| | | aivasryeenylspieinftpgyggttdvdlknnkyqtvnhvliaplsrgythinssniedpvvinpqyythpmdvdvhiast |
| | | klarrilgaepglasinsgetqpgsnitsdedvkqwladnvrsdyhpvgtcamlprelggvvdpnllvygtanlrvvdasimp |
| | | leisshlmqptygvaekaadiikmsrknnnn |
| 19031_C-His_339V5 | 4 | Mqktatsntydyvivgggvgglalasrlsedksvtvavleagpnadeqfvvyapgmygqavgtdlcplrptvpqeamnnr |
| | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsIPNPLLGLDvfqnetlaeeqrqqyynnktgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C-His_340V5 | 5 | Mqktatsntydyvivgggvgglalasrlsedksvtvavleagpnadeqfvvyapgmygqavgtdlcplrptvpqeamnnr |
| | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvIPNPLLGLDfqnetlaeeqrqqyynnktgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C-His_341V5 | 6 | Mqktatsntydyvivgggvgglalasrlsedksvtvavleagpnadeqfvvyapgmygqavgtdlcplrptvpqeamnnr |
| | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvfIPNPLLGLDqnetlaeeqrqqyynnktgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C-His_358V5 | 7 | Mqktatsntydyvivgggvgglalasrlsedksvtvavleagpnadeqfvvyapgmygqavgtdlcplrptvpqeamnnr |
| | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvfqnetlaeeqrqqyynnkIPNPLLGLDtgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C-His_339TN | 8 | Mqktatsntydyvivgggvgglalasrlsedksvtvavleagpnadeqfvvyapgmygqavgtdlcplrptvpqeamnnr |
| | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsISASRKLQSvfqnetlaeeqrqqyynnktgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C-His_340TN | 9 | Mqktatsntydyvivgggvgglalasrlsedksvtvavleagpnadeqfvvyapgmygqavgtdlcplrptvpqeamnnr |
| | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvISASRKLQSfqnetlaeeqrqqyynnktgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C-His_341TN | 10 | Mqktatsntydyvivgggvgglalasrlsedksvtvavleagpnadeqfvvyapgmygqavgtdlcplrptvpqeamnnr |
| | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvfISASRKLQSqnetlaeeqrqqyynnktgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C-His_342TN | 11 | Mqktatsntydyvivgggvgglalasrlsedksvtvavleagpnadeqfvvyapgmygqavgtdlcplrptvpqeamnnr |
| | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvfqISASRKLQSnetlaeeqrqqyynnktgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C-His_343TN | 12 | Mqktatsntydyvivgggvgglalasrlsedksvtvavleagpnadeqfvvyapgmygqavgtdlcplrptvpqeamnnr |
| | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvfqnISASRKLQSetlaeeqrqqyynnktgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C-His_344TN | 13 | Mqktatsntydyvivgggvgglalasrlsedksvtvavleagpnadeqfvvyapgmygqavgtdlcplrptvpqeamnnr |
| | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvfqneISASRKLQStlaeeqrqqyynnktgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C-His_356TN | 14 | Mqktatsntydyvivgggvgglalasrlsedksvtvavleagpnadeqfvvyapgmygqavgtdlcplrptvpqeamnnr |
| | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvfqnetlaeeqrqqyynISASRKLQSnktgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C-His_357TN | 15 | Mqktatsntydyvivgggvgglalasrlsedksvtvavleagpnadeqfvvyapgmygqavgtdlcplrptvpqeamnnr |
| | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvfqnetlaeeqrqqyynnISASRKLQSktgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C-His_358TN | 16 | Mqktatsntydyvivgggvgglalasrlsedksvtvavleagpnadeqfvvyapgmygqavgtdlcplrptvpqeamnnr |
| | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvfqnetlaeeqrqqyynnkISASRKLQStgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C- | 17 | Mqktatsntydyvivgggvgglalasrlsedksvtvavleagpnadeqfvvyapgmygqavgtdlcplrptvpqeamnnr |
| His_339HLTN | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsisasrklqseqrqqyynnktgiwttapnnlgypspsqlfngtsfesgqafanrirnstdqwaeyyastnatniellkkqya |
| | | ivasryeenylspieinftpgyggttdvdlknnkyqtvnhvliaplsrgythinssniedpvvinpqyythpmdvdvhiastkl |
| | | arrilgaepglasinsgetqpgsnitsdedvkqwladnvrsdyhpvgtcamlprelggvvdpnllvygtanlrvvdasimple |
| | | isshlmqptygvaekaadiikmsrknnnn |
| 19031_C- | 18 | mqktatsntydyvivgggvgglalasrlsedksvtvavleagpnadeqfvvyapgmygqavgtdlcplrptvpqeamnnrt |
| His_342HLTN | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqisasrklqsqqyynnktgiwttapnnlgypspsqlfngtsfesgqafanrirnstdqwaeyyastnatniellkkqy |
| | | aivasryeenylspieinftpgyggttdvdlknnkyqtvnhvliaplsrgythinssniedpvvinpqyythpmdvdvhiast |
| | | klarrilgaepglasinsgetqpgsnitsdedvkqwladnvrsdyhpvgtcamlprelggvvdpnllvygtanlrvvdasimp |
| | | leisshlmqptygvaekaadiikmsrknnnn |
| 19031_C- | 19 | mqktatsntydyvivgggvgglalasrlsedksvtvavleagpnadeqfvvyapgmygqavgtdlcplrptvpqeamnnrt |
| His_344HLTN | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqneisasrklqsyynnktgiwttapnnlgypspsqlfngtsfesgqafanrirnstdqwaeyyastnatniellkkqya |
| | | ivasryeenylspieinftpgyggttdvdlknnkyqtvnhvliaplsrgythinssniedpvvinpqyythpmdvdvhiastkl |
| | | arrilgaepglasinsgetqpgsnitsdedvkqwladnvrsdyhpvgtcamlprelggvvdpnllvygtanlrvvdasimple |
| | | isshlmqptygvaekaadiikmsrknnnn |
| 19031_C- | 20 | mqktatsntydyvivgggvgglalasrlsedksvtvavleagpnadeqfvvyapgmygqavgtdlcplrptvpqeamnnrt |
| His_338HLTN | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdgisasrklqsggrqqyynnktgiwttapnnlgypspsqlfngtsfesgqafanrirnstdqwaeyyastnatniellkkqy |
| | | aivasryeenylspieinftpgyggttdvdlknnkyqtvnhvliaplsrgythinssniedpvvinpqyythpmdvdvhiast |
| | | klarrilgaepglasinsgetqpgsnitsdedvkqwladnvrsdyhpvgtcamlprelggvvdpnllvygtanlrvvdasimp |
| | | leisshlmqptygvaekaadiikmsrknnnn |
| 19031_C- | 21 | mqktatsntydyvivgggvgglalasrlsedksvtvavleagpnadeqfvvyapgmygqavgtdlcplrptvpqeamnnrt |
| His_340HLTN | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvggisasrklqsggyynnktgiwttapnnlgypspsqlfngtsfesgqafanrirnstdqwaeyyastnatniellkkqy |
| | | aivasryeenylspieinftpgyggttdvdlknnkyqtvnhvliaplsrgythinssniedpvvinpqyythpmdvdvhiast |
| | | klarrilgaepglasinsgetqpgsnitsdedvkqwladnvrsdyhpvgtcamlprelggvvdpnllvygtanlrvvdasimp |
| | | leisshlmqptygvaekaadiikmsrknnnn |
| 19031_C- | 22 | mqktatsntydyvivgggvgglalasrlsedksvtvavleagpnadeqfvvyapgmygqavgtdlcplrptvpqeamnnrt |
| His_342bHLTN | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqggisasrklqsyynnktgiwttapnnlgypspsqlfngtsfesgqafanrirnstdqwaeyyastnatniellkkqy |
| | | aivasryeenylspieinftpgyggttdvdlknnkyqtvnhvliaplsrgythinssniedpvvinpqyythpmdvdvhiast |
| | | klarrilgaepglasinsgetqpgsnitsdedvkqwladnvrsdyhpvgtcamlprelggvvdpnllvygtanlrvvdasimp |
| | | leisshlmqptygvaekaadiikmsrknnnn |
| 19031_C- | 23 | mqktatsntydyvivgggvgglalasrlsedksvtvavleagpnadeqfvvyapgmygqavgtdlcplrptvpqeamnnrt |
| His_338bHLV5 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdipnpllgldggqrqqyynnktgiwttapnnlgypspsqlfngtsfesgqafanrirnstdqwaeyyastnatniellkkqy |
| | | aivasryeenylspieinftpgyggttdvdlknnkyqtvnhvliaplsrgythinssniedpvvinpqyythpmdvdvhiast |
| | | klarrilgaepglasinsgetqpgsnitsdedvkqwladnvrsdyhpvgtcamlprelggvvdpnllvygtanlrvvdasimp |
| | | leisshlmqptygvaekaadiikmsrknnnn |
| 19031_C- | 24 | mqktatsntydyvivgggvgglalasrlsedksvtvavleagpnadeqfvvyapgmygqavgtdlcplrptvpqeamnnrt |
| His_338cHLV5 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | Lkavgvewyttgdnsnkqtikarrevivssgsigspklleisgignkdivtaagvqslidlpgvgsnmqdhvhavtvsttnitg |
| | | fttdggipnpllgldggqqyynnktgiwttapnnlgypspsqlfngtsfesgqafanrirnstdqwaeyyastnatniellkkqy |
| | | aivasryeenylspieinftpgyggttdvdlknnkyqtvnhvliaplsrgythinssniedpvvinpqyythpmdvdvhiast |
| | | klarrilgaepglasinsgetqpgsnitsdedvkqwladnvrsdyhpvgtcamlprelggvvdpnllvygtanlrvvdasimp |
| | | leisshlmqptygvaekaadiikmsrknnnn |
| 19031_C- | 25 | mqktatsntydyvivgggvgglalasrlsedksvtvavleagpnadeqfvvyapgmygqavgtdlcplrptvpqeamnnrt |
| His_341TN1 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfisasrklqlqnetlaeeqrqqyynnktgiwttapnnlgypspsqlfngtsfesgqafanrirnstdqwaeyyastnatn |
| | | iellkkqyaivasryeenylspieinftpgyggttdvdlknnkyqtvnhvliaplsrgythinssniedpvvinpqyythpmdv |
| | | dvhiastklarrilgaepglasinsgetqpgsnitsdedvkqwladnvrsdyhpvgtcamlprelggvvdpnllvygtanlrvv |
| | | dasimpleisshlmqptygvaekaadiikmsrknnnn |
| 19031_C- | 26 | mqktatsntydyvivgggvgglalasrlsedksvtvavleagpnadeqfvvyapgmygqavgtdlcplrptvpqeamnnrt |
| His_341TN4 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfkkisasrklqlktqnetlaeeqrqqyynnktgiwttapnnlgypspsqlfngtsfesgqafanrirnstdqwaeyyast |
| | | natniellkkqyaivasryeenylspieinftpgyggttdvdlknnkyqtvnhvliaplsrgythinssniedpvvinpqyythp |
| | | mdvdvhiastklarrilgaepglasinsgetqpgsnitsdedvkqwladnvrsdyhpvgtcamlprelggvvdpnllvygta |
| | | nlrvvdasimpleisshlmqptygvaekaadiikmsrknnnn |
| 19031_C- | 27 | mqktatsntydyvivgggvgglalasrlsedksvtvavleagpnadeqfvvyapgmygqavgtdlcplrptvpqeamnnrt |
| His_341TN8 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfkkisasrklqlktlllqnetlaeeqrqqyynnktgiwttapnnlgypspsqlfngtsfesgqafanrirnstdqwaeyy |
| | | astnatniellkkqyaivasryeenylspieinftpgyggttdvdlknnkyqtvnhvliaplsrgythinssniedpvvinpqyy |
| | | thpmdvdvhiastklarrilgaepglasinsgetqpgsnitsdedvkqwladnvrsdyhpvgtcamlprelggvvdpnllvy |
| | | gtanlrvvdasimpleisshlmqptygvaekaadiikmsrknnnn |
| 19031_C- | 28 | mqktatsntydyvivgggvgglalasrlsedksvtvavleagpnadeqfvvyapgmygqavgtdlcplrptvpqeamnnrt |
| His_358TN1 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkisasrklqltgiwttapnnlgypspsqlfngtsfesgqafanrirnstdqwaeyyastnatn |
| | | iellkkqyaivasryeenylspieinftpgyggttdvdlknnkyqtvnhvliaplsrgythinssniedpvvinpqyythpmdv |
| | | dvhiastklarrilgaepglasinsgetqpgsnitsdedvkqwladnvrsdyhpvgtcamlprelggvvdpnllvygtanlrvv |
| | | dasimpleisshlmqptygvaekaadiikmsrknnnn |
| 19031_C- | 29 | mqktatsntydyvivgggvgglalasrlsedksvtvavleagpnadeqfvvyapgmygqavgtdlcplrptvpqeamnnrt |
| His_358TN4 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkkisasrklqlkttgiwttapnnlgypspsqlfngtsfesgqafanrirnstdqwaeyyastn |
| | | atniellkkqyaivasryeenylspieinftpgyggttdvdlknnkyqtvnhvliaplsrgythinssniedpvvinpqyythp |
| | | mdvdvhiastklarrilgaepglasinsgetqpgsnitsdedvkqwladnvrsdyhpvgtcamlprelggvvdpnllvygta |
| | | nlrvvdasimpleisshlmqptygvaekaadiikmsrknnnn |
| 19031_C- | 30 | mqktatsntydyvivgggvgglalasrlsedksvtvavleagpnadeqfvvyapgmygqavgtdlcplrptvpqeamnnrt |
| His_358TN8 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkkisasrklqlktllltgiwttapnnlgypspsqlfngtsfesgqafanrirnstdqwaeyyas |
| | | tnatniellkkqyaivasryeenylspieinftpgyggttdvdlknnkyqtvnhvliaplsrgythinssniedpvvinpqyyth |
| | | pmdvdvhiastklarrilgaepglasinsgetqpgsnitsdedvkqwladnvrsdyhpvgtcamlprelggvvdpnllvygt |
| | | anlrvvdasimpleisshlmqptygvaekaadiikmsrknnnn |
| 19031_C-His_341- | 31 | mqktatsntydyvivgggvgglalasrlsedksvtvavleagpnadeqfvvyapgmygqavgtdlcplrptvpqeamnnrt |
| V5FL | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfgkpipnpllgldstqnetlaeeqrqqyynnktgiwttapnnlgypspsqlfngtsfesgqafanrirnstdqwaeyy |
| | | astnatniellkkqyaivasryeenylspieinftpgyggttdvdlknnkyqtvnhvliaplsrgythinssniedpvvinpqyy |
| | | thpmdvdvhiastklarrilgaepglasinsgetqpgsnitsdedvkqwladnvrsdyhpvgtcamlprelggvvdpnllvy |
| | | gtanlrvvdasimpleisshlmqptygvaekaadiikmsrknnnn |
| 19031_C-His_341- | 32 | mqktatsntydyvivgggvgglalasrlsedksvtvavleagpnadeqfvvyapgmygqavgtdlcplrptvpqeamnnrt |
| VL1 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfGIPNPLLGLDqnetlaeeqrqqyynnktgiwttapnnlgypspsqlfngtsfesgqafanrirnstdqwaey |
| | | yastnatniellkkqyaivasryeenylspieinftpgyggttdvdlknnkyqtvnhvliaplsrgythinssniedpvvinpqy |
| | | ythpmdvdvhiastklarrilgaepglasinsgetqpgsnitsdedvkqwladnvrsdyhpvgtcamlprelggvvdpnllv |
| | | ygtanlrvvdasimpleisshlmqptygvaekaadiikmsrknnnn |
| 19031_C-His_341- | 33 | mqktatsntydyvivgggvgglalasrlsedksvtvavleagpnadeqfvvyapgmygqavgtdlcplrptvpqeamnnrt |
| VL2 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfGGIPNPLLGLDqnetlaeeqrqqyynnktgiwttapnnlgypspsqlfngtsfesgqafanrirnstdqwae |
| | | yyastnatniellkkqyaivasryeenylspieinftpgyggttdvdlknnkyqtvnhvliaplsrgythinssniedpvvinpq |
| | | yythpmdvdvhiastklarrilgaepglasinsgetqpgsnitsdedvkqwladnvrsdyhpvgtcamlprelggvvdpnll |
| | | vygtanlrvvdasimpleisshlmqptygvaekaadiikmsrknnnn |
| 19031_C-His_341- | 34 | mqktatsntydyvivgggvgglalasrlsedksvtvavleagpnadeqfvvyapgmygqavgtdlcplrptvpqeamnnrt |
| VL3 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfIPNPLLGLDGqnetlaeeqrqqyynnktgiwttapnnlgypspsqlfngtsfesgqafanrirnstdqwaey |
| | | yastnatniellkkqyaivasryeenylspieinftpgyggttdvdlknnkyqtvnhvliaplsrgythinssniedpvvinpqy |
| | | ythpmdvdvhiastklarrilgaepglasinsgetqpgsnitsdedvkqwladnvrsdyhpvgtcamlprelggvvdpnllv |
| | | ygtanlrvvdasimpleisshlmqptygvaekaadiikmsrknnnn |
| 19031_C-His_341- | 35 | mqktatsntydyvivgggvgglalasrlsedksvtvavleagpnadeqfvvyapgmygqavgtdlcplrptvpqeamnnrt |
| VL4 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfIPNPLLGLDGGqnetlaeeqrqqyynnktgiwttapnnlgypspsqlfngtsfesgqafanrirnstdqwae |
| | | yyastnatniellkkqyaivasryeenylspieinftpgyggttdvdlknnkyqtvnhvliaplsrgythinssniedpvvinpq |
| | | yythpmdvdvhiastklarrilgaepglasinsgetqpgsnitsdedvkqwladnvrsdyhpvgtcamlprelggvvdpnll |
| | | vygtanlrvvdasimpleisshlmqptygvaekaadiikmsrknnnn |
| 19031_C-His_341- | 36 | mqktatsntydyvivgggvgglalasrlsedksvtvavleagpnadeqfvvyapgmygqavgtdlcplrptvpqeamnnrt |
| VL5 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfGIPNPLLGLDGqnetlaeeqrqqyynnktgiwttapnnlgypspsqlfngtsfesgqafanrirnstdqwae |
| | | yyastnatniellkkqyaivasryeenylspieinftpgyggttdvdlknnkyqtvnhvliaplsrgythinssniedpvvinpq |
| | | yythpmdvdvhiastklarrilgaepglasinsgetqpgsnitsdedvkqwladnvrsdyhpvgtcamlprelggvvdpnll |
| | | vygtanlrvvdasimpleisshlmqptygvaekaadiikmsrknnnn |
| 19031_C-His_341- | 37 | mqktatsntydyvivgggvgglalasrlsedksvtvavleagpnadeqfvvyapgmygqavgtdlcplrptvpqeamnnrt |
| VL6 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfSGIPNPLLGLDGSqnetlaeeqrqqyynnktgiwttapnnlgypspsqlfngtsfesgqafanrirnstdq |
| | | waeyyastnatniellkkqyaivasryeenylspieinftpgyggttdvdlknnkyqtvnhvliaplsrgythinssniedpvvi |
| | | npqyythpmdvdvhiastklarrilgaepglasinsgetqpgsnitsdedvkqwladnvrsdyhpvgtcamlprelggvvd |
| | | pnllvygtanlrvvdasimpleisshlmqptygvaekaadiikmsrknnnn |
| 19031_C-His_341- | 38 | mqktatsntydyvivgggvgglalasrlsedksvtvavleagpnadeqfvvyapgmygqavgtdlcplrptvpqeamnnrt |
| VL7 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfGIPNPLLGLDGGqnetlaeeqrqqyynnktgiwttapnnlgypspsqlfngtsfesgqafanrirnstdqw |
| | | aeyyastnatniellkkqyaivasryeenylspieinftpgyggttdvdlknnkyqtvnhvliaplsrgythinssniedpvvin |
| | | pqyythpmdvdvhiastklarrilgaepglasinsgetqpgsnitsdedvkqwladnvrsdyhpvgtcamlprelggvvdp |
| | | nllvygtanlrvvdasimpleisshlmqptygvaekaadiikmsrknnnn |
| 19031_C-His_341- | 39 | mqktatsntydyvivgggvgglalasrlsedksvtvavleagpnadeqfvvyapgmygqavgtdlcplrptvpqeamnnrt |
| VL8 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfGGIPNPLLGLDGqnetlaeeqrqqyynnktgiwttapnnlgypspsqlfngtsfesgqafanrirnstdqw |
| | | aeyyastnatniellkkqyaivasryeenylspieinftpgyggttdvdlknnkyqtvnhvliaplsrgythinssniedpvvin |
| | | pqyythpmdvdvhiastklarrilgaepglasinsgetqpgsnitsdedvkqwladnvrsdyhpvgtcamlprelggvvdp |
| | | nllvygtanlrvvdasimpleisshlmqptygvaekaadiikmsrknnnn |
| 19031_C-His_341- | 40 | mqktatsntydyvivgggvgglalasrlsedksvtvavleagpnadeqfvvyapgmygqavgtdlcplrptvpqeamnnrt |
| VL9 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfGGIPNPLLGLDGGqnetlaeeqrqqyynnktgiwttapnnlgypspsqlfngtsfesgqafanrirnstdq |
| | | waeyyastnatniellkkqyaivasryeenylspieinftpgyggttdvdlknnkyqtvnhvliaplsrgythinssniedpvvi |
| | | npqyythpmdvdvhiastklarrilgaepglasinsgetqpgsnitsdedvkqwladnvrsdyhpvgtcamlprelggvvd |
| | | pnllvygtanlrvvdasimpleisshlmqptygvaekaadiikmsrknnnn |
| 19031_C-His_341- | 41 | mqktatsntydyvivgggvgglalasrlsedksvtvavleagpnadeqfvvyapgmygqavgtdlcplrptvpqeamnnrt |
| VL10 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfSGGGIPNPLLGLDGGGSqnetlaeeqrqqyynnktgiwttapnnlgypspsqlfngtsfesgqafanri |
| | | rnstdqwaeyyastnatniellkkqyaivasryeenylspieinftpgyggttdvdlknnkyqtvnhvliaplsrgythinssni |
| | | edpvvinpqyythpmdvdvhiastklarrilgaepglasinsgetqpgsnitsdedvkqwladnvrsdyhpvgtcamlprel |
| | | ggvvdpnllvygtanlrvvdasimpleisshlmqptygvaekaadiikmsrknnnn |
| 19031_C-His_341- | 42 | mqktatsntydyvivgggvgglalasrlsedksvtvavleagpnadeqfvvyapgmygqavgtdlcplrptvpqeamnnrt |
| VL11 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfSGGGGIPNPLLGLDGGGGSqnetlaeeqrqqyynnktgiwttapnnlgypspsqlfngtsfesgqaf |
| | | anrirnstdqwaeyyastnatniellkkqyaivasryeenylspieinftpgyggttdvdlknnkyqtvnhvliaplsrgythin |
| | | ssniedpvvinpqyythpmdvdvhiastklarrilgaepglasinsgetqpgsnitsdedvkqwladnvrsdyhpvgtcaml |
| | | prelggvvdpnllvygtanlrvvdasimpleisshlmqptygvaekaadiikmsrknnnn |
| 19031_C-His_358- | 43 | mqktatsntydyvivgggvgglalasrlsedksvtvavleagpnadeqfvvyapgmygqavgtdlcplrptvpqeamnnrt |
| V5FL | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkgkpipnpllgldsttgiwttapnnlgypspsqlfngtsfesgqafanrirnstdqwaeyy |
| | | astnatniellkkqyaivasryeenylspieinftpgyggttdvdlknnkyqtvnhvliaplsrgythinssniedpvvinpqyy |
| | | thpmdvdvhiastklarrilgaepglasinsgetqpgsnitsdedvkqwladnvrsdyhpvgtcamlprelggvvdpnllvy |
| | | gtanlrvvdasimpleisshlmqptygvaekaadiikmsrknnnn |
| 19031_C-His_358- | 44 | mqktatsntydyvivgggvgglalasrlsedksvtvavleagpnadeqfvvyapgmygqavgtdlcplrptvpqeamnnrt |
| VL1 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkGIPNPLLGLDtgiwttapnnlgypspsqlfngtsfesgqafanrirnstdqwaey |
| | | yastnatniellkkqyaivasryeenylspieinftpgyggttdvdlknnkyqtvnhvliaplsrgythinssniedpvvinpqy |
| | | ythpmdvdvhiastklarrilgaepglasinsgetqpgsnitsdedvkqwladnvrsdyhpvgtcamlprelggvvdpnllv |
| | | ygtanlrvvdasimpleisshlmqptygvaekaadiikmsrknnnn |
| 19031_C-His_358- | 45 | Mqktatsntydyvivgggvgglalasrlsedksvtvavleagpnadeqfvvyapgmygqavgtdlcplrptvpqeamnnr |
| VL2 | | tltiatgkllgggsainglvwtrgalkdfdaweelgnpgwngrtmfkyfkkverfhpptkaqvqygatyqkgvhgkngridi |
| | | sfpefqfpqsanwnaslatldfthqqdllngslhgysttpntldpkterrvdsytgyiapfvsrknlfvlanhtvsriqfkpkngte |
| | | llkavgvewyttgdnsnkqtikarrevivssgsigspklleisgignkdivtaagvqslidlpgvgsnmqdhvhavtvsttnitg |
| | | fttdsvfqnetlaeeqrqqyynnkGGIPNPLLGLDtgiwttapnnlgypspsqlfngtsfesgqafanrirnstdqwae |
| | | yyastnatniellkkqyaivasryeenylspieinftpgyggttdvdlknnkyqtvnhvliaplsrgythinssniedpvvinpq |
| | | yythpmdvdvhiastklarrilgaepglasinsgetqpgsnitsdedvkqwladnvrsdyhpvgtcamlprelggvvdpnll |
| | | vygtanlrvvdasimpleisshlmqptygvaekaadiikmsrknnnn |
| 19031_C-His_358- | 46 | mqktatsntydyvivgggvgglalasrlsedksvtvavleagpnadeqfvvyapgmygqavgtdlcplrptvpqeamnnrt |
| VL3 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkIPNPLLGLDGtgiwttapnnlgypspsqlfngtsfesgqafanrirnstdqwaey |
| | | yastnatniellkkqyaivasryeenylspieinftpgyggttdvdlknnkyqtvnhvliaplsrgythinssniedpvvinpqy |
| | | ythpmdvdvhiastklarrilgaepglasinsgetqpgsnitsdedvkqwladnvrsdyhpvgtcamlprelggvvdpnllv |
| | | ygtanlrvvdasimpleisshlmqptygvaekaadiikmsrknnnn |
| 19031_C-His_358- | 47 | mqktatsntydyvivgggvgglalasrlsedksvtvavleagpnadeqfvvyapgmygqavgtdlcplrptvpqeamnnrt |
| VL4 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkIPNPLLGLDGGtgiwttapnnlgypspsqlfngtsfesgqafanrirnstdqwae |
| | | yyastnatniellkkqyaivasryeenylspieinftpgyggttdvdlknnkyqtvnhvliaplsrgythinssniedpvvinpq |
| | | yythpmdvdvhiastklarrilgaepglasinsgetqpgsnitsdedvkqwladnvrsdyhpvgtcamlprelggvvdpnll |
| | | vygtanlrvvdasimpleisshlmqptygvaekaadiikmsrknnnn |
| 19031_C-His_358- | 48 | mqktatsntydyvivgggvgglalasrlsedksvtvavleagpnadeqfvvyapgmygqavgtdlcplrptvpqeamnnrt |
| VL5 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkGIPNPLLGLDGtgiwttapnnlgypspsqlfngtsfesgqafanrirnstdqwae |
| | | yyastnatniellkkqyaivasryeenylspieinftpgyggttdvdlknnkyqtvnhvliaplsrgythinssniedpvvinpq |
| | | yythpmdvdvhiastklarrilgaepglasinsgetqpgsnitsdedvkqwladnvrsdyhpvgtcamlprelggvvdpnll |
| | | vygtanlrvvdasimpleisshlmqptygvaekaadiikmsrknnnn |
| 19031_C-His_358- | 49 | mqktatsntydyvivgggvgglalasrlsedksvtvavleagpnadeqfvvyapgmygqavgtdlcplrptvpqeamnnrt |
| VL6 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkSGIPNPLLGLDGStgiwttapnnlgypspsqlfngtsfesgqafanrirnstdq |
| | | waeyyastnatniellkkqyaivasryeenylspieinftpgyggttdvdlknnkyqtvnhvliaplsrgythinssniedpvvi |
| | | npqyythpmdvdvhiastklarrilgaepglasinsgetqpgsnitsdedvkqwladnvrsdyhpvgtcamlprelggvvd |
| | | pnllvygtanlrvvdasimpleisshlmqptygvaekaadiikmsrknnnn |
| 19031_C-His_358- | 50 | mqktatsntydyvivgggvgglalasrlsedksvtvavleagpnadeqfvvyapgmygqavgtdlcplrptvpqeamnnrt |
| VL7 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkGIPNPLLGLDGGtgiwttapnnlgypspsqlfngtsfesgqafanrirnstdqw |
| | | aeyyastnatniellkkqyaivasryeenylspieinftpgyggttdvdlknnkyqtvnhvliaplsrgythinssniedpvvin |
| | | pqyythpmdvdvhiastklarrilgaepglasinsgetqpgsnitsdedvkqwladnvrsdyhpvgtcamlprelggvvdp |
| | | nllvygtanlrvvdasimpleisshlmqptygvaekaadiikmsrknnnn |
| 19031_C-His_358- | 51 | mqktatsntydyvivgggvgglalasrlsedksvtvavleagpnadeqfvvyapgmygqavgtdlcplrptvpqeamnnrt |
| VL8 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdlingslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkGGIPNPLLGLDGtgiwttapnnlgypspsqlfngtsfesgqafanrirnstdqw |
| | | aeyyastnatniellkkqyaivasryeenylspieinftpgyggttdvdlknnkyqtvnhvliaplsrgythinssniedpvvin |
| | | pqyythpmdvdvhiastklarrilgaepglasinsgetqpgsnitsdedvkqwladnvrsdyhpvgtcamlprelggvvdp |
| | | nllvygtanlrvvdasimpleisshlmqptygvaekaadiikmsrknnnn |
| 19031_C-His_358- | 52 | mqktatsntydyvivgggvgglalasrlsedksvtvavleagpnadeqfvvyapgmygqavgtdlcplrptvpqeamnnrt |
| VL9 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkGGIPNPLLGLDGGtgiwttapnnlgypspsqlfngtsfesgqafanrirnstdq |
| | | waeyyastnatniellkkqyaivasryeenylspieinftpgyggttdvdlknnkyqtvnhvliaplsrgythinssniedpvvi |
| | | npqyythpmdvdvhiastklarrilgaepglasinsgetqpgsnitsdedvkqwladnvrsdyhpvgtcamlprelggvvd |
| | | pnllvygtanlrvvdasimpleisshlmqptygvaekaadiikmsrknnnn |
| 19031_C-His_358- | 53 | mqktatsntydyvivgggvgglalasrlsedksvtvavleagpnadeqfvvyapgmygqavgtdlcplrptvpqeamnnrt |
| VL10 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdlingslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkSGGGIPNPLLGLDGGGStgiwttapnnlgypspsqlfngtsfesgqafanri |
| | | rnstdqwaeyyastnatniellkkqyaivasryeenylspieinftpgyggttdvdlknnkyqtvnhvliaplsrgythinssni |
| | | edpvvinpqyythpmdvdvhiastklarrilgaepglasinsgetqpgsnitsdedvkqwladnvrsdyhpvgtcamlprel |
| | | ggvvdpnllvygtanlrvvdasimpleisshlmqptygvaekaadiikmsrknnnn |
| 19031_C-His_358- | 54 | mqktatsntydyvivgggvgglalasrlsedksvtvavleagpnadeqfvvyapgmygqavgtdlcplrptvpqeamnnrt |
| VL11 | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkSGGGGIPNPLLGLDGGGGStgiwttapnnlgypspsqlfngtsfesgqaf |
| | | anrirnstdqwaeyyastnatniellkkqyaivasryeenylspieinftpgyggttdvdlknnkyqtvnhvliaplsrgythin |
| | | ssniedpvvinpqyythpmdvdvhiastklarrilgaepglasinsgetqpgsnitsdedvkqwladnvrsdyhpvgtcaml |
| | | prelggvvdpnllvygtanlrvvdasimpleisshlmqptygvaekaadiikmsrknnnn |
| 19031_C- | 55 | mqktatsntydyvivgggvgglalasrlsedksvtvavleagpnadeqfvvyapgmygqavgtdlcplrptvpqeamnnrt |
| His_341HA | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfYPYDVPDYAqnetlaeeqrqqyynnktgiwttapnnlgypspsqlfngtsfesgqafanrirnstdqwaeyy |
| | | astnatniellkkqyaivasryeenylspieinftpgyggttdvdlknnkyqtvnhvliaplsrgythinssniedpvvinpqyy |
| | | thpmdvdvhiastklarrilgaepglasinsgetqpgsnitsdedvkqwladnvrsdyhpvgtcamlprelggvvdpnllvy |
| | | gtanlrvvdasimpleisshlmqptygvaekaadiikmsrknnnn |
| 19031_C- | 56 | mqktatsntydyvivgggvgglalasrlsedksvtvavleagpnadeqfvvyapgmygqavgtdlcplrptvpqeamnnrt |
| His_358HA | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkYPYDVPDYAtgiwttapnnlgypspsqlfngtsfesgqafanrirnstdqwaeyy |
| | | astnatniellkkqyaivasryeenylspieinftpgyggttdvdlknnkyqtvnhvliaplsrgythinssniedpvvinpqyy |
| | | thpmdvdvhiastklarrilgaepglasinsgetqpgsnitsdedvkqwladnvrsdyhpvgtcamlprelggvvdpnllvy |
| | | gtanlrvvdasimpleisshlmqptygvaekaadiikmsrknnnn |
| 19031_C- | 57 | mqktatsntydyvivgggvgglalasrlsedksvtvavleagpnadeqfvvyapgmygqavgtdlcplrptvpqeamnnrt |
| His_341Myc | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfEQKLISEEDLqnetlaeeqrqqyynnktgiwttapnnlgypspsqlfngtsfesgqafanrirnstdqwaeyy |
| | | astnatniellkkqyaivasryeenylspieinftpgyggttdvdlknnkyqtvnhvliaplsrgythinssniedpvvinpqyy |
| | | thpmdvdvhiastklarrilgaepglasinsgetqpgsnitsdedvkqwladnvrsdyhpvgtcamlprelggvvdpnllvy |
| | | gtanlrvvdasimpleisshlmqptygvaekaadiikmsrknnnn |
| 19031_C- | 58 | mqktatsntydyvivgggvgglalasrlsedksvtvavleagpnadeqfvvyapgmygqavgtdlcplrptvpqeamnnrt |
| His_358Myc | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkEQKLISEEDLtgiwttapnnlgypspsqlfngtsfesgqafanrirnstdqwaeyy |
| | | astnatniellkkqyaivasryeenylspieinftpgyggttdvdlknnkyqtvnhvliaplsrgythinssniedpvvinpqyy |
| | | thpmdvdvhiastklarrilgaepglasinsgetqpgsnitsdedvkqwladnvrsdyhpvgtcamlprelggvvdpnllvy |
| | | gtanlrvvdasimpleisshlmqptygvaekaadiikmsrknnnn |
| 19031_C- | 59 | mqktatsntydyvivgggvgglalasrlsedksvtvavleagpnadeqfvvyapgmygqavgtdlcplrptvpqeamnnrt |
| His_341FLAG | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfDYKDDDDKqnetlaeeqrqqyynnktgiwttapnnlgypspsqlfngtsfesgqafanrirnstdqwaeyya |
| | | stnatniellkkqyaivasryeenylspieinftpgyggttdvdlknnkyqtvnhvliaplsrgythinssniedpvvinpqyyt |
| | | hpmdvdvhiastklarrilgaepglasinsgetqpgsnitsdedvkqwladnvrsdyhpvgtcamlprelggvvdpnllvyg |
| | | tanlrvvdasimpleisshlmqptygvaekaadiikmsrknnnn |
| 19031_C- | 60 | mqktatsntydyvivgggvgglalasrlsedksvtvavleagpnadeqfvvyapgmygqavgtdlcplrptvpqeamnnrt |
| His_358FLAG | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkDYKDDDDKtgiwttapnnlgypspsqlfngtsfesgqafanrirnstdqwaeyya |
| | | stnatniellkkqyaivasryeenylspieinftpgyggttdvdlknnkyqtvnhvliaplsrgythinssniedpvvinpqyyt |
| | | hpmdvdvhiastklarrilgaepglasinsgetqpgsnitsdedvkqwladnvrsdyhpvgtcamlprelggvvdpnllvyg |
| | | tanlrvvdasimpleisshlmqptygvaekaadiikmsrknnnn |
| 19031_C- | 61 | mqktatsntydyvivgggvgglalasrlsedksvtvavleagpnadeqfvvyapgmygqavgtdlcplrptvpqeamnnrt |
| His_341HNL | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfQPGEFTLGNqnetlaeeqrqqyynnktgiwttapnnlgypspsqlfngtsfesgqafanrirnstdqwaeyya |
| | | stnatniellkkqyaivasryeenylspieinftpgyggttdvdlknnkyqtvnhvliaplsrgythinssniedpvvinpqyyt |
| | | hpmdvdvhiastklarrilgaepglasinsgetqpgsnitsdedvkqwladnvrsdyhpvgtcamlprelggvvdpnllvyg |
| | | tanlrvvdasimpleisshlmqptygvaekaadiikmsrknnnn |
| 19031_C- | 62 | mqktatsntydyvivgggvgglalasrlsedksvtvavleagpnadeqfvvyapgmygqavgtdlcplrptvpqeamnnrt |
| His_358HNL | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkQPGEFTLGNtgiwttapnnlgypspsqlfngtsfesgqafanrirnstdqwaeyya |
| | | stnatniellkkqyaivasryeenylspieinftpgyggttdvdlknnkyqtvnhvliaplsrgythinssniedpvvinpqyyt |
| | | hpmdvdvhiastklarrilgaepglasinsgetqpgsnitsdedvkqwladnvrsdyhpvgtcamlprelggvvdpnllvyg |
| | | tanlrvvdasimpleisshlmqptygvaekaadiikmsrknnnn |
| 341BP1 | 63 | mqktatsntydyvivgggvgglalasrlsedksvtvavleagpnadeqfvvyapgmygqavgtdlcplrptvpqeamnnrt |
| | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfETSGLQEQqnetlaeeqrqqyynnktgiwttapnnlgypspsqlfngtsfesgqafanrirnstdqwaeyyast |
| | | natniellkkqyaivasryeenylspieinftpgyggttdvdlknnkyqtvnhvliaplsrgythinssniedpvvinpqyythp |
| | | mdvdvhiastklarrilgaepglasinsgetqpgsnitsdedvkqwladnvrsdyhpvgtcamlprelggvvdpnllvygta |
| | | nlrvvdasimpleisshlmqptygvaekaadiikmsrknnnn |
| 358BP1 | 64 | mqktatsntydyvivgggvgglalasrlsedksvtvavleagpnadeqfvvyapgmygqavgtdlcplrptvpqeamnnrt |
| | | ltiatgkllgggsainglvwtrgalkdfdaweelgnpgwngrtmfkyfkkverfhpptkaqvqygatyqkgvhgkngridis |
| | | fpefqfpqsanwnaslatldfthqqdllngslhgysttpntldpkterrvdsytgyiapfvsrknlfvlanhtvsriqfkpkngtel |
| | | lkavgvewyttgdnsnkqtikarrevivssgsigspklleisgignkdivtaagvqslidlpgvgsnmqdhvhavtvsttnitg |
| | | fttdsvfqnetlaeeqrqqyynnkETSGLQEQtgiwttapnnlgypspsqlfngtsfesgqafanrirnstdqwaeyyast |
| | | natniellkkqyaivasryeenylspieinftpgyggttdvdlknnkyqtvnhvliaplsrgythinssniedpvvinpqyythp |
| | | mdvdvhiastklarrilgaepglasinsgetqpgsnitsdedvkqwladnvrsdyhpvgtcamlprelggvvdpnllvygta |
| | | nlrvvdasimpleisshlmqptygvaekaadiikmsrknnnn |
| 19031_C- | 65 | mqktatsntydyvivgggvgglalasrlsedksvtvavleagpnadeqfvvyapgmygqavgtdlcplrptvpqeamnnrt |
| His_5xxHHAA | | ltiatgkllgggsainglvwtrgalkdfdaweelgnpgwngrtmfkyfkkverfhpptkaqvqygatyqkgvhgkngridis |
| (Catalytically-dead | | fpefqfpqsanwnaslatldfthqqdllngslhgysttpntldpkterrvdsytgyiapfvsrknlfvlanhtvsriqfkpkngtel |
| 19031 enzyme) | | lkavgvewyttgdnsnkqtikarrevivssgsigspklleisgignkdivtaagvqslidlpgvgsnmqdhvhavtvsttnitg |
| | | fttdsvfqnetlaeeqrqqyynnktgiwttapnnlgypspsqlfngtsfesgqafanrirnstdqwaeyyastnatniellkkqy |
| | | aivasryeenylspieinftpgyggttdvdlknnkyqtvnhvliaplsrgythinssniedpvvinpqyythpmdvdvhiast |
| | | klarrilgaepglasinsgetqpgsnitsdedvkqwladnvrsdyApvgtcamlprelggvvdpnllvygtanlrvvdasim |
| | | pleissAlmqptygvaekaadiikmsrknnnn |
| Mucor_prainii | 66 | MQDTNSSSTDTYDYVIVGGGVAGLALASRISENKDVTVAVLESGPNANDRFVVY |
| | | APGMYGQAVGTDLCPLIPTTPQENMGNRSLTIATGRLLGGGSAINGLVWTRGGLK |
| | | DYDAWEELGNPGWNGANLFKYFKKVENFTPPTPAQIEYGATYQKSAHGKKGPID |
| | | VSFTNYEFSQSASWNASLETLDFTALPDILNGTLAGYSTTPNILDPETVQRVDSYT |
| | | GYIAPYTSRNNLNVLANHTVSRIQFAPKNGSEPLKATGVEWYPTGNKNQKQIIKA |
| | | RYEVIISSGAIGSPKLLEISGIGNKDIVSAAGVESLIDLPGVGSNMQDHVHAITVSTT |
| | | NITGYTTNSVFVNETLAQEQREEYEANKTGIWATTPNNLGYPTPEQLENGTEFVS |
| | | GKEFADKIRNSTDEWANYYASTNASNVELLKKQYAIVASRYEENYLSPIEINFTPG |
| | | YEGSGNVDLQNNKYQTVNHVLIAPLSRGYTHINSSDVEDHSVINPQYYSHPMDID |
| | | VHIASTKLAREIITASPGLGDINSGEIEPGMNITSEDDLRSWLSNNVRSDWHPVGTC |
| | | AMLPKELGGVVSPALMVYGTSNLRVVDASIMPLEVSSHLMQPTYGIAEKAADIIK |
| | | NFYKTQHKNQN |
| Mucor_guilliermondii | 67 | MQSNTDTYDYVIVGGGVGGLALANRLSENKQVTVAVLEAGPNANDEFIVYAPG |
| | | MYGQAVGTYLAPLRPTVPQENMNNRSLSIATGKLLGGGSAVNGLVWTRGATKD |
| | | FDAWEELGNPGWNGASMFKYFKKVENFTAPTPYQVNYGATYQKNTHGYKGPV |
| | | QVSFTNYEFPQSAHWNQSLASLGFDHLPDLLNGTLSGYSTTPNILDPNTDQRCDA |
| | | YAAYIAPYTARTNLHVLANHTVSRIEFNQTNANQPLVASGVEWYPTGDNTKKQTI |
| | | KARLEVIVSSGSIGSPKLLEISGIGNKDIVTAAGVKSLLDLPGVGSNMQDHVHAVT |
| | | VSTTNITGYTTDSVFVNSTLASEQREQYEKDKSGIWTTTPNNLGYPTPAQLENGTE |
| | | FMDGKAFAARIRNSSQEWAQYYASKNASTVELLMKQYEIVASRYEENYLSPIEIN |
| | | LTPGYGGVGTVDKTKNKYQTVNHVLIAPLSRGFTHINSSDIEDPVNINPQYYSHPM |
| | | DIDVHVASTKLARRIINAPGLGDLNSGEVEPGMDITSDSDVRAWLANNVRSDWHP |
| | | VGTCAMLPKELGGVVDSSLKVYGTANLRVVDASIMPLEVSSHLMQPTFGVAEKA |
| | | ADIIKAEYKKQKAQ |
| Mucor_hiemalis | 68 | MQKTATSNTYDYVIVGGGVGGLALASRLSEDKSVTVAVLEAGPNADEQFVVYAP |
| | | GMYGQAVGTDLCPLRPTVPQEAMNNRTLTIATGKLLGGGSAINGLVWTRGALKD |
| | | FDAWEELGNPGWNGRTMFKYFKKVERFHPPTKAQVQYGATYQKGVHGKNGRI |
| | | DISFPEFQFPQSANWNASLATLDFTHQQDLLNGSLHGYSTTPNTLDPKTARRVDS |
| | | YTGYIAPFVSRKNLFVLANHTVSRIQFKPKNGTELLKAVGVEWYTTGDNSNKQTI |
| | | KARREVIVSSGSIGSPKLLEISGIGNKDIVTAAGVQSLIDLPGVGSNMQDHVHAVT |
| | | VSTTNITGFTTDSVFQNETLAEEQRQQYYNNKTGIWTTTPNNLGYPSPSQLFDGTS |
| | | FESGQAFANRIRNSTDQWAEYYASTNATNIELLKKQYAIVASRYEENYLSPIEINFT |
| | | PGYGGTTDVDLKNNKYQTVNHVLIAPLSRGYTHINSSNIEDPVVINPQYYTHPMD |
| | | VDVHIASTKLARRILGAEPGLASINSGEIQPGSNITSDEDVKQWLADNVRSDWHPV |
| | | GTCAMLPRELGGVVDPNLLVYGTANLRVVDASIMPLEISSHLMQPTYGVAEKAA |
| | | DIIKMSRKNNNN |
| Mucor_subtillisimus | 69 | MQQNGTSNDTYDYVIVGGGVGGLSLASRLSEDKGVTVAVLESGPYADDRFVVY |
| | | APGMYGQAVGTELCPLLPTVPQVGMNNRTITIATGRLLGGGSAVNGLVWTRGA |
| | | MKDFDAWEELGNPGWNGKTMFKYFKKIENFHPPTEEQVQYGATYQKNVHGSGG |
| | | PIDISFPVFEFPQSANWNASLAYLNFTHQQDLLNGSLHGYSTTPNTLNPETARRAD |
| | | AYAGYIQPNVNRTNLAVLANHTVSRIQFEKSNGSQPLKAIGVEWYTTGGDKSTK |
| | | QTIKARREVIISSGAIGSPKLLEVSGIGNKQIVTAAGVESLIDLPGVGSNMQDHVHA |
| | | VTVSTTNIEGYTTNSVFTNETLAQEQKDLYYNNKTGIWTTTPNNLGYPSPSQLFTN |
| | | TTFRSGKQFAAMIRNSTDKYAQYYASTKNATNIQLLKKQYAIVARRYEEDYISPIE |
| | | INFTPGYGGTGEVDLQNNKYQTVNHVLVAPLSRGYTHINSSDIEDPVVIDPQYYSH |
| | | PLDVDVHVASTQLARSILNAPALAAINSGEVEPGEKIQTDQDVRKWLSDNVRSD |
| | | WHPVGTCAMLPKGLGGVVDSNLKVYGTANLRVVDASIIPLEISSHLMQPVYAVS |
| | | ERAADIIKGSRN |
| Mucor_circinelloides | 70 | MQQDTNNTSSTDTYDYVIVGGGVAGLALASRISENKDVTVAVLESGPNAEDQFV |
| | | VYAPGMYGQAVGTELAPLVPTTPQENMGNRSLSIATGRLLGGGSAVNGLVWTR |
| | | GGLKDYDAWEELGNPGWNGSNLFKYFKKVENFHPPTPAQIEYGATYQKSAHGK |
| | | NGPIDVSFTNFEFPQSAKWNASLESLDFTALPDLLNGTLAGYSTTPNILDPETARR |
| | | VDAYAGYIVPYMGRNNLNVLANHTVSRIQFAPQNGSEPLKATGVEWYPTGNKD |
| | | QKQTIKARYEVIISSGAIGSPKLLEISGIGNKDIVTAAGVESLIDLPGVGANMQDHV |
| | | HAVTVSTTNIDGYTTNSVFTNETLAQEQREQYEANKTGIWTTTPNNLGYPTPEQL |
| | | FNGTEFVSGKEFAAKIRNSTDEWANYYASTNATNADLLKKQYAIVASRYEENYL |
| | | SPIEINLTPGYGGTGSPDLQNNKYQTVNHVLIAPLSRGYAHINSSDIEEPSVINPQY |
| | | YSHPLDIDVHVASTKLAREIITASPGLGDLNSGEVEPGMNVTSEDDLRSWLSNNV |
| | | RSDWHPVGTCAMLPQELGGVVSPALMVYGTSNLRVVDASIMPLEVSSHLMQPTY |
| | | GIAEKAADIIKNYYKSQYSGAGKN |
| Mucor_ambiguus_ | 71 | MQQDTNTSSTDTYDYVIVGGGVAGLALASRISENKDVTVAVLESGPNANDQFVV |
| GAN06663 | | YAPGMYGQAVGTDLCPLVPTTPQENMGNRSLSIATGRLLGGGSAVNGLVWTRG |
| | | GLKDYDAWEELGNPGWNGSNLFKYFKKVENFHPPTPAQIEYGATYQKNAHGKN |
| | | GPIDVSFTNYEFPQSAKWNASLSSLDFTALPDLLNGTLAGYSTTPNILDPETVQRV |
| | | DSYAGYIAPYTSRSNLNVLANHTVSRIQFAPQNGSEPLKATGVEWYPTGNKDQK |
| | | QTIKARYEVIISSGAIGSPKLLEISGIGSKDIVSAAGVESLIDLPGVGSNMQDHVHAV |
| | | TVSTTNITGYTTNSVFVNETLAQEQREEYETNKTGIWTTTPNNLGYPTPEQLENGT |
| | | EFVSGKEFADKIRNSTDEWANYYASTNATNVELLKKQYAIVASRYEENYLSPIEIN |
| | | LTPGYGGTGSPDLQNNKYQTVNHVLIAPLSRGYTHINSSDIEEPSVINPQYYSHPM |
| | | DIDVHVASTKLAREIITASPGLGDLNSGEVEPGMNITSEDDLRSWLSNNVRSDWHP |
| | | VGTCAMLPKELGGVVSPALMVYGTSNLRVVDASIMPLEVSSHLMQPTYGIAEKA |
| | | ADIIKNAYKNQYKN |
| Mucor_lusitanicus | 72 | Mqqdtnntsstdtydyvivgggvaglalasrisenkdvtvavlesgpnaedqfvvyapgmygqavgtelaplvpttpqen |
| | | mgnrslsiatgrllgggsavnglvwtrgglkdydaweelgnpgwngsnlfkyfkkvenfhpptpaqieygatyqksahgk |
| | | ngpidvsftnfefpqsakwnaslesldftalpdllngtlagysttpnildpetarrvdayagyivpymgrnnlnvlanhtvsriqf |
| | | apqngseplkatgvewyptgnkdqkqtikaryeviissgaigspklleisgignkdivtaagveslidlpgvganmqdhvha |
| | | vtvsttnidgyttnsvftnetlaqeqreqyeanktgiwtttpnnlgyptpeqlfngtefvsgkefaakirnstdewanyyastnat |
| | | nadllkkqyaivasryeenylspieinltpgyggtgspdlqnnkyqtvnhvliaplsrgyahinssdieepsvinpqyyshpld |
| | | idvhvastklareiitaspglgdlnsgevepgmnvtseddlrswlsnnvrsdwhpvgtcamlpqelggvvspalmvygtsn |
| | | lrvvdasimplevsshlmqptygiaekaadii |
| Aspergillus_flavus_ | 73 | Mlfslaflsalslataspagrakntttydyivvgggtsglvvanrlsenpdvsvllleagasvfnnpdvtnangyglafgsaidw |
| FAD-GDH | | qyqsinqsyaggkqqvlragkalggtstingmaytraedvqidvwqklgnegwtwkdllpyylksenltaptssqvaagaa |
| | | ynpavngkegplkvgwsgslasgnlsvalnrtfqaagvpwvedvnggkmrgfniypstldvdlnvredaarayyfpyddr |
| | | knlhllenttanrlfwkngsaeeaiadgveitsadgkvtrvhakkeviisagalrsplilelsgvgnptilkknnitprvdlptvge |
| | | nlqdqfnngmagegygvlagastvtypsisdvfgnetdsivaslrsqlsdyaaatvkvsnghmkqedlerlyqlqfdlivkd |
| | | kvpiaeilfhpgggnavssefwgllpfargnihissndptapaainpnyfmfewdgksqagiakyirkilrsaplnkliaketk |
| | | pglseipataadekwvewlkanyrsnfhpvgtaammprsiggvvdnrlrvygtsnvrvvdasvlpfqvcghlvstlyavae |
| | | rasdlikedaksa |
| Aspergillus_niger_ | 74 | Mtdpkdvsgrtvdyiiagggltglttaarltenpnisvlviesgsyesdrgpiiedlnaygdifgssvdhayetvelatnnqtalir |
| GOX | | sgnglggstlvnggtwtrphkaqvdswetvfgnegwnwdnvaayslqaerarapnakqiaaghyfnaschgvngtvhag |
| | | prdtgddyspivkalmsavedrgvptkkdfgcgdphgvsmfpntlhedqvrsdaarewllpnyqrpnlqvltgqyvgkvll |
| | | sqngttpravgvefgthkgnthnvyakhevllaagsavsptileysgigmksileplgidtvvdlpvglnlqdqttatvrsritsa |
| | | gagqgqaawfatfnetfgdysekahellntkleqwaeeavarggfhnttalliqyenyrdwivnhnvayselfldtagvasfd |
| | | vwdllpftrgyvhildkdpylhhfaydpqyflneldllgqaaatqlarnisnsgamqtyfagetipgdnlaydadlsawteyip |
| | | yhfrpnyhgvgtcsmmpkemggvvdnaarvygvqglrvidgsipptqmsshvmtvfyamalkisdailedyasmq |
| praHA341 | 75 | MQDTNSSSTDTYDYVIVGGGVAGLALASRISENKDVTVAVLESGPNANDRFVVY |
| | | APGMYGQAVGTDLCPLIPTTPQENMGNRSLTIATGRLLGGGSAINGLVWTRGGLK |
| | | DYDAWEELGNPGWNGANLFKYFKKVENFTPPTPAQIEYGATYQKSAHGKKGPID |
| | | VSFTNYEFSQSASWNASLETLDFTALPDILNGTLAGYSTTPNILDPETVQRVDSYT |
| | | GYIAPYTSRNNLNVLANHTVSRIQFAPKNGSEPLKATGVEWYPTGNKNQKQIIKA |
| | | RYEVIISSGAIGSPKLLEISGIGNKDIVSAAGVESLIDLPGVGSNMQDHVHAITVSTT |
| | | NITGYTTNSVFYPYDVPDYAVNETLAQEQREEYEANKTGIWATTPNNLGYPTPEQ |
| | | LENGTEFVSGKEFADKIRNSTDEWANYYASTNASNVELLKKQYAIVASRYEENYL |
| | | SPIEINFTPGYEGSGNVDLQNNKYQTVNHVLIAPLSRGYTHINSSDVEDHSVINPQY |
| | | YSHPMDIDVHIASTKLAREIITASPGLGDINSGEIEPGMNITSEDDLRSWLSNNVRS |
| | | DWHPVGTCAMLPKELGGVVSPALMVYGTSNLRVVDASIMPLEVSSHLMQPTYGI |
| | | AEKAADIIKNFYKTQHKNQN |
| praHA358 | 76 | MQDTNSSSTDTYDYVIVGGGVAGLALASRISENKDVTVAVLESGPNANDRFVVY |
| | | APGMYGQAVGTDLCPLIPTTPQENMGNRSLTIATGRLLGGGSAINGLVWTRGGLK |
| | | DYDAWEELGNPGWNGANLFKYFKKVENFTPPTPAQIEYGATYQKSAHGKKGPID |
| | | VSFTNYEFSQSASWNASLETLDFTALPDILNGTLAGYSTTPNILDPETVQRVDSYT |
| | | GYIAPYTSRNNLNVLANHTVSRIQFAPKNGSEPLKATGVEWYPTGNKNQKQIIKA |
| | | RYEVIISSGAIGSPKLLEISGIGNKDIVSAAGVESLIDLPGVGSNMQDHVHAITVSTT |
| | | NITGYTTNSVFVNETLAQEQREEYEANKYPYDVPDYATGIWATTPNNLGYPTPEQ |
| | | LENGTEFVSGKEFADKIRNSTDEWANYYASTNASNVELLKKQYAIVASRYEENYL |
| | | SPIEINFTPGYEGSGNVDLQNNKYQTVNHVLIAPLSRGYTHINSSDVEDHSVINPQY |
| | | YSHPMDIDVHIASTKLAREIITASPGLGDINSGEIEPGMNITSEDDLRSWLSNNVRS |
| | | DWHPVGTCAMLPKELGGVVSPALMVYGTSNLRVVDASIMPLEVSSHLMQPTYGI |
| | | AEKAADIIKNFYKTQHKNQN |
| praHNL341 | 77 | MQDTNSSSTDTYDYVIVGGGVAGLALASRISENKDVTVAVLESGPNANDRFVVY |
| | | APGMYGQAVGTDLCPLIPTTPQENMGNRSLTIATGRLLGGGSAINGLVWTRGGLK |
| | | DYDAWEELGNPGWNGANLFKYFKKVENFTPPTPAQIEYGATYQKSAHGKKGPID |
| | | VSFTNYEFSQSASWNASLETLDFTALPDILNGTLAGYSTTPNILDPETVQRVDSYT |
| | | GYIAPYTSRNNLNVLANHTVSRIQFAPKNGSEPLKATGVEWYPTGNKNQKQIIKA |
| | | RYEVIISSGAIGSPKLLEISGIGNKDIVSAAGVESLIDLPGVGSNMQDHVHAITVSTT |
| | | NITGYTTNSVFVNETLAQEQREEYEANKQPGEFTLGNTGIWATTPNNLGYPTPEQ |
| | | LENGTEFVSGKEFADKIRNSTDEWANYYASTNASNVELLKKQYAIVASRYEENYL |
| | | SPIEINFTPGYEGSGNVDLQNNKYQTVNHVLIAPLSRGYTHINSSDVEDHSVINPQY |
| | | YSHPMDIDVHIASTKLAREIITASPGLGDINSGEIEPGMNITSEDDLRSWLSNNVRS |
| | | DWHPVGTCAMLPKELGGVVSPALMVYGTSNLRVVDASIMPLEVSSHLMQPTYGI |
| | | AEKAADIIKNFYKTQHKNQN |
| praTNI341 | 78 | MQDTNSSSTDTYDYVIVGGGVAGLALASRISENKDVTVAVLESGPNANDRFVVY |
| | | APGMYGQAVGTDLCPLIPTTPQENMGNRSLTIATGRLLGGGSAINGLVWTRGGLK |
| | | DYDAWEELGNPGWNGANLFKYFKKVENFTPPTPAQIEYGATYQKSAHGKKGPID |
| | | VSFTNYEFSQSASWNASLETLDFTALPDILNGTLAGYSTTPNILDPETVQRVDSYT |
| | | GYIAPYTSRNNLNVLANHTVSRIQFAPKNGSEPLKATGVEWYPTGNKNQKQIIKA |
| | | RYEVIISSGAIGSPKLLEISGIGNKDIVSAAGVESLIDLPGVGSNMQDHVHAITVSTT |
| | | NITGYTTNSVFISASRKLQLVNETLAQEQREEYEANKTGIWATTPNNLGYPTPEQL |
| | | FNGTEFVSGKEFADKIRNSTDEWANYYASTNASNVELLKKQYAIVASRYEENYLS |
| | | PIEINFTPGYEGSGNVDLQNNKYQTVNHVLIAPLSRGYTHINSSDVEDHSVINPQY |
| | | YSHPMDIDVHIASTKLAREIITASPGLGDINSGEIEPGMNITSEDDLRSWLSNNVRS |
| | | DWHPVGTCAMLPKELGGVVSPALMVYGTSNLRVVDASIMPLEVSSHLMQPTYGI |
| | | AEKAADIIKNFYKTQHKNQN |
| praTNI358 | 79 | MQDTNSSSTDTYDYVIVGGGVAGLALASRISENKDVTVAVLESGPNANDRFVVY |
| | | APGMYGQAVGTDLCPLIPTTPQENMGNRSLTIATGRLLGGGSAINGLVWTRGGLK |
| | | DYDAWEELGNPGWNGANLFKYFKKVENFTPPTPAQIEYGATYQKSAHGKKGPID |
| | | VSFTNYEFSQSASWNASLETLDFTALPDILNGTLAGYSTTPNILDPETVQRVDSYT |
| | | GYIAPYTSRNNLNVLANHTVSRIQFAPKNGSEPLKATGVEWYPTGNKNQKQIIKA |
| | | RYEVIISSGAIGSPKLLEISGIGNKDIVSAAGVESLIDLPGVGSNMQDHVHAITVSTT |
| | | NITGYTTNSVFVNETLAQEQREEYEANKISASRKLQLTGIWATTPNNLGYPTPEQL |
| | | FNGTEFVSGKEFADKIRNSTDEWANYYASTNASNVELLKKQYAIVASRYEENYLS |
| | | PIEINFTPGYEGSGNVDLQNNKYQTVNHVLIAPLSRGYTHINSSDVEDHSVINPQY |
| | | YSHPMDIDVHIASTKLAREIITASPGLGDINSGEIEPGMNITSEDDLRSWLSNNVRS |
| | | DWHPVGTCAMLPKELGGVVSPALMVYGTSNLRVVDASIMPLEVSSHLMQPTYGI |
| | | AEKAADIIKNFYKTQHKNQN |
| guiHA341 | 80 | MQSNTDTYDYVIVGGGVGGLALANRLSENKQVTVAVLEAGPNANDEFIVYAPG |
| | | MYGQAVGTYLAPLRPTVPQENMNNRSLSIATGKLLGGGSAVNGLVWTRGATKD |
| | | FDAWEELGNPGWNGASMFKYFKKVENFTAPTPYQVNYGATYQKNTHGYKGPV |
| | | QVSFTNYEFPQSAHWNQSLASLGFDHLPDLLNGTLSGYSTTPNILDPNTDQRCDA |
| | | YAAYIAPYTARTNLHVLANHTVSRIEFNQTNANQPLVASGVEWYPTGDNTKKQTI |
| | | KARLEVIVSSGSIGSPKLLEISGIGNKDIVTAAGVKSLLDLPGVGSNMQDHVHAVT |
| | | VSTTNITGYTTDSVFVYPYDVPDYANSTLASEQREQYEKDKSGIWTTTPNNLGYP |
| | | TPAQLENGTEFMDGKAFAARIRNSSQEWAQYYASKNASTVELLMKQYEIVASRY |
| | | EENYLSPIEINLTPGYGGVGTVDKTKNKYQTVNHVLIAPLSRGFTHINSSDIEDPVN |
| | | INPQYYSHPMDIDVHVASTKLARRIINAPGLGDLNSGEVEPGMDITSDSDVRAWL |
| | | ANNVRSDWHPVGTCAMLPKELGGVVDSSLKVYGTANLRVVDASIMPLEVSSHL |
| | | MQPTFGVAEKAADIIKAEYKKQKAQ |
| guiHA358 | 81 | MQSNTDTYDYVIVGGGVGGLALANRLSENKQVTVAVLEAGPNANDEFIVYAPG |
| | | MYGQAVGTYLAPLRPTVPQENMNNRSLSIATGKLLGGGSAVNGLVWTRGATKD |
| | | FDAWEELGNPGWNGASMFKYFKKVENFTAPTPYQVNYGATYQKNTHGYKGPV |
| | | QVSFTNYEFPQSAHWNQSLASLGFDHLPDLLNGTLSGYSTTPNILDPNTDQRCDA |
| | | YAAYIAPYTARTNLHVLANHTVSRIEFNQTNANQPLVASGVEWYPTGDNTKKQTI |
| | | KARLEVIVSSGSIGSPKLLEISGIGNKDIVTAAGVKSLLDLPGVGSNMQDHVHAVT |
| | | VSTTNITGYTTDSVFVNSTLASEQREQYEKDKYPYDVPDYASGIWTTTPNNLGYP |
| | | TPAQLENGTEFMDGKAFAARIRNSSQEWAQYYASKNASTVELLMKQYEIVASRY |
| | | EENYLSPIEINLTPGYGGVGTVDKTKNKYQTVNHVLIAPLSRGFTHINSSDIEDPVN |
| | | INPQYYSHPMDIDVHVASTKLARRIINAPGLGDLNSGEVEPGMDITSDSDVRAWL |
| | | ANNVRSDWHPVGTCAMLPKELGGVVDSSLKVYGTANLRVVDASIMPLEVSSHL |
| | | MQPTFGVAEKAADIIKAEYKKQKAQ |
| guiHNL341 | 82 | MQSNTDTYDYVIVGGGVGGLALANRLSENKQVTVAVLEAGPNANDEFIVYAPG |
| | | MYGQAVGTYLAPLRPTVPQENMNNRSLSIATGKLLGGGSAVNGLVWTRGATKD |
| | | FDAWEELGNPGWNGASMFKYFKKVENFTAPTPYQVNYGATYQKNTHGYKGPV |
| | | QVSFTNYEFPQSAHWNQSLASLGFDHLPDLLNGTLSGYSTTPNILDPNTDQRCDA |
| | | YAAYIAPYTARTNLHVLANHTVSRIEFNQTNANQPLVASGVEWYPTGDNTKKQTI |
| | | KARLEVIVSSGSIGSPKLLEISGIGNKDIVTAAGVKSLLDLPGVGSNMQDHVHAVT |
| | | VSTTNITGYTTDSVFVQPGEFTLGNNSTLASEQREQYEKDKSGIWTTTPNNLGYPT |
| | | PAQLENGTEFMDGKAFAARIRNSSQEWAQYYASKNASTVELLMKQYEIVASRYE |
| | | ENYLSPIEINLTPGYGGVGTVDKTKNKYQTVNHVLIAPLSRGFTHINSSDIEDPVNI |
| | | NPQYYSHPMDIDVHVASTKLARRIINAPGLGDLNSGEVEPGMDITSDSDVRAWLA |
| | | NNVRSDWHPVGTCAMLPKELGGVVDSSLKVYGTANLRVVDASIMPLEVSSHLM |
| | | QPTFGVAEKAADIIKAEYKKQKAQ |
| guiHNL358 | 83 | MQSNTDTYDYVIVGGGVGGLALANRLSENKQVTVAVLEAGPNANDEFIVYAPG |
| | | MYGQAVGTYLAPLRPTVPQENMNNRSLSIATGKLLGGGSAVNGLVWTRGATKD |
| | | FDAWEELGNPGWNGASMFKYFKKVENFTAPTPYQVNYGATYQKNTHGYKGPV |
| | | QVSFTNYEFPQSAHWNQSLASLGFDHLPDLLNGTLSGYSTTPNILDPNTDQRCDA |
| | | YAAYIAPYTARTNLHVLANHTVSRIEFNQTNANQPLVASGVEWYPTGDNTKKQTI |
| | | KARLEVIVSSGSIGSPKLLEISGIGNKDIVTAAGVKSLLDLPGVGSNMQDHVHAVT |
| | | VSTTNITGYTTDSVFVNSTLASEQREQYEKDKQPGEFTLGNSGIWTTTPNNLGYPT |
| | | PAQLENGTEFMDGKAFAARIRNSSQEWAQYYASKNASTVELLMKQYEIVASRYE |
| | | ENYLSPIEINLTPGYGGVGTVDKTKNKYQTVNHVLIAPLSRGFTHINSSDIEDPVNI |
| | | NPQYYSHPMDIDVHVASTKLARRIINAPGLGDLNSGEVEPGMDITSDSDVRAWLA |
| | | NNVRSDWHPVGTCAMLPKELGGVVDSSLKVYGTANLRVVDASIMPLEVSSHLM |
| | | QPTFGVAEKAADIIKAEYKKQKAQ |
| guiTNI341 | 84 | MQSNTDTYDYVIVGGGVGGLALANRLSENKQVTVAVLEAGPNANDEFIVYAPG |
| | | MYGQAVGTYLAPLRPTVPQENMNNRSLSIATGKLLGGGSAVNGLVWTRGATKD |
| | | FDAWEELGNPGWNGASMFKYFKKVENFTAPTPYQVNYGATYQKNTHGYKGPV |
| | | QVSFTNYEFPQSAHWNQSLASLGFDHLPDLLNGTLSGYSTTPNILDPNTDQRCDA |
| | | YAAYIAPYTARTNLHVLANHTVSRIEFNQTNANQPLVASGVEWYPTGDNTKKQTI |
| | | KARLEVIVSSGSIGSPKLLEISGIGNKDIVTAAGVKSLLDLPGVGSNMQDHVHAVT |
| | | VSTTNITGYTTDSVFVISASRKLQLNSTLASEQREQYEKDKSGIWTTTPNNLGYPT |
| | | PAQLENGTEFMDGKAFAARIRNSSQEWAQYYASKNASTVELLMKQYEIVASRYE |
| | | ENYLSPIEINLTPGYGGVGTVDKTKNKYQTVNHVLIAPLSRGFTHINSSDIEDPVNI |
| | | NPQYYSHPMDIDVHVASTKLARRIINAPGLGDLNSGEVEPGMDITSDSDVRAWLA |
| | | NNVRSDWHPVGTCAMLPKELGGVVDSSLKVYGTANLRVVDASIMPLEVSSHLM |
| | | QPTFGVAEKAADIIKAEYKKQKAQ |
| guiTNI358 | 85 | MQSNTDTYDYVIVGGGVGGLALANRLSENKQVTVAVLEAGPNANDEFIVYAPG |
| | | MYGQAVGTYLAPLRPTVPQENMNNRSLSIATGKLLGGGSAVNGLVWTRGATKD |
| | | FDAWEELGNPGWNGASMFKYFKKVENFTAPTPYQVNYGATYQKNTHGYKGPV |
| | | QVSFTNYEFPQSAHWNQSLASLGFDHLPDLLNGTLSGYSTTPNILDPNTDQRCDA |
| | | YAAYIAPYTARTNLHVLANHTVSRIEFNQTNANQPLVASGVEWYPTGDNTKKQTI |
| | | KARLEVIVSSGSIGSPKLLEISGIGNKDIVTAAGVKSLLDLPGVGSNMQDHVHAVT |
| | | VSTTNITGYTTDSVFVNSTLASEQREQYEKDKISASRKLQLSGIWTTTPNNLGYPT |
| | | PAQLENGTEFMDGKAFAARIRNSSQEWAQYYASKNASTVELLMKQYEIVASRYE |
| | | ENYLSPIEINLTPGYGGVGTVDKTKNKYQTVNHVLIAPLSRGFTHINSSDIEDPVNI |
| | | NPQYYSHPMDIDVHVASTKLARRIINAPGLGDLNSGEVEPGMDITSDSDVRAWLA |
| | | NNVRSDWHPVGTCAMLPKELGGVVDSSLKVYGTANLRVVDASIMPLEVSSHLM |
| | | QPTFGVAEKAADIIKAEYKKQKAQ |
| hieHA341 | 86 | MQKTATSNTYDYVIVGGGVGGLALASRLSEDKSVTVAVLEAGPNADEQFVVYAP |
| | | GMYGQAVGTDLCPLRPTVPQEAMNNRTLTIATGKLLGGGSAINGLVWTRGALKD |
| | | FDAWEELGNPGWNGRTMFKYFKKVERFHPPTKAQVQYGATYQKGVHGKNGRI |
| | | DISFPEFQFPQSANWNASLATLDFTHQQDLLNGSLHGYSTTPNTLDPKTARRVDS |
| | | YTGYIAPFVSRKNLFVLANHTVSRIQFKPKNGTELLKAVGVEWYTTGDNSNKQTI |
| | | KARREVIVSSGSIGSPKLLEISGIGNKDIVTAAGVQSLIDLPGVGSNMQDHVHAVT |
| | | VSTTNITGFTTDSVFYPYDVPDYAQNETLAEEQRQQYYNNKTGIWTTTPNNLGYP |
| | | SPSQLFDGTSFESGQAFANRIRNSTDQWAEYYASTNATNIELLKKQYAIVASRYEE |
| | | NYLSPIEINFTPGYGGTTDVDLKNNKYQTVNHVLIAPLSRGYTHINSSNIEDPVVIN |
| | | PQYYTHPMDVDVHIASTKLARRILGAEPGLASINSGEIQPGSNITSDEDVKQWLAD |
| | | NVRSDWHPVGTCAMLPRELGGVVDPNLLVYGTANLRVVDASIMPLEISSHLMQP |
| | | TYGVAEKAADIIKMSRKNNNN |
| hieHA358 | 87 | MQKTATSNTYDYVIVGGGVGGLALASRLSEDKSVTVAVLEAGPNADEQFVVYAP |
| | | GMYGQAVGTDLCPLRPTVPQEAMNNRTLTIATGKLLGGGSAINGLVWTRGALKD |
| | | FDAWEELGNPGWNGRTMFKYFKKVERFHPPTKAQVQYGATYQKGVHGKNGRI |
| | | DISFPEFQFPQSANWNASLATLDFTHQQDLLNGSLHGYSTTPNTLDPKTARRVDS |
| | | YTGYIAPFVSRKNLFVLANHTVSRIQFKPKNGTELLKAVGVEWYTTGDNSNKQTI |
| | | KARREVIVSSGSIGSPKLLEISGIGNKDIVTAAGVQSLIDLPGVGSNMQDHVHAVT |
| | | VSTTNITGFTTDSVFQNETLAEEQRQQYYNNKYPYDVPDYATGIWTTTPNNLGYP |
| | | SPSQLFDGTSFESGQAFANRIRNSTDQWAEYYASTNATNIELLKKQYAIVASRYEE |
| | | NYLSPIEINFTPGYGGTTDVDLKNNKYQTVNHVLIAPLSRGYTHINSSNIEDPVVIN |
| | | PQYYTHPMDVDVHIASTKLARRILGAEPGLASINSGEIQPGSNITSDEDVKQWLAD |
| | | NVRSDWHPVGTCAMLPRELGGVVDPNLLVYGTANLRVVDASIMPLEISSHLMQP |
| | | TYGVAEKAADIIKMSRKNNNN |
| hieHNL341 | 88 | MQKTATSNTYDYVIVGGGVGGLALASRLSEDKSVTVAVLEAGPNADEQFVVYAP |
| | | GMYGQAVGTDLCPLRPTVPQEAMNNRTLTIATGKLLGGGSAINGLVWTRGALKD |
| | | FDAWEELGNPGWNGRTMFKYFKKVERFHPPTKAQVQYGATYQKGVHGKNGRI |
| | | DISFPEFQFPQSANWNASLATLDFTHQQDLLNGSLHGYSTTPNTLDPKTARRVDS |
| | | YTGYIAPFVSRKNLFVLANHTVSRIQFKPKNGTELLKAVGVEWYTTGDNSNKQTI |
| | | KARREVIVSSGSIGSPKLLEISGIGNKDIVTAAGVQSLIDLPGVGSNMQDHVHAVT |
| | | VSTTNITGFTTDSVFQPGEFTLGNQNETLAEEQRQQYYNNKTGIWTTTPNNLGYPS |
| | | PSQLFDGTSFESGQAFANRIRNSTDQWAEYYASTNATNIELLKKQYAIVASRYEEN |
| | | YLSPIEINFTPGYGGTTDVDLKNNKYQTVNHVLIAPLSRGYTHINSSNIEDPVVINP |
| | | QYYTHPMDVDVHIASTKLARRILGAEPGLASINSGEIQPGSNITSDEDVKQWLADN |
| | | VRSDWHPVGTCAMLPRELGGVVDPNLLVYGTANLRVVDASIMPLEISSHLMQPT |
| | | YGVAEKAADIIKMSRKNNNN |
| hieHNL358 | 89 | MQKTATSNTYDYVIVGGGVGGLALASRLSEDKSVTVAVLEAGPNADEQFVVYAP |
| | | GMYGQAVGTDLCPLRPTVPQEAMNNRTLTIATGKLLGGGSAINGLVWTRGALKD |
| | | FDAWEELGNPGWNGRTMFKYFKKVERFHPPTKAQVQYGATYQKGVHGKNGRI |
| | | DISFPEFQFPQSANWNASLATLDFTHQQDLLNGSLHGYSTTPNTLDPKTARRVDS |
| | | YTGYIAPFVSRKNLFVLANHTVSRIQFKPKNGTELLKAVGVEWYTTGDNSNKQTI |
| | | KARREVIVSSGSIGSPKLLEISGIGNKDIVTAAGVQSLIDLPGVGSNMQDHVHAVT |
| | | VSTTNITGFTTDSVFQNETLAEEQRQQYYNNKQPGEFTLGNTGIWTTTPNNLGYPS |
| | | PSQLFDGTSFESGQAFANRIRNSTDQWAEYYASTNATNIELLKKQYAIVASRYEEN |
| | | YLSPIEINFTPGYGGTTDVDLKNNKYQTVNHVLIAPLSRGYTHINSSNIEDPVVINP |
| | | QYYTHPMDVDVHIASTKLARRILGAEPGLASINSGEIQPGSNITSDEDVKQWLADN |
| | | VRSDWHPVGTCAMLPRELGGVVDPNLLVYGTANLRVVDASIMPLEISSHLMQPT |
| | | YGVAEKAADIIKMSRKNNNN |
| hieTNI341 | 90 | MQKTATSNTYDYVIVGGGVGGLALASRLSEDKSVTVAVLEAGPNADEQFVVYAP |
| | | GMYGQAVGTDLCPLRPTVPQEAMNNRTLTIATGKLLGGGSAINGLVWTRGALKD |
| | | FDAWEELGNPGWNGRTMFKYFKKVERFHPPTKAQVQYGATYQKGVHGKNGRI |
| | | DISFPEFQFPQSANWNASLATLDFTHQQDLLNGSLHGYSTTPNTLDPKTARRVDS |
| | | YTGYIAPFVSRKNLFVLANHTVSRIQFKPKNGTELLKAVGVEWYTTGDNSNKQTI |
| | | KARREVIVSSGSIGSPKLLEISGIGNKDIVTAAGVQSLIDLPGVGSNMQDHVHAVT |
| | | VSTTNITGFTTDSVFISASRKLQLQNETLAEEQRQQYYNNKTGIWTTTPNNLGYPS |
| | | PSQLFDGTSFESGQAFANRIRNSTDQWAEYYASTNATNIELLKKQYAIVASRYEEN |
| | | YLSPIEINFTPGYGGTTDVDLKNNKYQTVNHVLIAPLSRGYTHINSSNIEDPVVINP |
| | | QYYTHPMDVDVHIASTKLARRILGAEPGLASINSGEIQPGSNITSDEDVKQWLADN |
| | | VRSDWHPVGTCAMLPRELGGVVDPNLLVYGTANLRVVDASIMPLEISSHLMQPT |
| | | YGVAEKAADIIKMSRKNNNN |
| hieTNI358 | 91 | MQKTATSNTYDYVIVGGGVGGLALASRLSEDKSVTVAVLEAGPNADEQFVVYAP |
| | | GMYGQAVGTDLCPLRPTVPQEAMNNRTLTIATGKLLGGGSAINGLVWTRGALKD |
| | | FDAWEELGNPGWNGRTMFKYFKKVERFHPPTKAQVQYGATYQKGVHGKNGRI |
| | | DISFPEFQFPQSANWNASLATLDFTHQQDLLNGSLHGYSTTPNTLDPKTARRVDS |
| | | YTGYIAPFVSRKNLFVLANHTVSRIQFKPKNGTELLKAVGVEWYTTGDNSNKQTI |
| | | KARREVIVSSGSIGSPKLLEISGIGNKDIVTAAGVQSLIDLPGVGSNMQDHVHAVT |
| | | VSTTNITGFTTDSVFQNETLAEEQRQQYYNNKISASRKLQLTGIWTTTPNNLGYPS |
| | | PSQLFDGTSFESGQAFANRIRNSTDQWAEYYASTNATNIELLKKQYAIVASRYEEN |
| | | YLSPIEINFTPGYGGTTDVDLKNNKYQTVNHVLIAPLSRGYTHINSSNIEDPVVINP |
| | | QYYTHPMDVDVHIASTKLARRILGAEPGLASINSGEIQPGSNITSDEDVKQWLADN |
| | | VRSDWHPVGTCAMLPRELGGVVDPNLLVYGTANLRVVDASIMPLEISSHLMQPT |
| | | YGVAEKAADIIKMSRKNNNN |
| subHA341 | 92 | MQQNGTSNDTYDYVIVGGGVGGLSLASRLSEDKGVTVAVLESGPYADDRFVVY |
| | | APGMYGQAVGTELCPLLPTVPQVGMNNRTITIATGRLLGGGSAVNGLVWTRGA |
| | | MKDFDAWEELGNPGWNGKTMFKYFKKIENFHPPTEEQVQYGATYQKNVHGSGG |
| | | PIDISFPVFEFPQSANWNASLAYLNFTHQQDLLNGSLHGYSTTPNTLNPETARRAD |
| | | AYAGYIQPNVNRTNLAVLANHTVSRIQFEKSNGSQPLKAIGVEWYTTGGDKSTK |
| | | QTIKARREVIISSGAIGSPKLLEVSGIGNKQIVTAAGVESLIDLPGVGSNMQDHVHA |
| | | VTVSTTNIEGYTTNSVFYPYDVPDYATNETLAQEQKDLYYNNKTGIWTTTPNNLG |
| | | YPSPSQLFTNTTFRSGKQFAAMIRNSTDKYAQYYASTKNATNIQLLKKQYAIVAR |
| | | RYEEDYISPIEINFTPGYGGTGEVDLQNNKYQTVNHVLVAPLSRGYTHINSSDIEDP |
| | | VVIDPQYYSHPLDVDVHVASTQLARSILNAPALAAINSGEVEPGEKIQTDQDVRK |
| | | WLSDNVRSDWHPVGTCAMLPKGLGGVVDSNLKVYGTANLRVVDASIIPLEISSH |
| | | LMQPVYAVSERAADIIKGSRN |
| subHA358 | 93 | MQQNGTSNDTYDYVIVGGGVGGLSLASRLSEDKGVTVAVLESGPYADDRFVVY |
| | | APGMYGQAVGTELCPLLPTVPQVGMNNRTITIATGRLLGGGSAVNGLVWTRGA |
| | | MKDFDAWEELGNPGWNGKTMFKYFKKIENFHPPTEEQVQYGATYQKNVHGSGG |
| | | PIDISFPVFEFPQSANWNASLAYLNFTHQQDLLNGSLHGYSTTPNTLNPETARRAD |
| | | AYAGYIQPNVNRTNLAVLANHTVSRIQFEKSNGSQPLKAIGVEWYTTGGDKSTK |
| | | QTIKARREVIISSGAIGSPKLLEVSGIGNKQIVTAAGVESLIDLPGVGSNMQDHVHA |
| | | VTVSTTNIEGYTTNSVFTNETLAQEQKDLYYNNKYPYDVPDYATGIWTTTPNNLG |
| | | YPSPSQLFTNTTFRSGKQFAAMIRNSTDKYAQYYASTKNATNIQLLKKQYAIVAR |
| | | RYEEDYISPIEINFTPGYGGTGEVDLQNNKYQTVNHVLVAPLSRGYTHINSSDIEDP |
| | | VVIDPQYYSHPLDVDVHVASTQLARSILNAPALAAINSGEVEPGEKIQTDQDVRK |
| | | WLSDNVRSDWHPVGTCAMLPKGLGGVVDSNLKVYGTANLRVVDASIIPLEISSH |
| | | LMQPVYAVSERAADIIKGSRN |
| subHNL341 | 94 | MQQNGTSNDTYDYVIVGGGVGGLSLASRLSEDKGVTVAVLESGPYADDRFVVY |
| | | APGMYGQAVGTELCPLLPTVPQVGMNNRTITIATGRLLGGGSAVNGLVWTRGA |
| | | MKDFDAWEELGNPGWNGKTMFKYFKKIENFHPPTEEQVQYGATYQKNVHGSGG |
| | | PIDISFPVFEFPQSANWNASLAYLNFTHQQDLLNGSLHGYSTTPNTLNPETARRAD |
| | | AYAGYIQPNVNRTNLAVLANHTVSRIQFEKSNGSQPLKAIGVEWYTTGGDKSTK |
| | | QTIKARREVIISSGAIGSPKLLEVSGIGNKQIVTAAGVESLIDLPGVGSNMQDHVHA |
| | | VTVSTTNIEGYTTNSVFQPGEFTLGNTNETLAQEQKDLYYNNKTGIWTTTPNNLG |
| | | YPSPSQLFTNTTFRSGKQFAAMIRNSTDKYAQYYASTKNATNIQLLKKQYAIVAR |
| | | RYEEDYISPIEINFTPGYGGTGEVDLQNNKYQTVNHVLVAPLSRGYTHINSSDIEDP |
| | | VVIDPQYYSHPLDVDVHVASTQLARSILNAPALAAINSGEVEPGEKIQTDQDVRK |
| | | WLSDNVRSDWHPVGTCAMLPKGLGGVVDSNLKVYGTANLRVVDASIIPLEISSH |
| | | LMQPVYAVSERAADIIKGSRN |
| subHNL358 | 95 | MQQNGTSNDTYDYVIVGGGVGGLSLASRLSEDKGVTVAVLESGPYADDRFVVY |
| | | APGMYGQAVGTELCPLLPTVPQVGMNNRTITIATGRLLGGGSAVNGLVWTRGA |
| | | MKDFDAWEELGNPGWNGKTMFKYFKKIENFHPPTEEQVQYGATYQKNVHGSGG |
| | | PIDISFPVFEFPQSANWNASLAYLNFTHQQDLLNGSLHGYSTTPNTLNPETARRAD |
| | | AYAGYIQPNVNRTNLAVLANHTVSRIQFEKSNGSQPLKAIGVEWYTTGGDKSTK |
| | | QTIKARREVIISSGAIGSPKLLEVSGIGNKQIVTAAGVESLIDLPGVGSNMQDHVHA |
| | | VTVSTTNIEGYTTNSVFTNETLAQEQKDLYYNNKQPGEFTLGNTGIWTTTPNNLG |
| | | YPSPSQLFTNTTFRSGKQFAAMIRNSTDKYAQYYASTKNATNIQLLKKQYAIVAR |
| | | RYEEDYISPIEINFTPGYGGTGEVDLQNNKYQTVNHVLVAPLSRGYTHINSSDIEDP |
| | | VVIDPQYYSHPLDVDVHVASTQLARSILNAPALAAINSGEVEPGEKIQTDQDVRK |
| | | WLSDNVRSDWHPVGTCAMLPKGLGGVVDSNLKVYGTANLRVVDASIIPLEISSH |
| | | LMQPVYAVSERAADIIKGSRN |
| subTNI341 | 96 | MQQNGTSNDTYDYVIVGGGVGGLSLASRLSEDKGVTVAVLESGPYADDRFVVY |
| | | APGMYGQAVGTELCPLLPTVPQVGMNNRTITIATGRLLGGGSAVNGLVWTRGA |
| | | MKDFDAWEELGNPGWNGKTMFKYFKKIENFHPPTEEQVQYGATYQKNVHGSGG |
| | | PIDISFPVFEFPQSANWNASLAYLNFTHQQDLLNGSLHGYSTTPNTLNPETARRAD |
| | | AYAGYIQPNVNRTNLAVLANHTVSRIQFEKSNGSQPLKAIGVEWYTTGGDKSTK |
| | | QTIKARREVIISSGAIGSPKLLEVSGIGNKQIVTAAGVESLIDLPGVGSNMQDHVHA |
| | | VTVSTTNIEGYTTNSVFISASRKLQLTNETLAQEQKDLYYNNKTGIWTTTPNNLGY |
| | | PSPSQLFTNTTFRSGKQFAAMIRNSTDKYAQYYASTKNATNIQLLKKQYAIVARR |
| | | YEEDYISPIEINFTPGYGGTGEVDLQNNKYQTVNHVLVAPLSRGYTHINSSDIEDPV |
| | | VIDPQYYSHPLDVDVHVASTQLARSILNAPALAAINSGEVEPGEKIQTDQDVRKW |
| | | LSDNVRSDWHPVGTCAMLPKGLGGVVDSNLKVYGTANLRVVDASIIPLEISSHLM |
| | | QPVYAVSERAADIIKGSRN |
| subTNI358 | 97 | MQQNGTSNDTYDYVIVGGGVGGLSLASRLSEDKGVTVAVLESGPYADDRFVVY |
| | | APGMYGQAVGTELCPLLPTVPQVGMNNRTITIATGRLLGGGSAVNGLVWTRGA |
| | | MKDFDAWEELGNPGWNGKTMFKYFKKIENFHPPTEEQVQYGATYQKNVHGSGG |
| | | PIDISFPVFEFPQSANWNASLAYLNFTHQQDLLNGSLHGYSTTPNTLNPETARRAD |
| | | AYAGYIQPNVNRTNLAVLANHTVSRIQFEKSNGSQPLKAIGVEWYTTGGDKSTK |
| | | QTIKARREVIISSGAIGSPKLLEVSGIGNKQIVTAAGVESLIDLPGVGSNMQDHVHA |
| | | VTVSTTNIEGYTTNSVFTNETLAQEQKDLYYNNKISASRKLQLTGIWTTTPNNLGY |
| | | PSPSQLFTNTTFRSGKQFAAMIRNSTDKYAQYYASTKNATNIQLLKKQYAIVARR |
| | | YEEDYISPIEINFTPGYGGTGEVDLQNNKYQTVNHVLVAPLSRGYTHINSSDIEDPV |
| | | VIDPQYYSHPLDVDVHVASTQLARSILNAPALAAINSGEVEPGEKIQTDQDVRKW |
| | | LSDNVRSDWHPVGTCAMLPKGLGGVVDSNLKVYGTANLRVVDASIIPLEISSHLM |
| | | QPVYAVSERAADIIKGSRN |
| cirHA341 | 98 | MQQDTNNTSSTDTYDYVIVGGGVAGLALASRISENKDVTVAVLESGPNAEDQFV |
| | | VYAPGMYGQAVGTELAPLVPTTPQENMGNRSLSIATGRLLGGGSAVNGLVWTR |
| | | GGLKDYDAWEELGNPGWNGSNLFKYFKKVENFHPPTPAQIEYGATYQKSAHGK |
| | | NGPIDVSFTNFEFPQSAKWNASLESLDFTALPDLLNGTLAGYSTTPNILDPETARR |
| | | VDAYAGYIVPYMGRNNLNVLANHTVSRIQFAPQNGSEPLKATGVEWYPTGNKD |
| | | QKQTIKARYEVIISSGAIGSPKLLEISGIGNKDIVTAAGVESLIDLPGVGANMQDHV |
| | | HAVTVSTTNIDGYTTNSVFYPYDVPDYATNETLAQEQREQYEANKTGIWTTTPNN |
| | | LGYPTPEQLENGTEFVSGKEFAAKIRNSTDEWANYYASTNATNADLLKKQYAIVA |
| | | SRYEENYLSPIEINLTPGYGGTGSPDLQNNKYQTVNHVLIAPLSRGYAHINSSDIEE |
| | | PSVINPQYYSHPLDIDVHVASTKLAREIITASPGLGDLNSGEVEPGMNVTSEDDLRS |
| | | WLSNNVRSDWHPVGTCAMLPQELGGVVSPALMVYGTSNLRVVDASIMPLEVSS |
| | | HLMQPTYGIAEKAADIIKNYYKSQYSGAGKN |
| cirHA358 | 99 | MQQDTNNTSSTDTYDYVIVGGGVAGLALASRISENKDVTVAVLESGPNAEDQFV |
| | | VYAPGMYGQAVGTELAPLVPTTPQENMGNRSLSIATGRLLGGGSAVNGLVWTR |
| | | GGLKDYDAWEELGNPGWNGSNLFKYFKKVENFHPPTPAQIEYGATYQKSAHGK |
| | | NGPIDVSFTNFEFPQSAKWNASLESLDFTALPDLLNGTLAGYSTTPNILDPETARR |
| | | VDAYAGYIVPYMGRNNLNVLANHTVSRIQFAPQNGSEPLKATGVEWYPTGNKD |
| | | QKQTIKARYEVIISSGAIGSPKLLEISGIGNKDIVTAAGVESLIDLPGVGANMQDHV |
| | | HAVTVSTTNIDGYTTNSVFTNETLAQEQREQYEANKYPYDVPDYATGIWTTTPNN |
| | | LGYPTPEQLENGTEFVSGKEFAAKIRNSTDEWANYYASTNATNADLLKKQYAIVA |
| | | SRYEENYLSPIEINLTPGYGGTGSPDLQNNKYQTVNHVLIAPLSRGYAHINSSDIEE |
| | | PSVINPQYYSHPLDIDVHVASTKLAREIITASPGLGDLNSGEVEPGMNVTSEDDLRS |
| | | WLSNNVRSDWHPVGTCAMLPQELGGVVSPALMVYGTSNLRVVDASIMPLEVSS |
| | | HLMQPTYGIAEKAADIIKNYYKSQYSGAGKN |
| cirHNL341 | 100 | MQQDTNNTSSTDTYDYVIVGGGVAGLALASRISENKDVTVAVLESGPNAEDQFV |
| | | VYAPGMYGQAVGTELAPLVPTTPQENMGNRSLSIATGRLLGGGSAVNGLVWTR |
| | | GGLKDYDAWEELGNPGWNGSNLFKYFKKVENFHPPTPAQIEYGATYQKSAHGK |
| | | NGPIDVSFTNFEFPQSAKWNASLESLDFTALPDLLNGTLAGYSTTPNILDPETARR |
| | | VDAYAGYIVPYMGRNNLNVLANHTVSRIQFAPQNGSEPLKATGVEWYPTGNKD |
| | | QKQTIKARYEVIISSGAIGSPKLLEISGIGNKDIVTAAGVESLIDLPGVGANMQDHV |
| | | HAVTVSTTNIDGYTTNSVFQPGEFTLGNTNETLAQEQREQYEANKTGIWTTTPNN |
| | | LGYPTPEQLENGTEFVSGKEFAAKIRNSTDEWANYYASTNATNADLLKKQYAIVA |
| | | SRYEENYLSPIEINLTPGYGGTGSPDLQNNKYQTVNHVLIAPLSRGYAHINSSDIEE |
| | | PSVINPQYYSHPLDIDVHVASTKLAREIITASPGLGDLNSGEVEPGMNVTSEDDLRS |
| | | WLSNNVRSDWHPVGTCAMLPQELGGVVSPALMVYGTSNLRVVDASIMPLEVSS |
| | | HLMQPTYGIAEKAADIIKNYYKSQYSGAGKN |
| cirHNL358 | 101 | MQQDTNNTSSTDTYDYVIVGGGVAGLALASRISENKDVTVAVLESGPNAEDQFV |
| | | VYAPGMYGQAVGTELAPLVPTTPQENMGNRSLSIATGRLLGGGSAVNGLVWTR |
| | | GGLKDYDAWEELGNPGWNGSNLFKYFKKVENFHPPTPAQIEYGATYQKSAHGK |
| | | NGPIDVSFTNFEFPQSAKWNASLESLDFTALPDLLNGTLAGYSTTPNILDPETARR |
| | | VDAYAGYIVPYMGRNNLNVLANHTVSRIQFAPQNGSEPLKATGVEWYPTGNKD |
| | | QKQTIKARYEVIISSGAIGSPKLLEISGIGNKDIVTAAGVESLIDLPGVGANMQDHV |
| | | HAVTVSTTNIDGYTTNSVFTNETLAQEQREQYEANKQPGEFTLGNTGIWTTTPNN |
| | | LGYPTPEQLENGTEFVSGKEFAAKIRNSTDEWANYYASTNATNADLLKKQYAIVA |
| | | SRYEENYLSPIEINLTPGYGGTGSPDLQNNKYQTVNHVLIAPLSRGYAHINSSDIEE |
| | | PSVINPQYYSHPLDIDVHVASTKLAREIITASPGLGDLNSGEVEPGMNVTSEDDLRS |
| | | WLSNNVRSDWHPVGTCAMLPQELGGVVSPALMVYGTSNLRVVDASIMPLEVSS |
| | | HLMQPTYGIAEKAADIIKNYYKSQYSGAGKN |
| cirTNI341 | 102 | MQQDTNNTSSTDTYDYVIVGGGVAGLALASRISENKDVTVAVLESGPNAEDQFV |
| | | VYAPGMYGQAVGTELAPLVPTTPQENMGNRSLSIATGRLLGGGSAVNGLVWTR |
| | | GGLKDYDAWEELGNPGWNGSNLFKYFKKVENFHPPTPAQIEYGATYQKSAHGK |
| | | NGPIDVSFTNFEFPQSAKWNASLESLDFTALPDLLNGTLAGYSTTPNILDPETARR |
| | | VDAYAGYIVPYMGRNNLNVLANHTVSRIQFAPQNGSEPLKATGVEWYPTGNKD |
| | | QKQTIKARYEVIISSGAIGSPKLLEISGIGNKDIVTAAGVESLIDLPGVGANMQDHV |
| | | HAVTVSTTNIDGYTTNSVFISASRKLQLTNETLAQEQREQYEANKTGIWTTTPNNL |
| | | GYPTPEQLENGTEFVSGKEFAAKIRNSTDEWANYYASTNATNADLLKKQYAIVAS |
| | | RYEENYLSPIEINLTPGYGGTGSPDLQNNKYQTVNHVLIAPLSRGYAHINSSDIEEP |
| | | SVINPQYYSHPLDIDVHVASTKLAREIITASPGLGDLNSGEVEPGMNVTSEDDLRS |
| | | WLSNNVRSDWHPVGTCAMLPQELGGVVSPALMVYGTSNLRVVDASIMPLEVSS |
| | | HLMQPTYGIAEKAADIIKNYYKSQYSGAGKN |
| cirTNI358 | 103 | MQQDTNNTSSTDTYDYVIVGGGVAGLALASRISENKDVTVAVLESGPNAEDQFV |
| | | VYAPGMYGQAVGTELAPLVPTTPQENMGNRSLSIATGRLLGGGSAVNGLVWTR |
| | | GGLKDYDAWEELGNPGWNGSNLFKYFKKVENFHPPTPAQIEYGATYQKSAHGK |
| | | NGPIDVSFTNFEFPQSAKWNASLESLDFTALPDLLNGTLAGYSTTPNILDPETARR |
| | | VDAYAGYIVPYMGRNNLNVLANHTVSRIQFAPQNGSEPLKATGVEWYPTGNKD |
| | | QKQTIKARYEVIISSGAIGSPKLLEISGIGNKDIVTAAGVESLIDLPGVGANMQDHV |
| | | HAVTVSTTNIDGYTTNSVFTNETLAQEQREQYEANKISASRKLQLTGIWTTTPNNL |
| | | GYPTPEQLENGTEFVSGKEFAAKIRNSTDEWANYYASTNATNADLLKKQYAIVAS |
| | | RYEENYLSPIEINLTPGYGGTGSPDLQNNKYQTVNHVLIAPLSRGYAHINSSDIEEP |
| | | SVINPQYYSHPLDIDVHVASTKLAREIITASPGLGDLNSGEVEPGMNVTSEDDLRS |
| | | WLSNNVRSDWHPVGTCAMLPQELGGVVSPALMVYGTSNLRVVDASIMPLEVSS |
| | | HLMQPTYGIAEKAADIIKNYYKSQYSGAGKN |
| ambHA341 | 104 | MQQDTNTSSTDTYDYVIVGGGVAGLALASRISENKDVTVAVLESGPNANDQFVV |
| | | YAPGMYGQAVGTDLCPLVPTTPQENMGNRSLSIATGRLLGGGSAVNGLVWTRG |
| | | GLKDYDAWEELGNPGWNGSNLFKYFKKVENFHPPTPAQIEYGATYQKNAHGKN |
| | | GPIDVSFTNYEFPQSAKWNASLSSLDFTALPDLLNGTLAGYSTTPNILDPETVQRV |
| | | DSYAGYIAPYTSRSNLNVLANHTVSRIQFAPQNGSEPLKATGVEWYPTGNKDQK |
| | | QTIKARYEVIISSGAIGSPKLLEISGIGSKDIVSAAGVESLIDLPGVGSNMQDHVHAV |
| | | TVSTTNITGYTTNSVFYPYDVPDYAVNETLAQEQREEYETNKTGIWTTTPNNLGY |
| | | PTPEQLENGTEFVSGKEFADKIRNSTDEWANYYASTNATNVELLKKQYAIVASRY |
| | | EENYLSPIEINLTPGYGGTGSPDLQNNKYQTVNHVLIAPLSRGYTHINSSDIEEPSVI |
| | | NPQYYSHPMDIDVHVASTKLAREIITASPGLGDLNSGEVEPGMNITSEDDLRSWLS |
| | | NNVRSDWHPVGTCAMLPKELGGVVSPALMVYGTSNLRVVDASIMPLEVSSHLM |
| | | QPTYGIAEKAADIIKNAYKNQYKN |
| ambHA358 | 105 | MQQDTNTSSTDTYDYVIVGGGVAGLALASRISENKDVTVAVLESGPNANDQFVV |
| | | YAPGMYGQAVGTDLCPLVPTTPQENMGNRSLSIATGRLLGGGSAVNGLVWTRG |
| | | GLKDYDAWEELGNPGWNGSNLFKYFKKVENFHPPTPAQIEYGATYQKNAHGKN |
| | | GPIDVSFTNYEFPQSAKWNASLSSLDFTALPDLLNGTLAGYSTTPNILDPETVQRV |
| | | DSYAGYIAPYTSRSNLNVLANHTVSRIQFAPQNGSEPLKATGVEWYPTGNKDQK |
| | | QTIKARYEVIISSGAIGSPKLLEISGIGSKDIVSAAGVESLIDLPGVGSNMQDHVHAV |
| | | TVSTTNITGYTTNSVFVNETLAQEQREEYETNKYPYDVPDYATGIWTTTPNNLGY |
| | | PTPEQLENGTEFVSGKEFADKIRNSTDEWANYYASTNATNVELLKKQYAIVASRY |
| | | EENYLSPIEINLTPGYGGTGSPDLQNNKYQTVNHVLIAPLSRGYTHINSSDIEEPSVI |
| | | NPQYYSHPMDIDVHVASTKLAREIITASPGLGDLNSGEVEPGMNITSEDDLRSWLS |
| | | NNVRSDWHPVGTCAMLPKELGGVVSPALMVYGTSNLRVVDASIMPLEVSSHLM |
| | | QPTYGIAEKAADIIKNAYKNQYKN |
| ambHNL341 | 106 | MQQDTNTSSTDTYDYVIVGGGVAGLALASRISENKDVTVAVLESGPNANDQFVV |
| | | YAPGMYGQAVGTDLCPLVPTTPQENMGNRSLSIATGRLLGGGSAVNGLVWTRG |
| | | GLKDYDAWEELGNPGWNGSNLFKYFKKVENFHPPTPAQIEYGATYQKNAHGKN |
| | | GPIDVSFTNYEFPQSAKWNASLSSLDFTALPDLLNGTLAGYSTTPNILDPETVQRV |
| | | DSYAGYIAPYTSRSNLNVLANHTVSRIQFAPQNGSEPLKATGVEWYPTGNKDQK |
| | | QTIKARYEVIISSGAIGSPKLLEISGIGSKDIVSAAGVESLIDLPGVGSNMQDHVHAV |
| | | TVSTTNITGYTTNSVFQPGEFTLGNVNETLAQEQREEYETNKTGIWTTTPNNLGYP |
| | | TPEQLENGTEFVSGKEFADKIRNSTDEWANYYASTNATNVELLKKQYAIVASRYE |
| | | ENYLSPIEINLTPGYGGTGSPDLQNNKYQTVNHVLIAPLSRGYTHINSSDIEEPSVIN |
| | | PQYYSHPMDIDVHVASTKLAREIITASPGLGDLNSGEVEPGMNITSEDDLRSWLSN |
| | | NVRSDWHPVGTCAMLPKELGGVVSPALMVYGTSNLRVVDASIMPLEVSSHLMQ |
| | | PTYGIAEKAADIIKNAYKNQYKN |
| ambHNL358 | 107 | MQQDTNTSSTDTYDYVIVGGGVAGLALASRISENKDVTVAVLESGPNANDQFVV |
| | | YAPGMYGQAVGTDLCPLVPTTPQENMGNRSLSIATGRLLGGGSAVNGLVWTRG |
| | | GLKDYDAWEELGNPGWNGSNLFKYFKKVENFHPPTPAQIEYGATYQKNAHGKN |
| | | GPIDVSFTNYEFPQSAKWNASLSSLDFTALPDLLNGTLAGYSTTPNILDPETVQRV |
| | | DSYAGYIAPYTSRSNLNVLANHTVSRIQFAPQNGSEPLKATGVEWYPTGNKDQK |
| | | QTIKARYEVIISSGAIGSPKLLEISGIGSKDIVSAAGVESLIDLPGVGSNMQDHVHAV |
| | | TVSTTNITGYTTNSVFVNETLAQEQREEYETNKQPGEFTLGNTGIWTTTPNNLGYP |
| | | TPEQLENGTEFVSGKEFADKIRNSTDEWANYYASTNATNVELLKKQYAIVASRYE |
| | | ENYLSPIEINLTPGYGGTGSPDLQNNKYQTVNHVLIAPLSRGYTHINSSDIEEPSVIN |
| | | PQYYSHPMDIDVHVASTKLAREIITASPGLGDLNSGEVEPGMNITSEDDLRSWLSN |
| | | NVRSDWHPVGTCAMLPKELGGVVSPALMVYGTSNLRVVDASIMPLEVSSHLMQ |
| | | PTYGIAEKAADIIKNAYKNQYKN |
| ambTNI341 | 108 | MQQDTNTSSTDTYDYVIVGGGVAGLALASRISENKDVTVAVLESGPNANDQFVV |
| | | YAPGMYGQAVGTDLCPLVPTTPQENMGNRSLSIATGRLLGGGSAVNGLVWTRG |
| | | GLKDYDAWEELGNPGWNGSNLFKYFKKVENFHPPTPAQIEYGATYQKNAHGKN |
| | | GPIDVSFTNYEFPQSAKWNASLSSLDFTALPDLLNGTLAGYSTTPNILDPETVQRV |
| | | DSYAGYIAPYTSRSNLNVLANHTVSRIQFAPQNGSEPLKATGVEWYPTGNKDQK |
| | | QTIKARYEVIISSGAIGSPKLLEISGIGSKDIVSAAGVESLIDLPGVGSNMQDHVHAV |
| | | TVSTTNITGYTTNSVFISASRKLQLVNETLAQEQREEYETNKTGIWTTTPNNLGYP |
| | | TPEQLENGTEFVSGKEFADKIRNSTDEWANYYASTNATNVELLKKQYAIVASRYE |
| | | ENYLSPIEINLTPGYGGTGSPDLQNNKYQTVNHVLIAPLSRGYTHINSSDIEEPSVIN |
| | | PQYYSHPMDIDVHVASTKLAREIITASPGLGDLNSGEVEPGMNITSEDDLRSWLSN |
| | | NVRSDWHPVGTCAMLPKELGGVVSPALMVYGTSNLRVVDASIMPLEVSSHLMQ |
| | | PTYGIAEKAADIIKNAYKNQYKN |
| ambTNI358 | 109 | MQQDTNTSSTDTYDYVIVGGGVAGLALASRISENKDVTVAVLESGPNANDQFVV |
| | | YAPGMYGQAVGTDLCPLVPTTPQENMGNRSLSIATGRLLGGGSAVNGLVWTRG |
| | | GLKDYDAWEELGNPGWNGSNLFKYFKKVENFHPPTPAQIEYGATYQKNAHGKN |
| | | GPIDVSFTNYEFPQSAKWNASLSSLDFTALPDLLNGTLAGYSTTPNILDPETVQRV |
| | | DSYAGYIAPYTSRSNLNVLANHTVSRIQFAPQNGSEPLKATGVEWYPTGNKDQK |
| | | QTIKARYEVIISSGAIGSPKLLEISGIGSKDIVSAAGVESLIDLPGVGSNMQDHVHAV |
| | | TVSTTNITGYTTNSVFVNETLAQEQREEYETNKISASRKLQLTGIWTTTPNNLGYP |
| | | TPEQLENGTEFVSGKEFADKIRNSTDEWANYYASTNATNVELLKKQYAIVASRYE |
| | | ENYLSPIEINLTPGYGGTGSPDLQNNKYQTVNHVLIAPLSRGYTHINSSDIEEPSVIN |
| | | PQYYSHPMDIDVHVASTKLAREIITASPGLGDLNSGEVEPGMNITSEDDLRSWLSN |
| | | NVRSDWHPVGTCAMLPKELGGVVSPALMVYGTSNLRVVDASIMPLEVSSHLMQ |
| | | PTYGIAEKAADIIKNAYKNQYKN |
| AflHA341 | 110 | mlfslaflsalslataspagrakntttydyivvgggtsglvvanrlsenpdvsvllleagasvfnnpdvtnangyglafgsaidw |
| | | qyqsinqsyaggkqqvlragkalggtstingmaytraedvqidvwqklgnegwtwkdllpyylksenltaptssqvaagaa |
| | | ynpavngkegplkvgwsgslasgnlsvalnrtfqaagvpwvedvnggkmrgfniypstldvdlnvredaarayyfpyddr |
| | | knlhllenttanrlfwkngsaeeaiadgveitsadgkvtrvhakkeviisagalrsplilelsgvgnptilkknnitprvdlptvge |
| | | nlqdqfnngmagegygvYPYDVPDYAlagastvtypsisdvfgnetdsivaslrsqlsdyaaatvkvsnghmkqedl |
| | | erlyqlqfdlivkdkvpiaeilfhpgggnavssefwgllpfargnihissndptapaainpnyfmfewdgksqagiakyirkil |
| | | rsaplnkliaketkpglseipataadekwvewlkanyrsnfhpvgtaammprsiggvvdnrlrvygtsnvrvvdasvlpfq |
| | | vcghlvstlyavaerasdlikedaksa |
| AflHA358 | 111 | mlfslaflsalslataspagrakntttydyivvgggtsglvvanrlsenpdvsvllleagasvfnnpdvtnangyglafgsaidw |
| | | qyqsinqsyaggkqqvlragkalggtstingmaytraedvqidvwqklgnegwtwkdllpyylksenltaptssqvaagaa |
| | | ynpavngkegplkvgwsgslasgnlsvalnrtfqaagvpwvedvnggkmrgfniypstldvdlnvredaarayyfpyddr |
| | | knlhllenttanrlfwkngsaeeaiadgveitsadgkvtrvhakkeviisagalrsplilelsgvgnptilkknnitprvdlptvge |
| | | nlqdqfnngmagegygvlagYPYDVPDYAastvtypsisdvfgnetdsivaslrsqlsdyaaatvkvsnghmkqedl |
| | | erlyqlqfdlivkdkvpiaeilfhpgggnavssefwgllpfargnihissndptapaainpnyfmfewdgksqagiakyirkil |
| | | rsaplnkliaketkpglseipataadekwvewlkanyrsnfhpvgtaammprsiggvvdnrlrvygtsnvrvvdasvlpfq |
| | | vcghlvstlyavaerasdlikedaksa |
| AflHNL341 | 112 | mlfslaflsalslataspagrakntttydyivvgggtsglvvanrlsenpdvsvllleagasvfnnpdvtnangyglafgsaidw |
| | | qyqsinqsyaggkqqvlragkalggtstingmaytraedvqidvwqklgnegwtwkdllpyylksenltaptssqvaagaa |
| | | ynpavngkegplkvgwsgslasgnlsvalnrtfqaagvpwvedvnggkmrgfniypstldvdlnvredaarayyfpyddr |
| | | knlhllenttanrlfwkngsaeeaiadgveitsadgkvtrvhakkeviisagalrsplilelsgvgnptilkknnitprvdlptvge |
| | | nlqdqfnngmagegygvQPGEFTLGNlagastvtypsisdvfgnetdsivaslrsqlsdyaaatvkvsnghmkqedl |
| | | erlyqlqfdlivkdkvpiaeilfhpgggnavssefwgllpfargnihissndptapaainpnyfmfewdgksqagiakyirkil |
| | | rsaplnkliaketkpglseipataadekwvewlkanyrsnfhpvgtaammprsiggvvdnrlrvygtsnvrvvdasvlpfq |
| | | vcghlvstlyavaerasdlikedaksa |
| AflHNL358 | 113 | mlfslaflsalslataspagrakntttydyivvgggtsglvvanrlsenpdvsvllleagasvfnnpdvtnangyglafgsaidw |
| | | qyqsinqsyaggkqqvlragkalggtstingmaytraedvqidvwqklgnegwtwkdllpyylksenltaptssqvaagaa |
| | | ynpavngkegplkvgwsgslasgnlsvalnrtfqaagvpwvedvnggkmrgfniypstldvdlnvredaarayyfpyddr |
| | | knlhllenttanrlfwkngsaeeaiadgveitsadgkvtrvhakkeviisagalrsplilelsgvgnptilkknnitprvdlptvge |
| | | nlqdqfnngmagegygvlagQPGEFTLGNastvtypsisdvfgnetdsivaslrsqlsdyaaatvkvsnghmkqedl |
| | | erlyqlqfdlivkdkvpiaeilfhpgggnavssefwgllpfargnihissndptapaainpnyfmfewdgksqagiakyirkil |
| | | rsaplnkliaketkpglseipataadekwvewlkanyrsnfhpvgtaammprsiggvvdnrlrvygtsnvrvvdasvlpfq |
| | | vcghlvstlyavaerasdlikedaksa |
| AfITNI341 | 114 | mlfslaflsalslataspagrakntttydyivvgggtsglvvanrlsenpdvsvllleagasvfnnpdvtnangyglafgsaidw |
| | | qyqsinqsyaggkqqvlragkalggtstingmaytraedvqidvwqklgnegwtwkdllpyylksenltaptssqvaagaa |
| | | ynpavngkegplkvgwsgslasgnlsvalnrtfqaagvpwvedvnggkmrgfniypstldvdlnvredaarayyfpyddr |
| | | knlhllenttanrlfwkngsaeeaiadgveitsadgkvtrvhakkeviisagalrsplilelsgvgnptilkknnitprvdlptvge |
| | | nlqdqfnngmagegygvISASRKLQLlagastvtypsisdvfgnetdsivaslrsqlsdyaaatvkvsnghmkqedle |
| | | rlyqlqfdlivkdkvpiaeilfhpgggnavssefwgllpfargnihissndptapaainpnyfmfewdgksqagiakyirkilr |
| | | saplnkliaketkpglseipataadekwvewlkanyrsnfhpvgtaammprsiggvvdnrlrvygtsnvrvvdasvlpfqv |
| | | cghlvstlyavaerasdlikedaksa |
| AfITNI358 | 115 | Mlfslaflsalslataspagrakntttydyivvgggtsglvvanrlsenpdvsvllleagasvfnnpdvtnangyglafgsaidw |
| | | qyqsinqsyaggkqqvlragkalggtstingmaytraedvqidvwqklgnegwtwkdllpyylksenltaptssqvaagaa |
| | | ynpavngkegplkvgwsgslasgnlsvalnrtfqaagvpwvedvnggkmrgfniypstldvdlnvredaarayyfpyddr |
| | | knlhllenttanrlfwkngsaeeaiadgveitsadgkvtrvhakkeviisagalrsplilelsgvgnptilkknnitprvdlptvge |
| | | nlqdqfnngmagegygvlagISASRKLQLastvtypsisdvfgnetdsivaslrsqlsdyaaatvkvsnghmkqedle |
| | | rlyqlqfdlivkdkvpiaeilfhpgggnavssefwgllpfargnihissndptapaainpnyfmfewdgksqagiakyirkilr |
| | | saplnkliaketkpglseipataadekwvewlkanyrsnfhpvgtaammprsiggvvdnrlrvygtsnvrvvdasvlpfqv |
| | | cghlvstlyavaerasdlikedaksa |
| HNL-358-H3 | 116 | MQKTATSNTYDYVIVGGGVGGLALASRLSEDKSVTVAVLEAGPNADEQFVVYAP |
| | | GMYGQAVGTDLCPLRPTVPQEAMNNRTLTIATGKLLGGGSAINGLVWTRGALKD |
| | | FDAWEELGNPGWNGRTMFKYFKKVERFHPPTKAQVQYGATYQKGVHGKNGRI |
| | | DISFPEFQFPQSANWNASLATLDFTHQQDLLNGSLHGYSTTPNTLDPKTERRVDSY |
| | | TGYIAPFVSRKNLFVLANHTVSRIQFKPKNGTELLKAVGVEWYTTGDNSNKQTIK |
| | | ARREVIVSSGSIGSPKLLEISGIGNKDIVTAAGVQSLIDLPGVGSNMQDHVHAVTVS |
| | | TTNITGFTTDSVFQNETLAEEQRQQYYNNKGSQPGEFTLGNIKTGIWTTAPNNLG |
| | | YPSPSQLFNGTSFESGQAFANRIRNSTDQWAEYYASTNATNIELLKKQYAIVASRY |
| | | EENYLSPIEINFTPGYGGTTDVDLKNNKYQTVNHVLIAPLSRGYTHINSSNIEDPVV |
| | | INPQYYTHPMDVDVHIASTKLARRILGAEPGLASINSGETQPGSNITSDEDVKQWL |
| | | ADNVRSDYHPVGTCAMLPRELGGVVDPNLLVYGTANLRVVDASIMPLEISSHLM |
| | | QPTYGVAEKAADIIKMSRKNNNN |
| HNL-358-H1 | 117 | MQKTATSNTYDYVIVGGGVGGLALASRLSEDKSVTVAVLEAGPNADEQFVVYAP |
| | | GMYGQAVGTDLCPLRPTVPQEAMNNRTLTIATGKLLGGGSAINGLVWTRGALKD |
| | | FDAWEELGNPGWNGRTMFKYFKKVERFHPPTKAQVQYGATYQKGVHGKNGRI |
| | | DISFPEFQFPQSANWNASLATLDFTHQQDLLNGSLHGYSTTPNTLDPKTERRVDSY |
| | | TGYIAPFVSRKNLFVLANHTVSRIQFKPKNGTELLKAVGVEWYTTGDNSNKQTIK |
| | | ARREVIVSSGSIGSPKLLEISGIGNKDIVTAAGVQSLIDLPGVGSNMQDHVHAVTVS |
| | | TTNITGFTTDSVFQNETLAEEQRQQYYNNKQPGEFTLGNTGIWTTAPNNLGYPSPS |
| | | QLFNGTSFESGQAFANRIRNSTDQWAEYYASTNATNIELLKKQYAIVASRYEENY |
| | | LSPIEINFTPGYGGTTDVDLKNNKYQTVNHVLIAPLSRGYTHINSSNIEDPVVINPQ |
| | | YYTHPMDVDVHIASTKLARRILGAEPGLASINSGETQPGSNITSDEDVKQWLADN |
| | | VRSDYHPVGTCAMLPRELGGVVDPNLLVYGTANLRVVDASIMPLEISSHLMQPT |
| | | YGVAEKAADIIKMSRKNNNN |
| 358-HA-CC | 118 | MQKTATSNTYDYVIVGGGVGGLALASRLSEDKSVTVAVLEAGPNADEQFVVYAP |
| | | GMYGQAVGTDLCPLRPTVPQEAMNNRTLTIATGKLLGGGSAINGLVWTRGALKD |
| | | FDAWEELGNPGWNGRTMFKYFKKVERFHPPTKAQVQYGATYQKGCHGKNGRI |
| | | DISFPEFQFPQSANWNASLATLDFTHQQDLLCGSLHGYSTTPNTLDPKTERRVDSY |
| | | TGYIAPFVSRKNLFVLANHTVSRIQFKPKNGTELLKAVGVEWYTTGDNSNKQTIK |
| | | ARREVIVSSGSIGSPKLLEISGIGNKDIVTAAGVQSLIDLPGVGSNMQDHVHAVTVS |
| | | TTNITGFTTDSVFQNETLAEEQRQQYYNNKYPYDVPDYATGIWTTAPNNLGYPSP |
| | | SQLFNGTSFESGQAFANRIRNSTDQWAEYYASTNATNIELLKKQYAIVASRYEEN |
| | | YLSPIEINFTPGYGGTTDVDLKNNKYQTVNHVLIAPLSRGYTHINSSNIEDPVVINP |
| | | QYYTHPMDVDVHIASTKLARRILGAEPGLASINSGETQPGSNITSDEDVKQWLAD |
| | | NVRSDYHPVGTCAMLPRELGGVVDPNLLVYGTANLRVVDASIMPLEISSHLMQP |
| | | TYGVAEKAADIIKMSRKNNNN |
| UCW69416.1 | 119 | MKITAAIITVATAFASFASAQQDTNSSSTDTYDYVIVGGGVAGLALASRISENKDV |
| | | TVAVLESGPNANDRFVVYAPGMYGQAVGTDLCPLIPTTPQENMGNRSLTIATGRL |
| | | LGGGSAINGLVWTRGGLKDYDAWEELGNPGWNGANLFKYFKKVENFTPPTPAQI |
| | | EYGATYQKSAHGKKGPIDVSFTNYEFSQSASWNASLETLDFTALPDILNGTLAGY |
| | | STTPNILDPETVQRVDSYTGYIAPYTSRNNLNVLANHTVSRIQFAPKNGSEPLKAT |
| | | GVEWYPTGNKNQKQIIKARYEVIISSGAIGSPKLLEISGIGNKDIVSAAGVESLIDLP |
| | | GVGSNMQDHVHAITVSTTNITGYTTNSVFVNETLAQEQREEYEANKTGIWATTPN |
| | | NLGYPTPEQLENGTEFVSGKEFADKIRNSTDEWANYYASTNASNVELLKKQYAIV |
| | | ASRYEENYLSPIEINFTPGYEGSGNVDLQNNKYQTVNHVLIAPLSRGYTHINSSDV |
| | | EDHSVINPQYYSHPMDIDVHIASTKLAREIITASPGLGDINSGEIEPGMNITSEDDLR |
| | | SWLSNNVRSDWHPVGTCAMLPKELGGVVSPALMVYGTSNLRVVDASIMPLEVSS |
| | | HLMQPTYGIAEKAADIIKNFYKTQHKNQN |
| UCW69417.1 | 120 | MKITAAIITVATAFASFASAQQDTNSSSTDTYDYVIVGGGVAGLALASRISENKDV |
| | | TVAVLESGPYAGDRFVVYAPGMYGQAVGTDLAPLIPTTPQENMGNRSLTIATGRL |
| | | LGGGSAINGLVWTRGGLKDYDAWEELGNPGWNGANLFKYFKKVENFTPPTPAQI |
| | | EYGATYQKSAHGKKGPIDVSFTNYEFSQSASWNASLETLDFTALPDILNGTLAGY |
| | | STTPNILDPETVRRVDSYTGYIAPYTSRNNLNVLANHTVSRIQFAPKNGSEPLKAT |
| | | GVEWYPTGNKNQKQIIKARYEVIISSGAIGSPKLLEISGIGNKDIVSAAGVESLIDLP |
| | | GVGSNMQDHVHAITVSTTNITGYTTNSVFVNETLAQEQREEYEANKTGIWATCPN |
| | | NLGYPTPEQLENGTEFVSGKEFADKIRNSTDEWANYYASTNASNVELLKKQYAIV |
| | | ASRYEENYLSPIEINFTPGYEGSGNVDLQNNKYQTVNHVLIAPLSRGYTHINSSDV |
| | | EDHSVINPQYYSHPMDIDVHIASTKLAREIITASPGLGDINSGEIEPGMNITSDDDVR |
| | | KWLSNNVRSDWHPVGTCAMLPKELGGVVSPALMVYGTSNLRVVDASIMPLEVS |
| | | SHLMQPTYGIAEKAADIIKNFYKTQHKNQN |
| UCW69418.1 | 121 | MKISAAIVTIATAFASLVSAQSNTDTYDYVIVGGGVGGLALANRLSENKQVTVAV |
| | | LEAGPNANDEFIVYAPGMYGQAVGTYLAPLRPTVPQENMNNRSLSIATGKLLGG |
| | | GSAVNGLVWTRGATKDFDAWEELGNPGWNGASMFKYFKKVENFTAPTPYQVN |
| | | YGATYQKNTHGYKGPVQVSFTNYEFPQSAHWNQSLASLGFDHLPDLLNGTLSGY |
| | | STTPNILDPNTDQRCDAYAAYIAPYTARTNLHVLANHTVSRIEFNQTNANQPLVA |
| | | SGVEWYPTGDNTKKQTIKARLEVIVSSGSIGSPKLLEISGIGNKDIVTAAGVKSLLD |
| | | LPGVGSNMQDHVHAVTVSTTNITGYTTDSVFVNSTLASEQREQYEKDKSGIWTTT |
| | | PNNLGYPTPAQLENGTEFMDGKAFAARIRNSSQEWAQYYASKNASTVELLMKQY |
| | | EIVASRYEENYLSPIEINLTPGYGGVGTVDKTKNKYQTVNHVLIAPLSRGFTHINSS |
| | | DIEDPVNINPQYYSHPMDIDVHVASTKLARRIINAPGLGDLNSGEVEPGMDITSDS |
| | | DVRAWLANNVRSDWHPVGTCAMLPKELGGVVDSSLKVYGTANLRVVDASIMPL |
| | | EVSSHLMQPTFGVAEKAADIIKAEYKKQKAQ |
| UCW69419.1 | 122 | MKISVAIVTIAAAFASFANAQKTATSNTYDYVIVGGGVGGLALASRLSEDKSVTV |
| | | AVLEAGPNADEQFVVYAPGMYGQAVGTDLCPLRPTVPQEAMNNRTLTIATGKLL |
| | | GGGSAINGLVWTRGALKDFDAWEELGNPGWNGRTMFKYFKKVERFHPPTKAQV |
| | | QYGATYQKGVHGKNGRIDISFPEFQFPQSANWNASLATLDFTHQQDLLNGSLHG |
| | | YSTTPNTLDPKTARRVDSYTGYIAPFVSRKNLFVLANHTVSRIQFKPKNGTELLKA |
| | | VGVEWYTTGDNSNKQTIKARREVIVSSGSIGSPKLLEISGIGNKDIVTAAGVQSLID |
| | | LPGVGSNMQDHVHAVTVSTTNITGFTTDSVFQNETLAEEQRQQYYNNKTGIWTT |
| | | TPNNLGYPSPSQLFDGTSFESGQAFANRIRNSTDQWAEYYASTNATNIELLKKQY |
| | | AIVASRYEENYLSPIEINFTPGYGGTTDVDLKNNKYQTVNHVLIAPLSRGYTHINSS |
| | | NIEDPVVINPQYYTHPMDVDVHIASTKLARRILGAEPGLASINSGEIQPGSNITSDE |
| | | DVKQWLADNVRSDWHPVGTCAMLPRELGGVVDPNLLVYGTANLRVVDASIMPL |
| | | EISSHLMQPTYGVAEKAADIIKMSRKNNNN |
| UCW69420.1 | 123 | MRLSVAILTLTSALASVTSAQQNNTDTYDYVIVGGGVGGLALASRLSEDKNVTV |
| | | AVLESGPYADDKFVVYAPGMYGQAVGTDLCPLLPTVPQPSMNNRTITIATGRLLG |
| | | GGSAVNGLVWTRGAMKDFDAWQELGNPGWNGTTMFKYFKKIENFHPPTEEQIQ |
| | | YGATYNKSVHGFNGPIDIAFPVFEFPQSANWNASLAHLNFTRRQDLLDGSLHGYS |
| | | TTPNTLNPQTARRADAYAGYIQPNVNRTNLAVLANHTVSRIQFEARNGSQPLKAI |
| | | GVEWYTTGGDKTSKQTIKARREIILSSGAIGSPKLLEVSGIGNKAIVTAAGVQSLID |
| | | LPGVGSNMQDHVHAVTVSTTNIDGYTTNSVFTNETLAQEQKDLYYNNKTGIWTT |
| | | TPNNLGYPSPSQLFTNTTFKSGKEFAAMIRNSTDKYAQYYAANNATNVELLKKQ |
| | | YSIVARRYEENYISPIEINFTPGYGGTGMADLQNKKYQTVNHVLVAPLSRGYTHIN |
| | | SSDIEDPVVIDPQYYSHPLDVDVHVASTQLARSILNAPGLASINSGEVEPGEKVQS |
| | | DEDVRKWLSDNVRSDWHPVGTCAMLPRKLGGVVDSKLKVYGTANLRIVDASIIP |
| | | LEISSHLMQPVYAVSERAADIIKSSSKK |
| UCW69421.1 | 124 | MRLSLAILSLTSALVTVTSAQQNGTSNDTYDYVIVGGGVGGLSLASRLSEDKGVT |
| | | VAVLESGPYADDRFVVYAPGMYGQAVGTELCPLLPTVPQVGMNNRTITIATGRL |
| | | LGGGSAVNGLVWTRGAMKDFDAWEELGNPGWNGKTMFKYFKKIENFHPPTEEQ |
| | | VQYGATYQKNVHGSGGPIDISFPVFEFPQSANWNASLAYLNFTHQQDLLNGSLHG |
| | | YSTTPNTLNPETARRADAYAGYIQPNVNRTNLAVLANHTVSRIQFEKSNGSQPLK |
| | | AIGVEWYTTGGDKSTKQTIKARREVIISSGAIGSPKLLEVSGIGNKQIVTAAGVESL |
| | | IDLPGVGSNMQDHVHAVTVSTTNIEGYTTNSVFTNETLAQEQKDLYYNNKTGIW |
| | | TTTPNNLGYPSPSQLFTNTTFRSGKQFAAMIRNSTDKYAQYYASTKNATNIQLLK |
| | | KQYAIVARRYEEDYISPIEINFTPGYGGTGEVDLQNNKYQTVNHVLVAPLSRGYT |
| | | HINSSDIEDPVVIDPQYYSHPLDVDVHVASTQLARSILNAPALAAINSGEVEPGEKI |
| | | QTDQDVRKWLSDNVRSDWHPVGTCAMLPKGLGGVVDSNLKVYGTANLRVVDA |
| | | SIIPLEISSHLMQPVYAVSERAADIIKGSRN |
| UCW69422.1 | 125 | MKISAAVVTIVTAFASVATAQQQNTSETNTYDYVIVGGGVGGLALASRLSENKG |
| | | VSVAVLEAGPYAGDQFVVYAPGMYGQAVGTDLCPLLPTTPQENMGNRSLSIATG |
| | | KLLGGGSSVNGLVWTRGGLKDFDAWEELGNPGWNGASMFNYFKKVENFTPPTP |
| | | AQAAYGATYQKNAHGTKGPMDVSFTNFEFPQSGNWNASLNAVGFTAVPDLLNG |
| | | TLHGYSTTPNILDPVNARRADAYAGYIKPYISRNNLAVLANHTVSRIQFAPQSGSQ |
| | | PLRATGVEWYPTGDKSQKQVLNARYEVILSSGAIGSPKLLELSGIGNKDIVAAAGI |
| | | QSLLDLPGVGSNMQDHVHAVTVSTTNITGYTTNSIFTNDALAAEERQEYDNNKT |
| | | GIYTTTPNNLGYPSPSQLFRGTSFVSGKQFAARIRNTTDEWAERYAADNATNAEL |
| | | LKKQYAIIASRYEEDYLSPIEINLTPGYGGTADVDLTNNKYQTVNHVLIAPLSRGY |
| | | THIKSADIEDAVDINPQYYSHPMDVDVHVASTKLAREIISASPGLGDINSGETEPGK |
| | | EITSDSDVRKWLADNVRSDWHPVGTCAMLPKELGGVVDPNLKVYGTSNLRVVD |
| | | ASVMPLEVSSHLMQPTFGIAEKAADIIKSANKKRSN |
| UCW69423.1 | 126 | MKISAAVVTIVTAFASVATAQQQNTSETNTYDYVIVGGGVGGLALASRLSENKG |
| | | VSVAVLEAGPYAGDQFVVYAPGMYGQAVGTDLCPLLPTTPQENMGNRSLSIATG |
| | | KLLGGGSSVNGLVWTRGGLKDFDAWEELGNPGWNGASMFNYFKKVENFTPPTP |
| | | AQAAYGATYQKNAHGTKGPMDVSFTNFEFPQSGNWNASLNAVGFTAVPDLLNG |
| | | TLHGYSTTPNILDPVNARRADAYAGYIKPYISRNNLAVLANHTVSRIQFAPQSGSQ |
| | | PLRATGVEWYPTGDKSQKQVLNARYEVILSSGAIGSPKLLELSGIGNKDIVAAAGI |
| | | QSLLDLPGVGSNMQDHVHAVTVSTTNITGYTTNSIFTNDALAAEERQEYDNNKT |
| | | GIYTTTPNNLGYPSPSQLFRGTSFVSGKQFAARIRNTTDEWAERYAADNATNAEL |
| | | LKKQYAIIASRYEEDYLSPIEINLTPGYGGTADVDLTNNKYQTVNHVLIAPLSRGY |
| | | THIKSADIEDAVDINPQYYSHPMDVDVHVASTKLAREIISASPGLGDINSGETEPGK |
| | | EITSDSDVRKWLADNVRSDWHPVGTCAMLPKELDGVVDPNLKVYGTSNLRVVD |
| | | ASVMPLEVSSHLMQPTFGIAEKAADIIKSANKKRSN |
| UCW69424.1 | 127 | MKISAAIITVVTAFASFASAQQDTNNTSSTDTYDYVIVGGGVAGLALASRISENKD |
| | | VTVAVLESGPNAEDQFVVYAPGMYGQAVGTELAPLVPTTPQENMGNRSLSIATG |
| | | RLLGGGSAVNGLVWTRGGLKDYDAWEELGNPGWNGSNLFKYFKKVENFHPPTP |
| | | AQIEYGATYQKSAHGKNGPIDVSFTNFEFPQSAKWNASLESLDFTALPDLLNGTL |
| | | AGYSTTPNILDPETARRVDAYAGYIVPYMGRNNLNVLANHTVSRIQFAPQNGSEP |
| | | LKATGVEWYPTGNKDQKQTIKARYEVIISSGAIGSPKLLEISGIGNKDIVTAAGVES |
| | | LIDLPGVGANMQDHVHAVTVSTTNIDGYTTNSVFTNETLAQEQREQYEANKTGI |
| | | WTTTPNNLGYPTPEQLENGTEFVSGKEFAAKIRNSTDEWANYYASTNATNADLL |
| | | KKQYAIVASRYEENYLSPIEINLTPGYGGTGSPDLQNNKYQTVNHVLIAPLSRGYA |
| | | HINSSDIEEPSVINPQYYSHPLDIDVHVASTKLAREIITASPGLGDLNSGEVEPGMN |
| | | VTSEDDLRSWLSNNVRSDWHPVGTCAMLPQELGGVVSPALMVYGTSNLRVVDA |
| | | SIMPLEVSSHLMQPTYGIAEKAADIIKNYYKSQYSGAGKN |
| AfWT | 131 | Mntttydyivvgggtsglvvanrlsenpdvsvllleagasvfnnpdvtnangyglafgsaidwqyqsinqsyaggkqqvlra |
| | | gkalggtstingmaytraedvqidvwqklgnegwtwkdllpyylksenltaptssqvaagaaynpavngkegplkvgws |
| | | gslasgnlsvalnrtfqaagvpwvedvnggkmrgfniypstldvdlnvredaarayyfpyddrknlhllenttanrlfwkngs |
| | | aeeaiadgveitsadgkvtrvhakkeviisagalrsplilelsgvgnptilkknnitprvdlptvgenlqdqfnngmagegygv |
| | | lagastvtypsisdvfgnetdsivaslrsqlsdyaaatvkvsnghmkqedlerlyqlqfdlivkdkvpiaeilfhpgggnavsse |
| | | fwgllpfargnihissndptapaainpnyfmfewdgksqagiakyirkilrsaplnkliaketkpglseipataadekwvewl |
| | | kanyrsnfhpvgtaammprsiggvvdnrlrvygtsnvrvvdasvlpfqvcghlvstlyavaerasdlikedaksa |
| Af328TNI | 132 | Mntttydyivvgggtsglvvanrlsenpdvsvllleagasvfnnpdvtnangyglafgsaidwqyqsinqsyaggkqqvlra |
| | | gkalggtstingmaytraedvqidvwqklgnegwtwkdllpyylksenltaptssqvaagaaynpavngkegplkvgws |
| | | gslasgnlsvalnrtfqaagvpwvedvnggkmrgfniypstldvdlnvredaarayyfpyddrknlhllenttanrlfwkngs |
| | | aeeaiadgveitsadgkvtrvhakkeviisagalrsplilelsgvgnptilkknnitprvdlptvgenlqdqfnngmagegygv |
| | | ISASRKLQLlagastvtypsisdvfgnetdsivaslrsqlsdyaaatvkvsnghmkqedlerlyqlqfdlivkdkvpiaeil |
| | | fhpgggnavssefwgllpfargnihissndptapaainpnyfmfewdgksqagiakyirkilrsaplnkliaketkpglseipa |
| | | taadekwvewlkanyrsnfhpvgtaammprsiggvvdnrlrvygtsnvrvvdasvlpfqvcghlvstlyavaerasdlike |
| | | daksa |
| Af328HA | 133 | Mntttydyivvgggtsglvvanrlsenpdvsvllleagasvfnnpdvtnangyglafgsaidwqyqsinqsyaggkqqvlra |
| | | gkalggtstingmaytraedvqidvwqklgnegwtwkdllpyylksenltaptssqvaagaaynpavngkegplkvgws |
| | | gslasgnlsvalnrtfqaagvpwvedvnggkmrgfniypstldvdlnvredaarayyfpyddrknlhllenttanrlfwkngs |
| | | aeeaiadgveitsadgkvtrvhakkeviisagalrsplilelsgvgnptilkknnitprvdlptvgenlqdqfnngmagegygv |
| | | YPYDVPDYAlagastvtypsisdvfgnetdsivaslrsqlsdyaaatvkvsnghmkqedlerlyqlqfdlivkdkvpia |
| | | eilfhpgggnavssefwgllpfargnihissndptapaainpnyfmfewdgksqagiakyirkilrsaplnkliaketkpglsei |
| | | pataadekwvewlkanyrsnfhpvgtaammprsiggvvdnrlrvygtsnvrvvdasvlpfqvcghlvstlyavaerasdli |
| | | kedaksa |
| Af328HNL | 134 | Mntttydyivvgggtsglvvanrlsenpdvsvllleagasvfnnpdvtnangyglafgsaidwqyqsinqsyaggkqqvlra |
| | | gkalggtstingmaytraedvqidvwqklgnegwtwkdllpyylksenltaptssqvaagaaynpavngkegplkvgws |
| | | gslasgnlsvalnrtfqaagvpwvedvnggkmrgfniypstldvdlnvredaarayyfpyddrknlhllenttanrlfwkngs |
| | | aeeaiadgveitsadgkvtrvhakkeviisagalrsplilelsgvgnptilkknnitprvdlptvgenlqdqfnngmagegygv |
| | | QPGEFTLGNlagastvtypsisdvfgnetdsivaslrsqlsdyaaatvkvsnghmkqedlerlyqlqfdlivkdkvpiae |
| | | ilfhpgggnavssefwgllpfargnihissndptapaainpnyfmfewdgksqagiakyirkilrsaplnkliaketkpglsei |
| | | pataadekwvewlkanyrsnfhpvgtaammprsiggvvdnrlrvygtsnvrvvdasvlpfqvcghlvstlyavaerasdli |
| | | kedaksa |

### EXAMPLES

### Example 1

### General Method for New Allosteric Site Discovery

Allostery is the means by which an effector binds to an enzyme at a site which is distal to the active site and transmits a signal that alters the enzymatic activity. The effector can be a small molecule, peptide, or antibody. Antibodies are significantly larger than small molecules and peptides and therefore may be more amenable to the discovery of allosteric effector sites of enzymes which use small molecule substrates.

Pools of potentially inhibitory antibodies can be generated in multiple ways, including immunization of animals or through screening synthetic antibody libraries using phage display. Once a pool of antibodies specific to the enzyme of interest has been found, that pool can be screened for the ability to inhibit the target. The pool of antibodies could take the form of serum from an immunized animal or the form of a pool of phage enriched for the target of interest. If the appropriate controls are used (i.e., pools of antibodies generated towards a different target), the pool of antibodies can be used in the enzyme assay of choice to determine if the pool contains a significant amount of inhibitory antibodies. If the pool of antibodies shows inhibition above control, it can be concluded that the pool contains antibodies that will inhibit the enzyme of interest. If the enzyme of interest uses a small molecule as a substrate, it is probable that some of the antibodies inhibit the enzyme in an allosteric manner.

The pool of antibodies can then be separated into individual clones and screened using the enzyme assay of choice in a high throughput manner to find the individual clones that inhibit the enzyme. Once the clones have been identified, they may be screened for mode of inhibition using a Lineweaver-Burk analysis described below. Any antibodies determined to inhibit in a noncompetitive or uncompetitive manner can be considered allosteric inhibitors. Confirmation of the binding site is facilitated by a crystal structure or a similarly conclusive structural analysis of the epitope-paratope interaction.

### General FAD-GDH Assay Protocol

The FAD-GDH (flavin adenine dinucleotide—glucose dehydrogenase) activity assay measures the enzyme activity by monitoring the reaction mix's optical absorbance change at 600 nm. The reaction mixture contains enzyme (FAD-GDH), substrate (glucose), electron mediator (phenazine ethosulfate, PES) and a color report reagent (2,6-dichlorophenolindophenol, DCPIP). While FAD-GDH converts one molecule of glucose to gluconolactone, PES mediate two electrons to DCPIP, which as a final electron receptor, are reduced to colorless DCPIPH₂.

In the assay design, assay mixture contains three components, which includes 10 μL of the 10× enzyme solution (purified FAD-GDH or Pichia expression supernatant solution), 10 μL of 10× substrate (D-glucose) solution, and 80 μL of the 1.25× reaction master mix (electron mediator PES and color report reagent DCPIP). The total assay volume is 100 μl with final concentration of 1× enzyme (final concentration varies depending on experiment design), 1× substrate (100 mM) and 1× reaction master mix (2 mM PES and 0.5 mM DCPIP).

### Materials

Phenazine Ethosulfate (PES), Sigma (P4544-5G)
2,6-dichlorophenol-indophenol (DCPIP), Sigma, (D1878-5G)
Triton X-100, Sigma (T9284-100ML)
PIPES, Sigma (P6757-25G)
Costar Assay Plate, 96-well, black with clear, flat bottom, non-sterile, Corning (Cat #3631)
Millex-GV Sterile 33 mm Low Protein Binding Durapore PVDF Membrane 0.22 μm, Millipore (SLGV033RS)
BMG Plate Reader, AP20-506

### Methods

### Reagents Preparation

Prepare enzyme dilution buffer (50 mM potassium phosphate buffer pH 6.5): Make potassium phosphate pH 6.5 with mono- and dibasic potassium phosphate. Add about 35 mL of 1 M monobasic potassium phosphate to 15 mL of 1 M dibasic potassium phosphate. Titrate to pH 6.5 with mono- or dibasic potassium phosphate. Dilute this stock to 1 L to make 50 mM potassium phosphate pH 6.5. Filter with 0.22 μm filter.

Prepare assay buffer (50 mM PIPES-NaOH Buffer pH 6.5 with 0.1% Triton X-100 Solution): Add 1.51 g of PIPES into 60 mL of water and stir. Add 1.0 mL of 10% Triton solution and adjust the pH to 6.5+0.05 with 6N NaOH. Transfer to a 100 mL graduated cylinder and fill up to 100 mL mark with distilled water. Use 0.22 μm filter to filter sterilize the solution.

Prepare 1M D-Glucose Solution: Add 9.0 grams of D-Glucose to a 50 mL conical. Add distilled water until solution reaches 40 mL mark. Gently mix end-over-end until completely dissolved. Allow the solution to stir for at least 16 hours at room temperature (to allow time for mutarotation of the glucose). Transfer solution to 50 mL graduated cylinder and add distilled water to 50 mL mark. Use 0.22 μm filter to filter sterilize the solution.

### Assay Plate Preparation and Reading

Prepare the 10× enzyme diluent. Dilute enzyme stock solution or Pichia expression supernatant to 10× concentration with enzyme dilution buffer.

Prepare reaction mix. Add 357 μL of 80 mM PES solution and 893 μL 8 mM DCPIP solution in 8.6 mL of assay buffer. Add 179 μL of DI water to make total volume 10 mL. Vortex and mix well.

Transfer 10 μL of the 10× enzyme solution to a 96-well assay plate. Add 80 μL of the reaction master mix using multi-channel pipette. Incubate the plate at r.t for 5-10 mins. Load the plate on the BMG plate reader. Inject 10 μL of 1M D-Glucose solution in each well and shake 30 seconds at 500 rpm. Read the plate for 15 reading cycles. The time interval in between each reading cycles is 87 seconds. Total reading time is 20 mins.

### Data Processing

Analyze the data in MARS software. Use linear regression from GraphPad Prism8 to calculate the slope and R square of each reaction well. Use the slope from each individual sample well and control well (wild-type FAD-GDH enzyme) to calculate the relative activity using the following equation:

$$Activity \% = {Slope}_{sample} / {Slope}_{control} * 100 \%$$

### Example 2

### Discovery of Allosteric Sites on First Mucor FAD-GDH and Identification of Inhibitory Antibody 1-286

Mice immunizations Five CAF1/J, SJL/J, and RBF/DnJ female mice were inoculated with a Mucor mutant FAD-GDH. Thirty-five μg of FAD-GDH (ungrafted; 19031 FAD-GDH) was diluted in potassium phosphate pH 5.5, 0.1% (v/v) Triton X-100, 0.1 ml of Adjulite Complete Freund's adjuvant, and sterile 0.9% NaCl to a final volume of 0.2 ml per animal. An additional five animals were inoculated similarly, except 0.2 ml of AddaVax adjuvant and 0.005 mg of human/mouse CpG DNA was substituted for Adjulite, and 0.4 ml inoculum was used per mouse. Inoculum was administered to the 2 axillary and 2 inguinal sites on the animal's ventral side. After a period of six weeks, a secondary immunization was performed according to the same protocol. After an additional 4 weeks, another immunization was administered. The animals were allowed to develop immune responses for an additional 5 months, at which point serum was collected and screened for inhibition of the FAD-GDH enzyme activity in vitro.

Screening of mouse sera for inhibitory antibodies Sera were labeled #71-99 with each tube corresponding to a different mouse. Normal mouse serum (NMS) was used as control. Serial dilutions of each serum were prepared in phosphate buffered saline, pH 7.2 (PBS). Colorimetric GDH assays were performed in 96-well plates according to the General FAD-GDH Assay Protocol of Example 1, with each reaction containing 12.5 μl diluted serum in 50 mM PIPES/Triton buffer, 2 mM phenazine methosulfate (PMS) and 0.17 mM dichlorophenol indophenol (DCPIP), 0.04 μg FAD-GDH, and 12.5 μl of a 400 mM D-glucose solution added last to initiate each reaction. The final volume of reaction wells was 125 μl. The absorbance at 600 nm was read continuously over 30 min at 37° C. in a spectrophotometer.

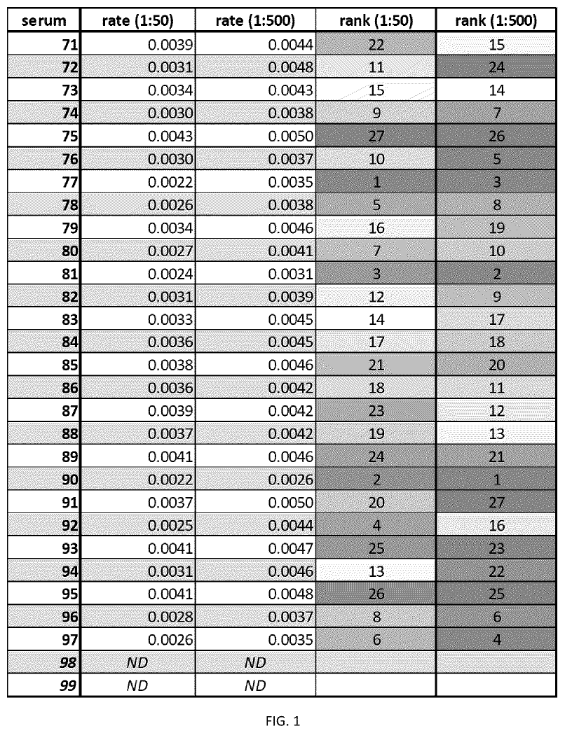

Resulting data were plotted and the slope of the linear portion of the curve was used to calculate a rate value (−ΔA600/min). Comparisons were made between different sera dilutions (1:50 or 1:500) and ranked by extent of inhibition (FIG. 1). Results for control reactions included: PBS+FAD-GDH+40 mM glucose: 0.0053; normal mouse serum (NMS)+enzyme+glucose rate was 0.0052 or 0.0051 for 1:50 and 1:500, respectively; PBS +enzyme+water (in lieu of glucose) rate was 0.0016. Several sera were found to inhibit the FAD-GDH enzyme reaction rate using this screening methodology. None of the sera showed an effect of GDH stimulation in these assays. Mice #80, 81, and 90 were selected for producing hybridoma fusions, and their spleens were harvested and perfused, and splenic cells were harvested, frozen, and stored in liquid nitrogen until use.

Fusion of human myeloma with mouse B cells Splenic cell recovery samples were thawed, combined, and rinsed extensively with HSFM culture media (Gibco; Cat. #: ME130092L1) supplemented with 10% (v/v) FBS, 10 ml/L L-glutamine, and 24 μg/ml CpG DNA and transferred to a culture flask. The cells were recovered in a 37° C. incubator with 5% CO2 overnight. B-cells were enriched using the EasySep Mouse B-Cell Isolation Kit (Stemcell Technologies) per manufacturer's instructions yielding a count of 1.4*10⁶ B cells.

Early passage human myeloma cells were thawed from cryostorage to 37° C. and recovered in supplemented HSFM culture media. After several days of expansion in growth flasks, cells were counted and 1.47*10⁷ mouse B cells and 1.47*10⁷ myeloma cells were fused using Cytofusion medium C (BTX; Cat. #: 47-0001) in a Microslide electrofusion chamber by standard methods. Fusion bulk culture was cultured in HSFM media supplemented with HAT (Sigma; Cat. #: H0262) for selection. Cells were then pelleted and resuspended in a mixture of HSFM and CloneMatrix semi-solid media with goat anti-mouse DyLight 488, and plated. Individual hybridoma colonies showing enrichment of fluorescent signal were picked using a Clonepix 2 and arrayed into 96-well culture plates. Ten plates of anti-FAD-GDH hybridoma clones were picked and grown to density.

ELISA screening of hybridoma supernatants A 1 μg/ml dilution of FAD-GDH was prepared in PBS buffer and passively coated onto 96-well ELISA plates (BrandTech; Cat. #: 781722). After washing plates with water, they were then blocked with a blocking buffer (PBS supplemented with 5% (w/v) BSA and 0.1% (v/v) Tween-20), washed again, and then incubated with hybridoma supernatants (1 clone per well). Plates were washed again and next incubated with Affinipure sheep anti-mouse peroxidase-conjugated antibody (Jackson ImmunoResearch; Cat. #: 515-035-062) for detection. After washing, the plates were reacted with colorimetric peroxide development solution, reactions were terminated by addition of 1 N sulfuric acid, and absorbance of each well read at 492 nm. Any well having an absorbance value above a nominal cut-off value of 0.2 was considered positive for binding. In total, 89 clones were identified as binding to FAD-GDH in the ELISA assay and carried forward.

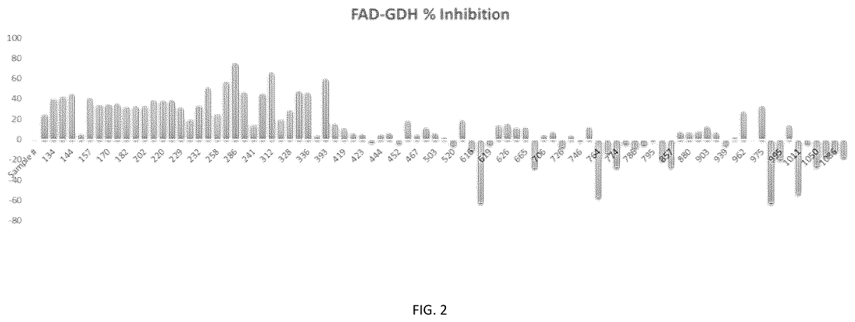

FAD-GDH inhibition assays with selected hybridoma clones Colorimetric FAD-GDH assays were performed in the presence of the selected 89 hybridoma clone supernatants (60 μl/well), with reagents and methods consistent with the General FAD-GDH Assay Protocol of Example 1. Absorbance was read at 600 nm for 30 min. Percent inhibition was calculated for each clone by comparing it to a media-only control by the following equation: ((Slope_{Media-only}−Slope_{Antibody})/Slope_{Media-only})*100 (FIG. 2).

Isotype determination of selected clones A panel of clones which exhibited the highest inhibition or activation of FAD-GDH were selected for isotype testing. ELISA plates were passively coated with sheep anti-mouse IgG antibody and washed. Antibody-containing hybridoma supernatants were screened using the SBA-Clonetyping System-HRP kit (Southern Biotech; Cat. #: 5300-05). Table 1 lists the identified isotype(s) detected in each clone together with its percent inhibition measured in the FAD-GDH assay.

TABLE 1
Listing of calculated percent inhibition in FAD-GDH colorimetric
screening assays with hybridoma supernatants and isotype
of antibodies detected in each clonal supernatant.

| Clone # | Percent Inhibition* | Isotype(s) |
|---|---|---|
| 134 | 40 | G1, kappa |
| 236 | 51 | G1, kappa |
| 228 | 38 | G1, kappa |
| 275 | 56 | G1, kappa |
| 286 | 75 | G1, kappa |
| 618 | −62 | G2b, kappa |
| 312 | 66 | G1, G2a, M, kappa |
| 336 | 46 | G1, G2a, kappa |
| 393 | 59 | G1, G2a, kappa |
| 978 | −62 | G1, G2b, M, kappa |
| 143 | 42 | G1, G2b, M, kappa |
| 144 | 45 | G1, M, kappa |
| 157 | 41 | G1, M kappa |
| 158 | 34 | G1, M, kappa |
| 170 | 34 | G1, M, kappa |
| 179 | 35 | G2b, M, kappa |
| 182 | 32 | G1, M, kappa |
| 196 | 32 | M |
| 202 | 32 | G1, G2a, M, kappa |
| 203 | 38 | G1, M, kappa |
| 220 | 38 | G1, M, kappa |
| 229 | 31 | G2b, M, kappa |
| 232 | 33 | G1, M, kappa |
| 292 | 46 | G, M, kappa |
| 311 | 45 | G1, M, kappa |
| 334 | 47 | G2a, M, kappa |
| 962 | 27 | G1, M, kappa |
| 703 | −28 | G1, G2b, kappa |
| 774 | −28 | G1, G2b, kappa |
| 857 | −27 | G1, kappa |
| 995 | −19 | G, M, kappa |
| 1050 | −26 | G, M, kappa |

*negative values of percent inhibition indicate observed stimulation of GDH activity with these samples.

Data were evaluated and clones meeting the following conditions were selected for scaling to 500 ml expression and purification experiments: change in enzyme activity greater than 30% in the colorimetric screening assay, IgG isotype, and clonal. Clones meeting these criteria were #134, 236, 228, 275, 286, and 618.

Culture of hybridoma clones and purification of anti-FAD-GDH antibodies FAD-GDH clones 1-134, 1-228, 1-236, 1-275, 1-286, and 1-618 were seeded into 500 ml of supplemented HSFM and cultured for two weeks. Culture supernatants were filtered through 0.45 μm then purified using a HiPrep Protein A column (Cytiva; Cat. #: 28-4082-61) and subsequently desalted into PBS using HiPrep 26/10 Desalting (Cytiva; Cat. #: 17-5087-01).

Absorbance of the purified protein at 280 nm was measured and a protein concentration determined using 1.38 AU for a 1 mg/ml solution measured in a 1 cm path length. Concentration and yield calculations are provided in Table 2.

TABLE 2
Results of purification of anti-FAD-GDH
antibodies from six hybridoma clones.

| | supernatant | volume of | mAb | Yield |
|---|---|---|---|---|
| Clone | volume | purified mAb | concentration | amount |
| ID | (ml) | (ml) | (mg/ml) | (mg) |
| 1-134 | 500 | ^{~}10 | 4.09 | 40.9 |
| 1-228 | 500 | ^{~}10 | 4.21 | 42.1 |
| 1-236 | 500 | ^{~}10 | 4.71 | 47.1 |
| 1-275 | 500 | ^{~}10 | 3.45 | 34.5 |
| 1-286 | 500 | ^{~}10 | 3.78 | 37.8 |
| 1-618 | 500 | ^{~}10 | 0.13 | 1.3 |

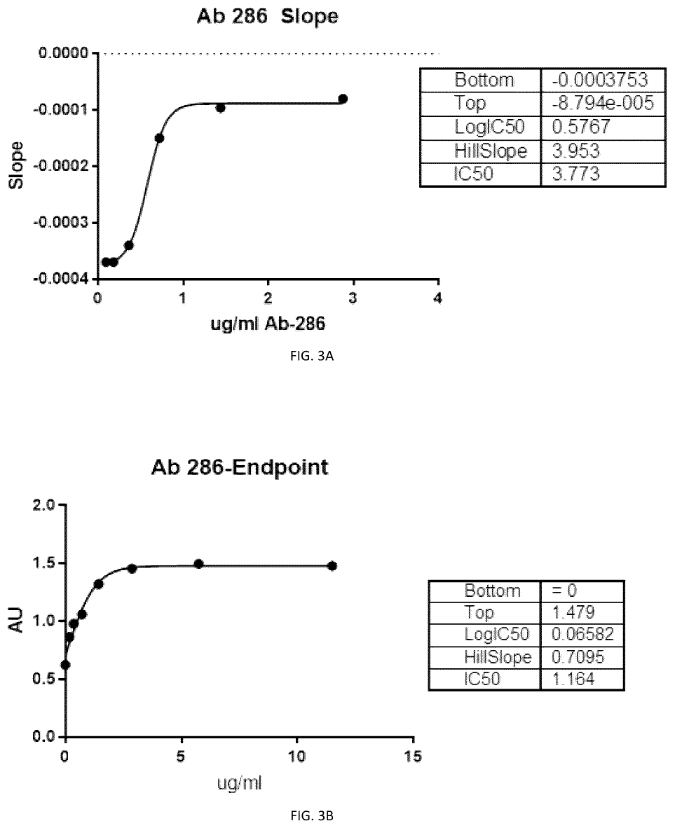

Enzyme assay screening of purified anti-FAD-GDH IgG clones Purified antibodies from clones 1-134, 1-286, 1-228, 1-275, 1-236, and 1-618 were tested for inhibition or stimulation of FAD-GDH enzyme activity. Only 1-286 showed inhibition in screening assays; no other clones showed inhibition. An 8-point, 2-fold dilution series of 1-286 was prepared and tested for dose-dependent inhibition of FAD-GDH using reagents and methods described the General FAD-GDH Assay Protocol of Example 1. Absorbance at 600 nm was measured for 30 min and the linear portion of each of the curves and linear regression was used to determine rates (slope of the line) and plotted against antibody concentration. FIG. 3A shows the dose-dependent relationship between measured slope and antibody concentration. The IC50 of mAb 1-286 was measured as 3.7 μg/ml under these experimental conditions. After reactions proceeded for a total of 45 min, absorbance at 600 nm was read again, and residual absorbance was plotted against antibody concentration. Again, higher absorbance values correlated with higher extents of inhibition at higher concentrations of mAb 1-286, showing a saturable and dose-dependent inhibition response (FIG. 3B).

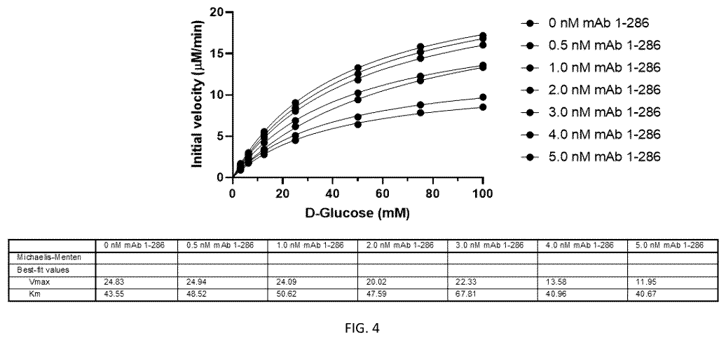

Enzyme assay for determining the allosteric mechanism of mAb 1-286 inhibition Using reagents and methods described in the General FAD-GDH Assay Protocol of Example 1, the initial velocity of FAD-GDH reaction was measured under conditions of serial dilution of D-glucose from 100 mM to 0 mM and a serial dilution of mAb 1-286 from 5 nM to 0 nM. Reactions proceeded for 30 min and absorbance read continuously at 600 nm. The initial velocity was calculated as μM/min and plotted against the concentration of glucose in mM (FIG. 4).

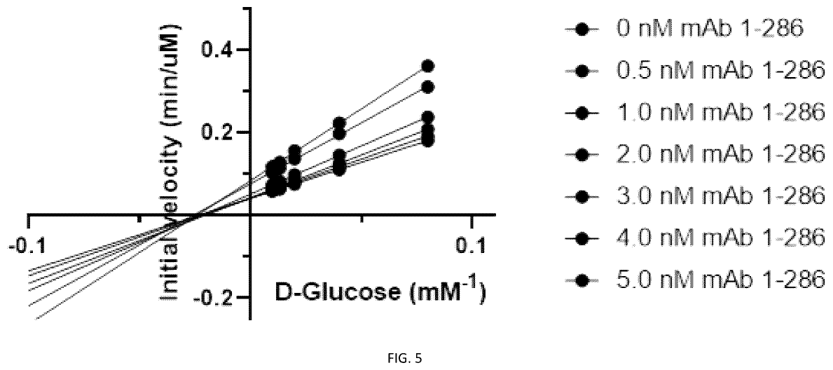

A double-reciprocal (Lineweaver-Burk) plot was generated from the data in FIG. 4 to determine the mechanism of enzyme inhibition by mAb 1-286. The data, presented in FIG. 5, show the intersection of the various lines at a common intercept point on the x-axis left of the origin, with varying y-intercepts measured. The data indicate an allosteric mechanism of inhibition and exclude a competitive inhibitory mechanism.

### Example 3

### Characterization of Allosteric Site Via Crystal Structure Analysis

To reduce the heterogeneity of the recombinant FAD-GDH protein for crystallization, DNA encoding non-glycosylated FAD-GDH was designed which removes the amino-terminal signal sequence directing the nascently folded protein to the secretion pathway. The protein is thus expressed recombinantly without attachment of glycan by the expression host organism. Following nominal expression conditions for glycosylated FAD-GDH, expression of non-glycosylated FAD-GDH protein was induced with methanol in Pichia pastoris clonal transformants. Cell pellets resulting from 2 L of expression culture were harvested by centrifugation and stored at −20° C. until purification.

Cytoplasmic proteins were liberated upon resuspension of the cells in 200 ml Yeastbuster reagent (EMD/Millipore), supplemented with 1×THP (Millipore), 1 mM MgCl2 (Sigma), and 400 U/ml OmniCleave endonuclease (Lucigen), and incubation at 22° C. with constant stirring for 1-2 hr. The lysate was then centrifuged at 18,000 rpm for 30 min at 8° C. in a JA-20 rotor (Beckman) to pellet insoluble material. The supernatants were pooled and filtered using a 0.22 μm, cellulose acetate vacuum filtration unit (Corning), diluted using 800 ml of Buffer A (20 mM potassium phosphate, pH 7.0), and mixed with constant stirring for 15 min. The mixture was filtered using a 0.45 μm, cellulose acetate vacuum filtration unit (Corning), and loaded onto a 5 ml HiTrap SP HP cation exchange column (GE/Cytiva) using an AKTA Pure FPLC (GE/Cytiva). After loading, the column is washed with 50 ml of Buffer A, then the protein eluted in a gradient of 0-700 mM NaCl using Buffer B (20 mM potassium phosphate, pH 7.0,1 M NaCl). The protein elutes between 200-300 mM NaCl as a sharp peak having characteristic absorbance at both 280 and 450 nm.

Peak eluate fractions containing non-glycosylated FAD-GDH were pooled and concentrated to ≤5 ml using Amicon-15 concentrators having a 30 kDa MWCO membrane (Millipore). The sample was then filtered using a MILLEX GV syringe-driven filter unit (Millipore) and injected onto a HiLoad 26/600 Superdex 200 pg column equilibrated in Buffer C (20 mM sodium phosphate, pH 7.2, 150 mM NaCl) using a 10 ml Superloop (GE/Cytiva). FAD-GDH was eluted as a single symmetric peak typically observed between 180-220 ml. The peak eluates were again pooled and concentrated to ≥20 mg/ml, frozen, and stored at −80° C. By SDS-PAGE and Coomassie Brilliant Blue staining analysis, purified, non-glycosylated FAD-GDH protein appears as a distinct band of approximately 70 kDa.

Crystal structures of FAD-GDH were solved either alone or in a 1:1 complex with rFab 286. For the enzyme-only structure, the complex of non-glycosylated FAD-GDH and rFab 286 was subjected to sitting drop sparse matrix screening of JCSG Core Suites I-IV (Nextal Biotech). After three days, crystals were observed in Suite I condition F10 (0.1 M phosphate-citrate, pH 4.2, 5% PEG 1000, 40% ethanol). A grid screen of the F10 hit condition was performed in 0.1 M sodium acetate, pH 4.2 with varying PEG 1000 concentrations from 3-8% and ethanol from 30-45%. Crystals grew to their maximum dimensions after one week of incubation at 20° C. Crystals were harvested by loops, passed through fresh drops of mother liquor containing 20% glycerol for cryoprotection, and flash-frozen in liquid nitrogen. Loops containing crystals were transferred to Uni Pucks and x-ray diffraction experiments were conducted. The crystal structure of FAD-GDH was solved from diffraction data extending to 1.94 Å using molecular replacement with Aspergillus flavus FAD-GDH (Protein Data Bank ID: 4YNT) as a search model. The structure revealed the presence of the FAD-GDH enzyme in the packed crystal lattice with no density for rFab 286. It was suspected that ethanol present in the mother liquor disrupted the interaction of the FAD-GDH with rFab 286, yielding crystals of FAD-GDH alone.

The 1:1 complex structure of FAD-GDH and rFab 286 was formed by mixing a molar ratio of 1:1.3 enzyme:Fab and development over a HiLoad 16/600 Superdex 200 pg column in Buffer D (20 mM Tris, pH 7.5, 100 mM NaCl, 1 mM TCEP). Peak fractions corresponding to the complex were collected and concentrated to ˜ 30 mg/ml and screening of the JCSG Core Suites I-IV (Nextal Biotech) in a sitting drop vapor diffusion format. Plates were incubated at 20° C. and initial hits were observed after 2 days. Four initial hits were identified with buffer pH ranging from 6.0-7.5, 10% PEG as precipitant (6K or 8K), and additives 8% ethylene glycol or 5% MPD. The best crystals were harvested by loop and passed through fresh drops of mother liquor containing 35% PEG 600 as cryoprotectant and flash-frozen in liquid nitrogen. Loops containing crystals were transferred to Uni Pucks and x-ray diffraction experiments were conducted at beamline BL13-XALOC, ALBA, Barcelona, Spain.

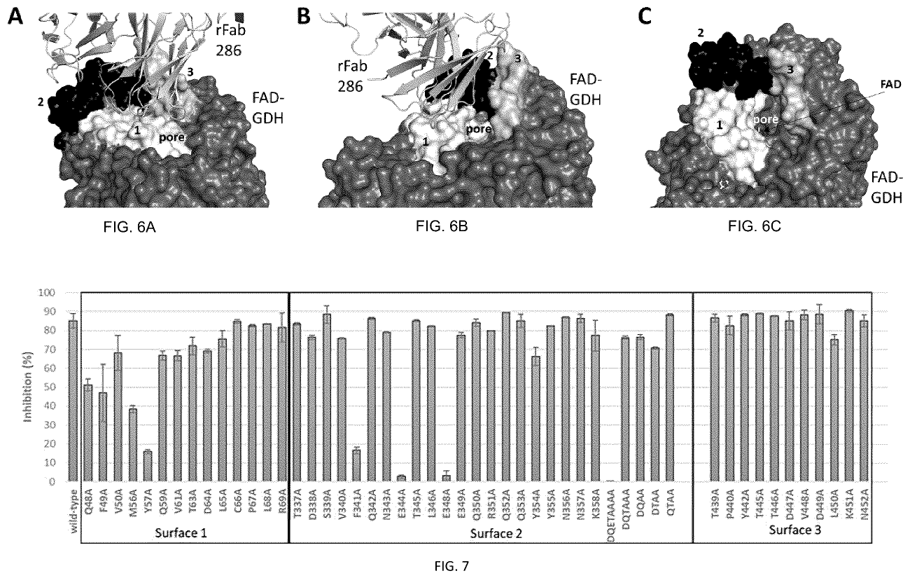

The interface formed between the enzyme and rFab spanned three non-contiguous segments of the enzyme sequence that are in proximity to one another in the folded enzyme (FIGS. 6A-6C). These regions were termed Surface 1, Surface 2, and Surface 3, and together form a conformational epitope for mAb 1-286 (rFab 286). Although the rFab was observed to bind adjacent to the presumed substrate entry channel of the enzyme, it does not appear to sterically occlude it to an appreciable extent. Direct contacts were formed between residues in the complementarity-determining regions (CDRs) of the rFab heavy chain and both Surface 2 (primarily) and Surface 1 of FAD-GDH. Direct interactions between rFab 286 and Surface 3 were less supported by the structure, although the wall-like structure formed by Surface 3 residues may assist in orienting and/or facilitating interactions of the rFab with the other two surfaces. Forty-six FAD-GDH residues within 4 Å distance of rFab 286 were selected for detailed epitope characterization via alanine scanning mutagenesis as described in the subsequent Example. Comparing FAD-GDH in the apo structure to that in a complex with rFab 286, no large changes in the FAD-GDH structure were detected.

### Example 4

### Characterization of Allosteric Site Via Surface Mapping

Alanine scanning mutagenesis was used to evaluate the specific importance of amino acid residues within the three Surfaces for the binding and functional responses to rFab 286. Amino acid residues within Surfaces 1, 2, or 3 of non-glycosylated FAD-GDH (SEQ ID NO: 1), were mutated from their native residue to alanine, except in the case where the native amino acid was either alanine or glycine. The panel of 46 mutant enzymes was expressed as non-glycosylated, His-tagged recombinant proteins in Pichia pastoris cultured in 24-well plates. Protein expression was induced by addition of 0.5% methanol, the cells were pelleted and lysed with YeastBuster™ Master Mix (Novagen/EMD Millipore), and soluble cytoplasmic proteins were isolated by centrifugation according to manufacturer's instructions. FAD-GDH variant activity in the soluble lysates was measured either in the absence or presence of mAb 1-286. Non-glycosylated FAD-GDH lacking any alanine mutations consistently showed greater than 80% inhibition with 1 nM mAb 1-286.

### Alanine Mutants' Activity Comparison

The purified, non-glycosylated FAD-GDH and alanine mutant FAD-GDH enzymes were diluted to final concentration of 100 nM, which were in turn serially diluted across wells of a 96-well plate using enzyme dilution buffer (50 mM Potassium Phosphate Buffer pH 6.5).

The Enzyme Solution (10 μL) was transferred from the Enzyme Dilution Plate to a 96-well Assay Plate. Reaction Master Mix (80 μL; 0.6 mM DCPIP and 2.5 mM PES in 50 mM PIPES buffer, pH 6.5, 0.1% v/v Triton) was added prior to incubation at ambient room temperature for 10 min. Following loading into the plate reader, 10 μL of 1 M D-Glucose solution was injected into each reaction well with shaking (30 s at 500 rpm). The plate was read for 15 reading cycles with an 87 s time interval between each reading cycle. Data from wells that exhibited too fast or too slow reaction rates were excluded from activity calculations. Data were trimmed to include only the linear portion of the reaction and linear regression from GraphPad Prism8 was used to calculate the slope and R-squared value of each reaction. The relative activity was calculated using the using the following equation:

$$Activity \% = {Slope}_{Alanine Mutant} / {Slope}_{Wild - type} * 100 \%$$

### Antibody 1-286 IC50 Measurement

Prepare the 10× enzyme solution. The 100 nM enzyme solutions, as prepared above, were diluted to final concentration of 6.25 nM. The 1-286 antibody stock solution was diluted to a final concentration 10 μM, which was then serially diluted (in 4-fold steps) in a 96-well plate using assay buffer.

The enzyme solution (10 μL) (unmutated FAD-GDH or alanine mutants) was transferred to the assay plate by row, along with 10 μL of the 1-286 antibody diluents. Reaction mix (70 μL) was added to the enzyme and incubated at ambient room temperature for 10 min. Following loading into the plate reader, 10 μL of 1M D-Glucose solution was injected in each well with shaking (30 s at 500 rpm). The plate was read for 30 reading cycles with a time interval between each reading cycle of 87 s. The dataset was trimmed to include only the linear portion of each reaction and linear regression was used to calculate the slope and R square of each reaction well. The percent inhibition was calculated using the slope of each well and the slope of the negative control wells (buffer only, no antibody) by using the following equation:

$$Inhibition \% = ( {Slope}_{antibody} - {Slope}_{Neg} ) / {Slope}_{Neg} * 100 \%$$

A four-parameter, non-linear regression analysis on the log antibody concentration vs. the inhibition percentage was performed to calculate the IC50 values.

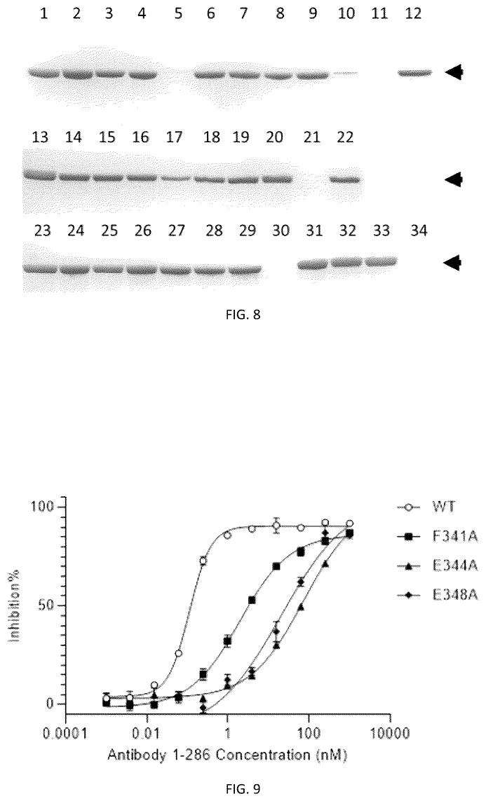

The evaluation of the alanine scanning mutants confirmed F341, E344, and E348 within Surface 2 as the most critical residues for inhibitory responses of FAD-GDH to mAb 1-286. Each of these residues formed contacts with the CDRs of rFab 286. Several mutations in the first half of Surface 1 manifested in changes in GDH activity as well as a diminished extent of inhibition by 1-286 (FIG. 7). Consistent with the lack of polar contacts observed between Surface 3 of FAD-GDH and rFab 286 in the structure, no alanine mutations introduced into Surface 3 were observed to alter the mutant enzymes' ability to respond to mAb 1-286.

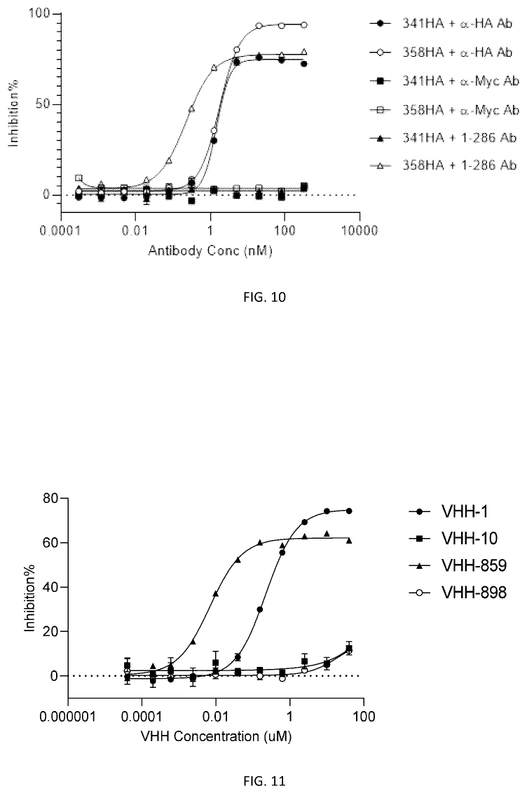

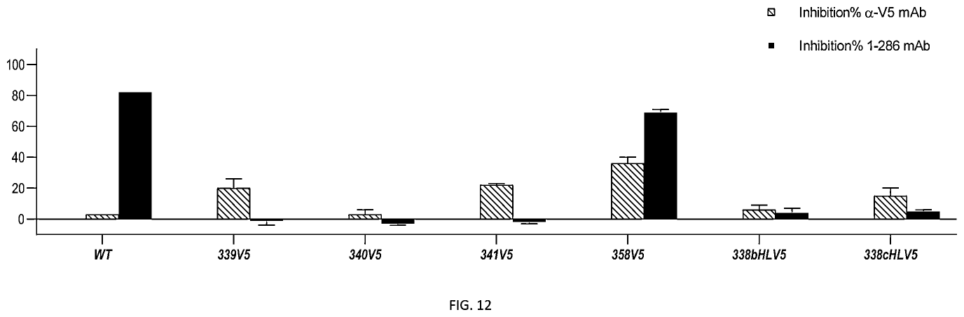

Select C-terminal His-tagged, non-glycosylated FAD-GDH mutants were scaled and purified from Pichia expression shake flask culture pellets by IMAC and size exclusion chromatography. The purified proteins were visualized by SDS-PAGE and Coomassie Brilliant Blue staining (FIG. 8). The mutant enzymes were next tested for in vitro GDH activity. To determine the IC50 value of mAb 1-286 when compared to the wild-type, the antibody was titrated against a set amount of non-glycosylated FAD-GDH and the resulting GDH activity was measured (Table 3). Most notably, the titration curves of mAb 1-286 and inhibition of wild-type (non-glycosylated, not mutated) FAD-GDH, F341A, E344A, and E348A are presented in FIG. 9. The IC50 values were calculated from the sigmoidal curve fitting: wild-type (WT), 0.1 nM; F341A, 2.0 nM; E344A, 71.4 nM; E348A, 20.2 nM. Since larger IC50 values correlate with more defective responses, by order of severity: E344A >E348A >>F341A. Differences between the inhibition of DQETAAAA and DQTAAA combination mutants in FIG. 7 also highlight the importance of E344 of FAD-GDH in responding to mAb 1-286. Based on these data it was concluded that Surface 2 is the main surface contributing to enzyme-antibody interactions resulting in the inhibitory function.

TABLE 3
Alanine mutants within Surfaces 1, 2, or 3 were produced as recombinant
proteins, purified, and tested for in vitro GDH activity. Activity
of each enzyme was measured relative to non-mutated, wild-type,
non-glycosylated FAD-GDH. Enzyme rates were measured with a range
of antibody concentrations and the IC50 calculated from the resulting
plot. Shifts in the IC50 values measured are presented as fold
of change from wild-type. NA, the measured enzyme activity was
judged too low for reliable IC50 determination.

| | | Relative | 1-286 IC50 | 1-286 IC50 Shift |
|---|---|---|---|---|
| Surface | Mutant | Activity % | (nM) | (−fold) |
| | Wild-type | 100 | 0.1 | 1 |
| Surface 1 | Q48A | 111 | 0.2 | 2 |
| | F49A | 87 | 0.1 | 2 |
| | V50A | 126 | 0.2 | 4 |
| | M56A | 111 | 0.6 | 9 |
| | Y57A | 14 | NA | NA |
| | Q59A | 118 | 0.1 | 1 |
| | V61A | 76 | 0.1 | 2 |
| | T63A | 102 | 0.1 | 2 |
| | D64A | 92 | 0.2 | 3 |
| | L65A | 99 | 0.2 | 3 |
| | C66A | 78 | 0.1 | 2 |
| | R69A | 110 | 0.1 | 2 |
| Surface 2 | T337A | 54 | 0.1 | 2 |
| | D338A | 58 | 0.2 | 3 |
| | V340A | 95 | 0.2 | 3 |
| | F341A | 97 | 2.0 | 30 |
| | N343A | 94 | 0.1 | 2 |
| | E344A | 92 | 71.4 | 1103 |
| | L346A | 84 | 0.1 | 2 |
| | E348A | 106 | 20.2 | 312 |
| | E349A | 100 | 0.1 | 2 |
| | Y354A | 15 | NA | NA |
| | K358A | 124 | 0.1 | 2 |
| Surface 3 | Y422A | 44 | 0.1 | 2 |
| | T446A | 83 | 0.1 | 1 |
| | D447A | 90 | 0.1 | 1 |
| | L450A | 118 | 0.2 | 2 |
| | N452A | 82 | 0.1 | 2 |

### Example 5

### Characterization of Inhibition of Epitope-Grafted FAD-GDH

Epitope-grafted FAD-GDH were used to evaluate the inhibition of enzyme activity in the presence of antibodies or antibody fragments specific for particular epitopes.

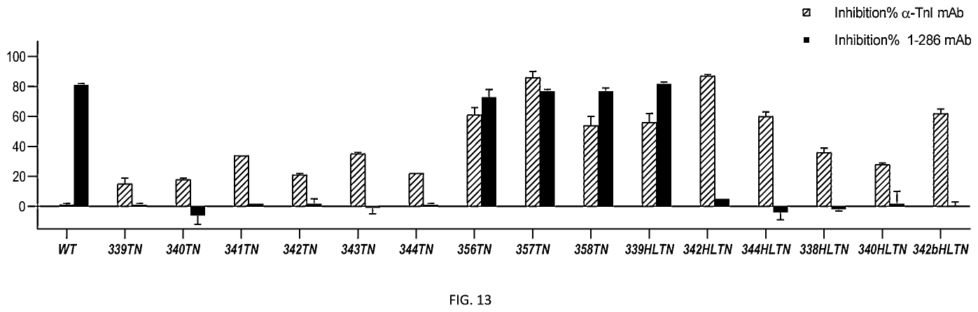

An HA epitope (YPYDVPDYA) was inserted at positions 341 (341HA) and 358 (358HA) of FAD-GDH (19031) (SEQ ID NOs: 55 and 56) and constructs were purified as described elsewhere herein. Following the General FAD-GDH Assay Protocol of Example 1, 1.5 nM of enzyme was treated with antibody concentrations from 320 nM to 0 nM. Both 341HA and 358HA showed no response to an irrelevant control antibody (α-Myc Ab). However, both 341HA and 358HA showed dose-dependent inhibition by α-HA Ab. 341HA was not inhibited by 1-286 Ab while 358HA shows inhibition by 1-286 Ab in a dose-dependent fashion (FIG. 10). Since the 341 site of FAD-GDH is central to the binding site for 1-286 and 358 is at the periphery of the interaction, the inserted epitope at 341 disrupts the 1-286 binding whereas the epitope at 358 does not appear to.

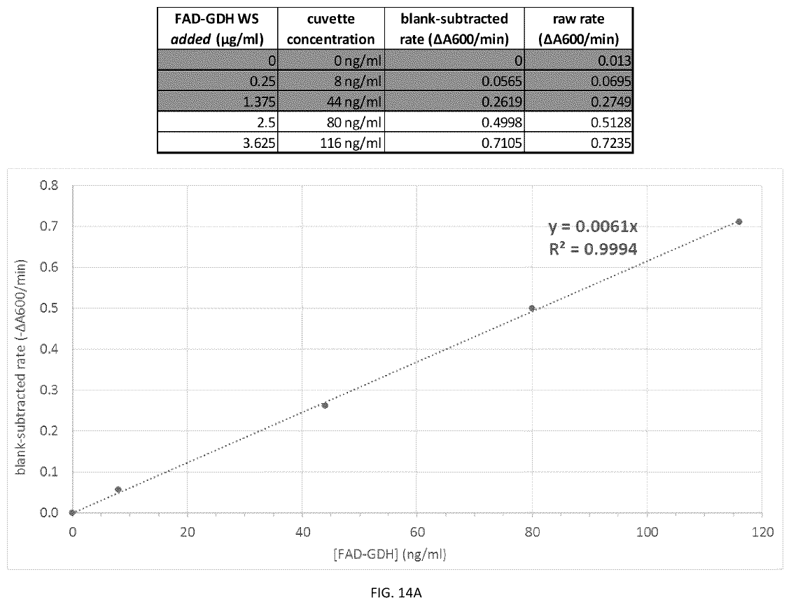

VHHs for FAD-GDH (19031) were identified through phage display. Following the General FAD-GDH Assay Protocol of Example 1, 1.5 nM of enzyme was treated with VHH concentrations from 40 μM to 0 nM. Both VHH-1 and VHH-859 epitope grafts showed dose-dependent inhibition to FAD-GDH, analogous to 1-286 Ab (FIG. 11). Based on these results, a small VHH format specific binding protein was able to bind and inhibit enzyme activity as efficiently as larger format IgG and Fab.

V5 (IPNPLLGLD) and TnI (ISASRKLQS) epitopes were inserted at various positions in FAD-GDH (19031) (See SEQ ID NOs: 5-31 and 43 and Table 4) and constructs expressed in Pichia pastoris as described elsewhere herein. Following the General FAD-GDH Assay Protocol of Example 1, 10× enzyme diluent were prepared by 2-fold serial dilution of the Pichia expression supernatant of each epitope graft construct.

Supernatant diluent concentration with the optimal reaction rate were identified, showing approximately 1.0 AU linear absorbance decrease in 15 reading cycles (or 20 mins reading window). The identified supernatant diluents (50 μL) were transferred to a fresh 96-well plate and used in the General FAD-GDH Assay Protocol of Example 1. Final antibody concentration (1×) was 50 nM for both α-V5 and 1-286 antibody.

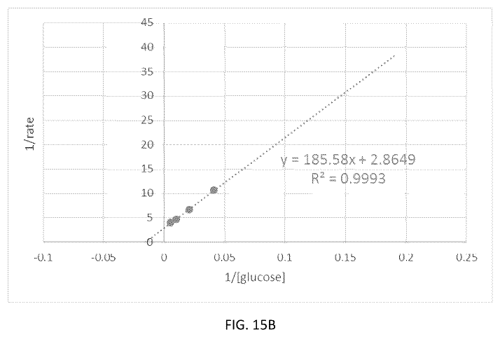

Various TnI epitope grafts (See Table 4) were screened using both 1-286 and α-TnI mAb at 50 nM final concentration. The calculated percent inhibition is summarized in the graph below. FAD-GDH (19031) does not respond to α-TnI antibody at 50 nM concentration while the TnI epitope grafts show various degrees of inhibition by α-TnI antibody (FIG. 13). An inhibition response to the anti-TnI Ab was observed in 339TN, 340TN, 341TN, 342TN, 343TN, 344TN, 356TN, 357TN, and 358TN; all but 356TN, 357TN, and 358TN exhibited poor responses to the 1-286 mAb since the epitope grafting site is located within the binding site for 1-286. In constructs 356TN, 357TN, and 358TN, the epitope is inserted towards the periphery of the 1-286 binding site and thus, the response to 1-286 was largely preserved.

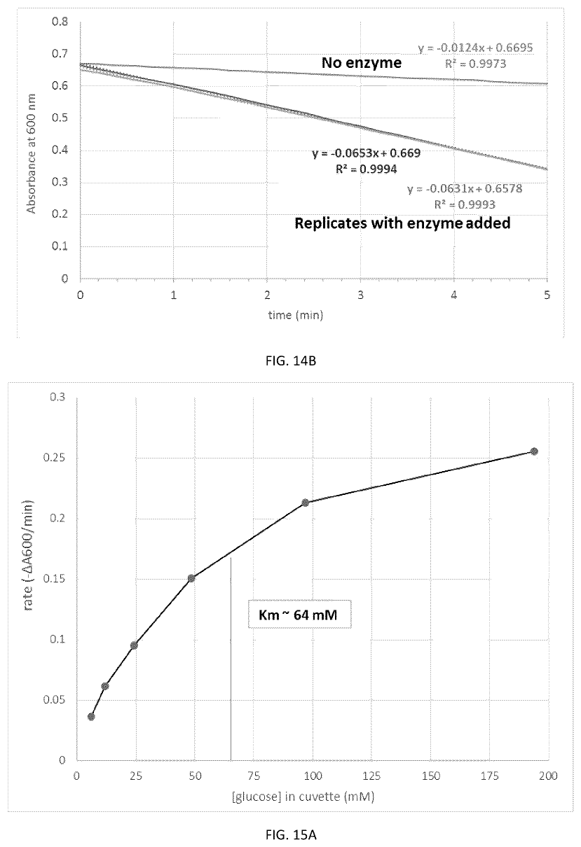

Various V5 epitope grafts grafts (See Table 4) were screened using both 1-286 and α-V5 mAb at 50 nM final concentration. The calculated percent inhibition is summarized in the graph below. FAD-GDH (19031) does not have respond to α-V5 antibody at 50 nM concentration while the V5 epitope grafts has various degrees of inhibition by α-V5 antibody (FIG. 12).

TABLE 4
Descriptions of Graft Constructs by position
and type of epitope grafted into FAD-GDH.

| Graft Construct | SEQ ID NO: |
|---|---|
| 339V5 | 4 |
| 340V5 | 5 |
| 341V5 | 6 |
| 358V5 | 7 |
| 339TN | 8 |
| 340TN | 9 |
| 341TN | 10 |
| 342TN | 11 |
| 343TN | 12 |
| 344TN | 13 |
| 356TN | 14 |
| 357TN | 15 |
| 358TN | 16 |
| 339HLTN | 17 |
| 342HLTN | 18 |
| 344HLTN | 19 |
| 338HLTN | 20 |
| 340HLTN | 21 |
| 342bHLTN | 22 |
| 338bHLV5 | 23 |
| 338cHLV5 | 24 |

### FAD-GDH Assays Using Mouse Serum

To evaluate antibodies raised against 19031 FAD-GDH for inhibitory activity, FAD-GDH activity is measured by spectrophotometry (2,6-dichloroindophenolate hydrate (DCPIP) assay) in the absence or presence of immunized animal sera.

Samples are prepared as 1 ml of Assay Reaction Mixture (ARM) (0.1 M D-glucose, 34.9 mM PIPES/Triton buffer, 0.14 mM Phenazine methosulfate (PMS), 0.68 mM DCPIP) in a quartz cuvette with stir bar, pre-warmed to 37° ° C. for 35-45 sec. The reaction is initiated by the addition of 0.25 μg/mL enzyme (33.3 μl) in ED buffer. The amount of enzyme for a linear response was tritrated with saturating glucose, final concentration of 194 mM.

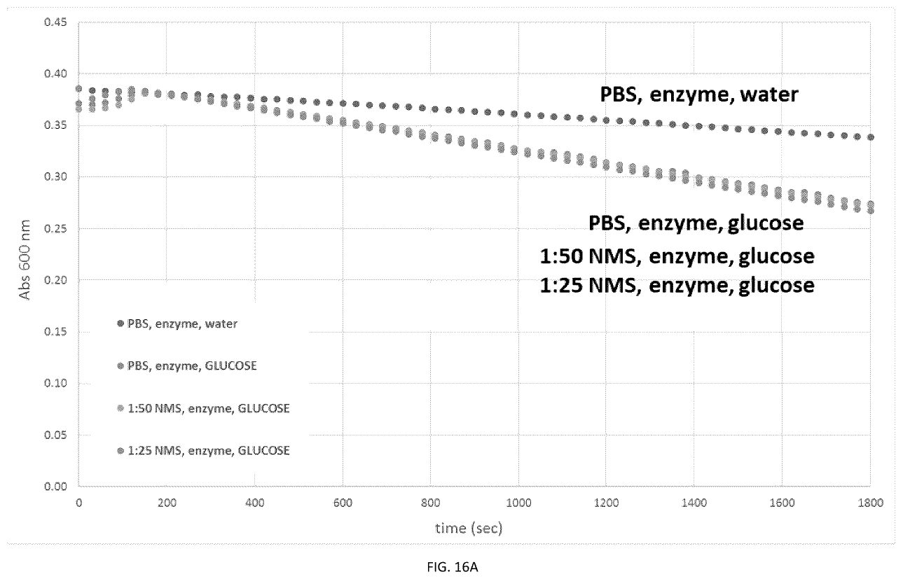

FIG. 14A shows the reaction rates of DCPIP reduction by FAD-GDH in initial testing. These were calculated using linear regression. The three rows highlighted in gray are illustrated as gray-filled circles in the graph. The DCPIP assay shows linear response across the amounts of FAD-GDH added to the cuvette. There is satisfactory linear fit up to and including 116 ng/ml final concentration in the cuvette. Choose an intermediate enzyme concentration on the linear portion of the curve and titrate glucose concentration. Proceed with 1.25 μg/ml FAD-GDH (40 ng/ml in the cuvette). The average specific activity was determined as 99% of the label claim of specific activity from the per min blank-subtracted rate as shown below:

$$[ [ average enzyme Δ A 600 / min ) - ( blank Δ A 600 / min ) ] * 3.1 * 1000 ] / [ ( 16.8 * 1 * 0.1 ) * 0.25 m \text{g/} ml ] = \underline{382.33 U / mg}$$

To determine ranges for robust output with sub-saturating substrate concentrations, FAD-GDH assays were performed while titrating glucose. According to the General FAD-GDH Assay Protocol, each reaction contained 12.5 μl diluted serum in 50 mM PIPES/Triton buffer, 2 mM phenazine methosulfate (PMS) and 0.17 mM dichlorophenol indophenol (DCPIP), 0.04 μg FAD-GDH. Various concentrations of D-glucose solution (12.5-100 mM) were added last to initiate each reaction. Absorbance at 600 nm was measured over the course of 30 minutes at 37° C. Enzymatic rates were calculated using linear regression. The DCPIP assay shows near-linear response for 1.25 μg/ml FAD-GDH (40 ng/ml in cuvette) between 12.1-48.5 mM glucose. Apparent Km for glucose is 64.7 mM under these conditions.

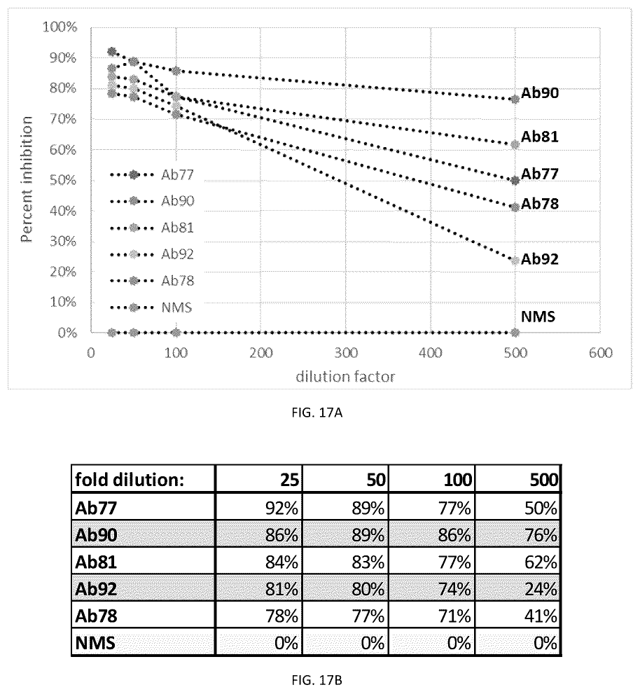

GDH assays were performed on polyclonal sera from normal mouse serum for modulation of FAD-GDH activity in 96-well plates according to the General FAD-GDH Assay Protocol, with each reaction containing 12.5 μl diluted serum in 50 mM PIPES/Triton buffer, 2 mM phenazine methosulfate (PMS) and 0.17 mM dichlorophenol indophenol (DCPIP), 0.04 μg FAD-GDH, and the equivalent of 20 mM D-glucose solution added last to initiate each reaction. Absorbance at 600 nm was measured over the course of 30 minutes at 37° C. Enzymatic rates were calculated using linear regression (FIGS. 16A and 16B). The DCPIP assay was used to determine the extent of inhibition of the activity of normal mouse serum. Negligible interference was observed for 1:25 or 1:50 diluted normal mouse serum, far exceeding range of serum in diagnostic assays. To increase the dynamic range to detect inhibition, the amount of glucose in the assay was increased to 40 mM.

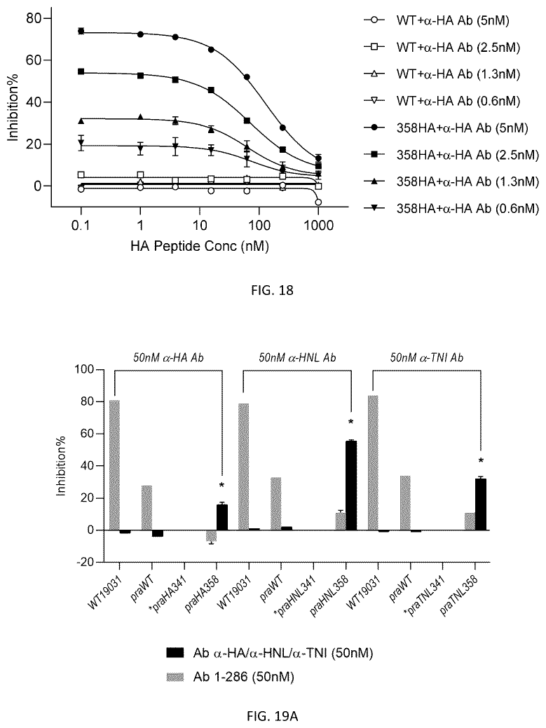

GDH assays were performed on polyclonal sera from mice immunized with FAD-GDH for modulation of FAD-GDH activity in 96-well plates according to the General FAD-GDH Assay Protocol, with each reaction containing 12.5 μl diluted serum in 50 mM PIPES/Triton buffer, 2 mM phenazine methosulfate (PMS) and 0.17 mM dichlorophenol indophenol (DCPIP), 0.04 μg FAD-GDH, and 12.5 μl of a 40 mM D-glucose solution added last to initiate each reaction. Absorbance at 600 nm was measured over the course of 30 minutes at 37° C. Enzymatic rates were calculated using linear regression (FIGS. 17A, 17B and 1). The DCPIP assay was used to determine the extent of inhibition of the activity of FAD-GDH by the various samples of FAD-GDH immunized mouse serum. Of these, the best-inhibiting sera consistently include Ab77, 90, 81, 92, and 78.

### Example 6

### De-inhibition Assay Using 358HA Construct

This example assays enzyme de-inhibition with a 358HA epitope grafted construct and an HA peptide. Using 1.5 nM for both control enzyme (ungrafted FAD-GDH) and 358HA, a final antibody concentration from 5 nM to 0 nM, 2-fold serial dilution, and a final HA peptide concentration from 1 μM to 0 nM, 4-fold serial dilution, the General FAD-GDH Assay Protocol of Example 1 was followed.

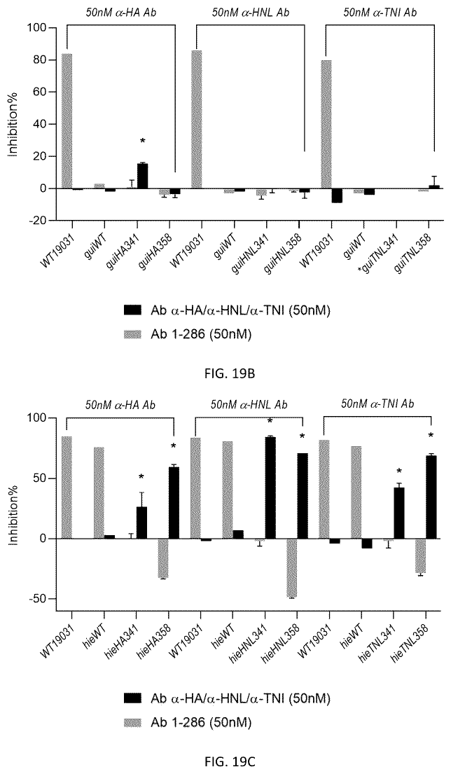

The percentage of inhibition decreased while antigen (HA) peptide concentration increased, indicating the successful competition of the antigen to the enzyme bound antibody resulting in release of antibody-bound enzyme and re-activation of the enzyme catalytic function. De-inhibition was observed at various antibody concentrations (5 nM/2.5 nM/1.3 nM/0.6 nM) in a dose-dependent fashion (FIG. 18). The inhibition/de-inhibition was not observed with ungrafted FAD-GDH.

### Example 7

Epitope Grafting Into Six Mucor Genus FAD-GDH sequences (M. prainii, M. guilliermondii, M. hiemalis, M. subtillissimus, M. circinelloides, and M. ambiguus)

Six Mucor epitope grafted constructs were expressed in Pichia pastoralis. Enzyme diluents were prepared for us in the by 2-fold serial diluting the Pichia expression supernatant of each epitope graft construct. The General FAD-GDH Assay Protocol of Example 1 was used as described in Example 5. An antibody inhibition test was also completed as in Example 5. Final antibody concentration was 50 nM for all four antibodies tested (α-HA, α-HNL α-TnI, and 1-286).

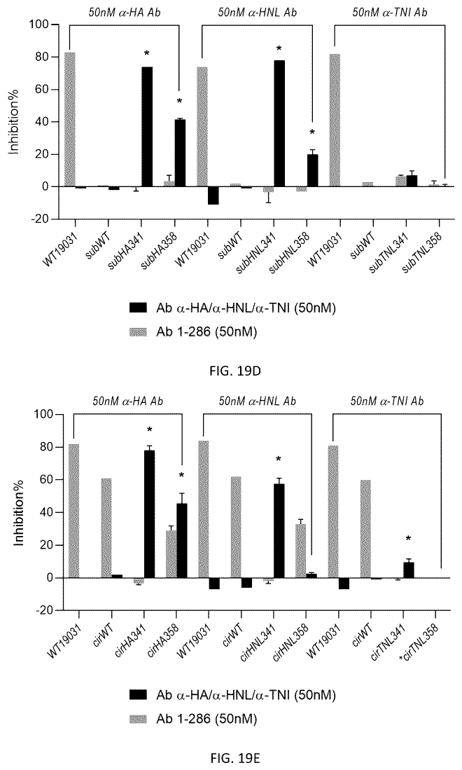

The percent of inhibition of Mucor (M. prainii, M. guilliermondii, M. hiemalis, M. subtillissimus, and M. ambiguus) epitope grafts by 1-286 antibody and anti-epitope antibodies (α-HA/α-HNL/α-TNI) is shown in FIGS. 19A-F. Epitope graft that labeled with “*” indicates no viable enzyme activity, due to which reason the percent of inhibition were not measured. Both in-house, ungrafted FAD-GDH (19031) and wild-type Mucor FAD-GDHs did not show response to anti-epitope antibodies (α-HA/α-HNL/α-TNI). Six Mucor epitope graft panels had at least one epitope graft that responded to the anti-epitope antibodies. These epitope grafts were indicated by the “*” above the percent of inhibition bar.

### Example 8

### VHH and Fab Inhibitors

### Inhibition by VHH-1 and VHH-859

### Materials

19031 FAD-GDH
Purified VHH-1, VHH-10, VHH-859, and VHH-898

### Methods

10× substrate diluent made by serial diluting (2/3 fold) 1M glucose with DI water.

Final substrate concentration (1×) ranges from 100M to 0 mM, 2/3-fold serial dilution. Final VHH-1 concentration (1×) ranges from 2 μM to 0 uM, 2-fold serial dilution. Final VHH-859 concentration (1×) ranges from 1 uM to 0 uM, 2-fold serial dilution.

Linear regression analysis from GraphPad Prism8 was used to calculate the slope and R square of each reaction well. The slope and the substrate concertation was plotted for each reaction well and the Michaelis-Menten equation was applied to fit the data.

$$v = \frac{V max [ S ]}{K m + [ S ]}$$

Additionally, the 1/slope and 1/[S] value was calculated. A Lineweaver-Burk plot was generated using linear regression fitting the double-reciprocal dataset.

### Results

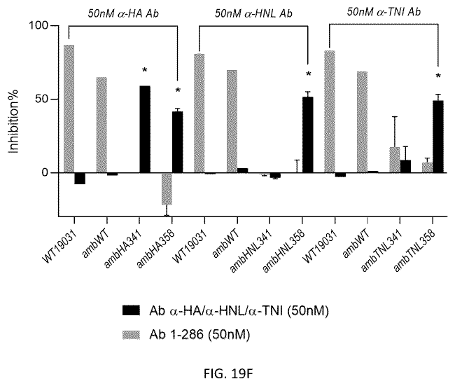

Based on the observation that all linear regression lines at various VHH doses were merged on the x-axis in the Lineweaver-Burk plot, both VHH-1 and VHH-859's inhibition modes are determined to be non-competitive inhibition (FIGS. 20A and 20B).

### Inhibition Test of Fab IO3

### Materials

19031 FAD-GDH
Purified IgG 103 (4.7 mg/mL) and Purified Fab IO3 (enzyme digested from IgG IO3, 5.4 mg/mL)

### Methods

Final glycosylated FAD-GDH concentration (1×) 1.0 nM. Final non-glycosylated FAD-GDH concentration (1×) 0.7 nM. Final antibody 1-286, IgG IO3 and FabIO3 concentrations (1×) are 100 nM, 100 nM, and 1.5 μM, respectively

### Results

Both IgG IO3 and its digested antibody fragment Fab IO3 inhibits the FAD-GDH (FIG. 20C).

### Example 9

### V5 Epitope Graft Antibody Inhibition Titration and V5 Antigen Detection

### Step 1. Range-Finding

### Materials

Ungrafted FAD-GDH (19031) and V5 epitope grafts (339V5 (SEQ ID NO: 4)/340V5 (SEQ ID NO: 5)/341V5 (SEQ ID NO: 6)) FAD-GDH constructs

Anti-V5 antibody, Mouse monoclonal 1 mg/mL, Sigma (cat #V8012-50UG)

### Methods

Final enzyme concentration (1×, ungrafted FAD-GDH (19031) and three V5 epitope graft FAD-GDHs) 0.625 nM. Final antibody (α-V5 mAb) concentration (1×) from 650 nM to 0 nM, 2-fold serial dilution.

### Results

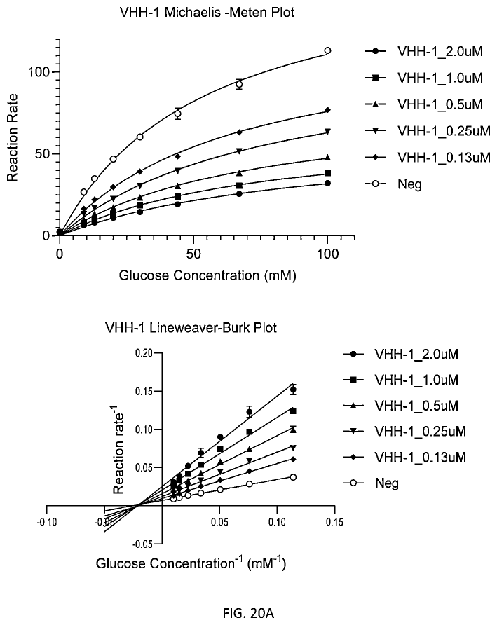

The calculated percent of inhibition at various α-V5 antibody concentrations are plotted in FIG. 21A. All V5 epitope grafts showed dose-dependent inhibition by α-V5 antibody. Ungrafted FAD-GDH (19031) did not show inhibition at any given α-V5 antibody concentration.

### Step 2. V5 Epitope Graft Enzyme De-inhibition by V5 Peptide—Single Dose

### Materials

α-V5 antibody, mouse monoclonal 1 mg/mL, Sigma (cat #V8012-50UG)
Ungrafted and 341V5 epitope grafted FAD-GDH
V5 peptide (CGKPIPDPLLGLDST), 10 mg/mL, Sigma (cat #V7754-4MG); resuspended in water at 10 mg/mL

### Methods

Final enzyme concentration (1×) 3 nM for V5 epitope graft (341V5) and 1.5 nM for control enzyme (ungrafted FAD-GDH). Final α-V5 antibody concentration (1×) 300 nM. Final V5 peptide concentration (1×) 3 μM.

Percent inhibition was calculated using the slope of each sample well and the slope of the negative control wells (buffer only, no antibody or V5 peptide) by using the following equation:

$$Inhibition \% = ( {Slope}_{Sample} - {Slope}_{Neg} ) / {Slope}_{Neg} * 100 \%$$

### Results

The α-V5 antibody (Enzyme+Ab+Buffer) has approximately 50% inhibition for the 341V5 FAD-GDH while it has no inhibition on the ungrafted FAD-GDH.

The percent of inhibition reduced to about 9% for the 341V5 epitope graft in the presence of V5 peptide (Enzyme+Ab+Ag) while the V5 peptide has no impact on the ungrafted enzyme (indicated by arrows in FIG. 21B).

### Step 3. V5 Epitope Graft Enzyme De-inhibition by V5 Peptide—Titration

Materials (same as above)

### Methods

Final enzyme concentration (1×) 3 nM for V5 epitope graft (341V5) and 1.5 nM for control enzyme (ungrafted FAD-GDH). Final α-V5 antibody concentration (1×) 300 nM. Final V5 peptide concentration (1×) from 3 μM to 0 uM, 2-fold serial dilution.

Percent inhibition was calculated using the slope of each sample well and the slope of the negative control wells (buffer only, no antibody or V5 peptide) by using the following equation:

$$Inhibition \% = ( {Slope}_{Sample} - {Slope}_{Neg} ) / {Slope}_{Neg} * 100 \%$$

Plot the log V5 peptide concentration and the inhibition percentage.

### Results

The V5-peptide showed de-inhibition of the α-V5 antibody to V5 epitope graft FAD-GDH (341V5) in a dose-dependent fashion (FIG. 21C).

### Example 10

### TnI Epitope Graft Antibody Inhibition Titration

### Materials

Purified TnI epitope graft enzymes (358TN1 (SEQ ID NO: 28), 358TN4 (SEQ ID NO: 29), and 358TN8 (SEQ ID NO: 30))
α-TnI 19C7 mouse IgG, 2 mg/mL, Abcam, mouse mAb to cardiac Troponin 19C7

### Methods

Final enzyme concentration (1×) 1.5 nM, 3.5 nM and 3.5 nm for 358TN1, 358TN4 and 358TN8 enzyme, respectively. Final antibody (α-TnI mAb) concentration (1×) from 1.0 μM to 0 nM, 4-fold serial dilution.

### Results

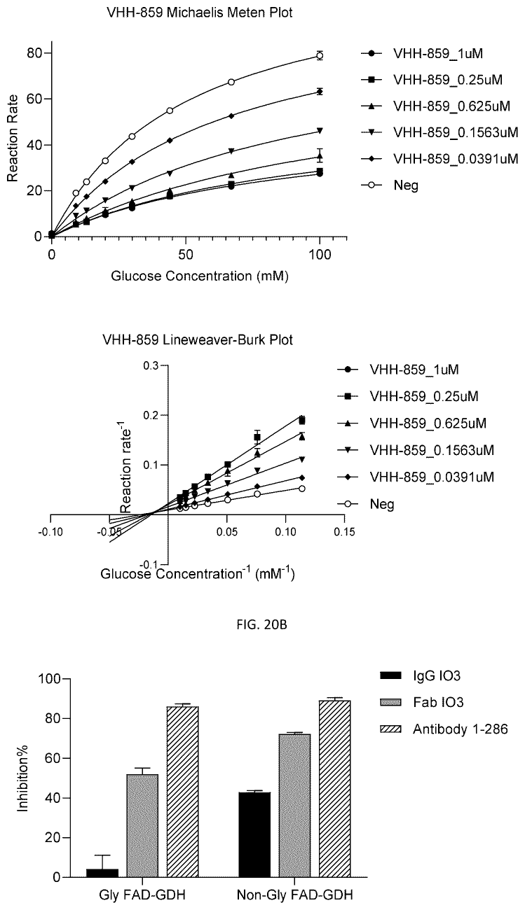

The calculated percent of inhibition at various α-TnI antibody concentrations are plotted in FIG. 22. All three TnI epitope graft (358TN1, 358TN4, and 358TN8) showed dose-dependent inhibition by α-TnI antibody.

### Example 11

Cardiac Troponin I (TNI), V5, HA, c-Myc, and FLAG Antibody Inhibition

### Materials

Epitope graft constructs from Pichia expression supernatant, including six TnI (19C7) epitope grafts (341TN1 (SEQ ID NO: 25), 341TN4 (SEQ ID NO: 26), 341TN8 (SEQ ID NO: 27), 358TN1 (SEQ ID NO: 28), 358TN4 (SEQ ID NO: 29), and 358TN8 (SEQ ID NO: 30)), twenty-four V5 epitope grafts (341VL1-341VL11, 341VLFL, 358VL1-358VL11, and 358VLFL (SEQ ID Nos: 31-54), two FLAG epitope grafts (341FLAG (SEQ ID NO: 59) and 358FLAG (SEQ ID NO: 60)), two c-Myc epitope grafts (341Myc (SEQ ID NO: 57) and 358Myc (SEQ ID NO: 58)), and two hemagglutinin (HA) epitope grafts (341HA (SEQ ID NO: 55) and 358HA (SEQ ID NO: 56)).
α-c-Myc 9E10 mouse mAb, 1 mg/mL, Millipore (Cat #: MABE282)
α-HA mouse mAb, 0.5 mg/mL, Sigma (Cat #: SAB1305536-400UL)
α-Flag M2 9E10 mouse mAb, 1 mg/mL, Millipore (Cat #: F1804-200UG)
α-V5 antibody, mouse mAb 1 mg/mL, Sigma (cat #V8012-50UG)
α-TnI 19C7 mouse IgG, 2 mg/mL, Abcam, mouse mAb to cardiac Troponin 19C7
Final antibody concentration (1×) was 100 nM for all antibodies.

### Results

Ungrafted FAD-GDH showed no inhibition response to any of the anti-epitope antibodies.

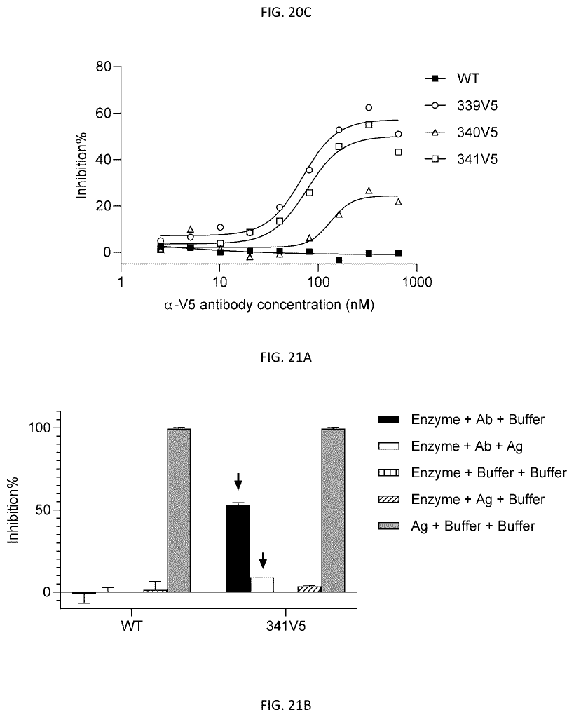

All FAD-GDH grafted enzymes with various epitopes (V5/TnI/Flag/HA/Myc) showed inhibition response to the corresponding anti-epitope antibody at 100 nM concentration as shown in FIG. 23.

### Example 12

### HNL Analyte Detection

### Materials

Purified 358HNL-H3 enzyme (SEQ ID NO:116)
Rabbit α-HNL mAb, 0.75 mg/mL, Abcam (cat #ab206427)

### Methods

Final enzyme concentration (1×) 1.25 nM for 358HNL-H3. Final antibody (α-HNL) concentration (1×) from 450 nM to 0 nM, 4-fold serial dilution.

### Results

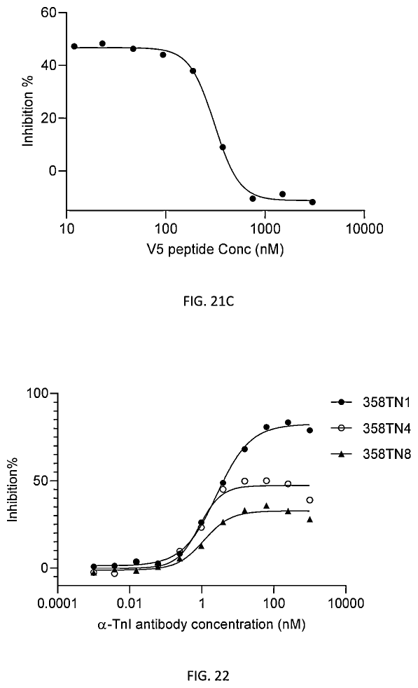

Percent of inhibition was calculated at various α-HNL antibody concentrations and shown in FIG. 24A. HNL epitope graft 358HNL-H3 showed dose-dependent inhibition by α-HNL antibody.

### HNL Epitope Graft Antibody De-Inhibition by HNL Peptide—Single Dose

### Materials

Purified 358HNL-H1 (SEQ ID NO:117) and 358HNL-H3 (SEQ ID NO:116)
Rabbit α-HNL mAb, 0.75 mg/mL, Abcam (cat #ab206427) 340V5Peptide SGSGPGSQPGEFTLGNIKS (SEQ ID NO: 128) (reconstituted to 20 mg/mL with DI water)
341V5 Peptide SGSGQPGEFTLGNIKSYPG (SEQ ID NO:129) (reconstituted to 20 mg/mL with DI water)

### Methods

Final enzyme concentration (1×) 0.5 nM for 358HNL-H1 and 1.5 nM for 358HNL-H3. Final α-HNL antibody concentration (1×) 25 nM. Final 340V5 and 341V5peptide concentration (1×) 4 μM. Reactions were conducted in wells of a multi-well plate.

Percent inhibition was calculated using the slope of each sample well and the slope of the negative control wells (buffer only, no antibody or HNL peptide) by using the following equation:

$$Inhibition \% = ( {Slope}_{Sample} - {Slope}_{Neg} ) / {Slope}_{Neg} * 100 \%$$

### Results

The α-HNL antibody has approximately 20% inhibition for 358HNL-H1 and 60% inhibition for 358HNL-H3 (Enzyme+Ab+Buffer). The percent of inhibition reduced to about 0% in the presence of 340V5 and 341V5 peptide for 358HNL-H1. Similarly, the percent of inhibition reduced to about 30% in the presence of 340V5 and 341V5 peptide for 358HNL-H3 (Enzyme+Ab+Ag). See FIG. 24B.

### Example 13

### NTproBNP Antigen Detection

### Materials

Purified NTproBNP epitope graft 341BP (SEQ ID NO:63) and 358BP (SEQ ID NO:64)
Mouse α-NTproBNP antibody, 1 mg/mL, Biorad (cat #MCA2641)
Mouse α-NTproBNP antibody, 3.5 mg/mL, Novus (cat #NB200-439)
Purified NTproBNP antigen 4.5 mg/mL (E248770251-22-005)

### Methods

FAD-GDH inhibition assays were conducted using epitope grafted enzymes 341BP and 358BP. Final enzyme concentration (1×) 1 nM for both 341BP and 358BP. Final α-NTproBNP antibody concentration (1×) 500 nM. Final NTproBNP antigen concentration (1×) 5 μM. Reactions were conducted in wells of a multi-well plate.

Percent inhibition was calculated using the slope of each sample well and the slope of the negative control wells (buffer only, no antibody or NTproBNP antigen) by using the following equation:

$$Inhibition \% = ( {Slope}_{Sample} - {Slope}_{Neg} ) / {Slope}_{Neg} * 100 \%$$

### Results

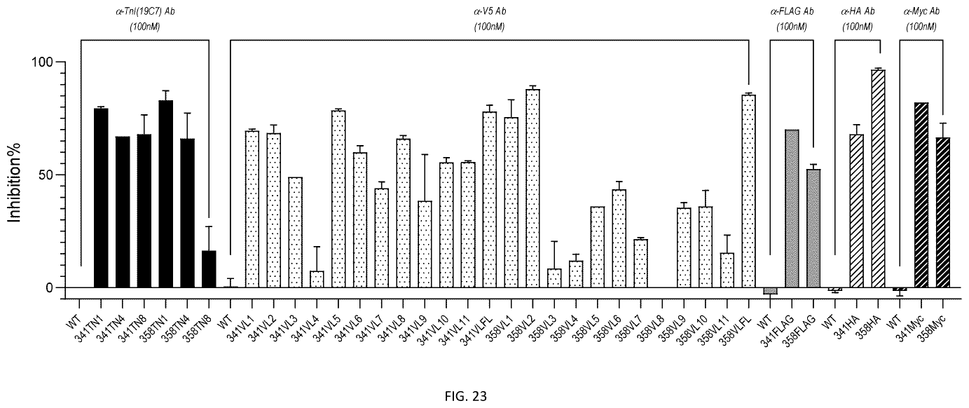

The Biorad α-NTproBNP antibody has approximately 25% inhibition for 341BP and 7% inhibition for 358BP (Enzyme+Ab+Buffer). The percent of inhibition reduced to about 0% in the presence of 5 μM of NTproBNP antigen. Similarly, the Novus α-NTproBNP antibody has approximately 7% inhibition for 358BP (Enzyme+Ab+Buffer). The percent of inhibition reduced to about 0% in the presence of 5 μM of NTproBNP antigen (FIG. 25). The Novus antibody has very minor inhibition to 358BP therefore the de-inhibition was not conclusive.

### Example 14

### De-inhibition Titration

This example assessed enzyme de-inhibition using differing concentrations of inhibitor and antigen. In these experiments, ungrafted FAD-GDH (Purified 19031 FAD-GDH 38 mg/mL (E239543171-22-011)) is used as the enzyme and an activity disabled FAD-GDH is used as the analyte (Purified 19031HHAA FAD-GDH 63 mg/mL (E247909068-22-015)) to assess inhibition and de-inhibition using mouse 1-286 mAb (Mouse 1-286 mAb, 8 mg/mL (E241086302-18-013)) as the inhibitor, which binds to an allosteric site on ungrafted FAD-GDH and inhibits FAD-GDH activity.

Ungrafted enzyme was used at a final enzyme concentration (1×) of 0.5 nM. 1-286 Ab was used at a concentration titrated from 4 nM to 0 nM by 2-fold serial dilution. Reactions were conducted in wells of a multi-well plate. Antigen was used at a concentration titrated from 25 nM to 0 nM by 2-fold serial dilution. Percent inhibition was calculated using the slope of each sample well and the slope of the negative control wells (buffer only, no antibody antigen) by using the following equation:

$$Inhibition \% = ( {Slope}_{Sample} - {Slope}_{Neg} ) / {Slope}_{Neg} * 100 \%$$

### Results

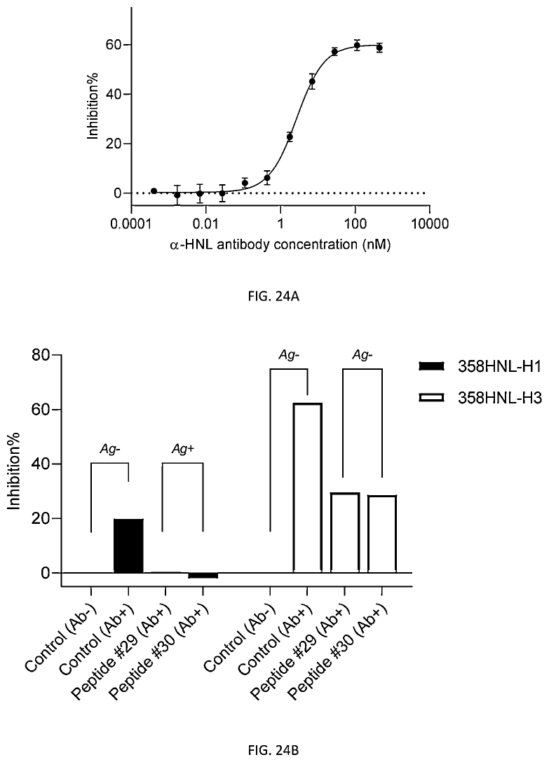

The antigen titration curve at 0 nM of antibody showed no increased or decreased inhibition %, which confirms the activity disabled FAD-GDH used as antigen does not have detectable residual enzyme activity within the tested concentration. Percent of inhibition drops while increasing the antigen concentration. This dose-dependent decrease of inhibition is repeatedly observed at various concentrations of antibody. This demonstrates the successful competition of the antigen to the enzyme bound antibody, which released antibody-bound enzyme and re-activated the enzyme catalytical function (FIG. 26).

### Example 15

### Stability Improvement by Introducing Disulfide Bond

This example demonstrated that introduction of a disulfide bond to FAD-GDH resulted in significant stability improvement in both time and temperature stress tests.

### Materials

Purified WT 19031 (SEQ ID NO:1), 358HA (SEQ ID NO:56) and 358HACC (SEQ ID NO:118)

### Methods

Enzymes were diluted to 1 mg/mL with PBS buffer. Diluted enzyme samples were aliquoted and frozen in −80 degree first. At each time point, one aliquot of each sample was thawed and stored in 37-degree incubator. Sample activities were measured at final 0.5 nM concentration by FAD-GDH activity assay.

### Results

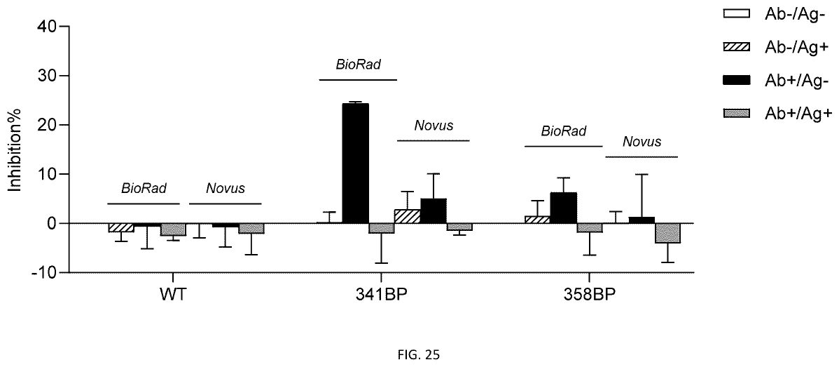

FIG. 27 shows the measured enzyme sample activity at each time point. Compared with the ungrafted 19031 enzyme, 358HA has significant activity loss over extended time point, which indicates less optimal stability. 358HACC, the disulfide bond containing construct using the 358HA as the parent sequence, displays significant stability improvement.

### Example 16

### Discovery of 2″d Allosteric Site on FAD-GDH

Using a phage display of non-glycosylated FAD-GDH, a second allosteric site of FAD-GDH was identified. An aliquot of Superhuman 2.0 Library from Distributed Bio was panned against non-glycosylated FAD-GDH 19031. Blocked, neutravidin coated beads were used for deselection. Selection was done in solution phase using the biotinylated non-glycosylated FAD-GDH and then captured on magnetic neutravidin coated particles. The non-glycosylated FAD-GDH bait was decreased over rounds 1˜4 from a top concentration of 100 nM, down to 5 nM. The wash stringency was increased from 6×, 30 seconds washes in Rounds 1, to 2× 30 minute washes, 4× 30 second washes in round 4. After each round, phage was rescued and amplified to a titer of at least 1*10¹¹ phage. Output titers are shown in Table 5 for rounds 1, 2, and 4.

TABLE 5

| Out | Colonies | Dilution | Total Phage Output |
|---|---|---|---|
| Rd 1 | 4 | 1.00E+04 | 8.80E+07 |
| Rd 2 | 3 | 1.00E+03 | 6.60E+06 |
| Rd 4 | 5 | 1.00E+05 | 1.10E+09 |

To confirm scFV enrichment against non-glycosylated FAD-GDH target, an ELISA assay was completed. A 96 well streptavidin coated plate was blocked and coated with non-glycosylated FAD-GDH. The phage output from rounds 2-4 was diluted in PBST in a 1:1,12-point dilution series. The phage was added to the FAD-GDH coated wells, followed by an anti-M13 phage HRP conjugate. The activity of HRP was read at 492 nm absorbance. The absorbance was then plotted against phage dilution. An increasing signal over rounds indicated that the phage output contained scFVs specific to the FAD-GDH target.

ER2738 cells containing the phagemids selected from the anti-FAD-GDH SuperHuman 2.0 campaign round 3 and 4 were streaked onto 225 mm×225 mm 2XYT agar plates with 2% glucose, Carb-100, and Tet-20 at a 1:100,000× and 1:1,000,000× fold dilution of the original glycerol stocks and incubated at 30° C. overnight. Individual colonies, representing a single antibody clone on a phagemid, were separated into individual wells of 96-deep well plates prepared with 500 μl per well of 2XYT media, 2% glucose, and Carb-100. The plates were covered with breathable lids and grown at 900 RPM in a short throw shaking incubator at 37° C. overnight. After overnight growth the wells were dense with cells. A fresh set of 96 deep well plates were prepared with 1 ml of 2XYT media and Carb-100. The dense cultures (20 μL) were transferred to the new 96-deep well plates and put back into the shaking incubator at 37° C. and 900 RPM for 2.5 hours. Storage media (20% glycerol+2XYT) was added at a volume of 500 μl to the remaining culture in the dense 96-well plates to create a temporary glycerol stock for storage at −80° C.

Activity Assay Screen of scFVs for FAD-GDH Inhibition

An activity assay mixture was prepared with the final concentrations: 80 mM PIPES+0.2% Triton; 5.36 mM PES; 0.68 mM DCPIP; and 57.14 pM enzyme, non-glycosylated. Into a Nunc clear bottom, black sided plate 25 μl of prepared periplasmic extracts (PPE), 85 μl of potassium phosphate monobasic, pH 6.5, 0.1% Triton X-100, and 75 μl of reaction mix prepared above. The final concentration of enzyme in the reaction is 42 pM. The reaction was initiated with 20 μl of 1 M glucose and the plate was read every 5 minutes at 600 nm for two hours. Percent inhibition was calculated for each clone.

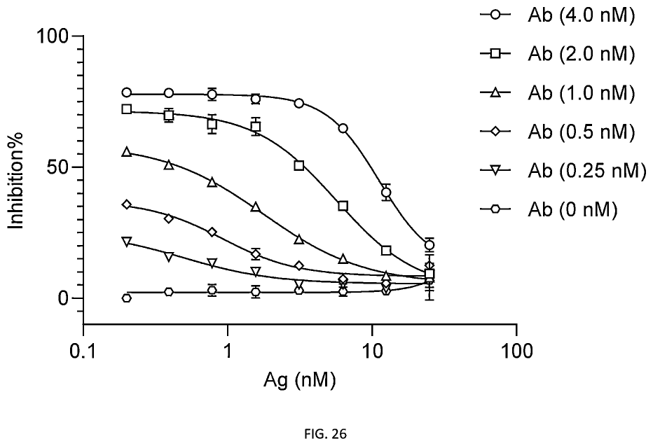

Streptavidin coated plates were blocked with 200 μl of blocking solution. Then 2 μg/ml of biotinylated, non-glycosylated FAD-GDH was diluted in PBS and 100 μl added to each well. After coating, the plates were washed and then 50 μl of PPE and 50 μl of PBS were added to each well and incubated for 1 hour. After incubation, the plates were washed and anti-V5-HRP antibody diluted to 1:5000 in block and 100 μl added to each well. After 1 hour incubation, the plates were washed and OPD substrate was prepared. 100 μl of substrate was added to each well and the plates developed for 4 minutes. After 4 minutes, 100 μl of 1 N Sulfuric Acid was added to each well and then absorbance read at 492 nm (FIG. 28).

Clones that showed binding and inhibition towards FAD-GDH were selected and grown up in 3 ml cultures containing LB plus Carb-100. The dense cultures were spun down and plasmid prepped to extract the phagemid. The phagemids were then used as templates for Sanger sequencing. Thirteen unique clones were shown to inhibit and bind to FAD-GDH.

In order to evaluate whether or not the thirteen identified anti-FAD-GDH scFV inhibitors were binding to a site which was distinct from the 1-286 antibody epitope, a competition ELISA was used. ELISA plates were coated with 1-286 anti-FAD-GDH antibody by diluting the 1-286 antibody to a concentration of 2 μg/ml in PBS and dispensing 100 μl of the diluted antibody into each well. The plates were allowed to incubate for two hours, washed, and blocked with blocking buffer by adding 200 μl of blocking buffer to each well and allowing them to incubate for 1 hour. The plates were washed and 2 μg/ml of non-glycosylated FAD-GDH diluted in block was added to each well at a volume of 100 μl and allowed to incubate for 1 hour. After incubation, the plates were washed and the scFV PPEs from each of the identified 13 FAD-GDH inhibitors were added to the wells. The plates were incubated for 1 hour, washed, and then 100 μl of anit-V5-HRP conjugate at 0.1 μg/ml was added. The plates were incubated for 1 hour and then read by adding 100 μl of prepared OPD substrate to each well and allowing them to develop for 3 minutes. After which, 100 μl of 1 N Sulfuric Acid was added to each well and then plates were read at 492 nm. An absence of signal indicated that the antibodies are competing for the same site on FAD-GDH and that they bind the same or similar epitope. The presence of a signal indicated that the scFV is still able to bind to FAD-GDH even in the presence of 1-286 antibody and thus, the binding site that is distinct from the 1-286 epitope (Table 6). scFVs #3, 6, 7, and 13 appeared to bind to an epitope distinct from 1-286.

TABLE 6

| | % | Same/similar epitope |
|---|---|---|
| Clone | Inhibition | as mAb 1-286 |
| 1 | 39.7 | Yes |
| 2 | 39.5 | Yes |
| 3 | 26.3 | No |
| 4 | 24 | Yes |
| 5 | 17.5 | Yes |
| 6 | 9.5 | No |
| 7 | 10 | No |
| 8 | 87 | Yes |
| 9 | 51 | Yes |
| 10 | 24 | Yes |
| 11 | 17 | Yes |
| 12 | 14 | Yes |
| 13 | 6 | No |

scFVs #3, 6, 7, and 13 were reformatted for expression as IgGs in CHO. Abbott pHybe vectors were used for expression and the DNA was synthesized and sequence verified by a third party. Expi-CHO cells were cultured to a cell density of approximately 6.0 ×10⁶ cells per ml and a total volume of 1 L per construct. The ThermoFisher Expi-CHO transfection kit and protocol were used to transfect 1 μg/ml of DNA of both heavy and light chain plasmids expressing inhibitory anti-FAD-GDH IgGs 3, 6, 7, and 13 to each litre of Expi-CHO cells. The cells were returned to the incubator and allowed to shake at 140 RPM, 8% CO2, 80% humidity and 37° C. The viability of the cells was measured over the next 10 days and once viability dipped below 80%, all cultures were harvested by spinning in a floor standing centrifuge and retaining the supernatant. The supernatant was filtered through a 0.4 μm filter and stored at 4° C.

Supernatants were purified using a HiTrap 5 ml MAbSelect Xtra column on an AKTA Pure. Each supernatant was flowed over the MAbSelect column at flow rate of 5 ml/min. After the supernatant was completed loaded, the column was washed with PBS and then the protein was eluted with a citric acid pH gradient ranging from 0.1 M Citric acid at pH 4.0, 3.6, 3.3, and 2.8. Fractions were eluted into 1 M Tris Tris-HCL pH 9.0 for neutralization. Peak fractions were pooled and run over a HiLoad 26/600 Superdex 200 pg gel filtration column. Peak fractions were collected and pooled.

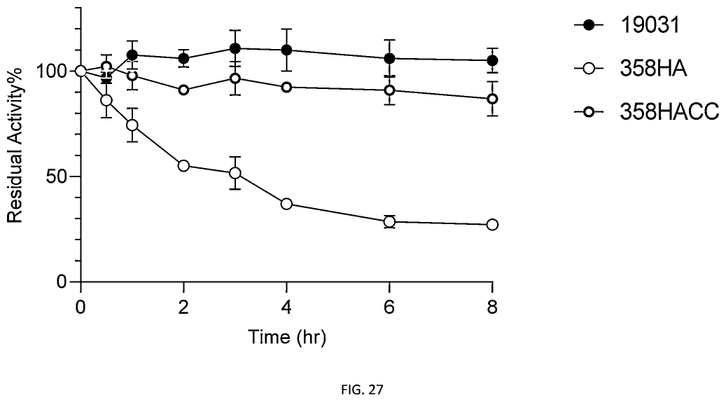

In order to evaluate whether the four identified and re-formatted anti-FAD-GDH IgG inhibitors were binding to a site which was distinct from the 1-286 antibody epitope, a competition ELISA was used. BRAND plastic ELISA plates were coated with 1-286 anti-FAD-GDH antibody by diluting the 1-286 antibody to a concentration of 2 μg/ml in PBS and dispensing 100 μl of the diluted antibody into each well. The plates were allowed to incubate for two hours, washed, and then blocked with blocking buffer by adding 200 μl of blocking buffer to each well and allowing them to incubate for 1 hour. The plates were washed and 2 μg/ml of non-glycosylated FAD-GDH diluted in block was added to each well at a volume of 100 μl and allowed to incubate for 1 hour. After incubation, the plates were washed and a serial dilution of the inhibitory IgGs (IO-3, IO6, IO-7, and IO-13) prepared in block was added to the wells. The plates were incubated for 1 hour, washed, and then 100 μl of Donkey anti-human (H+L)—HRP conjugate at 0.1 μg/ml was added. The plates were incubated for 1 hour and then read by adding 100 μl of prepared OPD substrate to each well and allowing them to develop for 3 minutes. After which, 100 μl of IN Sulfuric Acid was added to each well and then plates were read at 492 nm. An absence of a signal indicated that the antibodies are competing for the same site on FAD-GDH and that they bind the same or similar epitope. The presence of a signal indicated that the antibody was still able to bind to FAD-GDH in the presence of 1-286 antibody. IO-3 was the only full-length IgG which did not compete with 1-286 indicating binding to a site different from that of 1-286 (FIG. 29).

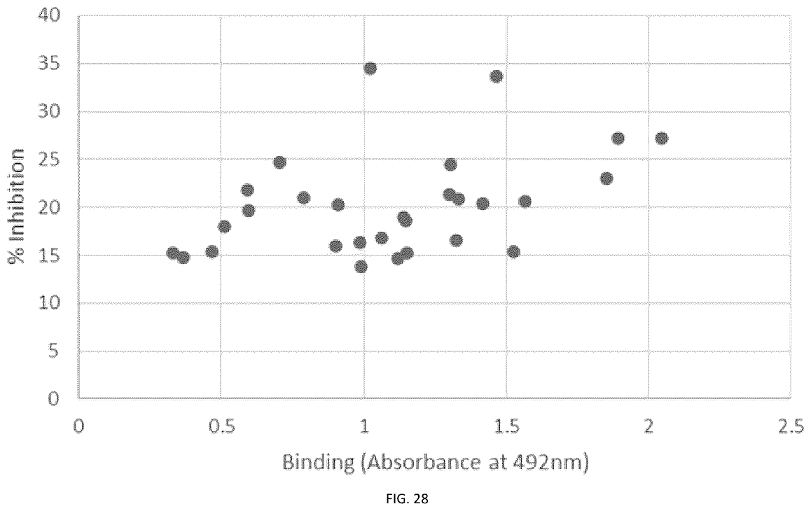

To further confirm the result that IO-3 did not compete with 1-286 and to test whether IO-3 bound to the non-glycosylated 19031 FAD-GDH or the glycosylated 19031 FAD-GDH (WT-FAD-GDH), two different ELISA formats were designed. A diagram summarizing the different assay formats is shown in FIG. 30A. The first ELISA coated BRAND plastic plates with 1-286 anti-FAD-GDH antibody by diluting the 1-286 antibody to a concentration of 2 μg/ml in PBS and dispensing 100 μl of the diluted antibody into each well (FIG. 30A, left). The second ELISA coated BRAND plastic plates with IO-3 anti-FAD-GDH antibody by diluting the IO-3 antibody to a concentration of 2 μg/ml in PBS and dispensing 100 μl of the diluted antibody into each well (FIG. 30A, right). The plates were allowed to incubate for two hours, washed, and blocked with blocking buffer by adding 200 μl of blocking buffer to each well and allowing them to incubate for 1 hour. The plates were washed and 2 μg/ml of non-glycosylated FAD-GDH or glycosylated FAD-GDH (WT FAD-GDH) were diluted and added to each well at a volume of 100 μl and allowed to incubate for 1 hour. After incubation, the plates were washed and a serial dilution of either IO-3 (on the 1-286 coated plates) or 1-286 (on the IO-3 coated plates were prepared in block and 100 μl added to the wells. The plates were incubated for 1 hour, washed, and then 100 μl of Donkey anti-human (H+L) —HRP conjugate at 0.1 μg/ml was added to the ELISA format 1 or 100 μl of Goat Anti-mouse (H+L) HRP conjugate was added to ELISA format 2. The plates were incubated for 1 hour and then read by adding 100 μl of prepared substrate to each well and allowing them to develop for 3 minutes. After which, 100 μl of 1 N Sulfuric Acid was added to each well and then plates were read at 492 nm.

IO-3 did not compete with 1-286 using the non-glycosylated FAD-GDH. However, the glycosylated WT FAD-GDH, showed no binding of IO-3 (FIGS. 30B and 30C) indicating that the glycosylation somehow interfered with the binding of IO-3 to FAD-GDH.

Colorimetric FAD-GDH Activity Assay with Inhibitory IgGs 3, 6, 7, and 13.

A dilution series was prepared for each of the inhibitory IgGs 3, 6, 7, and 13 in assay buffer (50 mM PIPES-NaOH and 0.1 mM Triton X-100). The dilution series was prepared such that the top final concentration in the assay of IgG was 400 nM and a 4-fold serial dilution was prepared down to 0.024 nM final concentration of IgG in the assay. Assays were assembled in clear bottom, black sided 96 well plates and contain the diluted antibody and a final concentration of 30 mM PIPES, 2 mM PES, 0.5 mM DCPIP, and 1 μM FAD-GDH. Reactions were started with 10 μl of 1 M Glucose and absorbance was measured at 600 nm for 40 minutes.

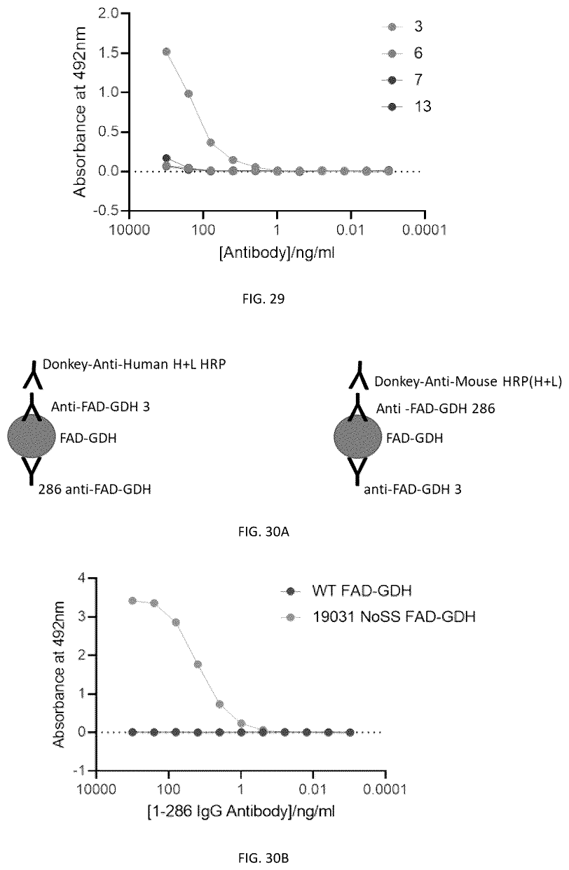

Each of the inhibitory IgGs showed at least some inhibition. IgG 3 and IgG 6 showed the highest percentage of inhibition overall (FIG. 31).

### Example 17

Demonstration of Anti-Epitope Antibody Inhibition of Aspergillus flavus FAD-GDH

The FAD-GDH of A. flavus (SEQ ID NO: 131) was modified with epitopes in a region of the enzyme corresponding to a region successfully modified in the above Mucor FAD-GDH enzymes. The Mucor FAD-GDH insertion including Surface 2 is a large protruding structure on the surface of the enzyme, having both unstructured and helical secondary structure segments. A. flavus FAD-GDH naturally lacks the insertion sequence and instead folds as a short connector without a defined secondary structure. The N-terminal (N′—) distal end of the short connector in A. flavus FAD-GDH was selected for epitope grafting with several epitopes. Specifically, position 328 of SEQ ID NO:131 was chosen for insertion of cardiac troponin I (TNI), hemagglutinin (HA), or human neutrophil lipocalin (HNL) epitopes, resulting in the proteins of SEQ ID NOs: 132 to 134.

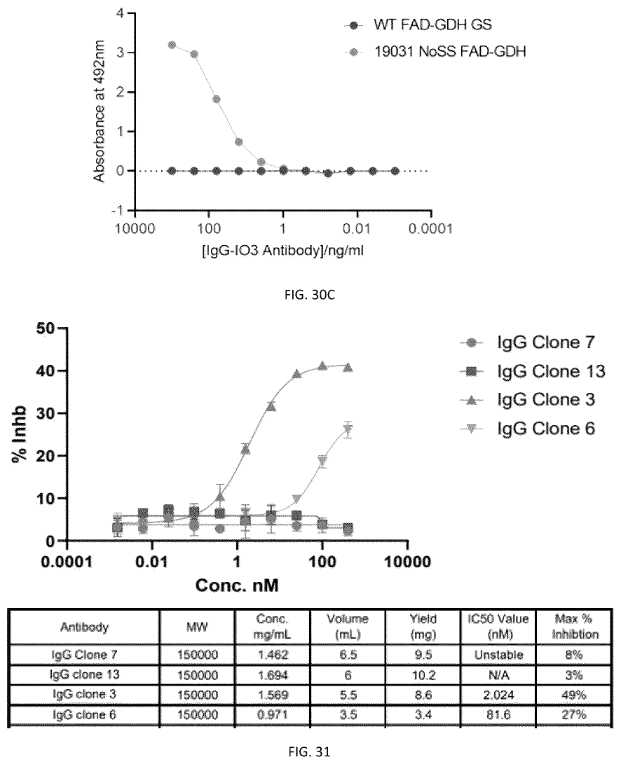

The panel of three grafted proteins and wild-type A. flavus FAD-GDH were expressed as secreted proteins in Pichia pastoris and purified from their supernatants using IMAC and preparative sizing chromatography steps. All three purified proteins exhibited activity in DCPIP assays indicating they are likely well-folded and functional enzymes in these preparations. A sample gel of the purified A. flavus FAD-GDH proteins with grafting at the 328 amino acid residue position is shown in FIG. 32.

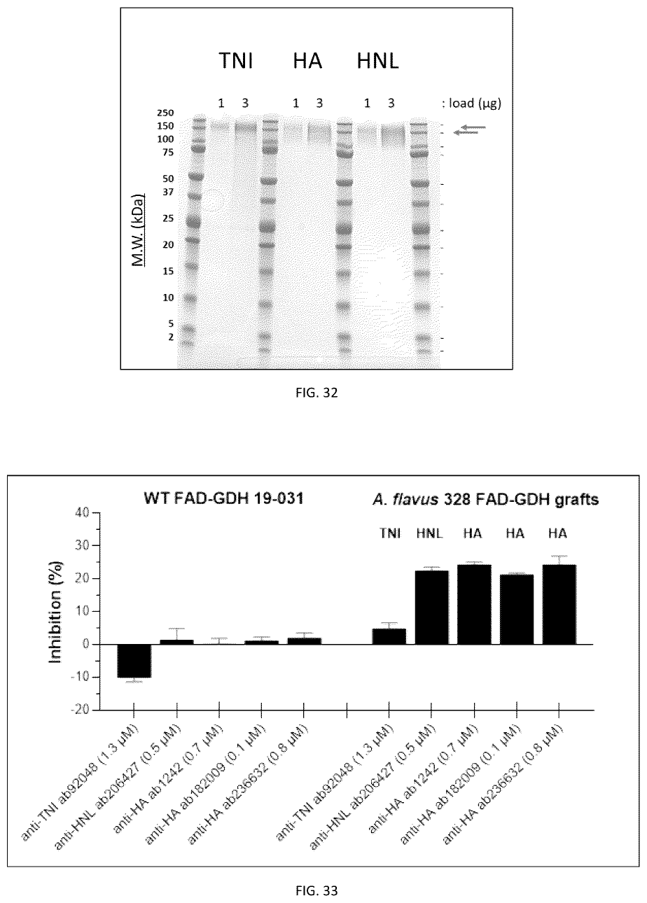

Anti-epitope antibodies were used to observe inhibition of the various A. flavus FAD-GDH 328 grafts. As shown in FIG. 33, antibodies were ineffective at inhibiting wild-type Mucor 19-031 as an experimental negative control. At the recited antibody concentrations, inhibition was observed, most notably with anti-HNL antibody ab206427, and the three anti-HA antibodies tested. The inhibition observed for the matched epitope-antibody pairs exceeded the nonspecific inhibition of wild-type A. flavus FAD-GDH in each case, indicating the inhibition of each of the grafts is specific.

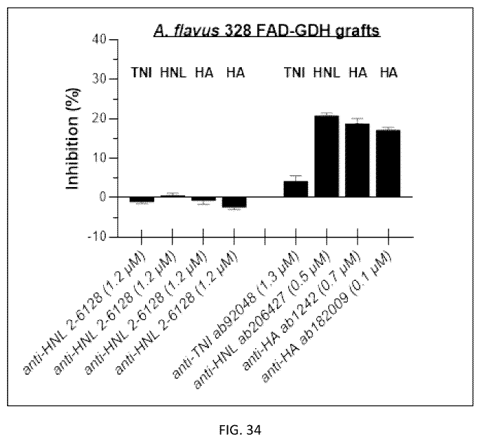

The three A. flavus FAD-GDH epitope grafts at position 328 were further tested using a negative control anti-HNL antibody 2-6128 that does not recognize the HNL sequence used for the epitope grafting. In FIG. 34, only the matched anti-HNL antibody ab206427 inhibited the A. flavus grafted enzyme 328HNL. Similar extent of inhibition of the 328HA graft was achieved using different concentrations of the two different antibodies tested.

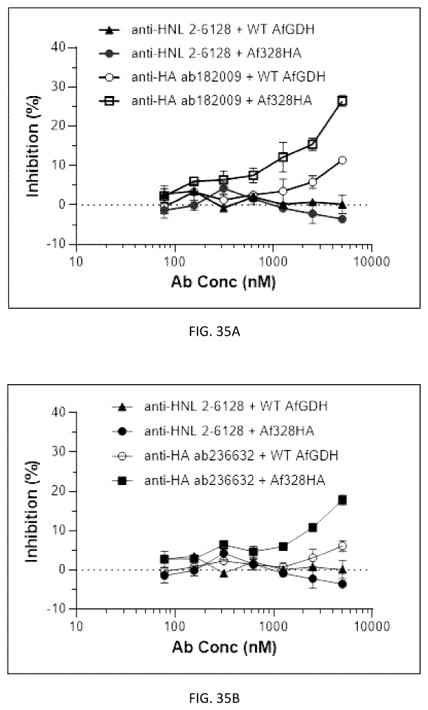

Dose-response inhibition experiments were conducted for each of two antibodies, ab182009 and ab236632 (FIGS. 35A and 35B). In FIG. 35A, anti-HA antibody ab182009 showed dose-dependent inhibition for the grafted enzyme (open squares) consistently above the background, nonspecific inhibition of the ungrafted enzyme (open circles). The unrelated antibody (anti-HNL 2-6128) was used as negative control for inhibition of either ungrafted or grafted A. flavus FAD-GDH (closed triangles and closed circles). In FIG. 35B, samples were compared in dose-response inhibition assays as in FIG. 35A, except the anti-HA antibody was ab236632. The measured inhibition was dose-responsive and consistently higher for the grafted enzyme (closed squares) than the ungrafted enzyme control (open circles).

1.-123. (canceled)

124. An enzyme comprising an epitope-grafted allosteric site that is inhibited by contact with an inhibitor and is de-inhibited in the presence of an analyte that binds to said inhibitor.

125. The enzyme of claim 124, wherein the enzyme is a glucose-metabolizing enzyme.

126. The enzyme of claim 125, wherein the glucose-metabolizing enzyme is an FAD dependent glucose dehydrogenase (FAD-GDH) enzyme.

127. The enzyme of claim 126, wherein the FAD-GDH enzyme is a genus Mucor FAD-GDH or genus Aspergillus FAD-GDH.

128. The enzyme of claim 127, wherein said enzyme is an FAD-GDH from an organism selected from the group consisting of: M. hiemalis, M. circinelloides, M. ambiguus, M. lusitanicus, M. guilliermondii, M. subtillissimus, M. prainii, and A. flavus.

129. The enzyme of claim 124, wherein the enzyme comprises a sequence selected from the group consisting of: SEQ ID NOS: 1-64, 65-72, 75-127, and 132-134 or a sequence at least 70% identical thereto.

130. The enzyme of claim 124, wherein said enzyme is an FAD-GDH enzyme and the allosteric site is located on a surface region corresponding to residue ranges 45-70, 335-362, and 439-457 of SEQ ID NO:1.

131. The enzyme of claim 124, wherein said epitope-grafted sequence comprises an epitope sequence corresponding to an analyte.

132. The enzyme of claim 131, wherein said analyte is a protein.

133. The enzyme of claim 131, wherein said epitope-grafted sequence comprises from 3 to 30 amino acids.

134. A system comprising an enzyme of claim 124 and an inhibitor that binds to said analyte and to said epitope-grafted sequence.

135. The system of claim 135, wherein the inhibitor binds to said analyte with a greater affinity than the inhibitor binds to said epitope-grated sequence.

136. The system of claim 135, wherein said inhibitor is an antibody or antibody fragment.

137. The system of claim 135, further comprising a substrate for said enzyme.

138. The system of claim 135, further comprising a sensor.

139. The system of claim 139, wherein said sensor is an electrochemical sensor.

140. The system of claim 139, wherein said sensor detects a product of said enzyme reacting with a substrate.

141. A method of detecting analyte, comprising: a) contacting a sample suspected of containing an analyte to an enzyme of claim 1; and b) detecting, directly or indirectly, activity of said enzyme.

142. The method of claim 141, wherein said detecting comprising electrochemical measurement of a byproduct of said enzyme reacting with a substrate.

143. The method of claim 141, wherein said sample comprises a biological sample selected from blood, serum, plasma, interstitial fluid, saliva, or urine.
